SEQUENCE LISTING (93 sequences)

SEQ ID NO: 1 - protein, 262 aa, Bemisia tabaci
MEDISSSGEEISMKAISKVIGVPDIRDGPTLTKCIVAEFVGTLLLVLIGCMSVAFVHQDNFVDVVKIAMAFGLIIASMVQAIGHVSGCHINPAVTCGLAVSGHVSIIKGMLYIVAQCLGAICGAIILNEITPKTGYTAAGNLGVTTLSTGVSDLQGVAIEALITFVLLLVVQSVCDGKRTDIKGSIGVAIGFAIACCHLAAIKYTGASMNPARSLGPAFVSGIWDKHWVYWAGPILGGVTASLLYAITFKAKKRSDESSYDF

SEQ ID NO: 2 - DNA, 789 bp, Bemisia tabaci
  source 1..789: mol_type = genomic DNA
atggaggacatatcatcttccggcgaagaaatcagcatgaaagcgatctcaaaagtgattggagtgcccgatattcgagatgggcccactctcacaaaatgcatagttgctgagttcgttgggactttgctgttagtgctcataggatgcatgtcggtagcatttgtccatcaggacaacttcgttgacgttgtgaaaattgccatggctttcgggctcatcatcgcctctatggtccaggcaataggtcacgttagtggttgtcacatcaatccggctgtaacttgtggactagctgtgtcgggacatgttagcataataaaaggtatgctgtacattgtcgcgcaatgccttggagccatctgtggagcaatcattttgaatgaaatcacgccaaaaacaggttacacggctgctggtaatctcggagtaacgacactgtctacaggagtttccgacctgcagggtgtggcgatagaggcactaatcacatttgtgctgcttttagttgtccagtccgtctgcgatgggaagcggaccgacatcaaaggatctatcggtgttgcgataggattcgcaattgcttgctgccatctcgccgcgatcaagtacaccggtgctagtatgaaccctgctagatcattaggccctgcattcgtcagtggaatttgggacaaacattgggtctactgggccggtccaatactcggtggagtcactgccagtctgctttacgccatcactttcaaagccaaaaaaagatcggatgagagctcctatgatttctga

SEQ ID NO: 3 - protein, 588 aa, Bemisia tabaci
MEKLQMKIAVLLFLVFPTAWCREAMEEQPQRDLAWWEKGVIYQIYPRSFKDSDGDGVGDLKGIAEKIDYLSKLGVEAVWISPIFRSPMADFGYDISDFRAIEPMFGTMEDFERLKRLFHKNGLKMILDFVPNHTSDEHDWFKKSVARVDPYTDYYNWVNGKMSENGTIQPPNNWLSYFGGSAWTWNEKRKQYYLHQFHPKQPDLNYRNPLVVQEMKDVLTYWMDKGVDGFRMDAVMTIMEDIKLRDEPLSGKTDVLPTDEEYLNHIYTRDQPGTYEIIKQFRKHLDDYSRKTHTVKFMATEAYSNLTSTMKYYGTKDNPGAHFTFNFETIEALTPQSNAADFKEVIEDWYAALPAGKWSNWVLGNHDKRRVGSRYGIDMLDGLHMLQMCLHGTSVTYAGDEIGMVDTFIRWDQTKDPPALNVGPERYQRFTRDPARTPFQWNASTSAGFSTNPKTWLPVNPDYWSHNIVTEKKKTSSHFKNYRRLLALKKSPVIQFGSVNVYTLSDWVLVITRTLKDHPTYIVILNLGTELEDTKGLRTLANLPEQIRLHTCSINCGYSPGTQLRTDEIQLRPKAGFVCIARDGLKAS

SEQ ID NO: 4 - DNA, 1695 bp, Bemisia tabaci
  source 1..1695: mol_type = genomic DNA
atggaagagcaaccgcaacgcgatctagcttggtgggagaaaggagttatctatcaaatctatccgcgatcattcaaagactcggatggcgacggagtcggcgatttgaaaggaattgcggagaaaattgattacctatcaaaactaggcgtcgaagcagtttggatttctccgatttttcgctccccgatggcagattttggttacgatatatcggatttcagagcgatcgagccaatgtttggcacaatggaagattttgaaaagctgaaaagattattccataaaaatggattaaaaatgattctggacttcgtgccaaaccataccagtgacgaacatgattggtttaagaaatcagtcgcaaaagttgatccgtacacagattattacaactgggtcaacgggaagatgagtgagaatggaaccatgcaaccaccgaataactggctttcatattttggtgggtcagcttggacttggaatgaaaaacgaaagcagtactatctgcatcagttccatccgaaacaaccagatttgaactataggaaccctctcgttgttcaagagatgaaggatgttttgacttattggatggacaaaggagtagatggattcaggatggacgctgtgatgaccatcatggaggacattaaactgagggacgaaccactctcaggcaaaacggacgtcctgccaactgatgaggaataccttaaccacatttataccagagaccaaccggggacctacgagataattaaacagtttcgaaaacatctcgatgattattccaggaaaactcacacggtcaagttcatggcgacagaggcatattcgaatttgaccagcacgatgaaatattacggcacaaaagataatcctggagcccactttacgttcaattttgagaccatcgaagcgctaaccccacaatcgaacgcggcagatttcaaagaggtcatcgaagactggtatgctgctctccctgccggcaagtggtctaattgggtgttgggaaaccacgacaagcggcgggtaggctcgcgctacgggatcgacatgttggacgggctgcacatgcttcagatgtgcctgcacggtacctcggtgacgtatgccggggacgagatcggaatggtggacacgttcatccgctgggatcaaaccaaagatccgcccgctctgaacgtcggccctgagaggtatcaaagattcacccgggaccccgccagaactccgttccagtggaatgcgtccacaagtgcaggtttttcgacgaatccaaaaacttggcttccagtcaatcctgattattggtcacacaatctagttacagaaaagaaaaaggccagtagccatttcaaaaattaccgaagattgttggctctgaaaaagtcgcccgttattcaatttggcagtgtaaatgtctacaccctatcagactgggtccttgtcataaccagaactctcaaagaccacccgacatacattgtcattctgaaccttggcacagaattagaggatacgaaagggttgagaactatcgcgaacttgccagaccaaatcaaactgcatacgtgcagcatcaactgtggctacacgcctggagctcaacttcggacggatgaaatccagcttcgaccgaaggccggctttgtgtgcatcgctcggggtggtctaaaagcctcttga

SEQ ID NO: 5 - protein, 676 aa, Bemisia tabaci
MTVLFVNIFAAAIVATCFVTVTCAVAGADNSKTEDCDDTALFEQSMATHPKQSKLGAGSERKADEKLEWWQTSVFYQIYPMSFKDANGDGKGDLQGIASKADHFVDIGVGAVWLSPIYSSPMADFGYDISNYTEINEIFGNLDDLVHLREKLHARGVKLILDFVPNHSSNEHPWFLNSVKKIDPYTNYYVWRDPVIDANGKKSPPNNWLSIFNSGSAWEWNEERGQYYLHQFAVQQPDLNYNCKELVEEMKEVLRFWLSHGVDGFRMDAVPYLFEDSLFRNEPYVDENIKNSKMYDSLIHIYSMDRPKTYEMIGQFRKVLDDFAKEHNTPPKVILSEAYTSLNYTMKYYGDAKNPGSHMPFNFLMINECHKDSSANDFNAAIHNWMDNMPDDHWANWVMGNHDQPRIATRYGPELVDAINMLVMLLPGTAITYNGEEIGMSNGYIRWDQTIDPAGINVGPKEYEKYSRDGCRTPFQWDDSISAGFSSNSRTWLPVNPNSYYLNLKNQKEESYSHYHIYKRLTTLRKTRTFQRGAWKTYVLSNWVLAIERMMEGEDSYVLVINLGSEYEPIKLSDQIKSLPDTMKVHTASVNSAIKNGESVNTASSGFLMRPKSALVLTTSKEDPPNPAPASSSVSLKTSLCVLVFGILFAILSVKAICYCRTSKSSTVITDLKSGQ

SEQ ID NO: 6 - DNA, 2031 bp, Bemisia tabaci
  source 1..2031: mol_type = genomic DNA
atgaccgttttatttgtgaatattttcgctgctgctattgttgctacctgtttcgtgacggtaacatgcgcagtagcaggagcagataattctaaaacggaggactgtgacgatacggccttatttgagcaatcaatggctactcacccaaaacaatcgaaattgggtgcaggatcggaacggaaggctgatgagaaacttgaatggtggcagacaagtgttttctatcaaatctaccccatgtctttcaaagacgcgaacggggatggaaaaggagatttacaagggatcgcatccaaggccgatcactttgtagacattggcgtgggagcagtatggctgtctcccatttattcctctccaatggccgattttgggtacgacatctcaaattacaccgagataaacgaaatttttggcaacctggatgacttggttcacctcagagaaaaactgcatgcccgtggagtcaagctgattttggattttgtgcccaatcattccagcaatgaacacccttggttcctcaattctgtaaagaaaattgacccttacactaactactacgtatggagggatccagtgattgacgctaacggcaagaaaagccctccaaacaactggttgagtattttcaacagtggatctgcatgggaatggaacgaagagagaggacaatactacctgcatcaatttgcagttcagcaacctgatttgaactacaattgtaaggagctcgtagaggaaatgaaggaagttctcagattttggcttagtcatggcgtggacggttttcgtatggatgcagtaccttacttgtttgaggattctttgttccgtaatgagccgtacgtcgacgagaatattaaaaactccaaaatgtacgactctcttattcacatttactccatggatcgaccgaagacttacgaaatgatcggccagttcagaaaagttctagacgatttcgccaaagaacataacacacctccaaaggttattttgagcgaggcttacacatctttgaactatacaatgaaatactacggagacgctaaaaatcccgggagccacatgcccttcaacttcttgatgatcaacgaatgtcataaagattcgagtgccaacgatttcaatgccgccattcacaactggatggacaatatgcctgatgatcattgggccaactgggtgatgggaaaccatgatcaacctcgaattgcgacacgatacggacctgaattagtagatgctattaatatgcttgtgatgctactaccaggaacggccatcacctataacggcgaagaaattggcatgtcaaatggttacattcgatgggatcaaactattgatcctgcggggataaatgtcggaccgaaagaatatgagaaatactcgcgtgatggatgcaggaccccctttcaatgggacgattcaattagtgcaggattttcttctaacagccggacttggttacctgtcaaccctaattcctactatttgaatttaaagaaccaaaaagaggaaagttacagccactaccacatttacaaaaggctcacaactcttcgtaagacgagaacattccaaagaggggcatggaagacttatgtcctttccaactgggtcttagctattgaaaggatgatggaaggagaggactcttacgttctggtcataaatcttggaagtgaatatgaaccaataaaactttccgatcaaattaagtccttaccagacacgatgaaagtccatacggccagtgtcaactccgcgattaaaaatggcgaaagcgtgaacacggcgagttccggctttttaatgcgccctaagtcagctctagttctgacgacatccaaggaggacccaccgaaccccgctccggccagcagttccgtttccttgaagacaagtctatgtgtcctcgtttttggaattctcttcgcaatattgagtgtcaaagccatttgttattgtcgaacatccaaatccagcactgtcataactgatttaaaatcgggacaatga

SEQ ID NO: 7 - protein, 559 aa, Bemisia tabaci
MQGFQMALWGGRFSEAADQRFKLFNDSLRFDYRLAEQDILGSIAWSKALVTVNVLTSQEQKELESALNELLAEVRSHPEQIPQSDAEDIHSWVESRLIEKVGALGKKLHTGRSRNDQVATDLKLWCKAQVKDLILATRGLQQALITTAEATQDILMPGYTHLQRAQPISFAHWCLAYVEMLARDETRLRDTLKRLDVSPLGCGALAGTAYPIDREQLAGWLGFASATRNSLDTVSDRDHVLEILSNAAIGMVHLSRFAEDLIFFNTGEASFVELSDKVTSGSSLMPQKKNPDALELIRGKCGRVQGALTGMLTTLKALPLAYNKDMQEDKEGLFDAVDTWLGCLQMAALVLDGIKVKKSRCREAAEQGYANSTELADYLVGKGLPFREAHHIVGRVVVEAIAQGVPLGAMSLQELKKFSDVIEGDVYPVLELQSCLEKRCAKGGVAPAQVALAIAEAKERLLGSEPKAFSLDEAPLKRFCAFLRTQESLRHLDRVKVIQWTPCFLSGRHAIYRINWFHFFRYSSFFSKFEIAILLSGDQRTHLPILTNKFNVIKAWFFL

SEQ ID NO: 8 - DNA, 1686 bp, Bemisia tabaci
  source 1..1686: mol_type = genomic DNA
atgcaagggtttcagatggcgctctggggcgggcgattctcggaggcggcggaccagcgcttcaagctcttcaacgactcgctgcgcttcgactaccgcctcgcggagcaggacatcctcggctccatcgcttggtccaaggctctcgtcaccgtcaacgtcttgaccagccaagagcagaaggaactcgagagcgccctcaacgagcttctggccgaggtccggagccaccccgagcagatacctcaaagcgacgccgaggacatacactcctgggtcgagtctcggctcatcgagaaagtcggggctttgggcaagaagctccacaccggccgcagccgcaatgatcaggtggcgaccgacctaaagctctggtgtaaggcacaagtcaaggatctgatcctggcgacgcgcgggctacagcaagcgctcatcaccaccgccgaagcgacccaagacatcctcatgcccggctacacccacctgcagcgcgctcagcctatcagcttcgcgcactggtgcctggcctacgtggagatgctggcgcgggatgagacccgtctaagggacaccctcaagcggctggacgtgagtcctctgggatgcggggccctcgccggcactgcctaccccatcgaccgcgagcagctcgcaggctggctcggcttcgcttcggccacgaggaatagtctcgacacggtctcggatcgggaccatgtcttggagattctgtccaacgccgccatcggcatggtccatctctcgcgcttcgccgaggatctcattttcttcaacacgggcgaggcgtccttcgtggaactgtcggacaaggtgacctcgggctcctcgctgatgccgcagaagaagaacccagacgccctggagttgatccgcgggaagtgcgggcgggttcagggggcgctgacggggatgctgacaacgctgaaggcgctcccgctggcctacaacaaggacatgcaggaagacaaggaagggctcttcgacgccgtcgacacgtggctagggtgcctccagatggcagcactcgtccttgacggcatcaaggtgaaaaaatctcgttgccgagaggcggccgagcaaggctacgccaactcgacggagctggccgactacctggtgggcaaagggctccccttccgcgaggcccaccacatcgtcggccgggttgtcgtcgaggccatcgcccaaggcgtgcccctcggggcgatgtccctgcaggagctcaagaaattctccgacgtcatcgagggagacgtctaccccgtcctcgagctccagtcctgcctcgagaagcggtgcgccaagggaggcgtcgcgcccgcccaagtggcgctggccatcgccgaggcaaaagaacgtctcctcggaagtgaaccgtgaaaagcgttctctctggactgagaggctcccttaaaaaggtaattctgcgcatttctgagaactcaggagtctcttcgacatttggaccgcgttaaggtgattcaatggacgccatgttttttgtcaggacggcatgcgatatatcgcatcaattggttccattttttccgctactcgtcatttttctccaaatttgagatcgcaattctgttgtcaggagaccaaagaactcacttaccaattttaacaaataaattcaacgtaataaaggcgtggtttttttta

SEQ ID NO: 9 - protein, 437 aa, Bemisia tabaci
MAGKAPLWNHFTYRNNELYCEDIPVANLAEKYGTPLYVYSQAAILKQLQALQEAFSELKPLVCYSVKANSNLSILRLLAEHGSGFDVVSGGELKRVSVAGGQARQTVFAGVGKTDEEIAAGLTAGVLMFNVESEEELDAIVRVAQSKKIQAPVALRINPDVDPKTHRYISTGHKESKFGMDIQRSLLLAEKFKGSESIKMIGIHMHIGSQITTTTPYSSAAAKAVDIITRLREIGHPIDWFNMGGGFGISYDGKTSPSIAEFAAAVVPSLRKTGCRLVMEPGRVIVGNAGILISRVIYTKVSGEKQFLIQDAAMNDLIRPSLYESFHGIWPVKVPAGFPSPPDDFETSRINGTKLCDVVGPVCESGDFLAQDRALPPLNRGELLAIFSVGAYGMTMASNYNSRPRPAELLVNGSGVVVARRRETFDDLIAEELNPVS

SEQ ID NO: 10 - DNA, 1314 bp, Bemisia tabaci
  source 1..1314: mol_type = genomic DNA
atggccggaaaagcgccactttggaatcacttcacgtatcgtaataatgaactctactgcgaagatattccagtcgcaaatcttgctgaaaaatatggcacaccattgtacgtatacagccaggcagcaattctcaagcagcttcaggcactccaggaggcattctctgagctgaagccactagtctgttattcagtaaaggcaaattccaatctatcaattcttcgcttgttggcggaacatggaagtggatttgatgtggtatctggtggtgagttgaagcgcgtcagtgtcgctggaggccaggctagacaaactgtctttgctggagttggcaaaaccgatgaggagattgctgctggcctgaccgctggggtcctaatgttcaacgtggagagtgaggaggaattggatgctattgtccgagtagctcaatccaaaaaaatccaggcacctgtggctctccgaatcaatcctgacgttgaccctaaaacgcatcggtatatttcgacaggccataaggagtcaaagttcggcatggatattcaacgatcattattgcttgcggaaaaattcaaaggctcagaatctataaagatgattggcattcatatgcatatagggtcgcagattacaacaacgaccccttacagcagtgcagcagctaaggctgttgatattattactcgtctgcgggaaataggccacccaatcgactggtttaacatgggtggcggctttggaatcagttacgacgggaagacgagtccatcaattgcagaatttgctgctgctgttgtacccagcttaaggaaaacggggtgccgtctggtgatggaacctggtcgggtgattgttggtaatgccggcattcttatcagtcgagtaatttacacaaaggtatcaggggaaaaacagtttctgatccaggacgccgcaatgaatgatctaatccgtccatctttatacgagtcgtttcatgggatttggccagtcaaagtccctgctggctttccttccccaccggatgattttgaaacatcgcgtattaatggaacaaaattatgcgatgtggttggcccagtatgtgaaagcggtgacttcctggcgcaagatcgagctttgccacccttaaatcggggtgagctcctagccattttttccgttggggcatacggaatgacaatggcctccaactacaattctcgccctcgccctgctgagctgttggttaatggatcaggcgttgttgtggctcgccggcgggagacctttgatgacttaattgccgaggagctaaatcctgttagttaa

SEQ ID NO: 11 - protein, 524 aa, Bemisia tabaci
MARNVPSSLLKLLKRQKLAVKQIVRRQVTAPATAPAYHNPHQSFQFSETSVRQCLPSQLKPKPPVSELGFGTQFTDHMLRVEYDVDKGGWQSPLIVPLEPLTLHPAAKGLHYALQLFEGMKAYRGVDGRIRVFRPDLNMARMNSSALRISLPQFDGEELLKCIKKLVSLDQEWVPISETASLYIRPTLVATDPVLGVNAPSSALLYVILSPVGPYFQTMKALTLLAEPNYVRAWPGGSGSTKVGANYGPTIYVQSVAKEVGAHQILWLFGENHAVTEAGAMNVFCVFINDKGRIELSTPPLNGLILPGVTRASILDLAREWNEFDVSERFLTMSDIVKLRSENRLLEFFGSGTAVLVNPVERLIYNNQVIDIPTMDQPDPIFLRLLRTLTDIQYGRLKHPWAEEIDDALKFHSTSMLLYSHRKQYLREFEINGLTRFLLKFQKEPLNHMCYLIYTPGQFLRETLEVRSNLFLWKNKFSVLGSASVQALFLPDLYIQMKFCTLNLYSLICNVFVTFNKSFLRKTL

SEQ ID NO: 12 - DNA, 1594 bp, Bemisia tabaci
  source 1..1594: mol_type = genomic DNA
atggcgcgcaacgtgccctcttccttactcaagctcttgaagcggcagaagctcgctgtcaagcagatagttcgtcgacaagtcactgccccggcaactgcgcctgcgtaccataatccacatcaaagttttcagttttcagagacctcagtgcgacaatgccttccgtcgcagctgaagccgaagccgccagtgtcggaactgggcttcggcacccaattcaccgaccacatgctcagagtcgagtatgacgtcgacaaaggcggctggcaatctcccctcatagttcctctggagcccctcacccttcatccggctgctaaagggctccactacgccctccagctgttcgaggggatgaaggcgtaccgcggcgtggacggtcggatccgggtgttccggccggacttgaacatggcgcggatgaacagctcggcgctgcggatcagcctgccccagttcgacggcgaggagctcctgaaatgcatcaagaaattggtcagtctcgaccaggagtgggtgcccatctcggagacggccagcctctacatccgacccacgctcgtcgccaccgatccggtgctgggtgtgaacgcgccctccagcgccctactctacgtgatcctcagcccagtgggcccgtatttccagacgatgaaggccctcacgctactcgccgagcccaactacgtccgcgcttggcccggcggcagtggctccaccaaagtcggcgccaactacggcccaaccatctacgtccagtcagtggcaaaagaagtcggtgctcatcaaattttatggctattcggagaaaatcatgctgtcactgaagccggagccatgaacgttttttgcgtcttcatcaacgacaaaggaagaatcgagttgagtaccccgcctttgaatggtctcatcctgccgggcgtaacccgtgcctcgattttggacctggcgcgggagtggaacgaattcgacgtcagcgagcggttcctcacaatgagcgacatcgtcaaactgaggtcggagaataggttactcgaattttttgggtctggaacagctgttctagtaaatccagtggagcgtctcatctacaacaaccaagtgatcgacattccgaccatggaccaaccagatcctatcttcttacggcttctcagaactctcacagacatccagtatggaaggcttaaacacccttgggcagaggagatcgattgagatgcattgaaatttcattctacatctatgttactataatactcacatcgcaaacaatatctgagggaattcgagataaatggtttgaccagattcctcctaaaatttcaaaaagaacctctcaatcatatgtaatgttatttaatttacactccaggtcagtttttgagggaaactttagaagtaaggtcaaacctatttttgtggaaaaacaagttttctgtgctgggttctgcgagcgttcaagcattgtttttaccagacctgtattagatacaaatgtgataaaaattttgtaccttaaacctgtaatattcgttgatttgcaatgtttttgttacttttaataaatcatttttacgtaaaacactga

SEQ ID NO: 13 - protein, 243 aa, Bemisia tabaci
MINIGVCGSTGKMGKMIIERIGSFDSCKISSTFSRQTNDSDEFCRNCQVIIDFSNGDLLEKLLKSAIKYKNKLVIGTTGFESKHSDLMHSASKEIAILYSANMSLGINLLSILTEKAAKILNKNYDIEILESFHRMKLDAPSGTALMFGKTLAEARNLDSNKVVIHNRHGKRLPGEIGIASIRGGQVCGEHEIMFLGDNEALSCKHQALNRESFADGAIKAAIWIASKETGFYSMRDVLGQLG

SEQ ID NO: 14 - DNA, 732 bp, Bemisia tabaci
  source 1..732: mol_type = genomic DNA
atgataaatattggagtctgtggttctaccggtaaaatgggaaaaatgataattgaaagaataggaagttttgattcttgtaaaatttcatcaacattttccaggcaaactaatgattcagatgaattttgtcgtaattgtcaggtaatcatagatttttcaaatggcgacttattggaaaaattgctcaaatcagcaataaaatacaaaaataaattggttattggtactaccggttttgagtcaaaacactcagatcttatgcattctgcttcaaaggaaatagctatattatattctgctaatatgagtctagggataaatttactttccatactaactgaaaaagcagcaaaaattctgaataaaaattatgatatagagatcttagagtcctttcacaggatgaagttagatgctccgtctggtactgctcttatgtttggcaaaacattagcggaagccagaaatttagattctaacaaggttgtaattcataaccgccatggtaaaagactaccaggcgaaattggtattgcttcaatcagaggaggacaggtgtgcggagagcatgagataatgtttctaggtgataatgaagctctcagctgtaaacaccaagctcttaatagagaatcttttgctgatggggcaattaaggctgcaatttggattgctagtaaagaaactggtttttattctatgcgtgatgtacttggtcaactcggttaa

SEQ ID NO: 15 - DNA, 20 bp, synthetic construct
  source 1..20: mol_type = other DNA
ttgtttcgcaagtttgccgt

SEQ ID NO: 16 - DNA, 20 bp, synthetic construct
  source 1..20: mol_type = other DNA
gactgattgacgccctggat

SEQ ID NO: 17 - DNA, 20 bp, synthetic construct
  source 1..20: mol_type = other DNA
aacactgcgaatagcgcatc

SEQ ID NO: 18 - DNA, 20 bp, synthetic construct
  source 1..20: mol_type = other DNA
cgccactctagatgttcgca

SEQ ID NO: 19 - DNA, 22 bp, synthetic construct
  source 1..22: mol_type = other DNA
cggtaaggtctgaaactgcgat

SEQ ID NO: 20 - DNA, 22 bp, synthetic construct
  source 1..22: mol_type = other DNA
gttttgctagatgtgcaaggca

SEQ ID NO: 21 - DNA, 20 bp, synthetic construct
  source 1..20: mol_type = other DNA
actacaattctcgccctcgc

SEQ ID NO: 22 - DNA, 20 bp, synthetic construct
  source 1..20: mol_type = other DNA
gtcatcaaaggtctcccgcc

SEQ ID NO: 23 - DNA, 20 bp, synthetic construct
  source 1..20: mol_type = other DNA
aagctctggtgtaaggcaca

SEQ ID NO: 24 - DNA, 20 bp, synthetic construct
  source 1..20: mol_type = other DNA
aggatgtcttgggtcgcttc

SEQ ID NO: 25 - DNA, 19 bp, synthetic construct
  source 1..19: mol_type = other DNA
cgtccagagtcagtggcaa

SEQ ID NO: 26 - DNA, 20 bp, synthetic construct
  source 1..20: mol_type = other DNA
gttcatggctccggcttcag

SEQ ID NO: 27 - DNA, 20 bp, synthetic construct
  source 1..20: mol_type = other DNA
ggtcctaccagttgtgcgaa

SEQ ID NO: 28 - DNA, 20 bp, synthetic construct
  source 1..20: mol_type = other DNA
agaatggagccgaacccaag

SEQ ID NO: 29 - DNA, 20 bp, synthetic construct
  source 1..20: mol_type = other DNA
cattccactacagagctcca

SEQ ID NO: 30 - DNA, 20 bp, synthetic construct
  source 1..20: mol_type = other DNA
tttcaggtttcggatggctt

SEQ ID NO: 31 - DNA, 23 bp, synthetic construct
  source 1..23: mol_type = other DNA
tgtcaggagtaacgacgtgtttg

SEQ ID NO: 32 - DNA, 20 bp, synthetic construct
  source 1..20: mol_type = other DNA
ttcgggaacggtaagtgctc

SEQ ID NO: 33 - DNA, 22 bp, synthetic construct
  source 1..22: mol_type = other DNA
tggtaaaagactaccaggcgaa

SEQ ID NO: 34 - DNA, 23 bp, synthetic construct
  source 1..23: mol_type = other DNA
agcttggtgtttacagctgagag

SEQ ID NO: 35 - protein, 7 aa, Bemisia tabaci
DAPGWLM

SEQ ID NO: 36 - protein, 7 aa, Bemisia tabaci
DAVEYLY

SEQ ID NO: 37 - protein, 7 aa, Bemisia tabaci
DAVNHLL

SEQ ID NO: 38 - protein, 7 aa, Bemisia tabaci
DAARHFF

SEQ ID NO: 39 - protein, 7 aa, Bemisia tabaci
DAPEFIF

SEQ ID NO: 40 - protein, 7 aa, Bemisia tabaci
DAVTYMY

SEQ ID NO: 41 - protein, 7 aa, Bemisia tabaci
DAVMTIM

SEQ ID NO: 42 - protein, 7 aa, Bemisia tabaci
DAVQILF

SEQ ID NO: 43 - protein, 7 aa, Bemisia tabaci
DAVTYMY

SEQ ID NO: 44 - protein, 7 aa, Bemisia tabaci
DAVPHLI

SEQ ID NO: 45 - protein, 7 aa, Bemisia tabaci
DAAKWLF

SEQ ID NO: 46 - protein, 7 aa, Bemisia tabaci
DAVPWLY

SEQ ID NO: 47 - protein, 7 aa, Bemisia tabaci
DAIKHLV

SEQ ID NO: 48 - protein, 7 aa, Bemisia tabaci
DAVVCLY

SEQ ID NO: 49 - protein, 7 aa, Bemisia tabaci
DAVPYLF

SEQ ID NO: 50 - protein, 7 aa, Bemisia tabaci
ETVSYLF

SEQ ID NO: 51 - protein, 7 aa, Bemisia tabaci
DAIKHVY

SEQ ID NO: 52 - protein, 7 aa, Bemisia tabaci
DAINFMF

SEQ ID NO: 53 - protein, 7 aa, Bemisia tabaci
DAVAYLF

SEQ ID NO: 54 - protein, 7 aa, Bemisia tabaci
DAVHTMF

SEQ ID NO: 55 - protein, 7 aa, Bemisia tabaci
DAIPILF

SEQ ID NO: 56 - protein, 7 aa, Bemisia tabaci
DAVNYLF

SEQ ID NO: 57 - DNA, 534 bp, Bemisia tabaci
  source 1..534: mol_type = genomic DNA
atgcaacgcgtactttattttgtcatttttgtcctttttctattcggccagctgggcagccgacaaactgcaaaggcagttgactggtgggaaaataccataatctaccagatatatccaaggtcattcaaggattcgaacggtgatggcctcggagacttgaaaggtatttacgagaaactggattatgtaaaagaaataggagtcgggaccatttggattcaatcattctataaatctccgatggttgatttggggtacgatatcaccgatttcagagctgtagatccaatatttggaaccatggatgacttcaagagagtagtgcaggggatccatgaaaggggtatgaggattatcctagacttcgtgccaaaccatacaagtaacacctgtgaatggtttaaactttctgaacgtcgcatcgaaccatatacaaatttttatgtttggcaagatccaaagcagttaaatgattcccactggacatatccgaataactgggtagctatacctaccgatgttatttattga

SEQ ID NO: 58 - protein, 177 aa, Bemisia tabaci
MQRVLYFVIFVLFLFGQLGSRQTAKAVDWWENTIIYQIYPRSFKDSNGDGLGDLKGIYEKLDYVKEIGVGTIWIQSFYKSPMVDLGYDITDFRAVDPIFGTMDDFKRVVQGIHERGMRIILDFVPNHTSNTCEWFKLSERRIEPYTNFYVWQDPKQLNDSHWTYPNNWVAIPTDVIY

SEQ ID NO: 59 - DNA, 1773 bp, Bemisia tabaci
  source 1..1773: mol_type = genomic DNA
atgtttaaggtaatcactgtgtgcttatggctattcgccttcaacagtcttaatgtgtcgtccgagtatgtttatgagggtttgaagagcgattcggtcgaaccagactggtggcagacggaaattatctatcaaatatacgtaagatcgtttaaagacagcgatggtgacggaatcggagacctgaacggtataacagagaaagtcccatattttaaaactatagatgttggcgccgtttggttgtcgccaatattcctctcaccacaaaatgatttcggatacgatatatcagactacaaagagatcgatcctatttatggctcaatggcagactttgaacggatgagggatgagtttcacaagcatggcataaaggtcttattggactttgtgccaaatcacacgagtgacgaacacgaatggttccaaaagtcaattaagaaaatagagcccttttcagactattacgtatggaaagatccgatccgtgatgtacatggaaataacacgcctccgagtaattggttaggtgtgttcaacagtgggtctgcgtgggaatggaatgaagaacgtcaacaatattatttacatcagttccaagtgaaacaacccgacttgaactacagaaatccgtcggttagagaagaaataaagaacacgctgttatattggttgggacgtggcgtcgacgggttcagattcgacgcggtgaactatctattcgaaagagaagatctagctgacgaacctaagtctaataaaattggctatttggacaccgattacgattctttaacgcatacgagcacacttgatcaacctgaaacttattccatcgttcgtcaatggaggcagatgctggacagttacaggactagggaaaagaaaaccaagtttatgatggtggaatgttactcaccatttgataaaactttgttgtactacggtagtaattcagagcctggcgctcattttccatttaactttttattcatcggaacgttcgatcaacagtctgatgctgctaaggtccataacatgatcaaatcatggattcgtggtatgcccaccggcatgtggcctaactgggtgttaggtaaccatgataacgcaagagtggcttcgaggagcaatccaatgttagttgatggactacacatgatccaacacctgttgccccgcacctctgtgacttattacggagacgaactaggtctgatcgacacaaccgttcgttgggaccaaacagttgatccagcggggcttaacgtgggcccctataggttcttgaaattcagcagagatcccgtgaggactccgttcccatgggacagttcgtataacgcaggtttttctaattcgtcttcgttgtggcttcctcttaacgccgattattggaaaaaaaatatggttgaagaatcaaggtttaaaagtaacctaaggtcatacaggcaattggctcggttaaggaggagtctcacatttgtcaaaggcgatttgcatttatacacactgtccaaatgggtgtttggattttcacggagtttttatgatcacccgacttacttcattgtagttaatttcgggagtgaaatagaaacagttaatttgatggaagctagaggtactttaccattaactatgaaagttaaggtatctagcattaactctggttttgttaccggaaatttagtacgtactgatagtgtattactgcgcccgaaagcagctcttgtacttacaacctcaagaatgaacgaagatgcataa

SEQ ID NO: 60 - protein, 590 aa, Bemisia tabaci
MFKVITVCLWLFAFNSLNVSSEYVYEGLKSDSVEPDWWQTEIIYQIYVRSFKDSDGDGIGDLNGITEKVPYFKTIDVGAVWLSPIFLSPQNDFGYDISDYKEIDPIYGSMADFERMRDEFHKHGIKVLLDFVPNHTSDEHEWFQKSIKKIEPFSDYYVWKDPIRDVHGNNTPPSNWLGVFNSGSAWEWNEERQQYYLHQFQVKQPDLNYRNPSVREEIKNTLLYWLGRGVDGFRFDAVNYLFEREDLADEPKSNKIGYLDTDYDSLTHTSTLDQPETYSIVRQWRQMLDSYRTREKKTKFMMVECYSPFDKTLLYYGSNSEPGAHFPFNFLFIGTFDQQSDAAKVHNMIKSWIRGMPTGMWPNWVLGNHDNARVASRSNPMLVDGLHMIQHLLPRTSVTYYGDELGLIDTTVRWDQTVDPAGLNVGPYRFLKFSRDPVRTPFPWDSSYNAGFSNSSSLWLPLNADYWKKNMVEESRFKSNLRSYRQLARLRRSLTFVKGDLHLYTLSKWVFGFSRSFYDHPTYFIVVNFGSEIETVNLMEARGTLPLTMKVKVSSINSGFVTGNLVRTDSVLLRPKAALVLTTSRMNEDA

SEQ ID NO: 61 - DNA, 1785 bp, Bemisia tabaci
  source 1..1785: mol_type = genomic DNA
atgaagctggtggcagtattttgtctcgttttggcttcggcctcctgcgaggtaatgaaagcataccctcaaagagacttagcgtggtgggaaaggggtgtcatttatcaaatttaccccaggtcgttcaaggactcaaacggagatggagtcggcgatctgaagggtatcgcagagaaaatcgattacctttcgaatttaggtgtcaaggcggtgtggctttctcctatttttcgttcaccgatggttgatttcggctacgatatctctgacttcagagctattgagcctatgttcggaaccatggaagattttgaagtactgaaagagttattacagaaaaatggactaaaaataattctggacttcgtgccgaaccatacaagtgatcaacacgactggtttatacgatccgtcgcaagagaggacccttacaccaattattacgtttgggttgatgccaagtacgtaaatgggaccagatcacccccaaacaactggctgtcagattttggaggctcagcttggacctggaatgaaaaacggaagcaatacttcttgcatcagttccaccctaaacaacccgatttaaactacagaaaccctctcgtggtgcaagaaatgaaggatgttttgacatactggatggacaaaggagtcgatggttttaggatggacgctgtgatgacgattatcgaagacgatcagctgagggacgagccgccgtccaacaaaccagacgttctgcccacagacgtagaattcctcacccacatccacactagggatcagccgggaacctatgatattattaaacagttccgaaagctcctggacgactattcggccaaaacaggcactgtcaagttcatggcgacagaagcgtactcaaatttaacaaacacgatgaaatactacggagacaaatattaccccggagctcacttcacgttcaatttcggaccaatcaccggactgactgctcaatcaagtgcaaagcaattcaaagaagtgatcgaaaactggtatagagagcttccagaaggcaaatggtctaactgggtgcttgggaaccacgaccaacaacgcgtggccactcgctacggtatcgacatgttggacggacttcacatgcttcaaatgtgccttcacggtacctccgtgtcgtattcgggtgatgagatcggaatgaccggtcccttcatccggtgggaccagactaaggacccgcccgccctcaacgttggacctgagaggtacctgagattcacaagggatcctgctaggactcccttccagtgggcttcatgcaccagtgcaggattttcgacgaaccctaagacttggctccctgtcaatcctaactactggtcgcacaacttggtcacagagaagaagaagaacagaagccacctgaaaaattatcagaagctcctggccttgaaagaatcaccagtcatacagtttggcaacttgaacgtttacacactatccgattgggtgcttgtcgtaacaagggctctggaaggacacccaacttatgtggtcgtactgaatataggttcagaaatagaatacacgacaaaactaagtgctgtcgcgaacctccccgaacaactcaaattacatatctgcagtcttaactgtgggtacacgcctggagttcaacttcatactgagaaaattcagctccgaccaaaagcgggtatggtcttatcaacccagaaaggtgttcaagctagcagagaagccgatgagtcaagttcagaacttcaaatggaataa

SEQ ID NO: 62 - protein, 594 aa, Bemisia tabaci
MKLVAVFCLVLASASCEVMKAYPQRDLAWWERGVIYQIYPRSFKDSNGDGVGDLKGIAEKIDYLSNLGVKAVWLSPIFRSPMVDFGYDISDFRAIEPMFGTMEDFEVLKELLQKNGLKIILDFVPNHTSDQHDWFIRSVAREDPYTNYYVWVDAKYVNGTRSPPNNWLSDFGGSAWTWNEKRKQYFLHQFHPKQPDLNYRNPLVVQEMKDVLTYWMDKGVDGFRMDAVMTIIEDDQLRDEPPSNKPDVLPTDVEFLTHIHTRDQPGTYDIIKQFRKLLDDYSAKTGTVKFMATEAYSNLTNTMKYYGDKYYPGAHFTFNFGPITGLTAQSSAKQFKEVIENWYRELPEGKWSNWVLGNHDQQRVATRYGIDMLDGLHMLQMCLHGTSVSYSGDEIGMTGPFIRWDQTKDPPALNVGPERYLRFTRDPARTPFQWASCTSAGFSTNPKTWLPVNPNYWSHNLVTEKKKNRSHLKNYQKLLALKESPVIQFGNLNVYTLSDWVLVVTRALEGHPTYVVVLNIGSEIEYTTKLSAVANLPEQLKLHICSLNCGYTPGVQLHTEKIQLRPKAGMVLSTQKGVQASREADESSSELQME

SEQ ID NO: 63 - DNA, 1506 bp, Bemisia tabaci
  source 1..1506: mol_type = genomic DNA
atgaccgttttatttgtgaatattttcgctgctgctattgttgctacctgtttcgtgacggtaacatgcgcagtagcaggagcagataattctaaaacggaggactgtgacgatacggccttatttgagcaatcaatggctactcacccaaaacaatcgaaattgggtgcaggatcggaacggaaggctgatgagaaacttgaatggtggcagacaagtgttttctatcaaatctaccccatgtctttcaaagacgcgaacggggatggaaaaggagatttacaagggatcgcatccaaggccgatcactttgtagacattggcgtgggagcagtatggctgtctcccatttattcctctccaatggccgattttgggtacgacatctcaaattacaccgagataaacgaaatttttggcaacctggatgacttggttcacctcagagaaaaactgcatgcccgtggagtcaagctgattttggattttgtgcccaatcattccagcaatgaacacccttggttcctcaattctgtaaagaaaattgacccttacactaactactacgtatggagggatccagtgattgacgctaacggcaagaaaagccctccaaacaactggttgagtattttcaacagtggatctgcatgggaatggaacgaagagagaggacaatactacctgcatcaatttgcagttcagcaacctgatttgaactacaattgtaaggagctcgtagaggaaatgaaggaagttctcagattttggcttagtcatggcgtggacggttttcgtatggatgcagtaccttacttgtttgaggattctttgttccgtaatgagccgtacgtcgacgagaatattaaaaactccaaaatgtacgactctcttattcacatttactccatggatcgaccgaagacttacgaaatgatcggccagttcagaaaagttctagacgatttcgccaaagaacataacacacctccaaaggttattttgagcgaggcttacacatctttgaactatacaatgaaatactacggagacgctaaaaatcccgggagccacatgcccttcaacttcttgatgatcaacgaatgtcataaagattcgagtgccaacgatttcaatgccgccattcacaactggatggacaatatgcctgatgatcattgggccaactgggtgatgggaaaccatgatcaacctcgaattgcgacacgatacggacctgaattagtagatgctattaatatgcttgtgatgctactaccaggaacggccatcacctataacggcgaagaaattggcatgtcaaatggttacattcgatgggatcaaactattgatcctgcggggataaatgtcggaccgaaagaatatgagaaatactcgcgtgatggatgcaggacccctttcaatgggacgattcaattagtgcaggattttcttctaacagccggacttggttacctgtcaaccctaattcctactatttga

SEQ ID NO: 64 - protein, 501 aa, Bemisia tabaci
MTVLFVNIFAAAIVATCFVTVTCAVAGADNSKTEDCDDTALFEQSMATHPKQSKLGAGSERKADEKLEWWQTSVFYQIYPMSFKDANGDGKGDLQGIASKADHFVDIGVGAVWLSPIYSSPMADFGYDISNYTEINEIFGNLDDLVHLREKLHARGVKLILDFVPNHSSNEHPWFLNSVKKIDPYTNYYVWRDPVIDANGKKSPPNNWLSIFNSGSAWEWNEERGQYYLHQFAVQQPDLNYNCKELVEEMKEVLRFWLSHGVDGFRMDAVPYLFEDSLFRNEPYVDENIKNSKMYDSLIHIYSMDRPKTYEMIGQFRKVLDDFAKEHNTPPKVILSEAYTSLNYTMKYYGDAKNPGSHMPFNFLMINECHKDSSANDFNAAIHNWMDNMPDDHWANWVMGNHDQPRIATRYGPELVDAINMLVMLLPGTAITYNGEEIGMSNGYIRWDQTIDPAGINVGPKEYEKYSRDGCRTPFNGTIQLVQDFLLTAGLGYLSTLIPTI

SEQ ID NO: 65 - DNA, 1860 bp, Bemisia tabaci
  source 1..1860: mol_type = genomic DNA
atgctgttagcgataatgtgcaaatttacggtagcattgatgattatcggtctagtgaccgaatttcacgcaaaatcggctgttttggagcaaaaacctcttgattggtggcaacgaggtgttttttaccaaatttaccctaggtccttcaaagacagtaacggcgatggtgttggagatctgcgaggtatagcggaaaaagtatggtatctaaaagatttaggcgtgtcagcgatatggttcaaccctatattcgattctcctggcttggatctgggatatgacatttccaactacacacgtatcgaccccctttttggaactttagaagatttggagtacttaagagacgctcttcataaagcaggtatacggcttctgctggattttgtcccaaatcatacaagtgatgagcatccatggtttcagtggtctatcaaaaaagtcgatccgtacacaaactattacgtttggaaggacgccaagatggtcaatggtcaaaggaaaccaccaaccaactggataagcgagtttggaggctccatgtggaaatggaacgacgaaagacagcaatacttcttacaccagtttcaccagaagcaaccagacttgaattatgaatgtaaagaactggtccaagagatgatggatgttgtcaggttttggctggaccgcggtgtagatgggctgcgtttagacgccatcaactttatgtttgaggatccgctatggagagatcaagagctcctagcgcctgatctggaccccaacttcttcttgagctacaatagatcaaggacgatggatcagccggccacatacgagatggtcacgagattcagagaaattttcgactcttacagtaaaaaagacggccgaaccagagtaatgatgactgaggcttatacaacgctggataagacactgggtttctatcagtacaacggcaagccgggcgctcacatgccatttaatttctttttcatcacgcatgttaatggacggtcacccgcaaaagattaccagagagctatccaaggctggatgcagggcatgccagaaggacactcggcgaactgggtgataggaaaccatgacaatcatcggatagcggcaagatacggcacggatctcgtagacggtatgaacatgatagcgctcttactcccagggactgggaacacgtactacggggatgaaatcggtatggacgacgcagctatcagattcgacgagggtgtcgacccacaggcctgccagttcgacaaggattggttcaatcgcgtcaccagggaccccgagagaacacctttccagtgggacacatccttgaacgcaggtttttcgacgagcatgaaaacttggctaccggtgaattccaacttttggcgtttaaacctgaaggaccagatgcaaagtaacgaaaaaagtcattacaaggtatacaagagactgatggatgttcgcaagacagacacgatgctgtatggggatgtggagacctacgtacttgccaaatgggtcttcgcctttactaggcgtttgactggcagtgacacttatgtggtggtcgtcaatcttgggagtgacactacatctgtcgatctctctgctttcatgagtgatctaccggagactttaacagtccacacgggcagtgtcaatacccagtatgatccaaatgacaaagtaccaactggtgtattttacatgcgaccaaaagcctccatccttttaactactgcccaagaagtgccgcctgctgcatattcggattccaaaactagtggagtcgcaacaaggacatccactgcaatcggagtaattgggttccttttcgctcataggttactcctatag

SEQ ID NO: 66 - protein, 619 aa, Bemisia tabaci
MLLAIMCKFTVALMIIGLVTEFHAKSAVLEQKPLDWWQRGVFYQIYPRSFKDSNGDGVGDLRGIAEKVWYLKDLGVSAIWFNPIFDSPGLDLGYDISNYTRIDPLFGTLEDLEYLRDALHKAGIRLLLDFVPNHTSDEHPWFQWSIKKVDPYTNYYVWKDAKMVNGQRKPPTNWISEFGGSMWKWNDERQQYFLHQFHQKQPDLNYECKELVQEMMDVVRFWLDRGVDGLRLDAINFMFEDPLWRDQELLAPDLDPNFFLSYNRSRTMDQPATYEMVTRFREIFDSYSKKDGRTRVMMTEAYTTLDKTLGFYQYNGKPGAHMPFNFFFITHVNGRSPAKDYQRAIQGWMQGMPEGHSANWVIGNHDNHRIAARYGTDLVDGMNMIALLLPGTGNTYYGDEIGMDDAAIRFDEGVDPQACQFDKDWFNRVTRDPERTPFQWDTSLNAGFSTSMKTWLPVNSNFWRLNLKDQMQSNEKSHYKVYKRLMDVRKTDTMLYGDVETYVLAKWVFAFTRRLTGSDTYVVVVNLGSDTTSVDLSAFMSDLPETLTVHTGSVNTQYDPNDKVPTGVFYMRPKASILLTTAQEVPPAAYSDSKTSGVATRTSTAIGVIGFLFAHRLLL

SEQ ID NO: 67 - DNA, 987 bp, Bemisia tabaci
  source 1..987: mol_type = genomic DNA
atggccctgagatggtcgctggaggaggctttgcgagtgttcaaattaccgatggcagacttgatgtatcaggctcagtctacccaccgggccaacttcaaccctaacgaaatgcaaatcagtacgcttctgagcatcaagacgggcgcttgcccagaaaactgctcgtactgtccccagtcagcctactacaaaacagagatcaagaaggagcctctgatggatttagcggaggttgttgccgccgcgaaggcagccaaagagagtggaagtacccgtttctgcatgggtgctgcttggcgtggacccacggacaggaatctcccgttggtctgtgatatggtgaaagaggtaaagaagttgggcttagagacttgcgtaacattagggcttctaaaggaccgccacgccgtacaactaaaagaagccggactggacttctacaaccacaacatcgacacgtcaccggagtactacaagaagatcatctcgacgaggactttcgaggatcgaatccggaccctggagcacgtgcgcaacgcagggatcaaggtctgctgcggcgggatcctaggaatgggtgagaacaccgaggaccggatcaagatgctcttggtattggcgaatctggaggagcctcctgaatcagtgccgatcaatcagctgattccgatccctgggactcctctcgccgacgcggaccctgtcgagggcaccgacttcgtgcggaccatcgctctcacccgggtcatgatgcccaaggcctacatccgactctccgctggccgggagaacatgtccgaggagatgcagacgctctgtttcttggcaggagtcaactcgattttctacggtgagaagctcctcacggccaagaacttccggccgagcaaggatgatcaattactcagtaagctcggcttcaagaagatggagattgatgaggcaccggccaacagtcagtgtaaagaggcggccattgggtga

SEQ ID NO: 68 - DNA, 1785 bp, Bemisia tabaci
  source 1..1785: mol_type = genomic DNA
cataaaaggagcttaaacttaaatgttttatgtattttccctttagaaagtagcgcatgaaagcaacttattttcctttttttttttttttttttaaattttaatttgtttgttctgtgtgtttcaggtatgtaatgacaactttaaattctctctccccatttactccagcccatttgctatcctgcgacacgagtttgacgatggaaacatttttttcaccctgtcagaagtgatgaggggagcgacgaggcaagagcggactcgctagtggatggattcggttgagcggaaagacaagtccaccgatgagaggagcggtggatgagcggatgagccctcggttcgactgcgatggatagaccttactcgtgggatcaatgcggatcagtgctgaaggttgaagcgggggtgggcggggtcggggtcgcgggggcgggggtaggaagcaaagccctggaggactcggacgacgggcccgggtgcttctgctgcgactcggacagcgacgacagcgccgagcaggtgcggatccgcaacacgaacaagaagtgcgcccgcgggtgcctcaacaacatcgtcgtctgctcggtcgtcatcttgtacaccttcttcggggccgtcctcttcctcctcatcgagggcggcggggtcttcaccgtcgacgacaagatcctcgacgagtccgaggagccctccttcctcaacgacgagaagatctcggccgtcaagcggaacctcggctccttcaacaacctcacgctccagtggatcgagaacatcaacgccgagaaccgtgagcgcactgtcgagaacatctgggacatcacggtgaacttgaacatcctctaccgggagaactggacccggttggccgccccggagatcaaccggttccagcagcagctcgtcgagcgcatcacgcgggaggtcaccgcctccggtccctccgagtccgagcttcaggtcccggtgcccgtgccggttcaggtcgaacgtcacctcgtcgactcttcctatcggatggatcagtatcctctggagtggacactcgccaaggctttcctcttctccctcaccatcatcacgacaataggtgagcattggacggccttcactcccgagcaatttttcaagccttggcataaaatcggagcgaaaatggggctcattgaatcagcattaatggccatggcggtatcactgtgcttattcgtcttcgcacactcgaaaagcgtggcggtatgccagccgcaaaccgcggaagtatgccacccgcaaacggcggaagcgaatgcggacgcttcgacaggagctatgatcatcctatccctggcctcgacgctgaacaatcggatggagtacatgaaagacgtggccgggaacaagttcaaagccaacaagccggtcgaggatctaccccgcgaggagaaggtcatgaactcggccgtcgccaacgcctcggccgaggggctggatccgaattcgatacggcccttcatccaggcgcagatggacgtggccaaggacatccagcgcgtctatattgccggctggaaggttaggccagagaagtgggatcccagggcgctcgaggaggtcagggagctcatctccgcggacgacgccaagatcctgaggctcagtcgacagcaacttacggttggagggttttgcgaggcctatagggagcagatctacaggagcctcacagcgccttacatcactgatgcgcatagaaaactcctcgtggatacgctgttttgcgttaggcttaagcagtag

SEQ ID NO: 69 - DNA, 801 bp, Bemisia tabaci
  source 1..801: mol_type = genomic DNA
catcttgaattttcacccatctataaaattacagacatgcacggcctgggcaatgacttcatggtcgtggacaccgtcacccagaacatctccttcacgacggaccagatccgtctcttggccgaccgacacctgggcgtcgggttcgaccagctcctggtcgtcgagccctcgtcggagcccgacgtggacttccactaccggatcttcaacgtggacggaagcgaggtggcccaatgcgggaacggcgcccggtgtttcgcccgcttcgtccggctccagcgcttgagccggaagcgcgacctccacgtcagcaccgcctccgggcgccgcatggtcctctccatcacgccggacgaccaggtccgcgtcaacatgggcgagcccactctttaccacatggagaacgtgacgttcggttgcgtgtcgctaggcaacccgcactgcgtggtgcaggtagagagtgttaagaccgcgcctgtggacacgctcggggccatgctggagacccacgagcgcttcccggagcgaaccaatgtgggctttatggaggtcgtcgatcccgggcatatccgccttcgcgtgttcgagcgtggtgtgggcgagacgagggcctgtggcagcaacgcctgtgctgctgttgtctgcggcgttcgccagggtgcgctgacgtcgccggtgcgcgtggatctccctggcggcacgctccgtgtcgcctgggcggggcttggacagccggtctacttgaccggtccggcggttcatgtttatgatggcttcgttgatttgggaaatacatga

SEQ ID NO: 70 - RNA, 370 nt, synthetic construct
  source 1..370: mol_type = other RNA; note = dsRNA
tgattttgttccaaaccacactagtgaccgtcacttgtggtttcgaaagtctgtcgccagggaagaaccttatacagatttctacgtttggagagatgccaaagtcgtcaatggtctacggttacccccaaataattggcggagtcagtttgccggatctgcgtggacttggaatgaagagagacagcaatatttctatcatcagttccatactaaacagccagatttgaattacaactgtaaagagctagtcagagaaatgatctcaacgttaaaattttggttggatcgaggtgtggatggcttccgtatggatgctgtgacgtacatgtacgaagacccgaactgggcggatgaggaattgctgaat

SEQ ID NO: 71 - RNA, 497 nt, synthetic construct
  source 1..497: mol_type = other RNA; note = dsRNA
tcgaaagacagacacgatgctgtacggagatgtggagacctacgtgctcgccaaatgggtcttcgcctttaccaggcgtttgactggaagtgacacttatgtggtggtcgttaatcttgggagcgacactacatctgtcgatctctctgctttcatgagtgatctaccggagactttaacagtccacacgggcagtgtcaatacccagtatgatccaaatgacaaagtaccaactggtgtattttacatgcgaccaaaagcctccatccttttaaccactgcccaagaagtgccgcctgctgcatattcggattcaaaaactagtggagtcgcaacgaggacatccacttcaatcggaataattgtatttcttttcactcataggttactcctataggctaatgtccattccttttatgtgtatttttattgtaatttatgataatgaaaattaaaaagatgaaacaaaagaagaaggaactacccgacggaaatca

SEQ ID NO: 72 - RNA, 501 nt, synthetic construct
  source 1..501: mol_type = other RNA; note = dsRNA
gagactactctactttgttgtgtgcgctaacatttggattggattttcggaaagccatcagattctaagcagcggcggaatgaaggagtggtgggagagcactctgatttatcagatttacccacgctcctttaaggattcaaatggggatggctacggtgatctaaatggtattcttgaaagactggattatgtcaaggagataggcgtggacacaatttggattcaaccgttctacaagtcgccgatggctgatatgggttacgacgttgctgattatcgagctgttgatccaatgttcggtaccatggatgattttaagaggttactaaaaaatagtcatgagaaagggatgaaagtgattgttgactttgtgccgaatcatagtagtgatcaaggtgagtggtttaagctctccgaacaacgtatcgaaccgttcacagatttctacgtttggaaggacggtaaacggattaacgaaactcacacaacacatccaaa

SEQ ID NO: 73 - RNA, 481 nt, synthetic construct
  source 1..481: mol_type = other RNA; note = dsRNA
cagcgaaaggagaaaggatgccaccgacgaactgggtcagtgaatttggaggctacacttggagctggaatgaggaaaggcaacaatattatcttcatattttccattacaagcaacctgacttaaactacaacagcaaagaactcgttagagagatgacggatgtgatcagattttggctagacagagggattgatggtctcagaatggatgctgttacctacatgtatgaagatccgcagtatagagatcaagaattattggccccagatttagatccatgggatttcaaaagctacaaccgctgccacacgatggaccaaccggcaacatacagattaataaccaagttcagggagatttttgacgcctactccaagagagatggcgagactaaggtcatgataacagaggcatacacatcattggataaaactatggaatactacaagttcggtgataaacctggtgctcatatgcc

SEQ ID NO: 74 - RNA, 481 nt, synthetic construct
  source 1..481: mol_type = other RNA; note = dsRNA
cggcgatctaaaaggaatcaaagagaaagtccactatctttgtgagctgggagtgaccgcaatttggataaacccgttctacgaatccccgctgaaagacaacggttatgacatctctaatttcaagaaaatccaccacaagttcggtaacatgtgtgactttaaggcactcatgaaaagacttgataataatccttgcggtgataacaaagagatcagagtgatcatggatttcgtacccaaccacacgagcgacgaacatgaatggtttcagaagtcagtcgatcgggtgcctccatacaatgattactacgtatggatggatcctaaaggatgggacaaaaatggaaatccaatccctcccaacaattgggcatcagtcttcagctacggagaggaagcgtctggatgggagtggaatgagaagagacaacaattttactttcacgctttcatgagggaacagccggaccttaacctt

SEQ ID NO: 75 - RNA, 897 nt, synthetic construct
  source 1..897: mol_type = other RNA; note = dsRNA
gagcaacaaacggacaaaggaaaagcgcctatggaatcgccaactgaattgaatcaatcagagaagtctgtgggtgaaagtgaggaaaaacataaaaagacaaaattcaaaataagagctggtggtggaaatgagaagagagatgatatagataaaattccaaccaacgctctagaattccggaagagcttcaaaccaccaaaagtgtcacaagcagcatatgtatggataccacgttcgcaaagagataatttgacacctgatgtcatacagaattttctagcgtacgtgcctccatcacatgctatagacaatcaattggcttctggagttgaagttgagaattgggccattgaggtttcaaaagcttatggagtcaccattcaagagttttatagaacgatcctaccagcttggattgttaattgtattgtgaatgggactagtgatgaaaggaaaaatgagaaatcgtggagagctgttgagctaaatacgcagggtgaggatattgatgattcagaataccctatggaaccaatgtacaaatttgctctcccgacaatgagaaaaataatgagaaatttttccagccaagcaatcctaatgtatcagaatagtgtgtctgctggaaaagcttttgtaataaaagcagcccgaaatgccgggtatacaagcattgaaaataagtggttgggtattgacttcctagccgaagcacaattgtcacaaagccaacttgatatcaaacatcaaatattggctgctaatgttggtagaagtaaaactaagttgtttgctttagctgctcctggtgacgataataatgtggataaagaaaggcacactacgcgggatgttagtgcaactaggcacagctacgcgggtgccgctatcgaa

SEQ ID NO: 76 - DNA, 20 bp, synthetic construct
  source 1..20: mol_type = other DNA
cattccactacagagctcca

SEQ ID NO: 77 - DNA, 20 bp, synthetic construct
  source 1..20: mol_type = other DNA
tttcaggtttcggatggctt

SEQ ID NO: 78 - DNA, 20 bp, synthetic construct
  source 1..20: mol_type = other DNA
ggcagtatcaacagtcgtca

SEQ ID NO: 79 - DNA, 20 bp, synthetic construct
  source 1..20: mol_type = other DNA
ggttcttgtagcctggagac

SEQ ID NO: 80 - DNA, 20 bp, synthetic construct
  source 1..20: mol_type = other DNA
atacggcttctgctggattt

SEQ ID NO: 81 - DNA, 20 bp, synthetic construct
  source 1..20: mol_type = other DNA
tggttggtggtttcctttga

SEQ ID NO: 82 - DNA, 19 bp, synthetic construct
  source 1..19: mol_type = other DNA
taacagccactacaaagta

SEQ ID NO: 83 - DNA, 18 bp, synthetic construct
  source 1..18: mol_type = other DNA
gacccatttagagagaac

SEQ ID NO: 84 - DNA, 20 bp, synthetic construct
  source 1..20: mol_type = other DNA
atctacctacacgctcactg

SEQ ID NO: 85 - DNA, 20 bp, synthetic construct
  source 1..20: mol_type = other DNA
ccaagtctggaatctcgtcc

SEQ ID NO: 86 - DNA, 20 bp, synthetic construct
  source 1..20: mol_type = other DNA
tgctggaggaaatggtgttc

SEQ ID NO: 87 - DNA, 20 bp, synthetic construct
  source 1..20: mol_type = other DNA
acagtgaggctgaatcttgg

SEQ ID NO: 88 - DNA, 19 bp, synthetic construct
  source 1..19: mol_type = other DNA
tttcttcaacacgggcgag

SEQ ID NO: 89 - DNA, 20 bp, synthetic construct
  source 1..20: mol_type = other DNA
gtctgggttcttcttctgcg

SEQ ID NO: 90 - DNA, 20 bp, synthetic construct
  source 1..20: mol_type = other DNA
aataggtgagcattggacgg

SEQ ID NO: 91 - DNA, 20 bp, synthetic construct
  source 1..20: mol_type = other DNA
gcgaagacgaataagcacag

SEQ ID NO: 92 - DNA, 20 bp, synthetic construct
  source 1..20: mol_type = other DNA
gccaacttcaaccctaacga

SEQ ID NO: 93 - DNA, 20 bp, synthetic construct
  source 1..20: mol_type = other DNA
aacaacctccgctaaatcca

(19) United States
(12) Patent Application Publication
(10) Pub. No.: US 2025/0027105 A1
(43) Pub. Date: Jan. 23, 2025

(54) COMPOSITIONS AND METHODS FOR MANAGEMENT OF WHITEFLY

(71) Applicants: Natural Resources Institute, Chatham Maritime (GB); Yissum Research Development Company of The Hebrew University of Jerusalem LTD, Jerusalem (IL); Cornell University, Ithaca, NY (US); Donald Danforth Plant Science Center, St. Louis, MO (US); National Agricultural Research Organization, Entebbe (UG)

(72) Inventors: John Colvin, Newington (GB); Sharon L. van Brunschot, Highgate Hill (AU); Rekha Swamy, Gillingham (GB); Susan Seal, Maidstone (GB); Shai Morin, Jerusalem (IL); Osnat Malka, Givatayim (IL); Dor Wintraube, Tel-Aviv (IL); Nigel Taylor, St. Louis, MO (US); Narayanan Narayanan, St. Louis, MO (US); Titus Alicai, Kampala (UG); Tadeo Kaweesi, Kampala (UG)

(21) Appl. No.: 18/773,683

(22) Filed: Jul. 16, 2024

Related U.S. Application Data
(60) Provisional application No. 63/514,082, filed on Jul. 17, 2023.

(51) Int. Cl. C12N 15/82; C12N 15/113

(52) U.S. Cl. CPC C12N 15/8286; C12N 15/113; C12N 2310/14

(57) ABSTRACT

The present disclosure is directed to controlling pest infestation by inhibiting one or more biological functions in an invertebrate pest. The disclosure discloses methods and compositions for use in controlling pest infestation by feeding one or more different recombinant double stranded RNA molecules to the pest in order to achieve a reduction in pest infestation through suppression of gene expression. The disclosure also discloses methods and compositions for targeted genome editing in the pest in order to achieve a reduction in pest infestation through disruption of protein activity. The disclosure is also directed to methods for making transgenic plants that express the double stranded RNA molecules and targeted genome editing constructs for use in protecting plants from pest infestation.

### REFERENCE TO RELATED APPLICATIONS

This application claims the benefit of U.S. Provisional Application No. 63/514,082 filed Jul. 17, 2023, which is incorporated herein by reference in its entirety.

### INCORPORATION OF SEQUENCE LISTING

A sequence listing containing the file named “AGOE011US_ST26.xml” which is 109 kilobytes (measured in MS-Windows®) and created on Jul. 11, 2024, and comprises 93 sequences, is incorporated herein by reference in its entirety.

### FIELD OF THE INVENTION

The present disclosure relates generally to genetic control of pest infestations in plants. More specifically, the present disclosure relates to the methods for modifying endogenous expression of coding sequences in the cell or tissue of a particular pest to achieve intended levels of pest control.

### BACKGROUND

Controlling pest infestation is an essential component of horticultural management. Many of the world's farmers face pressure from a wide variety of agricultural pests, which can result in low yield or plant death. Whitefly (e.g., Bemisia. tabaci (sensu latu)), is a globally distributed pest affecting agricultural production, both by direct damage and as a vector of plant viruses. In recent years, whitefly populations have risen to extreme levels, particularly in Africa. This has resulted in significant annual losses of important food security crops like cassava, which is a staple food for more than 400 million people in Sub-Saharan Africa. Therefore, the development of compositions and methods for controlling pest populations, including superabundant whitefly populations, is needed to reduce both the direct damage caused by pests and also the spread of devastating viral diseases transmitted by these pests.

### SUMMARY

In one aspect, the present disclosure provides a recombinant polynucleotide molecule comprising a first polynucleotide sequence having at least about 85% sequence identity to at least 18 contiguous nucleotides of a target nucleotide sequence, wherein said target nucleotide sequence: a) encodes a polypeptide having a sequence selected from the group consisting of SEQ ID NO: 1, SEQ ID NO:3, SEQ ID NO:5, SEQ ID NO:7, SEQ ID NO:9, SEQ ID NO: 11, and SEQ ID NO: 13; or b) has a sequence selected from the group consisting of SEQ ID NO:57, 59, 51, 63, 65, and 67-74; and wherein said recombinant polynucleotide molecule disrupts the activity of said polypeptide when provided in the diet of an invertebrate pest. In some embodiments, said first polynucleotide sequence is operably linked to a heterologous promoter; encodes an interfering RNA molecule; or encodes a ssRNA molecule or a dsRNA molecule. In other embodiments, the first polynucleotide sequence has at least about 90%, at least about 95%, or 100% sequence identity to at least 18 contiguous nucleotides of said target sequence. In certain embodiments, the first polynucleotide sequence has at least about 85% sequence identity to at least 19 contiguous nucleotides, at least 20 contiguous nucleotides, or at least 21 contiguous nucleotides of said target sequence. In some embodiments, the invertebrate pest is a pest of the order Hemiptera, e.g. a whitefly, Bemisia species pest such as Bemisia. tabaci. In another aspect, plants, plant parts, plant cells, seeds, or commodity products comprising the recombinant polynucleotide molecule provided herein are described. In yet another aspect, compositions comprising the recombinant polynucleotides provided herein are described. In some embodiments, the present disclosure provides a composition comprising a polynucleotide molecule comprising a first polynucleotide sequence having at least about 85% sequence identity to at least 18 contiguous nucleotides of a target nucleotide sequence, wherein said target nucleotide sequence a) encodes a polypeptide having a sequence selected from the group consisting of SEQ ID NO:1, SEQ ID NO:3, SEQ ID NO: 5, SEQ ID NO:7, SEQ ID NO:9, SEQ ID NO: 11, and SEQ ID NO: 13; or b) has a sequence selected from the group consisting of SEQ ID NO:57, 59, 51, 63, 65, and 67-74; and wherein said composition disrupts the activity of said polypeptide when provided in the diet of an invertebrate pest. In certain embodiments, the polynucleotide molecule is an interfering RNA molecule or encodes an interfering RNA molecule; or a ssRNA molecule or a dsRNA molecule or encodes a ssRNA molecule or a dsRNA molecule. Further provided are methods for controlling invertebrate pest infestation, the methods comprising providing a polynucleotide molecule comprising a first polynucleotide sequence having at least 85% sequence identity to at least 18 contiguous nucleotides of a target nucleotide sequence in the diet of an invertebrate pest, wherein said polynucleotide molecule disrupts the activity of a polypeptide encoded by said target nucleotide sequence, wherein said target nucleotide sequence: a) encodes a polypeptide having a sequence selected from the group consisting of SEQ ID NO:1, SEQ ID NO:3, SEQ ID NO:5, SEQ ID NO:7, SEQ ID NO:9, SEQ ID NO: 11, and SEQ ID NO:13; or b) has a sequence selected from the group consisting of SEQ ID NO:57, 59, 51, 63, 65, and 67-74. In certain embodiments of the methods described, the polynucleotide molecule is an interfering RNA molecule or encodes an interfering RNA molecule; or a ssRNA molecule or a dsRNA molecule or encodes a ssRNA molecule or a dsRNA molecule. In other embodiments, providing the polynucleotide molecule comprises providing a plant, plant part, plant cell, seed, or composition comprising said polynucleotide molecule in the diet of the invertebrate pest. In further embodiments, the invertebrate pest is a pest of the order Hemiptera, e.g. a Bemisia species pest. Throughout this specification and the claims, unless the context requires otherwise, the word “comprise” and its variations, such as “comprises” and “comprising,” will be understood to imply the inclusion of a stated composition, step, and/or value, or group thereof, but not the exclusion of any other composition, step, and/or value, or group thereof.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows differential gene expression in cassava B. tabaci (SSA1-SG1) gut. The volcano plot shows transcripts enriched in the B. tabaci SSA1-SG1 gut.

FIG. 2 shows differential gene expression in cassava B. tabaci (SSA1-SG1) gut. Panel A) is an MA-plot showing a global view of differential expression in the B. tabaci gut compared to whole-body. Panel B) shows dispersion estimates in gene expression in the gut.

FIG. 3 shows Tag distribution for the transcripts enriched in the cassava B. tabaci (SSA1-SG1) gut. A number of sequences were found to have hits in the NCBI non-redundant database and InterproScan database.

FIG. 4 shows species distribution of all the NCBI top blast-hits for the transcripts enriched in cassava B. tabaci (SSA1-SG1) gut.

FIG. 5 shows gene ontology (GO) annotation. Distribution of level 2 of the GO classification for biological process, molecular function and cellular component of the transcripts enriched in the cassava B. tabaci (SSA1-SG1) gut.

FIG. 6 shows direct GO count for biological process of transcripts enriched in cassava B. tabaci gut. Sucrose metabolic process was listed in the top 15 biological processes encoded by the sequences that are enriched in the cassava whitefly gut.

FIG. 7 shows gene ontology (GO) categorization, level 2 for Biological process of the transcripts enriched in the cassava B. tabaci (SSA1-SG1) gut. The largest proportion of transcripts enriched in the whitefly gut encode for metabolic processes.

FIG. 8 shows an InterProScan family distribution of the transcripts enriched in the cassava B. tabaci (SSA1-SG1) gut.

FIG. 9 shows an InterProScan domain distribution of the transcripts enriched in the cassava B. tabaci (SSA1-SG1) gut.

FIG. 10 shows the phylogenetic relationship of all aquaporin genes of cassava B. tabaci (SSA1-SG1) and experimentally validated aquaporins (Van Ekert et al., 2016). Phylogenetic analysis was done using Bayesian method implemented in Bayesian Evolutionary Analysis Sampling Trees (BEAST version 1.10.2). Scalebar represent branch length, an inference of genetic difference between the analysed sequences (number of substitutions per site)

FIG. 11 shows a multiple sequence alignment of selected α-glucosidase protein sequences, ENSSSA1UG % 028740 (Ssa12486) (SEQ ID NO: 58), NP_001119607.1_Aphid (SEQ ID NO: 60), ENSSSA1UGT002066 (Ssa05164) (SEQ ID NO: 62), ENSSSA1UGT002057 (Ssa05154) (SEQ ID NO: 64), and Ssa12230 (Bta15649) (SEQ ID NO: 66). The highlighted region is the partial sequence of conserved region II of α-glucosidase family 13. The function and substrate preference of α-glucosidase enzyme is determined by the amino acid residue at position 4 and 5 of the conserved region II. Protein sequences were aligned using Clustal Omega9 software.

FIG. 12 shows phylogenetic relationships of α-glucosidases protein sequences with both predicted signal peptides and catalytic site residue and experimentally validated Sucrase gene in Acyrthosiphon pisum (Price et. al., 2007). Genes in parenthesis are the orthologs of alpha-glucosidase family 13 in MEAM1 B. tabaci species. Phylogenetic analysis was done using Bayesian method implemented in Bayesian Evolutionary Analysis Sampling Trees (BEAST version 1.10.2). Scalebar represent branch length, an inference of genetic difference between the analysed sequences (number of substitutions per site).

FIG. 13 shows differential gene expression in cassava B. tabaci (SSA1-SG1) gut. Volcano plot showing genes enriched in the bacteriocyte.

FIG. 14 shows differential gene expression in cassava B. tabaci (SSA1-SG1) gut. A) MA-plot showing a global view of differential expression in the B. tabaci bacteriocytes compared to whole-body. B) Dispersion estimates of gene expression in cassava B. tabaci bacteriocytes.

FIG. 15 shows tag distribution for the transcripts enriched in the cassava B. tabaci (SSA1-SG1) bacteriocyte. Number of sequences blasted against NCBI non-redundant database and InterproScan database.

FIG. 16 shows top-hit species distribution for the differentially expressed transcripts in cassava B. tabaci (SSA1-SG1) bacteriocyte blasted against NCBI non-redundant database.

FIG. 17 shows gene ontology (GO) categorization, level 3 for the transcripts enriched in the cassava B. tabaci (SSA1-SG1) bacteriocyte.

FIG. 18 shows the distribution of transcripts enriched in the bacteriocyte as assigned to different categories of biological processes in the cassava B. tabaci (SSA1-SG1) bacteriocyte.

FIG. 19 shows direct gene ontology count for transcripts enriched in the cassava B. tabaci (SSA-SG1) bacteriocyte.

FIG. 20 shows quantitative real-time PCR gene expression of selected genes in the cassava whitefly (SSA1-SG1) gut and bacteriocytes relative to whole-body. Gene ENSSSA1UGT022145 (AQP1), ENSSSA1UGT002057 (SUC1) and ENSSSA1UGT002066 (SUC2) were selected as critical osmoregulation genes with enriched expression in the whitefly gut while ENSSSA1UGT008852 (AAT), ENSSSA1UGT003467 (ArgH), ENSSSA1UGT021004 (BCAT), ENSSSA1UGT000364 (dapB) and ENSSSA1UGT007134 (LysA) were selected as essential symbiosis genes that mediate terminal reaction in the phenylalanine, arginine, leucine, isoleucine, valine and lysine biosynthesis pathways. The relative gene expression of the selected genes was normalized relative to the relative expression of two reference genes (ribosomal protein L13a and β-tubulin) using normalized expression ΔΔCq method (Livak & Schmittgen, 2001).

FIG. 21 shows the distribution of sequences as assigned to different categories of biological processes encoded by genes in Portiera aleyrodidarum genome from B. tabaci SSA1-SG1

FIG. 22 shows direct GO counts for biological processes encoded by genes in Portiera aleyrodidarum genome from B. tabaci SSA1-SG1

FIG. 23 shows identification of secondary symbionts co-existing with Portiera aleyrodidarum in the cassava B. tabaci (SSA1-SG1) bacteriocyte. Restriction pattern of endosymbionts based on 16S for V6-V7 region generated using Mwo1 restriction enzyme and visualized using a 1 kb ladder (Quick-load, New England BioLabs Inc). Lane 1 and 2, positive control for Wolbachia-digested, lane 3 and 4, positive control for Wolbachia-undigested PCR product, lane 5 and 6, MED_ASL-digested, lane 7 and 8, MED-undigested PCR product, lane 9 and 10, SSA1-SG1 whole-body-digested, lane 11 and 12, SSA1-SG1 whole-body-undigested PCR product, lane 13 and 14 SSA1-SG1 bacteriocytes-digested and lane 15 and 16 SSA1-SG1 bacteriocyte-undigested PCR products.

FIG. 24 shows phylogenetic relationship of the 16S V6-V7 region of selected B. tabaci endosymbionts. Phylogenetic analysis was conducted using Bayesian methods implemented in Bayesian Evolutionary Analysis Sampling Trees (BEAST version 1.10.2) (Suchard et al., 2018).

FIG. 25 shows in silico prediction of production rates of essential amino acid of the two-compartment metabolic model of B. tabaci SSA1-SG1 and Portiera.

FIG. 26 shows robustness analysis measuring sensitivity of the metabolic object function (growth rate of B. tabaci SSA1-SG1) to the quantitative flux levels through terminal reactions for (A) arginine biosynthesis, (B) lysine biosynthesis, (C) histidine biosynthesis, (D) leucine biosynthesis, (E) isoleucine synthesis and (F) valine biosynthesis.

FIG. 27 shows robustness analysis measuring sensitivity of the metabolic object function (growth rate of B. tabaci SSA1-SG1) to the quantitative flux levels through terminal reactions for (A) phenylalanine biosynthesis (B) methionine biosynthesis (C) threonine biosynthesis and (D) tryptophan biosynthesis.

FIG. 28 shows robustness of the metabolic network “SSA1-SG1_Portiera” iKT420 showing the effect of single reaction deletion on the growth rate of B. tabaci SSA1-SG1 and Portiera mutant relative to the wild-type, simulated using flux balance analysis. A total of 270 reaction deletions affected growth, therefore, were deemed indispensable/essential for the growth and survival of cassava B. tabaci SSA1-SG1

FIG. 29 shows sucrose hydrolysis in whiteflies feeding on different sucrose sources. Amount of sucrose and free glucose in honeydew collected from whiteflies feeding on Arabidopsis, tomato, tobacco and artificial diet with 0.75 M sucrose. Treatments sharing the same compact letter display are not significantly different at 5% significance level.

FIG. 30 shows a comparison of sucrose hydrolysis in the aphid Aphis gossypii and the whitefly B. tabaci MEAM1 feeding on both tobacco and artificial diet with 0.75 M sucrose. Error bars represent standard deviation. Treatments sharing the same compact letter display are not significantly different at 5% significance level.

FIG. 31 shows the percentage mortality of whiteflies feeding on different tomato plants with dsRNA against RNase, GFP, AQP1 and SUC1 in the first experiment.

FIG. 32 shows the percentage mortality of whiteflies B. tabaci MEAM1 feeding on different transgenic tomato plants with dsRNA against RNase, GFP, AQP1 and SUC1 after six days in the second experiment. Both plants with empty vector construct and wildtype tomato were used as controls. Treatments sharing the same compact letter display are not significantly different at 5% significance level.

FIG. 33 shows relative gene expression of AQP1 genes in B. tabaci MEAM1 that survived after six days of feeding on different stably transformed tomato plants in the first experiment.

FIG. 34 shows relative gene expression of SUC 1 genes in B. tabaci MEAM1 that survived after six days of feeding on different stably transformed tomato plants in the first experiment.

FIG. 35 shows gene expression of AQP1 genes in B. tabaci MEAM1 that survived after six days of feeding on different stably transformed tomato plants in the second experiment.

FIG. 36 shows gene expression of SUC1 genes in B. tabaci MEAM1 that survived after six days of feeding on different stably transformed tomato plants in the second experiment. Plants with empty vector construct were used as control

FIG. 37 shows the effect of RNA interference on the development of B. tabaci MEAM1 feeding on different transgenic tomato plants in the first experiment. One-way ANOVA revealed that there was no significant difference between the number of 3rd instar nymphs and crawlers on different tomato transgenic plants.

FIG. 38 shows haemolymph osmotic pressure for B. tabaci feeding on different stably transformed tomato plants. Osmotic pressure in this study was used as a physiological index of B. tabaci osmoregulation to study the effect of feeding whiteflies on different stably transformed tomato plants with different dsRNA against osmoregulation genes in the B. tabaci.

FIG. 39 shows sucrose hydrolysis as a physiological change resulting from whiteflies feeding on different stably transformed tomato plants containing dsRNA against SUC1. Treatments sharing the same compact letter display are not significantly different at 5% significance level.

FIG. 40 shows the molar ratios of different sugars in honeydew samples collected from whiteflies feeding on different transgenic tomato plants. Error bars represent standard deviations.

FIG. 41 shows sugar concentration (hexose units per mg of honeydew) collected from whiteflies feeding on different transgenic tomato plants. Error bars represent standard deviation.

FIG. 42 shows a comparison of molar ratios of sugars in honeydew samples collected from aphids and whiteflies feeding on tobacco and artificial diet. Error bars represent standard deviation.

FIG. 43 shows a comparison of sugar concentration in different honeydew samples collected from aphids and whiteflies feeding on both tobacco and artificial diet. Error bars represent standard deviation.

FIG. 44 shows a maximum likelihood circular cladogram showing the phylogenetic relationships between glycoside hydrolase family 13 enzymes of B. tabaci. The tree was produced using protein sequences of 70 GH13 members. Shaded sequences indicate SUC3, SUC4, SUC7, and SUC12. The colours of the circle around the cladogram indicate the clade of the corresponding GH13 members. The protein sequences are named according to their NCBI accession numbers.

FIG. 45. Shows the effects of feeding on dsRNA diets targeting GH13 genes of B. tabaci adults. (Panel A) Relative survival of insects that were fed for five days on artificial diets containing dsSUC3, dsSUC4, dsSUC7, dsSUC12, and dsCBSV (n=15 replicates per treatment). Error bars represent SEM. ** correspond to a P value of <0.01. (Panel B) Relative expression levels, as determined using the 2\hat{ }-ΔΔCt method, of the SUC3, SUC4, SUC7, and SUC12 genes in B. tabaci adults after feeding for five days on dsRNA targeting the SUC3, SUC4, SUC7, and SUC12 genes or dsCBSV control diet (n=5 replicates per treatment). Error bars represent SEM. *, **, and ***, correspond to P values of ≤0.05, <0.01, and <0.001, respectively.

FIG. 46 shows plant performance experiments. (Panel A) Relative, mean survival proportion of seven pairs of B. tabaci adults after feeding for five days on artificial diets containing dsSUC3, dsSUC4, dsSUC7, dsSUC12, and dsCBSV, followed by 48 hours on Brussels sprout plants (n=6-8 replicates per treatment). Error bars represent SEM. Nearly significant differences between each treatment and the control are denoted with ˜*, corresponding to a P value of <0.1. (Panel B) Mean progeny numbers per seven pairs of B. tabaci adults (n=6-8 replicates per treatment) after feeding for five days on the aforementioned diets followed by 48 hours on Brussels sprout plants. Error bars represent SEM. Significant differences between each treatment to the control are denoted with **, corresponding to a P value of <0.01. (Panel C) The proportion of progeny of the silenced insects, that remained undeveloped (red), or reached early (green) or advanced (blue) nymph developmental stages after ˜3 weeks. Significant differences between each treatment and the control are denoted with ** and ***, corresponding to P values of <0.01 and <0.001, respectively. (Panel D) Abnormal phenotypes of progeny of silenced B. tabaci adults, observed at the end of the development period.

FIG. 47 shows relative sugar composition of honeydew secreted by B. tabaci adults during a five days period of feeding on artificial diets containing dsSUC3, dsSUC4, dsSUC7, dsSUC12, and dsCBSV (n=10 replicates per treatment). F+G—fructose and glucose, ISO—all sucrose isomers, OLIGO-all oligosaccharides. Each sugar/s fraction of each sample was normalized to the corresponding mean fraction of the dsCBSV treatment. Error bars represent SEM. Significantly differences between each treatment and the control are denoted with ˜*, *, and **, corresponding to P values of <0.1, <0.05, and <0.01, respectively.

FIG. 48 shows data from the Whitefly Resistance Trait Selection Trial I showing the mean number of adults (Panel A) and nymphs (Panel B) over five sampling periods (one to five months after planting) of WF-CFT-1 (DWF 10-15), after normalizing to the counts of NASE 13 WT. Lines within DWF 10, DWF 11, DWF 14 and DWF 15 were significantly different (having lower counts of adults and/or nymphs) from the control NASE 13 WT plants (P≤0.05, black “turn-left” arrow). Adult counts were lower than Mkumba, a highly whitefly resistant line. Analysis was conducted using an ANOVA one-way model (lines), followed by paired comparisons. The control cassava varieties were NASE 13 WT, NASE 13 expressing dsRNA against CBSV, NASE 12 (a whitefly susceptible line) and Mkumba (a whitefly resistant line).

FIG. 49 shows data from the Whitefly Resistance Trait Selection Trial II showing the mean number of adults (Panel A) and nymphs (Panel B) over five sampling periods (one to five months after planting) of WF-CFT-2 (including DWF 57, 61, 62), after normalizing to the counts of NASE 13 WT. Only one line, DWF62-N13004, had lower counts of adults and nymphs when compared to the control NASE 13 WT plants (P≤0.05, black arrow). Nymph counts were lower than Mkumba, a highly whitefly resistant line. Analysis was conducted using an ANOVA one-way model (lines), followed by paired comparisons. The control cassava varieties were NASE 13 WT, NASE 12 (a whitefly susceptible line) and Mkumba (a whitefly resistant line).

FIG. 50 shows data from laboratory adult survival assays showing the percent adult mortality on transgenic and control cassava plants after 7 days normalized to the mortality on the NASE 13 WT plants. Many lines within the DWF 10-15 technologies were found to cause significant higher mortality of adults when compared to the control plants (P<0.05). Analysis was conducted using one-way ANOVA model (lines), followed by paired comparisons. The control cassava varieties were NASE 13 WT and a line expressing dsRNA against the GFP gene (WF17-GFP004).

FIG. 51 shows data from laboratory nymph development assays showing the proportion of progeny that were in advanced development stage (red-eyed 4th nymphs) or completed development (emerged as adults leaving the remains of their exoskeleton (exuviae) behind) 25 days after egg laying normalized to the mortality on the NASE 13 WT plants. 16 lines were significantly different (causing lower development rate) from the control plants (P<0.05). Analysis was conducted using an ANOVA one-way model (lines), followed by paired comparisons. The control cassava varieties were NASE 13 WT and NASE 13 expressing dsRNA against the GFP gene (WF17-GFP004).

FIG. 52 shows data from laboratory adult survival assays showing percent adult mortality on transgenic and control cassava plants after 7 days normalized to the mortality on the NASE 13 WT plants. Most lines of DWF 61-65 technologies were found to cause significant higher mortality of adults when compared to the control plants (P<0.05). Analysis was conducted using one-way ANOVA model (lines), followed by paired comparisons. The control cassava varieties were NASE 13 WT and a line expressing dsRNA against the GFP gene (WF17-GFP004).

FIG. 53 shows data from laboratory nymph development assays showing the proportion of progeny that were in advanced development stage (red-eyed 4th nymphs) or completed development (emerged as adults leaving the remains of their exoskeleton (exuviae) behind) 25 days after egg laying normalized to the mortality on the NASE 13 WT plants. 9 lines were significantly different (causing lower development rate) from the control plants (P<0.05). Analysis was conducted using an ANOVA one-way model (lines), followed by paired comparisons. The control cassava varieties were NASE 13 WT and NASE 13 expressing dsRNA against the GFP gene (WF17-GFP004).

FIG. 54 shows data from adult gene silencing assays showing that relative expression levels of genes targeted by the dsRNA treatments normalized to the control (NASE 13 plants expressing dsGFP), as determined by the ΔΔCt method. Expression levels were calculated for 500 ng RNA samples extracted from 50 adults (n=4 replicates per each target gene, for both treatment and control). Values are presented as means±standard error, and significant differences are indicated by black arrows (P≤0.05).

FIG. 55 shows data from nymph gene silencing assays showing relative expression levels of genes targeted by the dsRNA treatments normalized to the control (NASE 13 plants expressing dsGFP), as determined by the ΔΔCt method. Expression levels were calculated for 500 ng RNA samples extracted from 50 nymphs (n=4 replicates per each target gene, for both treatment and control). Values are presented as means±standard error, and significant differences are indicated by a black “left-turn” arrow (P<0.05).

FIG. 56 shows the correlation between the relative expression levels of genes targeted by the dsRNA treatments and the performance of the insects (adult survival after 7 days and nymph development stage after 25 days) after normalization to the control (NASE 13 plants expressing dsGFP). Significant positive correlation indicates that reduced gene expression results in reduced performance, indicating the gene silencing is likely to be the mechanism causing lower performance.

### BRIEF DESCRIPTION OF THE SEQUENCES

SEQ ID NO:1 is the amino acid sequence of the B. tabaci water-carrying aquaporin, known as AQP1.

SEQ ID NO:2 is a nucleotide sequence encoding SEQ ID NO:1. Nucleotides 67-480 were identified as a target region for suppression using dsRNA as described herein.

SEQ ID NO:3 is the amino acid sequence of the B. tabaci α-glucosidase gene, known as SUC1.

SEQ ID NO:4 is a nucleotide sequence encoding SEQ ID NO:3. Nucleotides 61-416 were identified as a target region for suppression using dsRNA as described herein.

SEQ ID NO:5 is the amino acid sequence of the B. tabaci α-glucosidase gene, known as SUC2.

SEQ ID NO:6 is a nucleotide sequence encoding SEQ ID NO:5.

SEQ ID NO:7 is the amino acid sequence of the arginosuccinate lyase gene identified in the bacteriocyte of B. tabaci SSA1-SG1, known as argH.

SEQ ID NO:8 is a nucleotide sequence encoding SEQ ID NO:7. Nucleotides 362-715 were identified as a target region for suppression using dsRNA as described herein.

SEQ ID NO:9 is the amino acid sequence of the diaminopimelate decarboxylase gene identified in the bacteriocyte of B. tabaci SSA1-SG1, known as LysA.

SEQ ID NO:10 is a nucleotide sequence encoding SEQ ID NO:9. Nucleotides 128-472 were identified as a target region for suppression using dsRNA as described herein.

SEQ ID NO:11 is the amino acid sequence of the branched-chain-amino-acid aminotransferase gene identified in the bacteriocyte of B. tabaci SSA1-SG1, known as BCAT. SEQ ID NO:12 is a nucleotide sequence encoding SEQ ID NO:11.

SEQ ID NO:13 is the amino acid sequence of the 4-hydroxy-tetrahydrodipicolinate reductase gene identified in the bacteriocyte of B. tabaci SSA1-SG1, known as dapB.

SEQ ID NO:14 is a nucleotide sequence encoding SEQ ID NO:13.

SEQ ID NOs: 15-34 are primer sequences used in the validation of gene expression of selected gene.

SEQ ID NOs: 35-56 are characteristic catalytic site residue for α-glucosidase enzymes.

SEQ ID NO:57 is the nucleotide sequence encoding ENSSSA1UG % 028740 (Ssa12486).

SEQ ID NO:58 is the protein sequence encoded by SEQ ID NO:57.

SEQ ID NO:59 is the nucleotide sequence encoding NP_001119607.1_Aphid.

SEQ ID NO:60 is the protein sequence encoded by SEQ ID NO:59.

SEQ ID NO:61 is the nucleotide sequence encoding ENSSSA1UGT002066 (Ssa05164).

SEQ ID NO:62 is the protein sequence encoded by SEQ ID NO:61.

SEQ ID NO:63 is the nucleotide sequence encoding ENSSSA1UGT002057 (Ssa05154).

SEQ ID NO:64 is the protein sequence encoded by SEQ ID NO:63.

SEQ ID NO:65 is the nucleotide sequence encoding Ssa12230 (Bta15649).

SEQ ID NO:66 is the protein sequence encoded by SEQ ID NO:65.

SEQ ID NO:67 is the nucleotide sequence of ENSSSA1UGT008510 (BioB). Nucleotides 460-894 were identified as a target region for suppression using dsRNA as described herein.

SEQ ID NO:68 is the nucleotide sequence of ENSSSA1UGT027679 (CM). Nucleotides 1203-1663 were identified as a target region for suppression using dsRNA as described herein.

SEQ ID NO:69 is the nucleotide sequence of ENSSSA1UGT023765 (DapF). Nucleotides 198-565 were identified as a target region for suppression using dsRNA as described herein.

SEQ ID NO:70 is a dsRNA construct sequence directed against SUC3.

SEQ ID NO:71 is a dsRNA construct sequence directed against SUC4.

SEQ ID NO:72 is a dsRNA construct sequence directed against SUC5.

SEQ ID NO:73 is a dsRNA construct sequence directed against SUC7.

SEQ ID NO:74 is a dsRNA construct sequence directed against SUC12.

SEQ ID NO:75 is a dsRNA construct sequence directed against the coat protein of the Cassava Brown Streak virus CBSV.

SEQ ID NO:76 is the qRT-PCR: Bta04282 (RPL13A) Forward primer.

SEQ ID NO:77 is the qRT-PCR: Bta04282 (RPL13A) Reverse primer.

SEQ ID NO:78 is the qRT-PCR: Bta04298 (SUC3) Forward primer.

SEQ ID NO:79 is the qRT-PCR: Bta04298 (SUC3) Reverse primer.

SEQ ID NO:80 is the qRT-PCR: Bta15649 (SUC4) Forward primer.

SEQ ID NO:81 is the qRT-PCR: Bta15649 (SUC4) Reverse primer.

SEQ ID NO:82 is the qRT-PCR: Bta07453 (SUC7) Forward primer.

SEQ ID NO:83 is the qRT-PCR: Bta07453 (SUC7) Reverse primer.

SEQ ID NO:84 is the qRT-PCR: Bta12682 (SUC12) Forward primer.

SEQ ID NO:85 is the qRT-PCR: Bta12682 (SUC12) Reverse primer.

SEQ ID NO:86 is the DWF_57_F Forward primer.

SEQ ID NO:87 is the DWF_57_R Reverse primer.

SEQ ID NO:88 is the DWF_61_F Forward primer.

SEQ ID NO:89 is the DWF_61_R Reverse primer.

SEQ ID NO:90 is the DWF62_9_F Forward primer.

SEQ ID NO:91 is the DWF62_9_R Reverse primer.

SEQ ID NO:92 is the 65_B_Forward Forward primer.

SEQ ID NO:93 is the 65_B_Reverse Reverse primer.

### DETAILED DESCRIPTION

The global resurgence of whiteflies has led to increasing demand for novel management options that can selectively inhibit or downregulate target gene expression in hemipterans such as whitefly with high specificity and fidelity for use in integrated pest management programs. To date, more than 39 morphologically indistinguishable species within the whitefly species of Bemisia tabaci (B. tabaci) complex have been reported. Collectively, members of the B. tabaci species complex can transmit more than 300 plant viruses, some of which cause crop diseases that have been listed among the top 10 most economically damaging plant viruses. In Sub-Saharan Africa, African cassava whitefly (a.k.a. Bemisia tabaci, Sub-Saharan Africa 1-subgroup 1 (SSA1-SG1)) serves as a vector for two devastating cassava plant virus types, cassava mosaic viruses and cassava brown streak virus (CMVs and CBSVs, respectively). Increasing populations of B. tabaci SSA1-SG1, in combination with CMV and CBSV, have resulted in estimated annual losses of cassava between USD 1.9-2.7 billion in areas affected by B. tabaci-transmitted cassava mosaic virus diseases. Management of cassava whiteflies not only reduce the direct damage caused by high whitefly population but can also reduce the spread of devastating viral diseases transmitted by whiteflies.

B. tabaci SSA1-SG1 and other species of whitefly or B. tabaci species complex feed on plant phloem sap that contains high concentrations of soluble sugars, usually sucrose, at concentrations often exceeding 1 M. Due to its significant sugar content, the osmotic pressure of phloem sap is considerably higher than the body fluids of the whitefly. As a result, phloem sap-feeders, require specific genes to mediate phloem sap sugar transformations and ensure water recycling within the gut to maintain body fluid homeostasis.

In addition, whiteflies and other phloem-sap feeding insects depend on endosymbionts for essential amino acid and B vitamin biosynthesis. Whiteflies achieve this through an obligate interaction with intracellular symbiont, Portiera aleyrodidarum (henceforth referred to as Portiera), a vertically transmitted endosymbiont. Portiera is restricted to the cytoplasm of a specialized insect cell known as a bacteriocyte. Their major role is to provide several essential amino-acids or metabolites for intermediate reactions within different essential amino-acid biosynthesis pathways in the host.

The fact that whiteflies, and other phloem sap-feeders, require specific genes to mediate phloem sap sugar transformations and maintain body fluid homeostasis, and also depend on endosymbionts for amino acid biosynthesis, makes these systems and related pathways potential targets for whitefly management. While putative RNAi targets have previously been proposed for the management of whiteflies, these prior studies failed to yield beneficial results for a variety of reasons.

The present disclosure overcomes the limitations of the prior art by identifying and validating critical gene targets enriched in the whitefly gut and bacteriocyte, key components of osmoregulation and symbiosis, respectively, which can be used in the management of cassava whitefly. Importantly, the evaluation of these key target genes was further coupled with phylogenetic analysis of other insects, including other B. tabaci species for identification of osmoregulation genes and application of genome-scale metabolic reconstruction and constraint-based modelling to identify key symbiosis genes. As disclosed herein, the inventors identified candidate osmoregulation gene targets: two α-glucosidases, SUC 1 and SUC 2 with predicted function in sugar transformations that reduce osmotic pressure in the gut; and a water-specific aquaporin (AQP1), which mediates water cycling from the distal to the proximal end of the gut. Interestingly, expression of these genes in the gut was enriched 23.67-fold, 26.54-fold and 22.30-fold, respectively. Genome-wide metabolic reconstruction coupled with constraint based modeling further revealed four gene targets (argH, lysA, BCAT, and dapB) within the bacteriocytes that can be targeted for the management of cassava whiteflies. Further gene targets including BioB, CM, DapF, SUC3, SUC4, SUC5, SUC7, and SUC12 were also evaluated. The present disclosure therefore represents a significant advance in the art.

In particular, the present disclosure provides novel recombinant polynucleotide molecules comprising a first polynucleotide sequence having at least about 85% sequence identity to at least 18 contiguous nucleotides of a target nucleotide sequence, wherein said target nucleotide sequence encodes a polypeptide having a sequence provided herein, and wherein said recombinant polynucleotide molecule disrupts the activity of said polypeptide when provided in the diet of an invertebrate pest. As described herein, ingestion by a target pest of compositions containing one or more dsRNA's, at least one segment of which corresponds to at least a substantially identical segment of RNA produced in the cells of the target pest, resulted in death, stunting, or other inhibition of the target pest. These results indicated that a nucleotide sequence, either DNA or RNA, derived from an invertebrate pest can be used to construct a recombinant pest host (i.e. transgenic plant) that is a target for infestation by the pest. The pest host can be transformed to contain one or more of the nucleotide sequences derived from the invertebrate pest. The nucleotide sequence transformed into the pest host or symbiont encodes one or more RNAs that form into a dsRNA, or serve as a gRNA sequence for a site-specific nuclease, in the cells or biological fluids within the transformed host or symbiont, thus making the polynucleotide molecule available in the diet of the pest if/when the pest feeds upon the transgenic host, resulting in the suppression of expression of one or more genes in the cells of the pest and ultimately the death, stunting, or other inhibition of the pest.

The present disclosure therefore relates generally to genetic control of invertebrate pest infestations in host organisms. More particularly, the present disclosure includes the methods for delivery of pest control agents to an invertebrate pest. Such pest control agents cause, directly or indirectly, an impairment in the ability of the pest to maintain itself, grow or otherwise infest a target host or symbiont. The present disclosure provides methods for employing stabilized polynucleotide molecules, e.g. dsRNA and gRNA molecules, in the diet of the pest as a means for suppression of targeted genes in the pest, thus achieving the desired control of pest infestations in, or about the host or symbiont targeted by the pest. Transgenic plants can be produced using the methods of the present disclosure that express recombinant stabilized dsRNA and siRNA molecules; or gRNA in combination with a site-specific nuclease.

In accomplishing the foregoing, the present disclosure provides a method of inhibiting expression of a target gene in an invertebrate pest, and in particular, in African cassava whitefly or other Hemiptera insect species, resulting in the cessation of feeding, growth, development, reproduction, infectivity, and eventually may result in the death of the pest. The method comprises introducing partial or fully, stabilized guide RNA molecules (gRNA) in combination with a site-specific nuclease, double-stranded RNA (dsRNA) nucleotide molecules, or their modified forms such as small interfering RNA (siRNA) molecules into a nutritional composition that the pest relies on as a food source, and making the nutritional composition available to the pest for feeding. Ingestion of the nutritional composition containing the polynucleotide molecules results in the uptake of the molecules by the cells of the pest, resulting in the inhibition of expression of at least one target gene in the cells of the pest, or the modification of at least one target gene in the cells of the pest. Inhibition or modification of the target gene exerts a deleterious effect upon the pest. The polynucleotide molecules of the present disclosure may comprise a first polynucleotide sequence having at least about 85% sequence identity to at least 18 contiguous nucleotides of a target nucleotide sequence, wherein said target nucleotide sequence encodes a polypeptide having a sequence selected from the group consisting of SEQ ID NO:1, SEQ ID NO:3, SEQ ID NO:5, SEQ ID NO:7, SEQ ID NO:9, SEQ ID NO:11, and SEQ ID NO: 13, and wherein said recombinant polynucleotide molecule disrupts the activity of said polypeptide when provided in the diet of an invertebrate pest. The polynucleotide molecules of the present disclosure may comprise a first polynucleotide sequence having at least about 85% sequence identity to at least 18 contiguous nucleotides of a target nucleotide sequence, wherein said target nucleotide sequence has a sequence of any of SEQ ID NO:57, 59, 61, 63, 65, and 67-74, and wherein said recombinant polynucleotide molecule disrupts the activity of said polypeptide when provided in the diet of an invertebrate pest. In some embodiments, such disruption may include the reduction or removal of a protein or nucleotide sequence agent that is essential for the pests' growth and development or other biological function. The method is effective in disrupting and/or inhibiting the expression of at least one target gene and can be used to disrupt and/or inhibit many different types of target genes in the pest.

The present disclosure also provides different forms of the pest control agents to achieve the desired reduction in pest infestation. In one form, the pest control agents comprise dsRNA molecules. In another form, the pest control agents comprise siRNA molecules. In still another form, the pest control agents comprise gRNA molecules alone or in combination with a site-specific nuclease (e.g. a Cas nuclease, a Cpf1 nuclease, or a variant of either thereof). In still another form, the pest control agents comprise recombinant DNA constructs that can be used to stably transform microorganisms or plants, enabling the transformed microbes or plants to encode the dsRNA, siRNA, gRNA molecules. In another form, the pest control agents contain the recombinant DNA constructs encoding the dsRNA, siRNA, gRNA molecules.

The present disclosure provides recombinant DNA constructs for use in achieving stable transformation of particular host pest targets. Transformed host pest targets express pesticidally effective levels of preferred dsRNA, siRNA, gRNA molecules (alone or in combination with a site-specific nuclease) from the recombinant DNA constructs, and provide the molecules in the diet of the pest.

The present disclosure also provides, as an example of a transformed host pest target organism, transformed plant cells and transformed plants and their progeny. The transformed plant cells and transformed plants express one or more of the dsRNA or siRNA sequences, or a fragment of any of the disclosed sequences, of the present disclosure from one or more of the DNA sequences as set forth in, SEQ ID NO:2, 4, 6, 8, 10, 12, or 14 as set forth in the sequence listing, or the complement thereof.

In particular embodiments, the present disclosure provides methods of using the DNA molecule provided herein to control pests, including Whitefly (e.g., B. tabaci), a wide range of crops, including vegetables, fruits, and ornamental plants.

In certain examples DNA molecules and methods for controlling pests in tomato crops are provided, which reduce yield loss in tomato crops, as well as reduce transmission of viruses which can reduce fruit quality and yield.

In other examples DNA molecules and methods for controlling pests in field crops (e.g., cotton, soybean, tobacco) crops are provided, which reduce yield loss and increase quality in field crops. Whitefly also secrete honeydew, which can promote the growth of sooty mold that further damages cotton and other crop plants.

Whitefly can also cause significant damage to citrus crops, reducing yields and causing fruit drop. They can also transmit viruses that can affect the quality of the fruit. The DNA molecules and methods of the present disclosure can reduce or eliminate this loss in fruit quality or yield.

In some examples DNA molecules and methods for controlling pests in cucurbit crops are provided, which reduce yield loss and increase quality in cucurbit crops. Whiteflies (e.g., B. tabaci) can cause significant yield losses in cucurbit crops such as cucumbers, melons, and squash, as well as transmit viruses that can reduce fruit quality.

In other examples, DNA molecules and methods for controlling pests in vegetable crops (e.g., tomatoes, peppers, eggplants, cucumbers, squash, beans, sweet potatoes, cassava) are provided, which reduce yield loss and increase quality in vegetable crops. Whiteflies can cause significant damage to vegetable crops, reducing yields and quality. They can also transmit viruses that can affect the quality of the vegetables.

In other examples DNA molecules and methods for controlling pests in potato crops are provided, which reduce yield loss and increase quality in potato crops. Whitefly can cause significant damage to potato crops, reducing yields and quality. They can also transmit viruses that can affect the quality of the tubers.

In further examples DNA molecules and methods for controlling pests in ornamental plants are provided, which prevent loss of aesthetic value and marketability due to pests. Whitefly can cause significant damage to ornamental plants such as poinsettias, hibiscus, and petunias.

Overall, whiteflies can cause significant economic losses in a wide range of crops, and effective management strategies are essential to reduce their impact on agricultural production.

### I. Gene Suppression

As used herein the words “gene suppression”, when taken together, are intended to refer to any of the well-known methods for reducing the levels of protein produced as a result of gene transcription to mRNA and subsequent translation of the mRNA. Gene suppression is also intended to mean the reduction of protein expression from a gene or a coding sequence including posttranscriptional gene suppression and transcriptional suppression. Posttranscriptional gene suppression is mediated by the homology between all or a part of a mRNA transcribed from a gene or coding sequence targeted for suppression and the corresponding double stranded RNA used for suppression, and refers to the substantial and measurable reduction of the amount of available mRNA available in the cell for binding by ribosomes. The transcribed RNA can be in the sense orientation to effect what is called co-suppression, in the anti-sense orientation to effect what is called anti-sense suppression, or in both orientations producing a dsRNA to effect what is called RNA interference (RNAi). Transcriptional suppression is mediated by the presence in the cell of a dsRNA, a gene suppression agent, exhibiting substantial sequence identity to a promoter DNA sequence or the complement thereof to effect what is referred to as promoter trans suppression. Gene suppression may be effective against a native plant gene associated with a trait, e.g., to provide plants with reduced levels of a protein encoded by the native gene or with enhanced or reduced levels of an affected metabolite. Gene suppression can also be effective against target genes in plant pests that may ingest or contact plant material containing gene suppression agents, specifically designed to inhibit or suppress the expression of one or more homologous or complementary sequences in the cells of the pest.

Post-transcriptional gene suppression by anti-sense or sense oriented RNA to regulate gene expression in plant cells is disclosed in U.S. Pat. Nos. 5,107,065, 5,759,829, 5,283,184, and 5,231,020. The use of dsRNA to suppress genes in plants is disclosed in WO 99/53050, WO 99/49029, U.S. Patent Application Publication No. 2003/0175965, and 2003/0061626, U.S. patent application Ser. No. 10/465,800, and U.S. Pat. Nos. 6,506,559, and 6,326,193.

A preferred method of post transcriptional gene suppression in plants employs both sense-oriented and anti-sense-oriented, transcribed RNA which is stabilized, e.g., as a hairpin and stem and loop structure. A preferred DNA construct for effecting post transcriptional gene suppression one in which a first segment encodes an RNA exhibiting an anti-sense orientation exhibiting substantial identity to a segment of a gene targeted for suppression, which is linked to a second segment encoding an RNA exhibiting substantial complementarity to the first segment. Such a construct would be expected to form a stem and loop structure by hybridization of the first segment with the second segment and a loop structure from the nucleotide sequences linking the two segments (see WO94/01550, WO98/05770, US 2002/0048814, and US 2003/0018993).

As used herein, the term “nucleic acid” refers to a single or double-stranded polymer of deoxyribonucleotide or ribonucleotide bases read from the 5′ to the 3′ end. The “nucleic acid” may also optionally contain non-naturally occurring or altered nucleotide bases that permit correct read through by a polymerase and do not reduce expression of a polypeptide encoded by that nucleic acid. The term “nucleotide sequence” or “nucleic acid sequence” refers to both the sense and antisense strands of a nucleic acid as either individual single strands or in the duplex. The term “ribonucleic acid” (RNA) is inclusive of RNAi (inhibitory RNA), dsRNA (double stranded RNA), siRNA (small interfering RNA), mRNA (messenger RNA), miRNA (micro-RNA), tRNA (transfer RNA, guide RNA (gRNA), whether charged or discharged with a corresponding acylated amino acid), and cRNA (complementary RNA) and the term “deoxyribonucleic acid” (DNA) is inclusive of cDNA and genomic DNA and DNA-RNA hybrids. The words “nucleic acid segment”, “nucleotide sequence segment”, or more generally “segment” will be understood by those in the art as a functional term that includes both genomic sequences, ribosomal RNA sequences, transfer RNA sequences, messenger RNA sequences, operon sequences and smaller engineered nucleotide sequences that express or may be adapted to express, proteins, polypeptides or peptides.

As used herein, the term “pest” refers to insects, arachnids, crustaceans, fungi, bacteria, viruses, nematodes, flatworms, roundworms, pinworms, hookworms, tapeworms, trypanosomes, schistosomes, botflies, fleas, ticks, mites, and lice and the like that are pervasive in the human environment and that may ingest or contact one or more cells, tissues, or fluids produced by a pest host transformed to express or coated with a double stranded gene suppression agent or that may ingest plant material containing the gene suppression agent. As used herein, a “pest resistance” trait is a characteristic of a transgenic plant, transgenic host that causes the plant host to be resistant to attack from a pest that typically is capable of inflicting damage or loss to the plant host. Such pest resistance can arise from a natural mutation or more typically from incorporation of recombinant DNA that confers pest resistance. To impart insect resistance to a transgenic plant a recombinant DNA can be transcribed into a RNA molecule that forms a dsRNA molecule within the tissues or fluids of the recombinant plant. The dsRNA molecule is comprised in part of a segment of RNA that is identical to a corresponding RNA segment encoded from a DNA sequence within an insect pest that prefers to feed on the recombinant plant. Expression of the gene within the target insect pest, e.g. within the gut or bacteriocyte of said pest, is suppressed by the dsRNA, and the suppression of expression of the gene in the target insect pest results in the plant being insect resistant. Fire, et al. (U.S. Pat. No. 6,506,599) generically described inhibition of pest infestation, providing specifics only about several nucleotide sequences that were effective for inhibition of gene function in the nematode species Caenorhabditis elegans. Similarly, Plaetinck, et al. (US 2003/0061626) describe the use of dsRNA for inhibiting gene function in a variety of nematode pests. Mesa, et al. (US 2003/0150017) describe using dsDNA sequences to transform host cells to express corresponding dsRNA sequences that are substantially identical to target sequences in specific pathogens, and particularly describe constructing recombinant plants expressing such dsRNA sequences for ingestion by various plant pests, facilitating down-regulation of a gene in the genome of the pest and improving the resistance of the plant to the pest infestation.

The present disclosure provides for inhibiting gene expression of one or multiple target genes in a target insect pest using stabilized dsRNA methods. The disclosure is particularly useful in the modulation of gene expression in the gut and bacteriocytes of insect pests such as cassava whitefly. The modulatory effect is applicable to a variety of genes expressed in the pests including, for example, endogenous genes responsible for cellular metabolism or cellular transformation, including housekeeping genes, transcription factors and other genes which encode polypeptides involved in cellular metabolism. The modulatory effect is also applicable to cassava whitefly's intracellular symbiont, Portiera, which is restricted to the cytoplasm of insect cells known as bacteriocytes; and provides several essential amino-acids or metabolites for intermediate reactions within different essential amino-acid biosynthesis pathways in the insect pest.

As used herein, the term “expression” refers to the transcription and stable accumulation of sense or antisense RNA derived from the nucleic acids disclosed in the present disclosure. Expression may also refer to translation of mRNA into a polypeptide or protein. As used herein, the term “sense” RNA refers to an RNA transcript corresponding to a sequence or segment that, when produced by the target pest, is in the form of a mRNA that is capable of being translated into protein by the target pest cell. As used herein, the term “antisense RNA” refers to an RNA transcript that is complementary to all or a part of a mRNA that is normally produced in a cell of a target pest. The complementarity of an antisense RNA may be with any part of the specific gene transcript, i.e., at the 5′ non-coding sequence, 3′ non-translated sequence, introns, or the coding sequence. As used herein, the term “RNA transcript” refers to the product resulting from RNA polymerase-catalyzed transcription of a DNA sequence. When the RNA transcript is a perfect complementary copy of the DNA sequence, it is referred to as the primary transcript or it may be an RNA sequence derived from post-transcriptional processing of the primary transcript and is referred to as the mature RNA.

As used herein, the phrase “inhibition of gene expression” or “inhibiting expression of a target gene in the cell of an insect” refers to the absence (or observable decrease) in the level of protein and/or mRNA product from the target gene. Specificity refers to the ability to inhibit the target gene without manifest effects on other genes of the cell and without any effects on any gene within the cell that is producing the dsRNA molecule. The inhibition of gene expression of the target gene in the insect pest may result in novel phenotypic traits in the insect pest.

Without limiting the scope of the present invention, there is provided, in one aspect, a method for controlling infestation of a target insect using the stabilized dsRNA strategies. The method involves generating stabilized dsRNA molecules as one type of the insect control agents to induce gene silencing in an insect pest. The insect control agents of the present disclosure induce directly or indirectly post-transcriptional gene silencing events of target genes in the insect.

Down-regulation of expression of the target gene prevents or at least retards the insect's growth, development, reproduction and infectivity to hosts. As used herein, the phrase “generating stabilized dsRNA molecule” refers to the methods of employing recombinant DNA technologies readily available in the art (e.g., by Sambrook, et al., In: Molecular Cloning, A Laboratory Manual, 2nd Edition, Cold Spring Harbor Press, Cold Spring Harbor, New York, 1989) to construct a DNA nucleotide sequence that transcript the stabilized dsRNA. The detailed construction methods of the present disclosure are disclosed below in this disclosure. As used herein, the term “silencing” refers the effective “down-regulation” of expression of the targeted nucleotide sequence and, hence, the elimination of the ability of the sequence to cause an effect within the insect's cell.

The present disclosure provides in part a delivery system for the delivery of the insect control agents to insects through their exposure to a diet containing the insect control agents of the present disclosure. In accordance with one of the embodiments, the stabilized dsRNA or siRNA molecules may be incorporated in the insect diet or may be overlaid on the top of the diet for consumption by an insect.

The present disclosure also provides systems for the delivery of the insect control agents to insects through their exposure to a microorganism or a host such as a plant containing the insect control agents of the present disclosure by ingestion of the microorganism or the host cells or the contents of the cells. In accordance with other embodiments, the present disclosure involves generating a transgenic plant cell or a plant that contains a recombinant DNA construct transcribing the stabilized dsRNA molecules of the present disclosure. As used herein, the phrase “generating a transgenic plant cell or a plant” refers to the methods of employing the recombinant DNA technologies readily available in the art (e.g., by Sambrook, et al.) to construct a plant transformation vector transcribing the stabilized dsRNA molecules of the present disclosure, to transform the plant cell or the plant and to generate the transgenic plant cell or the transgenic plant that contain the transcribed, stabilized dsRNA molecules. In particular, the method of the present disclosure may comprise the recombinant construct in a cell of a plant that results in dsRNA transcripts that are substantially homologous to an RNA sequence encoded by a nucleotide sequence within the genome of an insect. Where the nucleotide sequence within the genome of an insect encodes a gene essential to the viability and infectivity of the insect, its down-regulation results in a reduced capability of the insect to survive and infect host cells. Hence, such down-regulation results in a “deleterious effect” on the maintenance viability and infectivity of the insect, in that it prevents or reduces the insect's ability to feed off and survive on nutrients derived from the host cells. By virtue of this reduction in the insect's viability and infectivity, resistance and/or enhanced tolerance to infection by an insect is facilitated in the cells of a plant. Genes in the insect may be targeted at the mature (adult), immature (larval), or egg stages.

In still another embodiment, non-pathogenic, attenuated strains of microorganisms may be used as a carrier for the insect control agents and, in this perspective, the microorganisms carrying such agents are also referred to as insect control agents. The microorganisms may be engineered to express a nucleotide sequence of a target gene to produce RNA molecules comprising RNA sequences homologous or complementary to RNA sequences typically found within the cells of an insect. Exposure of the insects to the microorganisms result in ingestion of the microorganisms and down-regulation of expression of target genes mediated directly or indirectly by the RNA molecules or fragments or derivatives thereof.

The present disclosure alternatively provides exposure of an insect to the insect control agents of the present disclosure incorporated in a spray mixer and applied to the surface of a host, such as a host plant. In an exemplary embodiment, ingestion of the insect control agents by an insect delivers the insect control agents to the gut of the insect and subsequently to the cells within the body of the insect. In another embodiment, infection of the insect by the insect control agents through other means such as by injection or other physical methods also permits delivery of the insect control agents. In yet another embodiment, the RNA molecules themselves are encapsulated in a synthetic matrix such as a polymer and applied to the surface of a host such as a plant. Ingestion of the host cells by an insect permits delivery of the insect control agents to the insect and results in down-regulation of a target gene in the host.

It is envisioned that the compositions of the present disclosure can be incorporated within the seeds of a plant species either as a product of expression from a recombinant gene incorporated into a genome of the plant cells, or incorporated into a coating or seed treatment that is applied to the seed before planting. The plant cell containing a recombinant gene is considered herein to be a transgenic event.

It is believed that a pesticidal seed treatment can provide significant advantages when combined with a transgenic event that provides protection from invertebrate pest infestation that is within the preferred effectiveness range against a target pest. In addition, it is believed that there are situations that are well known to those having skill in the art, where it is advantageous to have such transgenic events within the preferred range of effectiveness.

The present disclosure also includes seeds and plants having more than one transgenic event. Such combinations are referred to as “stacked” transgenic events. These stacked transgenic events can be events that are directed at the same target pest, or they can be directed to different target pests.

It is believed that the combination of a transgenic seed exhibiting bioactivity against a target pest as a result of the production of an insecticidal amount of an insecticidal dsRNA within the cells of the transgenic seed or plant grown from the seed coupled with treatment of the seed with certain chemical or protein pesticides, including insecticides, provides synergistic advantages to seeds having such treatment, including unexpectedly superior efficacy for protection against damage to the resulting transgenic plant by the target pest. The seeds of the present disclosure are also believed to have the property of decreasing the cost of pesticide use, because less of the pesticide can be used to obtain a required amount of protection than if the innovative composition and method is not used. Moreover, because less pesticide is used and because it is applied prior to planting and without a separate field application, it is believed that the subject method is therefore safer to the operator and to the environment, and is potentially less expensive than conventional methods.

When it is said that some effects are “synergistic”, it is meant to include the synergistic effects of the combination on the pesticidal activity (or efficacy) of the combination of the transgenic event and the pesticide. However, it is not intended that such synergistic effects be limited to the pesticidal activity, but that they should also include such unexpected advantages as increased scope of activity, advantageous activity profile as related to type and amount of damage reduction, decreased cost of pesticide and application, decreased pesticide distribution in the environment, decreased pesticide exposure of personnel who produce, handle and plant corn seeds, and other advantages known to those skilled in the art.

Pesticides and insecticides that are useful in compositions in combination with the methods and compositions of the present disclosure, including as seed treatments and coatings as well as methods for using such compositions can be found, for example, in U.S. Pat. No. 6,551,962, the entirety of which is incorporated herein by reference.

The subject pesticides can be applied to a seed as a component of a seed coating. Seed coating methods and compositions that are known in the art are useful when they are modified by the addition of one of the embodiments of the combination of pesticides of the present disclosure. Such coating methods and apparatus for their application are disclosed in, for example, U.S. Pat. Nos. 5,918,413, 5,891,246, 5,554,445, 5,389,399, 5,107,787, 5,080,925, 4,759,945 and 4,465,017. Seed coating compositions are disclosed, for example, in U.S. Pat. Nos. 5,939,356, 5,882,713, 5,876,739, 5,849,320, 5,834,447, 5,791,084, 5,661,103, 5,622,003, 5,580,544, 5,328,942, 5,300,127, 4,735,015, 4,634,587, 4,383,391, 4,372,080, 4,339,456, 4,272,417 and 4,245,432, among others.

As used herein, the term “insect control agent”, or “gene suppression agent” refers to a particular RNA molecule consisting of a first RNA segment and a second RNA segment linked by a third RNA segment. The first and the second RNA segments lie within the length of the RNA molecule and are substantially inverted repeats of each other and are linked together by the third RNA segment. The complementarity between the first and the second RNA segments results in the ability of the two segments to hybridize in vivo and in vitro to form a double stranded molecule, i.e., a stem, linked together at one end of each of the first and second segments by the third segment which forms a loop, so that the entire structure forms into a stem and loop structure, or even more tightly hybridizing structures may form into a stem-loop knotted structure. The first and the second segments correspond invariably and not respectively to a sense and an antisense sequence with respect to the target RNA transcribed from the target gene in the target insect pest that is suppressed by the ingestion of the dsRNA molecule. The insect control agent can also be a substantially purified (or isolated) nucleic acid molecule and more specifically nucleic acid molecules or nucleic acid fragment molecules thereof from a genomic DNA (gDNA) or cDNA library. Alternatively, the fragments may comprise smaller oligonucleotides having from about 15 to about 250 nucleotide residues, and more preferably, about 15 to about 30 nucleotide residues. The “insect control agent” may also refer to a DNA construct that comprises the isolated and purified nucleic acid molecules or nucleic acid fragment molecules thereof from a gDNA or cDNA library. The “insect control agent” may further refer to a microorganism comprising such a DNA construct that comprises the isolated and purified nucleic acid molecules or nucleic acid fragment molecules thereof from a gDNA or cDNA library. As used herein, the phrase “generating an insect control agent” refers to the methods of employing the recombinant DNA technologies readily available in the art (e.g., by Sambrook, et al.) to prepare a recombinant DNA construct transcribing the stabilized dsRNA or siRNA molecules, to construct a vector transcribing the stabilized dsRNA or siRNA molecules, and/or to transform and generate the cells or the microorganisms that contain the transcribed, stabilized dsRNA or siRNA molecules. The methods of the present disclosure provide for the production of a dsRNA transcript, the nucleotide sequence of which is substantially homologous to a targeted RNA sequence encoded by a target nucleotide sequence within the genome of a target insect pest.

As used herein, the term “genome” as it applies to cells of an insect or a host encompasses not only chromosomal DNA found within the nucleus, but organelle DNA found within subcellular components of the cell. The DNA of the present disclosure introduced into plant cells can therefore be either chromosomally integrated or organelle-localized. The term “genome” as it applies to bacteria encompasses both the chromosome and plasmids within a bacterial host cell. The DNA's of the present disclosure introduced into bacterial host cells can therefore be either chromosomally integrated or plasmid-localized.

Inhibition of target gene expression may be quantified by measuring either the endogenous target RNA or the protein produced by translation of the target RNA and the consequences of inhibition can be confirmed by examination of the outward properties of the cell or organism. Techniques for quantifying RNA and proteins are well known to one of ordinary skill in the art. Multiple selectable markers are available that confer resistance to ampicillin, bleomycin, chloramphenicol, gentamycin, hygromycin, kanamycin, lincomycin, methotrexate, phosphinothricin, puromycin, spectinomycin, rifampicin, and tetracyclin, and the like.

In certain preferred embodiments gene expression is inhibited by at least 10%, preferably by at least 33%, more preferably by at least 50%, and yet more preferably by at least 80%. In particularly preferred embodiments of the disclosure gene expression is inhibited by at least 80%, more preferably by at least 90%, more preferably by at least 95%, or by at least 99% within cells in the insect so a significant inhibition takes place. Significant inhibition is intended to refer to sufficient inhibition that results in a detectable phenotype (e.g., cessation of larval growth, paralysis or mortality, etc.) or a detectable decrease in RNA and/or protein corresponding to the target gene being inhibited. Although in certain embodiments of the disclosure inhibition occurs in substantially all cells of the insect, in other preferred embodiments inhibition occurs in only a subset of cells expressing the gene. For example, if the gene to be inhibited plays an essential role in cells in the insect alimentary tract, inhibition of the gene within these cells is sufficient to exert a deleterious effect on the insect.

The advantages of the present disclosure may include, but are not limited to, the following: the case of introducing dsRNA into the insect cells, the low concentration of dsRNA or siRNA which can be used, the stability of dsRNA or siRNA, and the effectiveness of the inhibition. The ability to use a low concentration of a stabilized dsRNA avoids several disadvantages of anti-sense interference. The present disclosure is not limited to in vitro use or to specific sequence compositions, to a particular set of target genes, a particular portion of the target gene's nucleotide sequence, or a particular transgene or to a particular delivery method, as opposed to the some of the available techniques known in the art, such as antisense and co-suppression. Furthermore, genetic manipulation becomes possible in organisms that are not classical genetic models.

In practicing the present disclosure, it is important that the presence of the nucleotide sequences that are transcribed from the recombinant construct are neither harmful to cells of the plant in which they are expressed in accordance with the disclosure, nor harmful to an animal food chain and in particular humans. Because the produce of the plant may be made available for human ingestion, the down-regulation of expression of the target nucleotide sequence occurs only in the insect.

Therefore, in order to achieve inhibition of a target gene selectively within an insect species that it is desired to control, the target gene should preferably exhibit a low degree of sequence identity with corresponding genes in a plant or a vertebrate animal. Preferably the degree of the sequence identity is less than approximately 80%. More preferably the degree of the sequence identity is less than approximately 70%. Most preferably the degree of the sequence identity is less than approximately 60%.

According to one embodiment of the present disclosure, there is provided a nucleotide sequence, for which in vitro expression results in transcription of a stabilized RNA sequence that is substantially homologous to an RNA molecule of a targeted gene in an insect that comprises an RNA sequence encoded by a nucleotide sequence within the genome of the insect. Thus, after the insect ingests the stabilized RNA sequence incorporated in a diet or sprayed on a plant surface, a down-regulation of the nucleotide sequence corresponding to the target gene in the cells of a target insect is affected. The down-regulated nucleotide sequence in the insect results in a deleterious effect on the maintenance, viability, proliferation, reproduction and infectivity of the insect. Therefore, the nucleotide sequence of the present disclosure may be useful in modulating or controlling infestation by a range of insects.

According to another embodiment of the present disclosure, there is provided a nucleotide sequence, the expression of which in a microbial cell results in a transcription of an RNA sequence which is substantially homologous to an RNA molecule of a targeted gene in an insect that comprises an RNA sequence encoded by a nucleotide sequence within the genome of the insect. Thus, after the insect ingests the stabilized RNA sequence contained in the cell of the microorganism, it will affect down-regulation of the nucleotide sequence of the target gene in the cells of the insect. The down-regulated nucleotide sequence in the insect results in a deleterious effect on the maintenance, viability, proliferation, reproduction and infestation of the insect. Therefore, the nucleotide sequence of the present disclosure may be useful in modulating or controlling infestation by a range of insects.

According to yet another embodiment of the present disclosure, there is provided a nucleotide sequence, the expression of which in a plant cell results in a transcription of an RNA sequence which is substantially homologous to an RNA molecule of a targeted gene in an insect that comprises an RNA sequence encoded by a nucleotide sequence within the genome of the insect. Thus, after the insect ingests the stabilized RNA sequence contained in the cell of the plant, it will affect down-regulation of the nucleotide sequence of the target gene in the cells of the insect. The down-regulated nucleotide sequence in the insect results in a deleterious effect on the maintenance, viability, proliferation, reproduction and infestation of the insect. Therefore, the nucleotide sequence of the present disclosure may be useful in modulating or controlling infestation by a range of insects in plants.

As used herein, the term “substantially homologous” or “substantial homology”, with reference to a nucleic acid sequence, refers to a nucleotide sequence that hybridizes under stringent conditions to a nucleotide sequence encoding a polypeptide having a sequence selected from the group consisting of SEQ ID NO:1, SEQ ID NO:3, SEQ ID NO:5, SEQ ID NO:7, SEQ ID NO:9, SEQ ID NO:11, and SEQ ID NO:13, and wherein said recombinant polynucleotide molecule disrupts the activity of said polypeptide when provided in the diet of a an invertebrate pest. Sequences that hybridize under stringent conditions to a nucleotide sequence encoding a polypeptide having a sequence as set forth in the sequence listing, or the complements thereof, are those that allow an antiparallel alignment to take place between the two sequences, and the two sequences are then able, under stringent conditions, to form hydrogen bonds with corresponding bases on the opposite strand to form a duplex molecule that is sufficiently stable under the stringent conditions to be detectable using methods well known in the art. Such substantially homologous sequences have preferably from about 65% to about 70% sequence identity, or more preferably from about 80% to about 85% sequence identity, or most preferable from about 90% to about 95% sequence identity, to about 99% sequence identity, to the referent nucleotide sequences, or the complements thereof.

As used herein, the term “sequence identity”, “sequence similarity” or “homology” is used to describe sequence relationships between two or more nucleotide sequences. The percentage of “sequence identity” between two sequences is determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. A sequence that is identical at every position in comparison to a reference sequence is said to be identical to the reference sequence and vice-versa. A first nucleotide sequence when observed in the 5′ to 3′ direction is said to be a “complement” of, or complementary to, a second or reference nucleotide sequence observed in the 3′ to 5′ direction if the first nucleotide sequence exhibits complete complementarity with the second or reference sequence. As used herein, nucleic acid sequence molecules are said to exhibit “complete complementarity” when every nucleotide of one of the sequences read 5′ to 3′ is complementary to every nucleotide of the other sequence when read 3′ to 5′. A nucleotide sequence that is complementary to a reference nucleotide sequence will exhibit a sequence identical to the reverse complement sequence of the reference nucleotide sequence. These terms and descriptions are well defined in the art and are easily understood by those of ordinary skill in the art.

As used herein, a “comparison window” refers to a conceptual segment of at least 6 contiguous positions, usually about 50 to about 100, more usually about 100 to about 150, in which a sequence is compared to a reference sequence of the same number of contiguous positions after the two sequences are optimally aligned. The comparison window may comprise additions or deletions (i.e. gaps) of about 20% or less as compared to the reference sequence (which does not comprise additions or deletions) for optimal alignment of the two sequences Those skilled in the art should refer to the detailed methods used for sequence alignment in the Wisconsin Genetics Software Package Release 7.0, Genetics Computer Group, 575 Science Drive Madison, Wis., USA) or refer to Ausubel, et al. (1998) for a detailed discussion of sequence analysis.

The target gene of the present disclosure is derived from an insect cell, e.g. a bacteriocyte, or alternatively, a foreign gene such as a foreign genetic sequence from a endosymbiont, a virus, a fungus, an insect or a nematode, among others. By “derived” it is intended that a sequence is all or a part of the naturally occurring nucleotide sequence of the target gene from the genome of an insect cell, particularly all or a part of the naturally occurring nucleotide sequence of the capped, spliced, and polyadenylated mRNA expressed from the naturally occurring DNA sequence as found in the cell if the gene is a structural gene, or the sequence of all or a part of an RNA that is other than a structural gene including but not limited to a tRNA, a catalytic RNA, a ribosomal RNA, a micro-RNA, and the like. A sequence is derived from one of these naturally occurring RNA sequences if the derived sequence is produced based on the nucleotide sequence of the native RNA, exhibits from about 80% to about 100% sequence identity to the native sequence, and hybridizes to the native sequence under stringent hybridization conditions. In one embodiment, the target gene comprises a nucleotide sequence as set forth in any of SEQ ID NO:2, 4, 6, 8, 10, 12, or 14 as set forth in the sequence listing, or fragments or complements thereof. Depending on the particular target gene and the dose of dsRNA molecules delivered, this process may provide partial or complete loss of function for the target gene, or any desired level of suppression in between.

The present disclosure also provides an artificial DNA sequence capable of being expressed in a cell or microorganism and which is capable of inhibiting target gene expression in a cell, tissue or organ of an insect, wherein the artificial DNA sequence at least comprises a dsDNA molecule coding for one or more different nucleotide sequences, wherein each of the different nucleotide sequences comprises a sense nucleotide sequence and an antisense nucleotide sequence connected by a spacer sequence coding for a dsRNA molecule of the present disclosure. The spacer sequence constitutes part of the sense nucleotide sequence or the antisense nucleotide sequence and will form within the dsRNA molecule between the sense and antisense sequences. The sense nucleotide sequence or the antisense nucleotide sequence is substantially identical to the nucleotide sequence of the target gene or a derivative thereof or a complementary sequence thereto. The dsDNA molecule is placed operably under the control of a promoter sequence that functions in the cell, tissue or organ of the host expressing the dsDNA to produce dsRNA molecules.

The disclosure also provides an artificial DNA sequence for expression in a cell of a plant, and that, upon expression of the DNA to RNA and ingestion by a target pest achieves suppression of a target gene in a cell, tissue or organ of an insect pest. The dsRNA at least comprises one or multiple structural gene sequences, wherein each of the structural gene sequences comprises a sense nucleotide sequence and an antisense nucleotide sequence connected by a spacer sequence that forms a loop within the complementary and antisense sequences. The sense nucleotide sequence or the antisense nucleotide sequence is substantially identical to the nucleotide sequence of the target gene, derivative thereof, or sequence complementary thereto. The one or more structural gene sequences is placed operably under the control of one or more promoter sequences, at least one of which is operable in the cell, tissue or organ of a prokaryotic or eukaryotic organism, particularly an insect.

As used herein, the term “non-naturally occurring gene”, “non-naturally occurring coding sequences”, “artificial sequence”, or “synthetic coding sequences” for transcribing the dsRNA or siRNA of the present disclosure or fragments thereof refers to those prepared in a manner involving any sort of genetic isolation or manipulation that results in the preparation of a coding sequence that transcribes a dsRNA or a siRNA of the present disclosure or fragments thereof. This includes isolation of the coding sequence from its naturally occurring state, manipulation of the coding sequence as by (1) nucleotide insertion, deletion, or substitution, (2) segment insertion, deletion, or substitution, (3) chemical synthesis such as phosphoramidite chemistry and the like, site-specific mutagenesis, truncation of the coding sequence or any other manipulative or isolative method.

The non-naturally occurring gene sequence or fragment thereof according to this aspect of the disclosure for cassava whitefly control may be cloned between two tissue specific promoters, such as two phloem specific promoters which are operable in a transgenic plant cell and therein expressed to produce mRNA in the transgenic plant cell that form dsRNA molecules thereto. The dsRNA molecules contained in plant tissues are ingested by an insect so that the intended suppression of the target gene expression is achieved.

The present disclosure also provides a method for obtaining a nucleic acid comprising a nucleotide sequence for producing a dsRNA or siRNA of the present disclosure. In a preferred embodiment, the method of the present disclosure for obtaining the nucleic acid comprising: (a) probing a cDNA or gDNA library with a hybridization probe comprising all or a portion of a nucleotide sequence or a homolog thereof from a targeted insect; (b) identifying a DNA clone that hybridizes with the hybridization probe; (c) isolating the DNA clone identified in step (b); and (d) sequencing the cDNA or gDNA fragment that comprises the clone isolated in step (c) wherein the sequenced nucleic acid molecule transcribes all or a substantial portion of the RNA nucleotide acid sequence or a homolog thereof.

In another preferred embodiment, the method of the present disclosure for obtaining a nucleic acid fragment comprising a nucleotide sequence for producing a substantial portion of a dsRNA or siRNA of the present disclosure comprising: (a) synthesizing a first and a second oligonucleotide primers corresponding to a portion of one of the nucleotide sequences from a targeted insect; and (b) amplifying a cDNA or gDNA insert present in a cloning vector using the first and second oligonucleotide primers of step (a) wherein the amplified nucleic acid molecule transcribes a substantial portion of the a substantial portion of a dsRNA or siRNA of the present disclosure.

In practicing the present disclosure, a target gene may be derived from a whitefly, such as an African cassava whitefly (e.g., B. tabaci SSA1-SG1), or any insect species that damage crop plants and subsequent yield losses or serves as a vector for plant virus transmission. The present inventors contemplate that several criteria may be employed in the selection of preferred target genes. The gene is one whose protein product has a rapid turnover rate, so that dsRNA inhibition will result in a rapid decrease in protein levels. In certain embodiments it is advantageous to select a gene for which a small drop in expression level results in deleterious effects for the insect. If it is desired to target a broad range of insect species a gene is selected that is highly conserved across these species. Conversely, for the purpose of conferring specificity, in certain embodiments of the disclosure, a gene is selected that contains regions that are poorly conserved between individual insect species, or between insects and other organisms. In certain embodiments it may be desirable to select a gene that has no known homologs in other organisms.

As used herein, the term “derived from” refers to a specified nucleotide sequence that may be obtained from a particular specified source or species, albeit not necessarily directly from that specified source or species.

In one embodiment, a gene is selected that is expressed in the insect gut. Targeting genes expressed in the gut avoids the requirement for the dsRNA to spread within the insect. Target genes for use in the present disclosure may include, for example, those that share substantial homologies to the nucleotide sequences of gut-expressed genes that encode protein components that mediate phloem sap sugar transformations or maintain optimum osmotic pressure within the gut. Among the osmoregulation genes, target genes include, but are not limited to, genes within the glucosyl hydrolase family 13.

In another embodiment, a gene is selected that is essentially involved in the growth, development, and reproduction of an insect. For example, a gene expressed by the intracellular symbiont, Portiera that mediate the terminal reaction of three essential amino acids, such as threonine, methionine and tryptophan.

Target genes may also include Portiera genes that contribute to the synthesis of metabolites for intermediate reactions of other essential amino acids. For example, the bulk of reactions of the biosynthesis pathways of seven essential amino acids (arginine, histidine, lysine, phenylalanine, isoleucine, valine and leucine) are mediated by genes in Portiera. Metabolites produced in these reactions are exported across a symbiosome membrane into the bacteriocyte for amino acid biosynthesis. Metabolites (intermediate precursors) exported across the symbiosome membrane include, N(Omega)-(L-Arginino) succinate for arginine biosynthesis, L-Histidinol phosphate for histidine biosynthesis, LL-2,6-Diaminoheptanedioate for lysine biosynthesis, phenylpyruvate for phenylalanine biosynthesis, (5)-3-methyl-2-oxopentanoate for isoleucine biosynthesis, 3-methyl-2-oxobutanoate for valine biosynthesis and 3-carboxy-4-methyl-2-oxopentanoate for leucine biosynthesis.

The present disclosure is not limited to the specific genes described herein but encompasses any gene, the inhibition of which exerts a deleterious effect on an insect pest. In order to obtain a DNA segment from the corresponding gene in an insect species, PCR primers may be designed based on the sequence as found in cassava whitefly or other insects from which the gene has been cloned. The primers are designed to amplify a DNA segment of sufficient length for use in the present disclosure. DNA (either genomic DNA or cDNA) is prepared from the insect species, and the PCR primers are used to amplify the DNA segment. Amplification conditions are selected so that amplification will occur even if the primers do not exactly match the target sequence. Alternately, the gene (or a portion thereof) may be cloned from a gDNA or cDNA library prepared from the insect pest species, using the cassava whitefly gene or another known insect gene as a probe. Techniques for performing PCR and cloning from libraries are known. Further details of the process by which DNA segments from target insect pest species may be isolated based on the sequence of genes previously cloned from cassava whitefly or other insect species are provided in the Examples.

It has been found that the present disclosure is particularly effective when the insect pest is a whitefly such as B. tabaci., and especially when the pest is B. tabaci SSA1-SG1. The present disclosure is also particularly effective for controlling species of insects that pierce and/or suck the fluids from the cells and tissues of plants, including but not limited to phloem-sap feeders. Modifications of the methods disclosed herein are also surprisingly particularly useful in controlling crop pests within the order Hemiptera.

The present disclosure provides stabilized dsRNA or siRNA molecules for control of insect infestations. The dsRNA or siRNA nucleotide sequences comprise double strands of polymerized ribonucleotide and may include modifications to either the phosphate-sugar backbone or the nucleoside. Modifications in RNA structure may be tailored to allow specific genetic inhibition.

In one embodiment, the dsRNA molecules may be modified through an enzymatic process so the siRNA molecules may be generated. The siRNA can efficiently mediate the down-regulation effect for some target genes in some insects. This enzymatic process may be accomplished by utilizing an RNAse III enzyme or a DICER enzyme, present in the cells of an insect, a vertebrate animal, a fungus or a plant in the eukaryotic RNAi pathway (Elbashir et al., 2002, Methods, 26 (2): 199-213; Hamilton and Baulcombe, 1999, Science 286:950-952). This process may also utilize a recombinant DICER or RNAse III introduced into the cells of a target insect through recombinant DNA techniques that are readily known to the skilled in the art. Both the DICER enzyme and RNAse III, being naturally occurring in an insect or being made through recombinant DNA techniques, cleave larger dsRNA strands into smaller oligonucleotides. The DICER enzymes specifically cut the dsRNA molecules into siRNA pieces each of which is about 19-25 nucleotides in length while the RNAse III enzymes normally cleave the dsRNA molecules into 12-15 base-pair siRNA. The siRNA molecules produced by the either of the enzymes have 2 to 3 nucleotide 3′ overhangs, and 5′ phosphate and 3′ hydroxyl termini. The siRNA molecules generated by RNAse III enzyme are the same as those produced by Dicer enzymes in the eukaryotic RNAi pathway and are hence then targeted and degraded by an inherent cellular RNA-degrading mechanism after they are subsequently unwound, separated into single-stranded RNA and hybridize with the RNA sequences transcribed by the target gene. This process results in the effective degradation or removal of the RNA sequence encoded by the nucleotide sequence of the target gene in the insect. The outcome is the silencing of a particularly targeted nucleotide sequence within the insect. Detailed descriptions of enzymatic processes can be found in Hannon (2002, Nature, 418:244-251).

Inhibition of a target gene using the stabilized dsRNA technology of the present disclosure is sequence-specific in that nucleotide sequences corresponding to the duplex region of the RNA are targeted for genetic inhibition. RNA containing a nucleotide sequences identical to a portion of the target gene is preferred for inhibition. RNA sequences with insertions, deletions, and single point mutations relative to the target sequence have also been found to be effective for inhibition. In performance of the present disclosure, it is preferred that the inhibitory dsRNA and the portion of the target gene share at least from about 80% sequence identity, or from about 90% sequence identity, or from about 95% sequence identity, or from about 99% sequence identity, or even about 100% sequence identity. Alternatively, the duplex region of the RNA may be defined functionally as a nucleotide sequence that is capable of hybridizing with a portion of the target gene transcript. A less than full length sequence exhibiting a greater homology compensates for a longer less homologous sequence. The length of the identical nucleotide sequences may be at least about 25, 50, 100, 200, 300, 400, 500 or at least about 1000 bases. Normally, a sequence of greater than 20-100 nucleotides should be used, though a sequence of greater than about 200-300 nucleotides would be preferred, and a sequence of greater than about 500-1000 nucleotides would be especially preferred depending on the size of the target gene. The disclosure has the advantage of being able to tolerate sequence variations that might be expected due to genetic mutation, strain polymorphism, or evolutionary divergence. The introduced nucleic acid molecule may not need to be absolute homology, may not need to be full length, relative to either the primary transcription product or fully processed mRNA of the target gene. Therefore, those skilled in the art need to realize that, as disclosed herein, 100% sequence identity between the RNA and the target gene is not required to practice the present disclosure.

The dsRNA molecules may be synthesized either in vivo or in vitro. The dsRNA may be formed by a single self-complementary RNA strand or from two complementary RNA strands. Endogenous RNA polymerase of the cell may mediate transcription in vivo, or cloned RNA polymerase can be used for transcription in vivo or in vitro. Inhibition may be targeted by specific transcription in an organ, tissue, or cell type; stimulation of an environmental condition (e.g., infection, stress, temperature, chemical inducers); and/or engineering transcription at a developmental stage or age. The RNA strands may or may not be polyadenylated; the RNA strands may or may not be capable of being translated into a polypeptide by a cell's translational apparatus.

The RNA, dsRNA, siRNA, or miRNA of the present disclosure may be produced chemically or enzymatically by one skilled in the art through manual or automated reactions or in vivo in another organism. RNA may also be produced by partial or total organic synthesis; any modified ribonucleotide can be introduced by in vitro enzymatic or organic synthesis. The RNA may be synthesized by a cellular RNA polymerase or a bacteriophage RNA polymerase (e.g., T3, T7, SP6). The use and production of an expression construct are known in the art (see, for example, WO 97/32016; U.S. Pat. Nos. 5,593,874, 5,698,425, 5,712,135, 5,789,214, and 5,804,693). If synthesized chemically or by in vitro enzymatic synthesis, the RNA may be purified prior to introduction into the cell. For example, RNA can be purified from a mixture by extraction with a solvent or resin, precipitation, electrophoresis, chromatography, or a combination thereof. Alternatively, the RNA may be used with no or a minimum of purification to avoid losses due to sample processing. The RNA may be dried for storage or dissolved in an aqueous solution. The solution may contain buffers or salts to promote annealing, and/or stabilization of the duplex strands.

For transcription from a transgene in vivo or an expression construct, a regulatory region (e.g., promoter, enhancer, silencer, and polyadenylation) may be used to transcribe the RNA strand (or strands). Therefore, in one embodiment, the nucleotide sequences for use in producing RNA molecules may be operably linked to one or more promoter sequences functional in a microorganism, a fungus or a plant host cell. Ideally, the nucleotide sequences are placed under the control of an endogenous promoter, normally resident in the host genome. The nucleotide sequence of the present disclosure, under the control of an operably linked promoter sequence, may further be flanked by additional sequences that advantageously affect its transcription and/or the stability of a resulting transcript. Such sequences are generally located upstream of the operably linked promoter and/or downstream of the 3′ end of the expression construct and may occur both upstream of the promoter and downstream of the 3′ end of the expression construct, although such an upstream sequence only is also contemplated.

In another embodiment, the nucleotide sequence of the present disclosure may comprise an inverted repeat separated by a “spacer sequence”. The spacer sequence may be a region comprising any sequence of nucleotides that facilitates secondary structure formation between each repeat, where this is required. In one embodiment of the present disclosure, the spacer sequence is part of the sense or antisense coding sequence for mRNA. The spacer sequence may alternatively comprise any combination of nucleotides or homologues thereof that are capable of being linked covalently to a nucleic acid molecule. The spacer sequence may comprise a sequence of nucleotides of at least about 10-100 nucleotides in length, or alternatively at least about 100-200 nucleotides in length, at least about 200-400 nucleotides in length, or at least about 400-500 nucleotides in length.

For the purpose of the present disclosure, the dsRNA or siRNA molecules may be obtained from the cassava whitefly by polymerase chain (PCR) amplification of a target gene sequences derived from a cassava whitefly gDNA or cDNA library or portions thereof. The whitefly pupa may be prepared using methods known to the ordinary skilled in the art and DNA/RNA may be extracted. Pupa with various sizes ranging from 1^{st} instars to fully-grown whitefly may be used for the purpose of the present disclosure for DNA/RNA extraction. Genomic DNA or cDNA libraries generated from whitefly may be used for PCR amplification for production of the dsRNA or siRNA.

The target genes may be then be PCR amplified and sequenced using the methods readily available in the art. One skilled in the art may be able to modify the PCR conditions to ensure optimal PCR product formation. The confirmed PCR product may be used as a template for in vitro transcription to generate sense and antisense RNA with the included minimal promoters.

The nucleic acids from whitefly or other insects that may be used in the present disclosure may also comprise isolated and substantially purified Unigenes and EST nucleic acid molecules or nucleic acid fragment molecules thereof. EST nucleic acid molecules may encode significant portions of, or indeed most of, the polypeptides. Alternatively, the fragments may comprise smaller oligonucleotides having from about 15 to about 250 nucleotide residues, and more preferably, about 15 to about 30 nucleotide residues. Alternatively, the nucleic acid molecules for use in the present disclosure may be from cDNA libraries from whitefly, or from any other invertebrate pest species.

As used herein, the phrase “a substantially purified nucleic acid”, “an artificial sequence”, “an isolated and substantially purified nucleic acid”, or “an isolated and substantially purified nucleotide sequence” refers to a nucleic acid that is no longer accompanied by some of the materials with which it is associated in its natural state or to a nucleic acid the structure of which is not identical to that of any of naturally occurring nucleic acid. Examples of a substantially purified nucleic acid include: (1) DNAs which have the sequence of part of a naturally occurring genomic DNA molecules but are not flanked by two coding sequences that flank that part of the molecule in the genome of the organism in which it naturally occurs; (2) a nucleic acid incorporated into a vector or into the genomic DNA of a prokaryote or eukaryote in a manner such that the resulting molecule is not identical to any naturally occurring vector or genomic DNA; (3) a separate molecule such as a cDNA, a genomic fragment, a fragment produced by polymerase chain reaction (PCR), or a restriction fragment; (4) recombinant DNAs; and (5) synthetic DNAs. A substantially purified nucleic acid may also be comprised of one or more segments of cDNA, genomic DNA or synthetic DNA.

Nucleic acid molecules and fragments thereof from cassava whitefly or other invertebrate pest species may be employed to obtain other nucleic acid molecules from other species for use in the present disclosure to produce desired dsRNA and siRNA molecules. Such nucleic acid molecules include the nucleic acid molecules that encode the complete coding sequence of a protein and promoters and flanking sequences of such molecules. In addition, such nucleic acid molecules include nucleic acid molecules that encode for gene family members. Such molecules can be readily obtained by using the above-described nucleic acid molecules or fragments thereof to screen cDNA or gDNA libraries obtained from the whitefly, such as B. tabaci species complex. Methods for forming such libraries are well known in the art.

Nucleic acid molecules and fragments thereof from whitefly, such as the B. tabaci species complex may also be employed to obtain other nucleic acid molecules such as nucleic acid homologues for use in the present disclosure to produce desired dsRNA and siRNA molecules. Such homologues include the nucleic acid molecules that encode, in whole or in part, protein homologues of other species, plants or other organisms. Such molecules can be readily obtained by using the above-described nucleic acid molecules or fragments thereof to screen EST, cDNA or gDNA libraries. Methods for forming such libraries are well known in the art. Such homologue molecules may differ in their nucleotide sequences disclosed herein, because complete complementarity is not needed for stable hybridization. These nucleic acid molecules also include molecules that, although capable of specifically hybridizing with the nucleic acid molecules may lack complete complementarity. In a particular embodiment, methods for 3′ or 5′ RACE may be used to obtain such sequences (Frohman, M. A. et al., Proc. Natl. Acad. Sci. (U.S.A.) 85:8998-9002 (1988); Ohara, O. et al., Proc. Natl. Acad. Sci. (U.S.A.) 86:5673-5677 (1989)). In general, any of the above described nucleic acid molecules or fragments may be used to generate dsRNAs or siRNAs that are suitable for use in a diet, in a spray-on mixer or in a recombinant DNA construct of the present disclosure.

As used herein, the phrase “coding sequence”, “structural nucleotide sequence” or “structural nucleic acid molecule” refers to a nucleotide sequence that is translated into a polypeptide, usually via mRNA, when placed under the control of appropriate regulatory sequences. The boundaries of the coding sequence are determined by a translation start codon at the 5′-terminus and a translation stop codon at the 3′-terminus. A coding sequence can include, but is not limited to, genomic DNA, cDNA, EST and recombinant nucleotide sequences.

The nucleic acid molecules or fragment of the nucleic acid molecules or other nucleic acid molecules from an invertebrate pest such as a cassava whitefly are capable of specifically hybridizing to other nucleic acid molecules under certain circumstances. As used herein, two nucleic acid molecules are said to be capable of specifically hybridizing to one another if the two molecules are capable of forming an anti-parallel, double-stranded nucleic acid structure. A nucleic acid molecule is said to be the complement of another nucleic acid molecule if they exhibit complete complementarity. Two molecules are said to be “minimally complementary” if they can hybridize to one another with sufficient stability to permit them to remain annealed to one another under at least conventional “low-stringency” conditions. Similarly, the molecules are said to be complementary if they can hybridize to one another with sufficient stability to permit them to remain annealed to one another under conventional “high-stringency” conditions. Conventional stringency conditions are described by Sambrook, et al., and by Haymes, et al. In: Nucleic Acid Hybridization, A Practical Approach, IRL Press, Washington, DC (1985).

Departures from complete complementarity are therefore permissible, as long as such departures do not completely preclude the capacity of the molecules to form a double-stranded structure. Thus, in order for a nucleic acid molecule or a fragment of the nucleic acid molecule to serve as a primer or probe it needs only be sufficiently complementary in sequence to be able to form a stable double-stranded structure under the particular solvent and salt concentrations employed.

Appropriate stringency conditions which promote DNA hybridization are, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

A nucleic acid for use in the present disclosure may specifically hybridize to one or more of nucleic acid molecules from cassava whitefly or complements thereof under moderately stringent conditions, for example at about 2.0×SSC and about 65° C. A nucleic acid for use in the present disclosure will include those nucleic acid molecules that specifically hybridize to one or more of the nucleic acid molecules disclosed in any of SEQ ID NO:2, 4, 6, 8, 10, 12, or 14 as set forth in the sequence listing, or fragments or complements thereof under high stringency conditions. Preferably, a nucleic acid for use in the present disclosure will exhibit at least from about 80%, or at least from about 90%, or at least from about 95%, or at least from about 98% or even about 100% sequence identity with one or more nucleic acid molecules as set forth in SEQ ID NO: 2, 4, 6, 8, 10, 12, or 14, or as disclosed herein; or a nucleic acid for use in the present disclosure will exhibit at from about 80%, or at least from about 90%, or at least from about 95%, or at least from about 98% or even about 100% sequence identity with one or more nucleic acid molecules as set forth in SEQ ID NO:2, 4, 6, 8, 10, 12, or 14 in the sequence listing isolated from the genomic DNA of an insect pest.

Nucleic acids of the present disclosure may also be synthesized, either completely or in part, especially where it is desirable to provide plant-preferred sequences, by methods known in the art. Thus, all or a portion of the nucleic acids of the present disclosure may be synthesized using codons preferred by a selected host. Species-preferred codons may be determined, for example, from the codons used most frequently in the proteins expressed in a particular host species. Other modifications of the nucleotide sequences may result in mutants having slightly altered activity.

The present disclosure provides in part a delivery system for the delivery of insect control agents to insects. The stabilized dsRNA or siRNA molecules of the present disclosure may be directly introduced into the cells of an insect, or introduced into an extracellular cavity, interstitial space, lymph system, digestive system, into the circulation of the insect through oral ingestion or other means that one skilled in the art may employ. Methods for oral introduction may include direct mixing of RNA with food of the insect, as well as engineered approaches in which a species that is used as food is engineered to express the dsRNA or siRNA, then fed to the insect to be affected. In one embodiment, for example, the dsRNA or siRNA molecules may be incorporated into, or overlaid on the top of, the insect's diet. In another embodiment, the RNA may be sprayed onto a plant surface. In still another embodiment, the dsRNA or siRNA may be expressed by microorganisms and the microorganisms may be applied onto a plant surface or introduced into a root, stem by a physical means such as an injection. In still another embodiment, a plant may be genetically engineered to express the dsRNA or siRNA in an amount sufficient to kill the insects known to infect the plant.

Specifically, in practicing the present disclosure in an invertebrate pest, such as a cassava whitefly, the stabilized dsRNA or siRNA may be introduced in the midgut or bacteriocyte inside the insect and achieve the desired inhibition of the targeted genes. The dsRNA or siRNA molecules may be incorporated into a diet or be overlaid on the diet as discussed above and may be ingested by the insects. In any event, the dsRNA's of the present disclosure are provided in the diet of the target pest. The digestive tract of a target pest is defined herein as the location within the pest where food that is ingested by the target pest is exposed to an environment that is favorable for the uptake of the dsRNA molecules of the present disclosure without suffering a pH so extreme that the hydrogen bonding between the double-strands of the dsRNA are caused to dissociate and form single stranded molecules.

Further, for the purpose of controlling insect infestations in plants, delivery of insect control dsRNAs to the surfaces of a plant via a spray-on application affords another means of protecting the plants. In this instance, a bacterium engineered to produce and accumulate dsRNAs may be fermented and the products of the fermentation formulated as a spray-on product compatible with common agricultural practices. The formulations may include the appropriate stickers and wetters required for efficient foliar coverage as well as UV protectants to protect dsRNAs from UV damage. Such additives are commonly used in the bioinsecticide industry and are well known to those skilled in the art. Likewise, formulations for soil application may include granular formulations that serve as a bait for insect pests such as the cassava whitefly.

It is also anticipated that dsRNA's produced by chemical or enzymatic synthesis may be formulated in a manner consistent with common agricultural practices and used as spray-on products for controlling insect infestations. The formulations may include the appropriate stickers and wetters required for efficient foliar coverage as well as UV protectants to protect dsRNAs from UV damage. Such additives are commonly used in the bioinsecticide industry and are well known to those skilled in the art. Such applications could be combined with other spray-on insecticide applications, biologically based or not, to enhance plant protection from insect feeding damage.

The present inventors contemplate that bacterial strains producing insecticidal proteins may be used to produce dsRNAs for insect control purposes. These strains may exhibit improved insect control properties. A variety of different bacterial hosts may be used to produce insect control dsRNAs. Exemplary bacteria may include E. coli, B. thuringiensis, Pseudomonas sp., Photorhabdus sp., Xenorhabdus sp., Serratia entomophila and related Serratia sp., B. sphaericus, B. cereus, B. laterosporus, B. popilliae, Clostridium bifermentans and other Clostridium species, or other spore-forming gram-positive bacteria.

The present disclosure also relates to recombinant DNA constructs for expression in a microorganism. Exogenous nucleic acids from which an RNA of interest is transcribed can be introduced into a microbial host cell, such as a bacterial cell or a fungal cell, using methods known in the art.

The nucleotide sequences of the present disclosure may be introduced into a wide variety of prokaryotic and eukaryotic microorganism hosts to produce the stabilized dsRNA or siRNA molecules. The term “microorganism” includes prokaryotic and eukaryotic microbial species such as bacteria and fungi. Fungi include yeasts and filamentous fungi, among others. Illustrative prokaryotes, both Gram-negative and Gram-positive, include Enterobacteriaceae, such as Escherichia, Erwinia, Shigella, Salmonella, and Proteus; Bacillaceae; Rhizobiceae, such as Rhizobium; Spirillaceae, such as Photobacterium, Zymomonas, Serratia, Aeromonas, Vibrio, Desulfovibrio, Spirillum; Lactobacilluseae; Pseudomonadaceae, such as Pseudomonas and Acetobacter; Azotobacteraceae, Actinomycetales, and Nitrobacteraceae. Among eukaryotes are fungi, such as Phycomycetes and Ascomycetes, which includes yeast, such as Saccharomyces and Schizosaccharomyces; and Basidiomycetes yeast, such as Rhodotorula, Aureobasidium, Sporobolomyces, and the like.

For the purpose of plant protection against insects, a large number of microorganisms known to inhabit the phylloplane (the surface of the plant leaves) and/or the rhizosphere (the soil surrounding plant roots) of a wide variety of important crops may also be desirable host cells for manipulation, propagation, storage, delivery and/or mutagenesis of the disclosed recombinant constructs. These microorganisms include bacteria, algae, and fungi. Of particular interest are microorganisms, such as bacteria, e.g., genera Bacillus (including the species and subspecies B. thuringiensis kurstaki HD-1, B. thuringiensis kurstaki HD-73, B. thuringiensis sotto, B. thuringiensis berliner, B. thuringiensis, B. thuringiensis tolworthi, B. thuringiensis dendrolimus, B. thuringiensis alesti, B. thuringiensis galleriae, B. thuringiensis aizawai, B. thuringiensis subtoxicus, B. thuringiensis entomocidus, B. thuringiensis tenebrionis and B. thuringiensis san diego); Pseudomonas, Erwinia, Serratia, Klebsiella, Zanthomonas, Streptomyces, Rhizobium, Rhodopseudomonas, Methylophilius, Agrobacterium, Acetobacter, Lactobacillus, Arthrobacter, Azotobacter, Leuconostoc, and Alcaligenes; fungi, particularly yeast, e.g., genera Saccharomyces, Cryptococcus, Kluyveromyces, Sporobolomyces, Rhodotorula, and Aureobasidium. Of particular interest are such phytosphere bacterial species as Pseudomonas syringae, Pseudomonas fluorescens, Serratia marcescens, Acetobacter xylinum, Agrobacterium tumefaciens, Rhodobacter sphaeroides, Xanthomonas campestris, Rhizobium melioti, Alcaligenes eutrophus, and Azotobacter vinlandii; and phytosphere yeast species such as Rhodotorula rubra, R. glutinis, R. marina, R. aurantiaca, Cryptococcus albidus, C. diffluens, C. laurentii, Saccharomyces rosei, S. pretoriensis, S. cerevisiae, Sporobolomyces roseus, S. odorus, Kluyveromyces veronae, and Aureobasidium pollulans.

The term “operably linked”, as used in reference to a regulatory sequence and a structural nucleotide sequence, means that the regulatory sequence causes regulated expression of the linked structural nucleotide sequence. “Regulatory sequences” or “control elements” refer to nucleotide sequences located upstream (5′ noncoding sequences), within, or downstream (3′ non-translated sequences) of a structural nucleotide sequence, and which influence the timing and level or amount of transcription, RNA processing or stability, or translation of the associated structural nucleotide sequence. Regulatory sequences may include promoters, translation leader sequences, introns, enhancers, stem-loop structures, repressor binding sequences, and polyadenylation recognition sequences and the like.

The present disclosure also contemplates transformation of a nucleotide sequence of the present disclosure into a plant to achieve pest inhibitory levels of expression of one or more dsRNA molecules. A transformation vector can be readily prepared using methods available in the art. The transformation vector comprises one or more nucleotide sequences that is/are capable of being transcribed to an RNA molecule and that is/are substantially homologous and/or complementary to one or more nucleotide sequences encoded by the genome of the insect, such that upon uptake of the RNA transcribed from the one or more nucleotide sequences molecules by the insect, there is down-regulation of expression of at least one of the respective nucleotide sequences of the genome of the insect.

### II. Genome Editing

The present disclosure provides, in certain embodiments, insect, insect parts, insect cells, and bacteriocytes produced through genome modification using site-specific integration or genome editing. Ingestion by a target pest of compositions containing one or more gRNAs in combination with a site-specific nuclease, resulting in targeted editing at least one gene of interest in the cells of the target pest, results in death, stunting, or other inhibition of the target pest. Genome editing can be used to make one or more edit(s) or mutation(s) at a desired target site in the genome of an insect, including symbiosis genes (amino acid synthesis, transport and horizontally transferred genes), to change expression and/or activity of one or more genes, or to integrate an insertion sequence or transgene at a desired location in a genome. Any site or locus within the genome of an insect may potentially be chosen for making a genomic edit (or gene edit) or site-directed integration of a transgene, construct, or transcribable DNA sequence. As used herein, a “target site” for genome editing or site-directed integration refers to the location of a polynucleotide sequence within an insect genome that is bound and cleaved by a site-specific nuclease to introduce a double-stranded break (DSB) or single-stranded nick into the nucleic acid backbone of the polynucleotide sequence and/or its complementary DNA strand within the genome. A target site may comprise, for example, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 29, or at least 30 consecutive nucleotides. A “target site” for an RNA-guided nuclease may comprise the sequence of either complementary strand of a double-stranded nucleic acid (DNA) molecule or chromosome at the target site. A site-specific nuclease may bind to a target site, such as via a non-coding guide RNA (e.g., without being limiting, a CRISPR RNA (crRNA) or a single-guide RNA (sgRNA) as described further herein). A non-coding guide RNA provided herein may be complementary to a target site (e.g., complementary to either strand of a double-stranded nucleic acid molecule or chromosome at the target site). It will be appreciated that perfect identity or complementarity may not be required for a non-coding guide RNA to bind or hybridize to a target site. For example, at least 1, at least 2, at least 3, at least 4, at least 5, at least 6, at least 7, or at least 8 mismatches (or more) between a target site and a non-coding RNA may be tolerated. A “target site” also refers to the location of a polynucleotide sequence within a genome that is bound and cleaved by any other site-specific nuclease that may not be guided by a non-coding RNA molecule, such as a zinc finger nuclease (ZFN), a transcription activator-like effector nuclease (TALEN), a meganuclease, etc., to introduce a DSB or single-stranded nick into the polynucleotide sequence and/or its complementary DNA strand. As used herein, a “target region” or a “targeted region” refers to a polynucleotide sequence or region that is flanked by two or more target sites. Without being limiting, in some embodiments a target region may be subjected to a mutation, deletion, insertion, substitution, inversion, or duplication. As used herein, “flanked” when used to describe a target region of a polynucleotide sequence or molecule, refers to two or more target sites of the polynucleotide sequence or molecule surrounding the target region, with one target site on each side of the target region.

As used herein, a “targeted genome editing technique” refers to any method, protocol, or technique that allows the precise and/or targeted editing of a specific location in a genome of an insect (i.e., the editing is largely or completely non-random) using a site-specific nuclease, such as a meganuclease, a zinc-finger nuclease (ZFN), an RNA-guided endonuclease (e.g., the CRISPR/Cas9 or Cas12a system), a TALE (transcription activator-like effector)-endonuclease (TALEN), a recombinase, or a transposase. In particular embodiments, a “targeted genome editing technique” refers to an RNA-guided Cas12a system. As used herein, “editing” or “genome editing” refers to generating a targeted mutation, deletion, insertion, substitution, inversion or duplication of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 1000, at least 2500, at least 5000, at least 10,000, or at least 25,000 nucleotides of an endogenous insect genome nucleic acid sequence. As used herein, “editing” or “genome editing” may also encompass the targeted insertion or site-directed integration of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 750, at least 1000, at least 1500, at least 2000, at least 2500, at least 3000, at least 4000, at least 5000, at least 10,000, or at least 25,000 nucleotides into the endogenous genome of an insect. An “edit” or “genomic edit” in the singular refers to one such targeted mutation, deletion, insertion, substitution, inversion, or duplication, whereas “edits” or “genomic edits” refers to two or more targeted mutation(s), deletion(s), insertion(s), substitution(s), inversion(s), and/or duplication(s), with each “edit” being introduced via a targeted genome editing technique.

According to some embodiments, a site-specific nuclease may be co-delivered with a donor template molecule to serve as a template for making a desired edit, mutation or insertion into the genome at the desired target site through repair of the double strand break (DSB) or nick created by the site-specific nuclease. According to some embodiments, a site-specific nuclease may be co-delivered with a DNA molecule comprising a selectable or screenable marker gene.

A site-specific nuclease may be an RNA-guided nuclease. According to some embodiments, an RNA-guided endonuclease may be selected from the group consisting of Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx 17, Csx14, Csx 10, Csx16, CsaX, Csx3, Csx1, Csx15, Csf1, Csf2, Csf3, Csf4, Cpf1, CasX, CasY, and homologs or modified versions of any thereof, as well as Argonaute proteins (non-limiting examples of Argonaute proteins include Thermus thermophilus Argonaute (TtAgo), Pyrococcus furiosus Argonaute (PfAgo), Natronobacterium gregoryi Argonaute (NgAgo), and homologs or modified versions of any thereof). According to some embodiments, an RNA-guided endonuclease is a Cas9 or Cpf1 (also referred to herein as Cas12a) enzyme. The RNA-guided nuclease may be delivered as a protein or a recombinant DNA construct comprising a polynucleotide sequence encoding said nuclease, with or without a guide RNA; or the guide RNA may be complexed with the RNA-guided nuclease enzyme and delivered as a ribonucleoprotein (RNP).

For RNA-guided endonucleases, a guide RNA molecule may be further provided to direct the endonuclease to a target site in the genome of the insect via base-pairing or hybridization to cause a DSB or nick at or near the target site. As described herein, the guide RNA may be transformed or introduced into an insect cell or tissue as a gRNA molecule, or as a recombinant DNA molecule, construct or vector comprising a transcribable DNA sequence encoding one or more guide RNAs operably linked to a single promoter or individual promoters. As understood in the art, a guide RNA may comprise, for example, a CRISPR RNA (crRNA), a single-chain guide RNA (sgRNA), or any other RNA molecule that may guide or direct an endonuclease to a specific target site in the genome. A prototypical CRISPR associated protein, Cas9 from S. pyogenes, naturally binds two RNAs, a CRISPR RNA (crRNA) guide and a trans-acting CRISPR RNA (tracrRNA), to assemble a CRISPR ribonucleoprotein (crRNP). In comparison, the CRISPR-Cas 12a system does not require a trans-activating crispr RNA (tracrRNA) for biogenesis of mature crRNA. Instead, the RuvC endonuclease domain of Cas 12a processes its mature crRNA directly. A “single-chain guide RNA” (or “sgRNA”) is an RNA molecule comprising a crRNA covalently linked a tracrRNA by a linker sequence, which may be expressed as a single RNA transcript or molecule. The guide RNA comprises a guide or targeting sequence (also referred to herein as a “spacer sequence”) that is identical or complementary to a target site within the insect genome, such as at or near a gene. The guide RNA is typically a non-coding RNA molecule that does not encode a protein. The guide sequence of the guide RNA may be at least 10 nucleotides in length, such as 12-40 nucleotides, 12-30 nucleotides, 12-20 nucleotides, 12-35 nucleotides, 12-30 nucleotides, 15-30 nucleotides, 17-30 nucleotides, or 17-25 nucleotides in length, or about 12, 13, 14, 15, 16, 17, 18, 19, 20, 21, 22, 23, 24, 25 or more nucleotides in length. The guide sequence may be at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of a DNA sequence at the genomic target site.

As mentioned above, a target gene for genome editing may be any insect gene of interest. For knockdown mutations of the gene of interest through genome editing, an RNA-guided endonuclease may be targeted to an upstream or downstream sequence, such as a promoter and/or enhancer sequence, or an intron, 5′UTR, and/or 3′UTR sequence of the gene to mutate one or more promoter and/or regulatory sequences of the gene to affect or reduce its level of expression. Similarly, mutations of the gene of interest through genome editing, an RNA-guided endonuclease may be targeted to a transcribable DNA sequence (i.e., a transcribable region) of said gene, such as a region of the gene comprising a coding sequence, a specific DNA sequence encoding a protein domain, an exon region, an intron region, or a combination thereof. For example, in certain embodiments a transcribable DNA sequence targeted for genome editing may comprise an exon/intron boundary or may be in close proximity to an exon/intron boundary. If the resulting modification spans an exon/intron boundary, the modification may be referred to as a modification in an exon region and an intron region. For genetic modification of the gene of interest, a guide RNA may be used, which comprises a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of any one of SEQ ID NO:2, 4, 6, 8, 10, 12, or 14 as set forth in the sequence listing, or a sequence complementary thereto, although alternative splicing and different exon/intron boundaries may occur. As used herein, the term “consecutive” in reference to a polynucleotide or protein sequence means without deletions or gaps in the sequence.

As used herein, with respective to a given sequence, a “complement”, a “complementary sequence” and a “reverse complement” are used interchangeably. All three terms refer to the inversely complementary sequence of a nucleotide sequence, i.e., to a sequence complementary to a given sequence in reverse order of the nucleotides.

Antisense RNA molecules are single-stranded nucleic acids which can combine with a sense RNA strand or sequence or mRNA to form duplexes due to complementarity of the sequences. The term “antisense strand” refers to a nucleic acid strand that is complementary to the “sense” strand. The “sense strand” of a gene or locus is the strand of DNA or RNA that has the same sequence as an RNA molecule transcribed from the gene or locus (with the exception of uracil in RNA and thymine in DNA).

A protospacer-adjacent motif (PAM) may be present in the genome immediately adjacent and upstream to the 5′ end of the genomic target site sequence complementary to the targeting sequence of the guide RNA—i.e., immediately downstream (3′) to the sense (+) strand of the genomic target site (relative to the targeting sequence of the guide RNA) as known in the art. See, e.g., Wu et al. (Quant Biol. 2 (2): 59-70, 2014). The genomic PAM sequence on the sense (+) strand adjacent to the target site (relative to the targeting sequence of the guide RNA) may comprise 5′-NGG-3′ for Cas9; or 5′-TTTN-3′ for Cas12a. However, the corresponding sequence of the guide RNA (i.e., immediately downstream (3′) to the targeting sequence of the guide RNA) may generally not be complementary to the genomic PAM sequence.

As used herein, a “donor molecule”, “donor template”, or “donor template molecule” (collectively a “donor template”), which may be a recombinant polynucleotide, DNA or RNA donor template or sequence, is defined as a nucleic acid molecule having a homologous nucleic acid template or sequence (e.g., homology sequence) and/or an insertion sequence for site-directed, targeted insertion or recombination into the genome of an insect cell via repair of a nick or DSB in the genome of an insect cell. A donor template may be a separate DNA molecule comprising one or more homologous sequence(s) and/or an insertion sequence for targeted integration, or a donor template may be a sequence portion (i.e., a donor template region) of a DNA molecule further comprising one or more other expression cassettes, genes/transgenes, and/or transcribable DNA sequences. For example, a “donor template” may be used for site-directed integration of a transgene or construct, or as a template to introduce a mutation, such as an insertion, deletion, substitution, etc., into a target site within the genome of an insect. A targeted genome editing technique provided herein may comprise the use of one or more, two or more, three or more, four or more, or five or more donor molecules or templates. A donor template provided herein may comprise at least one, at least two, at least three, at least four, at least five, at least six, at least seven, at least eight, at least nine, or at least ten gene(s) or transgene(s) and/or transcribable DNA sequence(s). Alternatively, a donor template may comprise no genes, transgenes or transcribable DNA sequences.

Any method known in the art for site-directed integration may be used with the present disclosure. In the presence of a donor template molecule with an insertion sequence, the DSB or nick can be repaired by homologous recombination between homology arm(s) of the donor template and the genome, or by non-homologous end joining (NHEJ), resulting in site-directed integration of the insertion sequence into the genome to create the targeted insertion event at the site of the DSB or nick. Thus, site-specific insertion or integration of a transgene, transcribable DNA sequence, construct, or sequence may be achieved if the transgene, transcribable DNA sequence, construct or sequence is located in the insertion sequence of the donor template.

The introduction of a DSB or nick may also be used to introduce targeted mutations in the genome of an insect. According to this approach, mutations, such as deletions, insertions, substitutions, inversions, and/or duplications may be introduced at a target site via imperfect repair of the DSB or nick to produce a genetic modification within a gene. Such mutations may be generated by imperfect repair of the targeted locus even without the use of a donor template molecule. A modification of a gene may be achieved by inducing a DSB or nick at or near the endogenous locus of the gene that results in expression of a non-functional protein, interfering protein, or a protein having reduced, disrupted, or altered activity as compared to a protein expressed from the gene lacking said modification.

Similarly, such targeted mutations of a gene may be generated with a donor template molecule to direct a particular or desired mutation at or near the target site via repair of the DSB or nick. The donor template molecule may comprise a homologous sequence with or without an insertion sequence and comprising one or more mutations, such as one or more deletions, insertions, substitutions, inversions, and/or duplications, relative to the targeted genomic sequence at or near the site of the DSB or nick. For example, targeted mutations of a gene may be achieved by deleting, inserting, substituting, inverting, or duplicating at least a portion of the gene, such as by introducing a frame shift or premature stop codon into the coding sequence of the gene or introducing a modification into a transcribable DNA sequence. A deletion of a portion of a gene may also be introduced by generating DSBs or nicks at two target sites and causing a deletion of the intervening target region flanked by the target sites. A modification of a targeted gene may result in expression of a non-functional protein, interfering protein, or a protein having reduced, disrupted, or altered activity as compared to a protein expressed from the gene lacking said modification.

### III. Constructs for Genome Editing

Recombinant DNA constructs and vectors are provided comprising a polynucleotide sequence encoding a site-specific nuclease, such as an RNA-guided endonuclease, wherein the coding sequence is operably linked to a plant expressible promoter. For RNA-guided endonucleases, recombinant DNA constructs and vectors are further provided comprising a polynucleotide sequence encoding one or more guide RNA(s), wherein the guide RNA(s) comprise a guide sequence of sufficient length having a percent identity or complementarity to a target site within the genome of an insect, such as at or near a targeted gene of interest. A polynucleotide sequence of a recombinant DNA construct and vector that encodes a site-specific nuclease or a guide RNA(s) may be operably linked to a plant expressible promoter, such as an inducible promoter, a constitutive promoter, a tissue-specific promoter, etc.

As used herein, a “gene” refers to a nucleic acid sequence forming a genetic and functional unit and coding for one or more sequence-related RNA and/or polypeptide molecules. A gene generally contains a coding region operably linked to appropriate regulatory sequences that regulate the expression of a gene product (e.g., a polypeptide or a functional RNA). A gene can have various sequence elements, including, but not limited to, a promoter, an untranslated region (UTR), exons, introns, and other upstream or downstream regulatory sequences.

As used herein, an “allele” refers to an alternative nucleic acid sequence of a gene or at a particular locus (e.g., a nucleic acid sequence of a gene or locus that is different than other alleles for the same gene or locus). Such an allele can be considered (i) wild-type or (ii) mutant if one or more mutations or edits are present in the nucleic acid sequence of the mutant allele relative to the wild-type allele. A mutant or edited allele for a gene may have reduced, disrupted, altered, or eliminated activity, or a reduced or eliminated expression level for the gene relative to the wild-type allele. For example, a mutant or edited allele for a gene of interest may have a deletion in the transcribable region of the endogenous gene that reduces, disrupts, or alters the activity of the protein encoded by the mutant allele as compared to the activity of the protein encoded by the wild-type allele in an otherwise identical insect. For diploid organisms, e.g., female whiteflies, a first allele can occur on one chromosome, and a second allele can occur at the same locus on a second homologous chromosome. If one allele at a locus on one chromosome of an insect is a mutant or edited allele and the other corresponding allele on the homologous chromosome of the insect is wild-type, then the insect is described as being heterozygous for the mutant or edited allele. However, if both alleles at a locus are mutant or edited alleles, then the insect is described as being homozygous for the mutant or edited alleles. An insect homozygous for mutant or edited alleles at a locus may comprise the same mutant or edited allele or different mutant or edited alleles if heteroallelic or biallelic.

As used herein, a “wild-type gene” or “wild-type allele” refers to a gene or allele having a sequence or genotype that is most common in a particular whitefly species, or another sequence or genotype having only natural variations, polymorphisms, or other silent mutations relative to the most common sequence or genotype that do not significantly impact the expression and activity of the gene or allele. Indeed, a “wild-type” gene or allele contains no variation, polymorphism, or any other type of mutation that substantially affects the normal function, activity, expression, or phenotypic consequence of the gene or allele relative to the most common sequence or genotype.

In general, the term “variant” refers to molecules with some differences, generated synthetically or naturally, in their nucleotide or amino acid sequences as compared to a reference (native) polynucleotides or polypeptides, respectively. These differences include substitutions, insertions, deletions, inversions, duplications, or any desired combinations of such changes in a native polynucleotide or amino acid sequence.

The term “recombinant” in reference to a polynucleotide (DNA or RNA) molecule, protein, construct, vector, etc., refers to a polynucleotide or protein molecule or sequence that is man-made and not normally found in nature, and/or is present in a context in which it is not normally found in nature, including a polynucleotide (DNA or RNA) molecule, protein, construct, etc., comprising a combination of two or more polynucleotide or protein sequences that would not naturally occur together in the same manner without human intervention, such as a polynucleotide molecule, protein, construct, etc., comprising at least two polynucleotide or protein sequences that are operably linked but heterologous with respect to each other. For example, the term “recombinant” can refer to any combination of two or more DNA or protein sequences in the same molecule (e.g., a plasmid, construct, vector, chromosome, protein, etc.) where such a combination is man-made and not normally found in nature. As used in this definition, the phrase “not normally found in nature” means not found in nature without human introduction. A recombinant polynucleotide or protein molecule, construct, etc., can comprise polynucleotide or protein sequence(s) that is/are (i) separated from other polynucleotide or protein sequence(s) that exist in proximity to each other in nature, and/or (ii) adjacent to (or contiguous with) other polynucleotide or protein sequence(s) that are not naturally in proximity with each other. Such a recombinant polynucleotide molecule, protein, construct, etc., can also refer to a polynucleotide or protein molecule or sequence that has been genetically engineered and/or constructed outside of a cell. For example, a recombinant DNA molecule can comprise any engineered or man-made plasmid, vector, etc., and can include a linear or circular DNA molecule. Such plasmids, vectors, etc., can contain various maintenance elements including a prokaryotic origin of replication and selectable marker, as well as one or more transgenes or expression cassettes perhaps in addition to a plant selectable marker gene, etc.

Reference in this application to an “isolated DNA molecule” or an “isolated polynucleotide”, or an equivalent term or phrase, is intended to mean that the DNA molecule or polynucleotide is one that is present alone or in combination with other compositions, but not within its natural environment. For example, nucleic acid elements such as a coding sequence, intron sequence, untranslated leader sequence, promoter sequence, transcriptional termination sequence, and the like, that are naturally found within the DNA of the genome of an organism are not considered to be “isolated” so long as the element is within the genome of the organism and at the location within the genome in which it is naturally found. However, each of these elements, and subparts of these elements, would be “isolated” within the scope of this disclosure so long as the element is not within the genome of the organism and at the location within the genome in which it is naturally found. Similarly, a nucleotide sequence encoding a protein or any naturally occurring variant of that protein would be an isolated nucleotide sequence so long as the nucleotide sequence was not within the DNA of the organism in which the sequence encoding the protein is naturally found. A synthetic nucleotide sequence encoding the amino acid sequence of the naturally occurring protein would be considered to be isolated for the purposes of this disclosure. For the purposes of this disclosure, any transgenic nucleotide sequence, i.e., the nucleotide sequence of the DNA inserted into the genome of the cells of a plant or bacterium, or present in an extrachromosomal vector, would be considered to be an isolated nucleotide sequence whether it is present within the plasmid or similar structure used to transform the cells, within the genome of the plant or bacterium, or present in detectable amounts in tissues, progeny, biological samples or commodity products derived from the plant or bacterium.

As commonly understood in the art, the term “promoter” can generally refer to a DNA sequence that contains an RNA polymerase binding site, transcription start site, and/or TATA box and assists or promotes the transcription and expression of an associated transcribable polynucleotide sequence and/or gene (or transgene). A promoter can be synthetically produced, varied or derived from a known or naturally occurring promoter sequence or other promoter sequence. A promoter can also include a chimeric promoter comprising a combination of two or more heterologous sequences. A promoter of the present disclosure can thus include variants or fragments of promoter sequences that are similar in composition, but not identical to, other promoter sequence(s) known or provided herein. A promoter provided herein, or variant or fragment thereof, may comprise a “minimal promoter” which provides a basal level of transcription and is comprised of a TATA box or equivalent DNA sequence for recognition and binding of the RNA polymerase II complex for initiation of transcription. A promoter can be classified according to a variety of criteria relating to the pattern of expression of an associated coding or transcribable sequence or gene (including a transgene) operably linked to the promoter, such as constitutive, developmental, tissue-specific, inducible, etc. Promoters that drive expression in all or most tissues of the plant are referred to as “constitutive” promoters. Promoters that drive expression during certain periods or stages of development are referred to as “developmental” promoters. Promoters that drive enhanced expression in certain tissues of the plant relative to other plant tissues are referred to as “tissue-enhanced” or “tissue-preferred” promoters. Thus, a “tissue-preferred” promoter causes relatively higher or preferential expression in a specific tissue(s) of the plant, but with lower levels of expression in other tissue(s) of the plant. Promoters that express within a specific tissue(s) of the plant, with little or no expression in other plant tissues, are referred to as “tissue-specific” promoters. An “inducible” promoter is a promoter that initiates transcription in response to an environmental stimulus such as cold, drought or light, or other stimuli, such as wounding or chemical application. A promoter can also be classified in terms of its origin, such as being heterologous, homologous, chimeric, synthetic, etc.

As used herein, a “plant-expressible promoter” refers to a promoter that can initiate, assist, affect, cause, and/or promote the transcription and expression of its associated transcribable DNA sequence, coding sequence or gene in a plant cell or tissue.

The term “heterologous” in reference to a promoter or other regulatory sequence in relation to an associated polynucleotide sequence (e.g., a transcribable DNA sequence or coding sequence or gene) is a promoter or regulatory sequence that is not operably linked to such associated polynucleotide sequence in nature without human introduction—e.g., the promoter or regulatory sequence has a different origin relative to the associated polynucleotide sequence and/or the promoter or regulatory sequence is not naturally occurring in a plant species to be transformed with the promoter or regulatory sequence. Similarly, “heterologous” in reference to a coding sequence may refer to the use of a recombinant DNA molecule codon-optimized for a different organism as compared to the organism said DNA molecule is being expressed in—e.g., the recombinant DNA sequence encoding a Cas 12a is codon-optimized for expression in humans, but is expressed in a plant cell.

As used herein, an “untranslated region (UTR)” of a gene refers to a segment of an RNA molecule or sequence (e.g., a mRNA molecule) expressed from a gene (or transgene), but excluding the exon and intron sequences of the RNA molecule. An “untranslated region (UTR)” also refers to a DNA segment or sequence encoding such a UTR segment of an RNA molecule. An untranslated region can be a 5′-UTR or a 3′-UTR depending on whether it is located at the 5′ or 3′ end of a DNA or RNA molecule or sequence relative to a coding region of the DNA or RNA molecule or sequence (i.e., upstream (5′) or downstream (3′) of the exon and intron sequences, respectively).

As used herein, a “transcribable region” or “transcribable DNA sequence” refers to a nucleic acid sequence expressed from a gene (or transgene).

As used herein, a “transcription termination sequence” refers to a nucleic acid sequence containing a signal that triggers the release of a newly synthesized transcript RNA molecule from an RNA polymerase complex and marks the end of transcription of a gene or locus.

The terms “percent identity,” “% identity” or “percent identical” as used herein in reference to two or more nucleotide or protein sequences is calculated by (i) comparing two optimally aligned sequences (nucleotide or protein) over a window of comparison, (ii) determining the number of positions at which the identical nucleic acid base (for nucleotide sequences) or amino acid residue (for proteins) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present application, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity can be adjusted upwards to correct for the conservative nature of the substitution. Sequences that differ by such conservative substitutions are said to have “sequence similarity” or “similarity.” Sequences having a percent identity to a base sequence may exhibit the activity of the base sequence.

Homologs are inferred from sequence similarity, by comparison of protein sequences, for example, manually or by use of a computer-based tool. For optimal alignment of sequences to calculate their percent identity, various pair-wise or multiple sequence alignment algorithms and programs are known in the art, such as ClustalW or Basic Local Alignment Search Tool® (BLAST), etc., that can be used to compare the sequence identity or similarity between two or more nucleotide or protein sequences. BLAST, can also be used, for example to search query protein sequences of a base organism against a database of protein sequences of various organisms, to find similar sequences. The generated summary Expectation value (E-value) can be used to measure the level of sequence similarity. Because a protein hit with the lowest E-value for a particular organism may not necessarily be an ortholog or be the only ortholog, a reciprocal query is used to filter hit sequences with significant E-values for ortholog identification. The reciprocal query entails search of the significant hits against a database of protein sequences of the base organism. A hit can be identified as an ortholog, when the reciprocal query's best hit is the query protein itself or a paralog of the query protein. With the reciprocal query process orthologs are further differentiated from paralogs among all the homologs, which allows for the inference of functional equivalence of genes.

The terms “percent complementarity” or “percent complementary”, as used herein in reference to two nucleotide sequences, is similar to the concept of percent identity but refers to the percentage of nucleotides of a query sequence that optimally base-pair or hybridize to nucleotides of a subject sequence when the query and subject sequences are linearly arranged and optimally base paired without secondary folding structures, such as loops, stems or hairpins. Such a percent complementarity may be between two DNA strands, two RNA strands, or a DNA strand and an RNA strand. The “percent complementarity” is calculated by (i) optimally base-pairing or hybridizing the two nucleotide sequences in a linear and fully extended arrangement (i.e., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences may be determined based on the known pairings of nucleotide bases, such as G-C, A-T, and A-U, through hydrogen bonding. If the “percent complementarity” is being calculated in relation to a reference sequence without specifying a particular comparison window, then the percent identity is determined by dividing the number of complementary positions between the two linear sequences by the total length of the reference sequence. Thus, for purposes of the present disclosure, when two sequences (query and subject) are optimally base-paired (with allowance for mismatches or non-base-paired nucleotides but without folding or secondary structures), the “percent complementarity” for the query sequence is equal to the number of base-paired positions between the two sequences divided by the total number of positions in the query sequence over its length (or by the number of positions in the query sequence over a comparison window), which is then multiplied by 100%.

As used herein, a “fragment” of a polynucleotide refers to a sequence comprising at least about 50, at least about 75, at least about 95, at least about 100, at least about 125, at least about 150, at least about 175, at least about 200, at least about 225, at least about 250, at least about 275, at least about 300, at least about 500, at least about 600, at least about 700, at least about 750, at least about 800, at least about 900, or at least about 1000 contiguous nucleotides, or longer, of a DNA molecule or protein as disclosed herein. Methods for producing such fragments from a starting promoter molecule are well known in the art. Fragments of a DNA molecule or protein may exhibit the activity of the DNA molecule or protein from which they are derived.

A plant selectable marker transgene in a transformation vector or construct of the present disclosure may be used to assist in the selection of transformed cells or tissue due to the presence of a selection agent, such as an antibiotic or herbicide, wherein the plant selectable marker transgene provides tolerance or resistance to the selection agent. Thus, the selection agent may bias or favor the survival, development, growth, proliferation, etc., of transformed cells expressing the plant selectable marker gene, such as to increase the proportion of transformed cells or tissues in the Ro plant. Commonly used plant selectable marker genes include, for example, those conferring tolerance or resistance to antibiotics, such as kanamycin and paromomycin (nptll), hygromycin B (aph IV), streptomycin or spectinomycin (aadA) and gentamycin (aac3 and aacC4), or those conferring tolerance or resistance to herbicides such as glufosinate (bar or pat), dicamba (DMO) and glyphosate (proA or EPSPS). Plant screenable marker genes may also be used, which provide an ability to visually screen for transformants, such as luciferase or green fluorescent protein (GFP), or a gene expressing a beta glucuronidase or uidA gene (GUS) for which various chromogenic substrates are known. Plant transformation may also be carried out in the absence of selection during one or more steps or stages of culturing, developing or regenerating transformed explants, tissues, plants and/or plant parts.

### IV. Transformation Methods

Methods and compositions are provided for transforming a plant cell, tissue or explant with a recombinant DNA molecule or construct encoding one or more molecules required for gene suppression, or targeted genome editing (e.g., guide RNA(s) and/or site-directed nuclease(s)) as described herein. Suitable methods for transformation of host plant cells include virtually any method by which DNA or RNA can be introduced into a cell (for example, where a recombinant DNA construct is stably integrated into a plant chromosome or where a recombinant DNA construct or an RNA is transiently provided to a plant cell) and are well known in the art. Two effective methods for cell transformation are bacterially-mediated transformation, such as Agrobacterium-mediated or Rhizobium-mediated transformation, and microprojectile or particle bombardment-mediated transformation. Microprojectile bombardment methods are illustrated, for example, in U.S. Pat. Nos. 5,550,318; 5,538,880; 6,160,208; and 6,399,861. Agrobacterium-mediated transformation methods are described, for example in U.S. Pat. No. 5,591,616, Hinchliffe and Harwood (2019), and Sparrow and Irwin (2015). Other methods for plant transformation, such as microinjection, electroporation, vacuum infiltration, pressure, sonication, silicon carbide fiber agitation, PEG-mediated transformation, etc., are also known in the art.

Any of the polynucleotide molecules of the present disclosure may be introduced into a plant cell in a permanent or transient manner in combination with other genetic elements such as promoters, introns, enhancers, and untranslated leader sequences, etc. Any of the nucleic acid molecules encoding an invertebrate pest, such as B. tabaci RNA or an RNA from a piercing and sucking insect species, or preferably a B. tabaci SSA1-SG1 RNA, may be fabricated and introduced into a plant cell in a manner that allows for production of the dsRNA molecules within the plant cell, providing an insecticidal amount of one or more particular dsRNAs in the diet of a target insect pest.

In one embodiment the plant transformation vector is an isolated and purified DNA molecule comprising a promoter operatively linked to one or more nucleotide sequences of the present disclosure. The nucleotide sequence is selected from the group consisting of SEQ ID NO: 2, 4, 6, 8, 10, 12, or 14 as set forth in the sequence listing, or fragments or complements thereof. The nucleotide sequence includes a segment coding all or part of an RNA present within a targeted pest RNA transcript and may comprise inverted repeats of all or a part of a targeted pest RNA. The DNA molecule comprising the expression vector may also contain a functional intron sequence positioned either upstream of the coding sequence or even within the coding sequence, and may also contain a five prime (5′) untranslated leader sequence (i.e., a UTR or 5′-UTR) positioned between the promoter and the point of translation initiation.

A plant transformation vector may contain sequences from more than one gene, thus allowing production of more than one dsRNA for inhibiting expression of two or more genes in cells of a target pest, such as an invertebrate pest. One skilled in the art will readily appreciate that segments of DNA whose sequence corresponds to that present in different genes can be combined into a single composite DNA segment for expression in a transgenic plant. Alternatively, a plasmid of the present disclosure already containing at least one DNA segment can be modified by the sequential insertion of additional DNA segments between the enhancer and promoter and terminator sequences. In the insect control agent of the present disclosure designed for the inhibition of multiple genes, the genes to be inhibited can be obtained from the same insect species in order to enhance the effectiveness of the insect control agent. In certain embodiments, the genes can be derived from different insects in order to broaden the range of insects against which the agent is effective. When multiple genes are targeted for suppression or a combination of expression and suppression, a polycistronic DNA element can be fabricated as illustrated and disclosed in Fillatti, Application Publication No. US 2004-0029283.

Where a nucleotide sequence of the present disclosure is to be used to transform a plant, a promoter exhibiting the ability to drive expression of the coding sequence in that particular species of plant is selected. Promoters that function in different plant species are also well known in the art. Promoters useful for expression of polypeptides in plants are those that are inducible, viral, synthetic, or constitutive as described in Odell et al. (1985, Nature 313:810-812), and/or promoters that are temporally regulated, spatially regulated, and spatio-temporally regulated. Preferred promoters include the enhanced CaMV35S promoters, and the FMV35S promoter. For the purpose of the present disclosure, e.g., for optimum control of species that feed on plant leaves, it is preferable to achieve the highest levels of expression of these genes within the leaves of plants. A number of phloem-specific promoters have been identified and are known in the art.

Transformation of plant material is practiced in tissue culture on nutrient media, for example a mixture of nutrients that allow cells to grow in vitro. Recipient cell targets include, but are not limited to, meristem cells, shoot tips, hypocotyls, calli, immature or mature embryos, and gametic cells such as microspores and pollen. Callus can be initiated from tissue sources including, but not limited to, immature or mature embryos, hypocotyls, seedling apical meristems, microspores and the like. Cells containing a transgenic nucleus are grown into transgenic plants. Any suitable method or technique for transformation of a plant cell known in the art may be used according to present methods. In transformation, DNA is typically introduced into only a small percentage of target plant cells in any one transformation experiment. Marker genes are used to provide an efficient system for identification of those cells that are stably transformed by receiving and integrating a recombinant DNA molecule into their genomes.

As used herein, the terms “regeneration” and “regenerating” refer to a process of growing or developing a plant from one or more plant cells through one or more culturing steps. Transformed or edited cells, tissues or explants containing a DNA sequence insertion or edit may be grown, developed or regenerated into transgenic plants in culture, plugs, or soil according to methods known in the art. Certain embodiments of the disclosure therefore relate to methods and constructs for regenerating a plant from a cell with modified genomic DNA resulting from genome editing. The regenerated plant can then be used to propagate additional plants, e.g. by vegetative propagation.

According to an aspect of the present disclosure, regenerated plants or a progeny plant, plant part or seed thereof can be screened or selected based on a marker, trait, or phenotype produced by the edit or mutation, or by the site-directed integration of an insertion sequence, transgene, etc., in the developed or regenerated plant, or a progeny plant, plant part or seed thereof. If a given mutation, edit, trait or phenotype is recessive, one or more generations or crosses (e.g., selfing) from the initial Ro plant may be necessary to produce a plant homozygous for the edit or mutation so the trait or phenotype can be observed. Progeny plants, such as plants grown from RI seed or in subsequent generations, can be tested for zygosity using any known zygosity assay, such as by using a single nucleotide polymorphism (SNP) assay, DNA sequencing, thermal amplification, or polymerase chain reaction (PCR), and/or Southern blotting that allows for the distinction between heterozygote, homozygote and wild-type plants.

Methods and techniques are provided for screening for, and/or identifying, cells or plants, etc., for the presence of targeted edits or transgenes, and selecting cells or plants comprising targeted edits or transgenes, which may be based on one or more phenotypes or traits, or on the presence or absence of a molecular marker or polynucleotide or protein sequence in the cells or plants. As used herein, a “molecular technique” refers to any method known in the fields of molecular biology, biochemistry, genetics, plant biology, or biophysics that involves the use, manipulation, or analysis of a nucleic acid, a protein, or a lipid. Without being limiting, molecular techniques useful for detecting the presence of a modified sequence in a genome include phenotypic screening; molecular marker technologies such as SNP analysis by TaqMan® or Illumina/Infinium technology; Southern blot; PCR; enzyme-linked immunosorbent assay (ELISA); and sequencing (e.g., Sanger, Illumina®, 454, Pac-Bio, Ion Torrent™). In one aspect, a method of detection provided herein comprises phenotypic screening. In another aspect, a method of detection provided herein comprises SNP analysis. In a further aspect, a method of detection provided herein comprises a Southern blot. In a further aspect, a method of detection provided herein comprises PCR. In an aspect, a method of detection provided herein comprises ELISA. In a further aspect, a method of detection provided herein comprises determining the sequence of a nucleic acid or a protein. Without being limiting, nucleic acids can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, NY).

Nucleic acids can be isolated using techniques routine in the art. For example, nucleic acids can be isolated using any method including, without limitation, recombinant nucleic acid technology, and/or PCR. General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides.

Detection (e.g., of an amplification product, of a hybridization complex, of a polypeptide) can be accomplished using detectable labels that may be attached or associated with a hybridization probe or antibody. The term “label” is intended to encompass the use of direct labels as well as indirect labels. Detectable labels include enzymes, prosthetic groups, fluorescent materials, luminescent materials, bioluminescent materials, and radioactive materials. The screening and selection of modified (e.g., edited) plants or plant cells can be through any methodologies known to those skilled in the art of molecular biology. Examples of screening and selection methodologies include, but are not limited to, Southern analysis, PCR amplification for detection of a polynucleotide, Northern blots, RNase protection, primer-extension, RT-PCR amplification for detecting RNA transcripts, Sanger sequencing, Next Generation sequencing technologies (e.g., Illumina®, PacBio®, Ion Torrent™, etc.) enzymatic assays for detecting enzyme or ribozyme activity of polypeptides and polynucleotides, and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are known in the art.

As used herein, the term “polypeptide” refers to a chain of at least two covalently linked amino acids. Polypeptides can be encoded by polynucleotides provided herein. An example of a polypeptide is a protein. Proteins provided herein can be encoded by nucleic acid molecules provided herein. Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

Polypeptides can be detected using antibodies. Techniques for detecting polypeptides using antibodies include enzyme linked immunosorbent assays (ELISAs), Western blots, immunoprecipitations and immunofluorescence. An antibody provided herein can be a polyclonal antibody or a monoclonal antibody. An antibody having specific binding affinity for a polypeptide provided herein can be generated using methods well known in the art. An antibody provided herein can be attached to a solid support such as a microtiter plate using methods known in the art.

The present disclosure can be, in practice, combined with other insect control traits in a plant to achieve desired traits for enhanced control of insect infestation. Combining insect control traits that employ distinct modes-of-action can provide insect-protected transgenic plants with superior durability over plants harboring a single insect control trait because of the reduced probability that resistance will develop in the field.

A plant that may be transformed with a recombinant DNA molecule or transformation vector comprising interfering RNA sequence(s) (e.g. ssRNA, dsRNA, siRNA, etc.), guide RNA(s), or combination thereof, may include a variety of flowering plants or angiosperms, which may be further defined as including various dicotyledonous (dicot) plant species or monocotyledonous (monocot) plant species. A dicot plant could be members of the Fabaceae family (such as legumes), sunflower (Helianthus annuus), safflower (Carthamus tinctorius), sesame (Sesamum spp.), tobacco (Nicotiana tabacum), potato (Solanum tuberosum), cotton (Gossypium barbadense, Gossypium hirsutum), sweet potato (Ipomoea batatas), cassava (Manihot esculenta), coffee (Coffea spp.), tea (Camellia spp.), fruit trees, such as apple (Malus spp.), Prunus spp., such as plum, apricot, peach, cherry, etc., pear (Pyrus spp.), fig (Ficus carica), etc., citrus trees (Citrus spp.), cocoa (Theobroma cacao), avocado (Persea americana), olive (Olea europaea), almond (Prunus amygdalus), walnut (Juglans spp.), strawberry (Fragaria spp.), watermelon (Citrullus lanatus), pepper (Capsicum spp.), eggplant, beet (Beta vulgaris), grape (Vitis, Muscadinia), tomato (Lycopersicon esculentum, Solanum lycopersicum), cucumber (Cucumis sativus), and members of the Brassicaceae family, such as thale cress (Arabidopsis thaliana) and Brassica sp. (e.g., B. napus, B. rapa, B. juncea), particularly those Brassica species useful as sources of seed oil. Legumes and leguminous plants include peas (Pisum sativum) alfalfa (Medicago sativa), barrel clover (Medicago truncatula), pigeon pea (Cajanus cajan) guar (Cyamopsis tetragonoloba), carob (Ceratonia siliqua), fenugreck (Trigonella foenum-graecum), soybean (Glycine max), common bean (Phaseolus vulgaris), cowpea (Vigna unguiculata), mung bean (Vigna radiata), lima bean (Phaseolus lunatus), fava bean (Vicia faba), lentil (Lens culinaris or Lens esculenta), peanut (Arachis hypogaea), licorice (Glycyrrhiza glabra), and chickpea (Cicer arietinum). A monocot plant could be oil palm (Elaeis spp.), coconut (Cocos spp.), banana (Musa spp.), and cereals such as corn (Zea mays), barley (Hordeum vulgare), sorghum (Sorghum bicolor), rice (Oryza sativa), and wheat (Triticum aestivum). Given that the present disclosure may apply to a broad range of plant species, the present disclosure further applies to other botanical structures analogous to pods of leguminous plants, such as bolls, siliques, fruits, nuts, tubers, etc.

### V. Plants Comprising DNA Molecules

The term “transgenic plant cell” or “transgenic plant” as used herein includes a plant cell or a plant that contains an exogenous nucleic acid, which can be derived from an invertebrate pest, such as whitefly, (e.g. B. tabaci), or from a different insect species. The transgenic plants are also meant to comprise progeny (decedent, offspring, etc.) of any generation of such a transgenic plant or a seed of any generation of all such transgenic plants wherein said progeny or seed comprises a DNA sequence encoding the RNA, SRNA, dsRNA, siRNA, gRNA, or fragment thereof of the present disclosure is also an important aspect of the disclosure.

A transgenic plant formed using Agrobacterium transformation methods typically contains a single simple recombinant DNA sequence inserted into one chromosome and is referred to as a transgenic event. Such transgenic plants can be referred to as being heterozygous for the inserted exogenous sequence. A transgenic plant homozygous with respect to a transgene can be obtained by sexually mating (selfing) an independent segregant transgenic plant that contains a single exogenous gene sequence to itself, for example an F0 plant, to produce F1 seed. One fourth of the F1 seed produced will be heterozygous with respect to the transgene. Germinating F1 seed results in plants that can be tested for heterozygosity, typically using a SNP assay or a thermal amplification assay that allows for the distinction between heterozygotes and homozygotes (i.e., a zygosity assay). Crossing a heterozygous plant with itself or another heterozygous plant results in only heterozygous progeny.

As used herein, the term “transformed” refers to a cell, tissue, organ, or organism into which a foreign DNA molecule, such as a construct, has been introduced. The introduced DNA molecule may be integrated into the genomic DNA of the recipient cell, tissue, organ, or organism such that the introduced DNA molecule is inherited by subsequent progeny. A “transgenic” or “transformed” cell or organism may also include progeny of the cell or organism and progeny produced from a breeding program employing such a transgenic organism as a parent in a cross and exhibiting an altered phenotype resulting from the presence of a foreign DNA molecule. The introduced DNA molecule may also be transiently introduced into the recipient cell such that the introduced DNA molecule is not inherited by subsequent progeny. The term “transgenic” refers to a bacterium, fungus, or plant containing one or more heterologous DNA molecules.

A transgenic plant subsequently may be regenerated from a transgenic plant cell of the present disclosure. Using conventional breeding techniques or self-pollination, seed may be produced from this transgenic plant. Such seed, and the resulting progeny plant grown from such seed, will contain the recombinant DNA molecule of the present disclosure, and therefore will be transgenic.

Transgenic plants of the disclosure can be self-pollinated to provide seed for homozygous transgenic plants of the disclosure (homozygous for the recombinant DNA molecule) or crossed with non-transgenic plants or different transgenic plants to provide seed for heterozygous transgenic plants of the present disclosure (heterozygous for the recombinant DNA molecule). Both such homozygous and heterozygous transgenic plants are referred to herein as “progeny plants.” Progeny plants are transgenic plants descended from the original transgenic plant and containing the recombinant DNA molecule of the present disclosure. Seeds produced using a transgenic plant of the present disclosure can be harvested and used to grow generations of transgenic plants, i.e., progeny plants of the present disclosure, comprising the construct of this disclosure and expressing a gene of agronomic interest. Descriptions of breeding methods that are commonly used for different crops can be found in one of several reference books, see, e.g., Allard, Principles of Plant Breeding, John Wiley & Sons, NY, U. of CA, Davis, CA, 50-98 (1960); Simmonds, Principles of Crop Improvement, Longman, Inc., NY, 369-399 (1979); Sneep and Hendriksen, Plant breeding Perspectives, Wageningen (ed), Center for Agricultural Publishing and Documentation (1979); Fehr, Soybeans: Improvement, Production and Uses, 2nd Edition, Monograph, 16:249 (1987); Fehr, Principles of Variety Development, Theory and Technique, (Vol. 1) and Crop Species Soybean (Vol. 2), Iowa State Univ., Macmillan Pub. Co., NY, 360-376 (1987).

The transformed plants may be analyzed for the presence of the gene or genes of interest and the expression level and/or profile conferred by the regulatory elements of the present disclosure. Those of skill in the art are aware of the numerous methods available for the analysis of transformed plants. For example, methods for plant analysis include, but are not limited to, Southern blots or northern blots, PCR-based approaches, biochemical analyses, phenotypic screening methods, field evaluations, and immunodiagnostic assays. The expression of a transcribable DNA molecule can be measured using TaqMan® (Applied Biosystems, Foster City, CA) reagents and methods as described by the manufacturer and PCR cycle times determined using the TaqMan® Testing Matrix. Alternatively, other methods and reagents for measuring expression of a transcribable DNA molecule are well known in the art. For example, the Invader® (Third Wave Technologies, Madison, WI) or SYBR Green (Thermo Fisher, A46012) reagents and methods as described by the manufacturer can be used to evaluate transgene expression.

The seeds of the plants of this disclosure can be harvested from fertile transgenic plants and be used to grow progeny generations of transformed plants of this disclosure including hybrid plant lines comprising the construct of this disclosure and expressing a gene of agronomic interest.

The present disclosure also provides for parts of the plants of the present disclosure. Plant parts, without limitation, include leaves, stems, roots, tubers, seeds, endosperm, ovule, and pollen. The disclosure also includes and provides transformed plant cells which comprise a nucleic acid molecule of the present disclosure.

The transgenic plant may pass along the transgenic polynucleotide molecule to its progeny. Progeny includes any regenerable plant part or seed comprising the transgene derived from an ancestor plant. The transgenic plant is preferably homozygous for the transformed polynucleotide molecule and transmits that sequence to all offspring as a result of sexual reproduction. Progeny may be grown from seeds produced by the transgenic plant. These additional plants may then be self-pollinated to generate a true breeding line of plants. Progeny from these plants are evaluated, among other things, for gene expression. The gene expression may be detected by several common methods such as western blotting, northern blotting, immuno-precipitation, and ELISA.

As an alternative to traditional transformation methods, a DNA molecule, such as a transgene, expression cassette(s), etc., may be inserted or integrated into a specific site or locus within the genome of a plant or plant cell via site-directed integration. Recombinant DNA construct(s) and molecule(s) of this disclosure may thus include a donor template sequence comprising at least one transgene, expression cassette, or other DNA sequence for insertion into the genome of the plant or plant cell. Such donor template for site-directed integration may further include one or two homology arms flanking an insertion sequence (i.e., the sequence, transgene, cassette, etc., to be inserted into the plant genome). The recombinant DNA construct(s) of this disclosure may further comprise an expression cassette(s) encoding a site-specific nuclease and/or any associated protein(s) to carry out site-directed integration. These nuclease expressing cassette(s) may be present in the same molecule or vector as the donor template (in cis) or on a separate molecule or vector (in trans). Several methods for site-directed integration are known in the art involving different proteins (or complexes of proteins and/or guide RNA) that cut the genomic DNA to produce a double strand break (DSB) or nick at a desired genomic site or locus.

Briefly as understood in the art, during the process of repairing the DSB or nick introduced by the nuclease enzyme, the donor template DNA may become integrated into the genome at the site of the DSB or nick. The presence of the homology arm(s) in the donor template may promote the adoption and targeting of the insertion sequence into the plant genome during the repair process through homologous recombination, although an insertion event may occur through non-homologous end joining (NHEJ). Examples of site-specific nucleases that may be used include zinc-finger nucleases, engineered or native meganucleases, TALE-endonucleases, and RNA-guided endonucleases (e.g., Cas9 or Cpf1). For methods using RNA-guided site-specific nucleases (e.g., Cas9 or Cpf1), the recombinant DNA construct(s) will also comprise a sequence encoding one or more guide RNAs to direct the nuclease to the desired site within the plant genome.

### VI. Invertebrate Pest

The present disclosure provides for a recombinant polynucleotide molecule that disrupts the activity of said polypeptide when provided in the diet of an invertebrate pest. The invertebrate pest can be a pest in the order of Hemiptera. For example, the Hemiptera can belong to the family Aleyrodidae, comprising over 1,200 known species of whitefly, many of which are pests to various plants and crops worldwide. For example, the whitefly can be of species Aleurocanthus spiniferus (Orange spiny whitefly), Aleuroclava lefroyi (Coconut whitefly), Aleuroclava manii, Aleurodicus dispersus (Spiralling whitefly), Aleurodicus rugioperculatus (Rugose spiralling whitefly), Aleurothrixus floccosus (Woolly whitefly), Aleurotrachelus atratus (Palm infesting whitefly), Aleyrodes proletella (Cabbage whitefly), Bemisia argentifolii (Silverleaf whitefly), Bemisia tabaci (Sweet potato whitefly/Cassava whitefly, SSA1, SSA2, MEAM1, also known as B biotype; MED, also known as Q biotype), Paraleyrodes bondari (Bondar's nesting whitefly), or Trialeurodes vaporariorum (Greenhouse whitefly). These species are known for their impact on agriculture and horticulture, causing significant damage to various crops through direct feeding and transmission of plant viruses. For example, cassava whitefly/sweet potato whitefly (Bemisia tabaci) is a significant pest for many agricultural crops, spreading several plant viruses; greenhouse whitefly (Trialeurodes vaporariorum) commonly affects a wide range of herbaceous plants in greenhouses and gardens; silverleaf whitefly (Bemisia argentifolii) is known for infesting numerous plants including tomatoes, beans, and various ornamentals, and causing specific damage symptoms such as silvering of leaves and irregular ripening of fruits; citrus blackfly (Aleurocanthus woglumi), despite its name, is a whitefly that targets citrus plants; cabbage whitefly (Aleyrodes proletella) is a pest of various Brassica crops. In addition to being vectors for plant viruses, which make them particularly dangerous to crops, whiteflies excrete a sticky substance called honeydew, which can lead to sooty mold growth, further harming the plants.

Current control methods can include regular inspection, biological control using natural predators, mechanical control with sticky traps, and chemical treatments, although resistance to traditional insecticides can be a problem. Integrated pest management strategies are often recommended for effective control of whitefly populations.

### VII. Commodity Products

The present disclosure provides a commodity product comprising DNA molecules according to the disclosure. As used herein, a “commodity product” refers to any composition or product which is comprised of material derived from a plant, seed, plant cell or plant part comprising a DNA molecule of the disclosure. Commodity products may be sold to consumers and may be viable or nonviable. Nonviable commodity products include but are not limited to nonviable seeds and grains; processed seeds, seed parts, and plant parts; dehydrated plant tissue, frozen plant tissue, and processed plant tissue; seeds and plant parts processed for animal feed for terrestrial and/or aquatic animal consumption, oil, meal, flour, flakes, bran, fiber, milk, cheese, paper, cream, wine, and any other food for human consumption; and biomasses and fuel products. Viable commodity products include but are not limited to seeds and plant cells. Plants comprising a DNA molecule according to the disclosure can thus be used to manufacture any commodity product typically acquired from plants or parts thereof.

### VIII. Definitions

The following definitions are provided to define and clarify the meaning of these terms in reference to the relevant embodiments of the present disclosure as used herein and to guide those of ordinary skill in the art in understanding the present disclosure. Unless otherwise noted, terms are to be understood according to their conventional meaning and usage in the relevant art, particularly in the field of molecular biology and plant transformation.

When introducing elements of the present disclosure or the embodiment(s) thereof, the articles “a”, “an”, “the”, and “said” are intended to mean that there are one or more of the elements.

The term “and/or”, when used in a list of two or more items, means any one of the items, any combination of the items, or all of the items with which this term is associated.

The terms “comprising”, “including”, and “having” are intended to be inclusive and mean that there may be additional elements other than the listed elements. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

As used herein, a “plant” includes a whole plant, explant, plant part, seedling, or plantlet at any stage of regeneration or development.

As used herein, a “plant part” can refer to any organ or intact tissue of a plant, such as a meristem, shoot organ/structure (e.g., leaf, stem or node), root, flower or floral organ/structure (e.g., bract, sepal, petal, stamen, carpel, anther and ovule), seed, embryo, endosperm, seed coat, fruit, the mature ovary, propagule, or other plant tissues (e.g., vascular tissue, dermal tissue, ground tissue, and the like), or any portion thereof. Plant parts of the present disclosure can be viable, nonviable, regenerable, and/or non-regenerable. A “propagule” can include any plant part that can grow into an entire plant.

As used herein, “genomic DNA” or “gDNA” refers to chromosomal DNA of an organism.

As used herein, a “genomic modification” (also referred to as “modification”) or “genomic edit” (also referred to as “edit”) refers to any modification to a genomic nucleotide sequence as compared to a wild-type or control plant. A genomic modification or genomic edit comprises a deletion, an insertion, a substitution, an inversion, a duplication, or any combination thereof.

As used herein, “T-DNA” or “transfer DNA” refers to the transferred DNA of the tumor-inducing (Ti) plasmid of some species of bacteria such as Agrobacterium tumefaciens.

As used herein, “a target structural motif,” or “target motif,” refers to any rationally selected sequence or combination of sequences in which the sequences or sequence(s) are chosen based on a three-dimensional configuration that is formed upon the folding of the target motif. There are a variety of target motifs known in the art. Protein target motifs include, but are not limited to, enzymatic active sites and signal sequences. Nucleic acid target motifs include, but are not limited to, promoter sequences, cis elements, hairpin structures and inducible expression elements (protein binding sequences). Specifically, a “target motif” may refer to a catalytic domain and/or a signal peptide required for an enzyme to function as an extracellular enzyme in the gut lumen of whitefly.

All methods described herein can be performed in any suitable order unless otherwise indicated herein or clearly contradicted by context. The use of any and all examples, or exemplary language (e.g., “such as”) provided with respect to certain embodiments herein is intended merely to illuminate the present disclosure and does not pose a limitation on the scope of the present disclosure otherwise claimed.

### EXAMPLES

The following examples are included to illustrate embodiments of the invention. It should be appreciated by those of skill in the art that the techniques disclosed in the examples that follow represent techniques discovered by the inventor to function well in the practice of the invention. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1: Identification of Key Osmoregulation and Symbiont Genes as Targets for Controlling Bemisia tabaci

In this example, the differential expression of genes in the cassava B. tabaci (SSA1-SG1) gut and bacteriocytes was analyzed to identify essential gene targets for the management of B. tabaci SSA1-SG1 population using RNA interference. Functional analysis of the genes enriched in gut and bacteriocytes was done to identify gene targets whose disruption would have a detrimental effect on B. tabaci SSA1-SG1 survival.

Dissection of Guts and Bacteriocytes from Cassava B. tabaci

Genes enriched in the gut and bacteriocytes were identified by comparing gene expression (i) in the B. tabaci gut and (ii) bacteriocyte with the whole-body. The cassava B. tabaci population (SSA1-SG1) used in this study was derived from a single virgin female and male originally collected from Namulonge, Uganda, an area with superabundant B. tabaci populations and a high incidence of both Cassava brown streak disease and Cassava mosaic disease. The B. tabaci population was maintained at the Natural Resources Institute, the University of Greenwich, on aubergine (Solanum melongena cv. Black Beauty) under insectary conditions (60% relative humidity, 28° C. and 12h light: 12h dark regime) with 500 μmol^{m-2s-1} PAR. light intensity. Approximately 300 guts and more than 10000 bacteriocytes were dissected in phosphate buffered saline (PBS, 1×) from 300 and 500 female adult whiteflies respectively, belonging to cassava B. tabaci species SSA1-SG1 using fine pins and a stercomicroscope. Both guts and bacteriocytes were maintained in RNAlater™ stabilization solution (Invitrogen™) to preserve their RNA quality. A total of 50 female adult whiteflies were also collected and immediately stored at −80° C. for analysis of gene expression in wholebody.

RNA Extraction from Dissected Guts and Bacteriocytes

Total RNA was extracted from B. tabaci guts, bacteriocytes and whole bodies using TRIzoL™ reagent (Invitrogen™), with slight modifications made in the manufacturer's instructions to ensure purity and quantity of RNA for downstream analysis for low tissue amounts. Briefly, the B. tabaci whole body, guts and bacteriocytes were homogenized with 500 μl TRIzoL™. Homogenized samples were incubated at room temperature for 5 minutes followed by adding 100 μl chloroform. The mixture was then incubated for 3 minutes at room temperature and centrifuged at 12,000 g for 15 minutes at 4° C. to separate the aqueous phase. The aqueous phase was then transferred to a clean tube and the second cleanup was done by adding 100 μl of chloroform. The mixture was vortexed for 15 seconds then incubated at room temperature for 3 minutes and thereafter centrifuged at 12,000 g for 10 minutes at 4° C. Phase separation was repeated and 1 μl of linear acrylamide was added to the aqueous phase to improve the precipitation of RNA. Ice cold isopropanol (250 μl) was then added and mixed by pipetting. The mixture was further incubated at room temperature for 10 minutes and then centrifuged at 12000 g for 15 minutes at 4° C. The supernatant was removed and discarded using a pipette and the RNA pellet washed twice with 500 μl of 75% ethanol. Finally, the RNA pellet was air dried for 5-10 minutes then dissolved in DNase/RNase free water followed by incubating at 60° C. for 10 minutes to ensure all the RNA had dissolved. Total RNA was further treated with ezDNase™ enzyme (Invitrogen™) according to the manufacturer's instructions, to remove contaminating genomic DNA. In brief, 1 μl 10× ezDNase buffer and 1 μl ezDNase™ enzyme were added to 8 μl RNA and mixed gently. RNA and ezDNase™ mixture were then incubated for 2 minutes at 37° C. followed by 5 minutes at 55° C. in presence of 10 mM DTT to inactivate the enzyme. RNA quantity and integrity were confirmed using a Qubit fluorometer (Life Technologies).

rRNA Depletion and RNAseg Library Preparation

Total RNA was depleted of cytoplasmic and mitochondrial rRNA using the Ribo-Zero Gold rRNA removal epidemiology kit (Illumina, San Diego, USA) according to manufacturer's instruction. For the whole-body samples, 1 μg total RNA was used for rRNA depletion, due to limited material only 367 ng total RNA was used for the bacteriocyte RNA sample and 230 ng for the gut sample.

RNAseq libraries were prepared with the NEBNext Ultra II RNA Library Prep Kit (New England Biolabs Inc), according to the manufacturer's instructions. For the whole-body samples, 15 ng rRNA-depleted RNA was used as input while for the bacteriocyte and gut samples, all the rRNA-depleted RNA (not quantified) was used as input. The final libraries were quantified with a Qubit HS DNA assay and the size distribution was determined on a Fragment Analyzer instrument (Agilent Technologies, Inc. CA, USA). Equimolar amounts of each library were pooled for sequencing.

### RNA Sequencing and RNAseg Analysis

The RNAseq libraries were sequenced and >40 million single-end 75 nt reads were generated on the NEXTseq500 sequencing platform at the Cornell Genomics Facility (Biotechnology Resource Center). Raw reads were filtered to remove low quality reads and adaptor sequences with Cutadapt (Martin, 2011). The cleaned RNAseq reads were aligned against the SSA1-SG1 genome using HISAT2 (Kim et al., 2015), which runs Bowtie2 in the background. The accepted hits were used, first to develop gene annotation file containing gene models based on RNAseq aligned reads for guts, bacteriocytes, and whole-body using cufflinks and cuffmerge version 2.2.1 (Trapnell et al., 2010). Second, the accepted hits were assembled and aligned reads counted using featureCounts (Liao et al., 2014). Differential gene expression in cassava B. tabaci (SSA1-SG1) gut and bacteriocytes relative to the whole-body was performed based feature counts using edgeR (Robinson et al., 2010) with false discovery rate controls based on the Benjamini & Hochberg, (1995) method and trimmed mean of M-values normalization to account for library size variation between the samples. Up-regulated genes with log 2 fold difference (log FC) based on general linear model (likelihood ratio test) greater than one in B. tabaci guts and bacteriocytes were selected for further analysis.

### Identification of Osmoregulation and Symbiosis Genes

Nucleotide sequences for the differentially expressed genes in both B. tabaci gut and bacteriocytes were extracted using Samtools faidx tool (Li et al., 2009). Blastx search (Altschul et al., 1997) with a cutoff E-value of 1e-3 against NCBI protein reference non-redundant database (nr), mapping and annotation of these sequences were done using Blast2GO suite (Conesa et al., 2005). The suite was also used to generate and characterize gene ontology terms based on BLAST output. Gene ontology annotation was characterized into three groups, namely molecular function, biological process and cellular component. Selected sequences with metabolic functions were further translated to protein sequences using the ExPASy molecular biology server of the Swiss Institute of Bioinformatics (Gasteiger et al., 2003). The translated protein sequences with Aamy domain (PF00128, IPR006047) for glycosyl hydrolases (family 13), aquaporin domain (PF00230, IPR000425) for osmoregulation genes and amino acid transporter domains (PF01490, IPR013057), amino acid/polyamine transporter I (IPR002293) and amino acid permease domain (IPR004841, IPR013612) for symbiont genes were selected from the blast2go output. The selected sequences were further analyzed using SignalP 4.0 server (Petersen et al., 2011), NCBI conserved domain database (Marchler-Bauer et al., 2015), HMMER V.3 (Eddy, 2011) and InterProScan (Zdobnov & Apweiler, 2001) to confirm the functional domain for sucrase, aquaporin and amino acid transport. The selected sequences were also analyzed using NCBI Batch web CD-search tool to determine if the conserved domains were complete or incomplete at N-terminus or C-terminus. Furthermore, a candidate-based search for horizontally transferred genes (HTGs) in SSA1-SG1 was conducted. Sequences of HTGs identified in MEAM1 (Luan et al., 2015; Chen et al., 2016), were identified from a whitefly genome database (Chen W, et al., BMC Biology 14:110, 2016). A reciprocal blast against SSA1-SG1 genome and transcriptome was done to identify genes that encode for HTGs in SSA1-SG1 and their expression level in the bacteriocytes.

### Phylogenetic Analysis

To determine the conservation of osmoregulation function of the selected genes, phylogenetic analyses were done to compare selected sequences to osmoregulation genes in other B. tabaci species and aphids (Jing et al., 2016). Protein sequences were aligned by ClustalW (Larkin et al., 2007) implemented in MEGA X using default parameters. The best-fit amino acid substitution model to describe the substitution pattern was selected based on Bayesian information criterion (BIC) scores, computed using MEGA X (Kumar et al., 2016; Kumar et al., 2018). The best amino acid substitution model with the lowest BIC score was selected and used in the phylogenetic reconstruction implemented in Bayesian Evolutionary Analysis Sampling Trees (BEAST version 1.10.2) (Suchard et al., 2018) with four gamma categories and strict molecular clock model at 1.0 clock rate. Yule process model (Gernhard, 2008) with uniform birth rate and inverse gamma as the gamma shape was used to provide prior on the tree. Markov Chain Monte Carlo (MCMC) were set with a chain length of 10,000,000. The generated tree was visualized using FigTree V1.4.3. The phylogenetic trees were used to study the relationship of selected genes with experimentally validated osmoregulation genes in other B. tabaci and aphid species (Price et al., 2007; Shakesby et al., 2009; Mathew et al., 2011) to select the most suitable gene target.

### Validation of Selected Genes Using Real-Time Quantitative PCR

Expression of the selected genes was validated using real-time quantitative PCR. In brief, the total RNA for whitefly gut, bacteriocytes whole-body samples were normalized to ensure an equal amount of starting RNA template (120 ng). Normalized RNAs were then treated with 1 μl of the ezDNase™ enzyme (Invitrogen™) for 2 minutes at 37° C. to remove genomic DNA. The RNA sample was further incubated at 55° C. for 5 minutes in the presence of 10 mM DTT to inactivate the enzyme. First-strand cDNA synthesis was carried out using SuperScript™ III Reverse Transcriptase (Invitrogen™) according to the manufacturer's instructions. A three-step thermocycling protocol was used with polymerase activation and DNA denaturation at 95° C. for 3 minutes followed by 40 cycles of both denaturation at 95° C. and annealing/extension at 55.2° C. for 45 seconds, performed on C1000 Thermocycler with CFX96 Real-time detection system (Bio-Rad), using 60S ribosomal protein L13a (RPL13) and β-tubulin as the internal controls. A total of 20 μl of reaction mix containing 10 μl of iQ™ SYBR® Green Supermix (2×), 1 μl of forward and reverse primer (Table 1), 1 μl of cDNA and 7 μl of water was used. Melting curve analysis at the end of the reaction was done by increasing temperature from 55-95° C. in increments of 0.5° C. every 5 seconds to assess the dissociation characteristics of double-stranded DNA during heating.

TABLE 1
Primers used in the validation of gene expression of selected genes

| | | SEQ ID | Amplicon | |
|---|---|---|---|---|
| Target gene | Primer sequence | NO. | length (bp) | Tm (° C.) |
| ENSSSA1UGT022145 | F - TTGTTTCGCAAGTTTGCCGT | 15 | 90 | 59.83 |
| (AQP1) | R - GACTGATTGACGCCCTGGAT | 16 | | 59.82 |
| ENSSSA1UGT002057 | F - AACACTGCGAATAGCGCATC | 17 | 86 | 59.35 |
| (SUC1) | R - CGCCACTCTAGATGTTCGCA | 18 | | 60.18 |
| ENSSSA1UGT002066 | F - CGGTAAGGTCTGAAACTGCGAT | 19 | 110 | 60.86 |
| (SUC2) | R - GTTTTGCTAGATGTGCAAGGCA | 20 | | 58.76 |
| Diaminopimelate | F - ACTACAATTCTCGCCCTCGC | 21 | 86 | 60.18 |
| decarboxylase | R - GTCATCAAAGGTCTCCCGCC | 22 | | 60.74 |
| (LysA) | | | | |
| Arginosuccinate lyase | F - AAGCTCTGGTGTAAGGCACA | 23 | 98 | 59.24 |
| (ArgH) | R - AGGATGTCTTGGGTCGCTTC | 24 | | 59.75 |
| Branched-chain-amino-acid | F - CGTCCAGAGTCAGTGGCAA | 25 | 93 | 59.63 |
| aminotransferase (BCAT) | R - GTTCATGGCTCCGGCTTCAG | 26 | | 61.37 |
| Aspartate aminotrans- | F - GGTCCTACCAGTTGTGCGAA | 27 | 95 | 59.97 |
| ferase (AAT) | R - AGAATGGAGCCGAACCCAAG | 28 | | 60.04 |
| 60S ribosomal protein | F - CATTCCACTACAGAGCTCCA | 29 | 101 | 60.00 |
| L13a (RPL13A) | R - TTTCAGGTTTCGGATGGCTT | 30 | | 60.00 |
| β-Tubulin (fl-Tub) | F - TGTCAGGAGTAACGACGTGTTTG | 31 | 150 | 60.00 |
| | R - TTCGGGAACGGTAAGTGCTC | 32 | | 60.00 |
| 4-hydroxy- | F - TGGTAAAAGACTACCAGGCGAA | 33 | 118 | 59.37 |
| tetrahydrodipicolinate | R - AGCTTGGTGTTTACAGCTGAGAG | 34 | | 60.81 |
| reductase (dapB) | | | | |

RNAseq Analysis for Gut and Bacteriocytes Samples from Cassava B. tabaci

A total of 144,377,532 sequencing reads were generated from guts, bacteriocytes and whole-body samples of cassava B. tabaci (SSA1-SG1) (Table 2). These samples had a high percentage mapping ranging from 78.0 to 83.8%. Of these, the bacteriocyte sample had the highest number of reads mapping on to the reference genome with a percentage mapping of 83.84%.

TABLE 2
Mapping quality of RNAseq reads from SSA1-SG1
whole-body, bacteriocytes and guts samples

| | | Mapping to SSA1-SG1 | |
|---|---|---|---|
| | | genome | |
| Sample | Mapped reads | Unmapped reads | Percentage mapping (%) |
| Whole-body | 37,170,765 | 10,475,500 | 78.01 |
| Bacteriocyte | 39,660,655 | 7,644,558 | 83.84 |
| Guts | 38,603,036 | 10,823,018 | 78.10 |
| Total | 115,434,450 | 28,943,076 | |

Identification of Transcripts Enriched in the Cassava B. tabaci (SSA1-SG1) Gut and Bacteriocytes

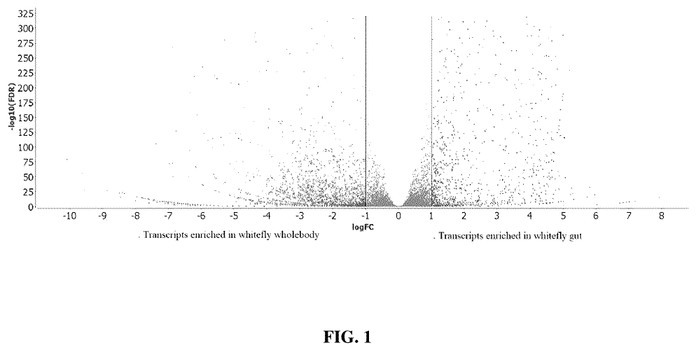

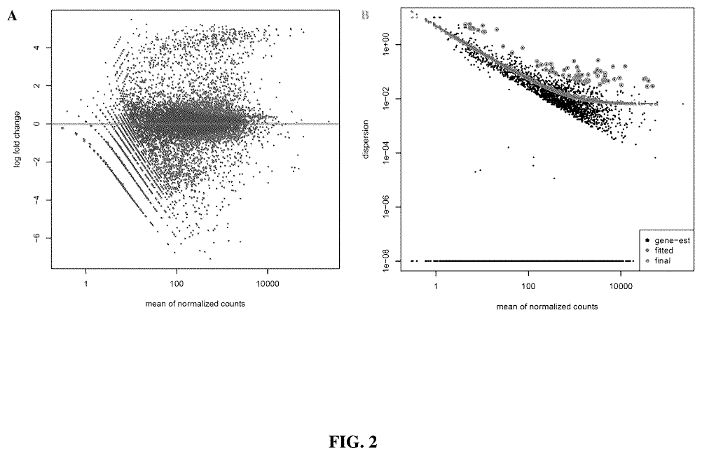

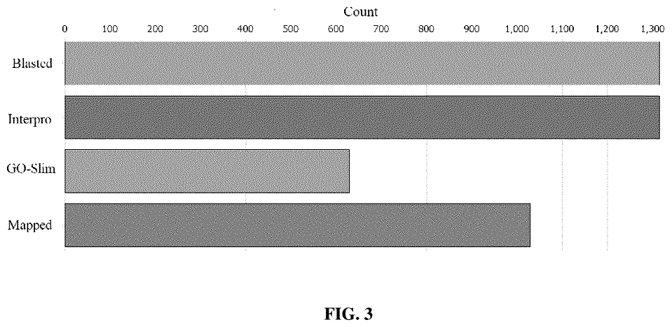

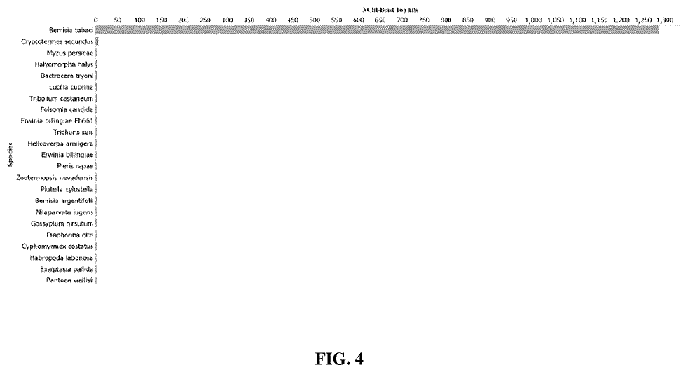

RNAseq analysis to identify genes with enriched expression in the gut and bacteriocytes relative to the whole-body revealed 3178 and 3212 transcripts that are differentially expressed (both up-regulated and down-regulated) in the gut and bacteriocytes respectively. From these, only transcripts with enriched expression (Log FC>1) in the gut (1316 transcripts) and bacteriocyte (1909 transcripts) were selected for further analysis (Table 3, FIG. 1). A considerable number of transcripts had a high fold difference (greater than 3) (FIG. 2, A). A blastx search against the NCBI non-redundant (nr) database was done to identify the selected transcripts enriched in the cassava B. tabaci gut and bacteriocyte. All gut transcripts selected for further analysis (1316 transcripts) had NCBI blast hits (FIG. 3). The species distribution analysis of all blastx top-hits revealed that 97.6% of the blastx top hits belonged to Bemisia tabaci. A few blast top hits belonged to other species which included; Cryptotermes secundus Hill, Myzus persicae Sulzer, Halyomorpha halys Stal and Bactrocera tryoni Froggatt (FIG. 4).

TABLE 3
Summary statistics of RNAseq analysis for genes
expressed in B. tabaci gut and bacteriocytes

| | Comparison | |
|---|---|---|
| | Gut/Whole-body Bacteriocyte/ | |
| Description | Whole-body | |
| Number of total transcripts tested | 10027 | 10092 |
| Number of transcripts with enriched | 3178 | 3212 |
| expression | | |
| Number of transcripts with LogFC > 1 | 1316 | 1909 |
| Number of transcripts with LogFC < −1 | 1862 | 1303 |

In addition, a total of 1316 sequences were successfully scanned against Interpro signatures to identify functional domains, Interpro IDs, gene ontology IDs and names. Gene ontology (GO) mapping of protein blast hits revealed that only 1030 sequences had functional labels. Of these, only 630 sequences had specific GO annotation terms. Based on these functional labels and Interpro IDs, functional osmoregulation genes enriched in the cassava B. tabaci (SSA1-SG1) gut were selected.

Gene Ontology Categorization for the Differentially Expressed Transcripts in B. tabaci Gut

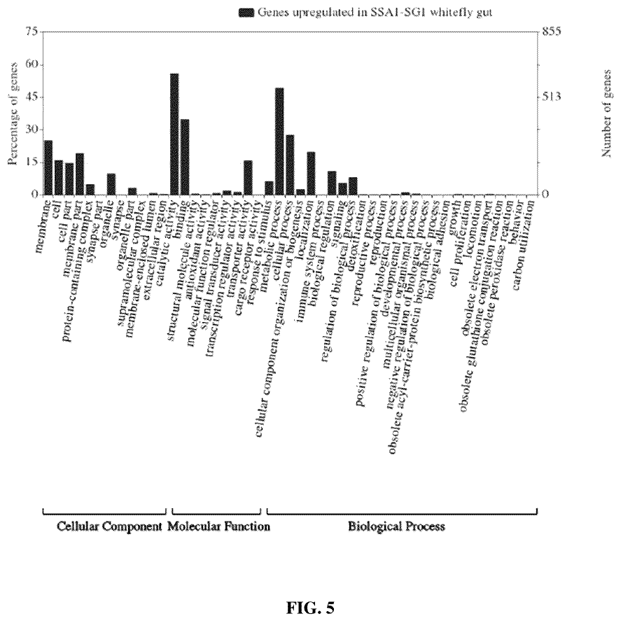

Annotation of differentially expressed transcripts in the cassava B. tabaci (SSA1-SG1) gut revealed roles and localization of these transcripts in terms of GO terms. The transcripts were characterized into three categories (i) cellular component (ii) molecular function and (iii) biological process. Based on the cellular component, the highest number of transcripts were expressed in the membrane (23%) and membrane part (20%). At the molecular level, a high percentage of transcripts (56% and 35%) in the B. tabaci (SSA1-SG1) gut encoded for proteins that mediate catalytic and binding activities respectively. These genes were mostly involved in metabolic and cellular biological processes (FIG. 5). In summary, most of the genes enriched in cassava B. tabaci (SSA1-SG1) gut encode for catalytic, binding and transporter activities especially for metabolic processes and they are more expressed in the membrane and intracellular component of the cassava B. tabaci (SSA1-SG1) gut.

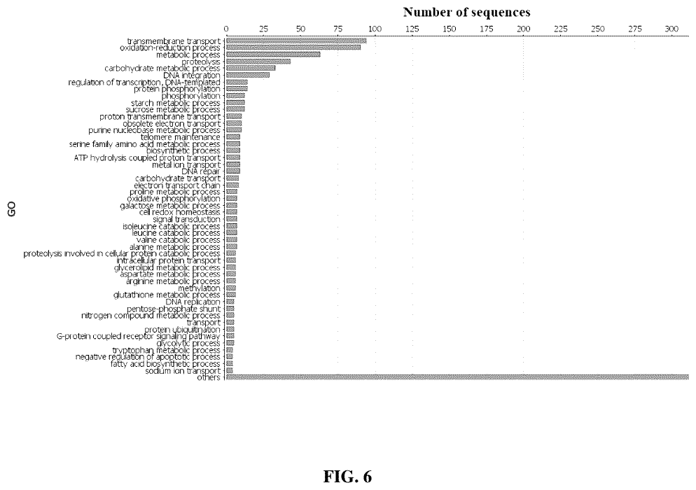

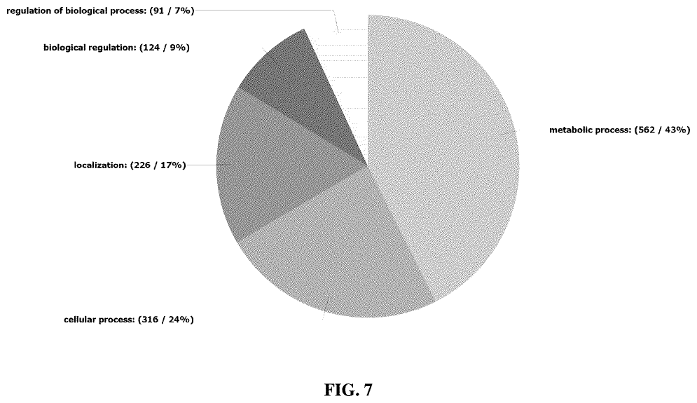

Direct GO count was also done to gain deeper insight into the biological roles of the transcripts enriched the cassava B. tabaci gut. The count showed that the highest number of transcripts encode for transmembrane transport proteins (95 transcripts), oxidation-reduction process (87 transcripts) and metabolic process (62 transcripts). This analysis also revealed a sucrose metabolic process as one of the top 15 biological processes in the cassava B. tabaci (SSA1-SG1) gut (FIG. 6). In summary, a large percentage of transcripts (43%) enriched in the cassava B. tabaci (SSA1-SG1) gut encode for proteins that are involved in metabolic processes (FIG. 6 & FIG. 7).

### Categorization of Transcripts Based on Superfamilies and Functional Domains

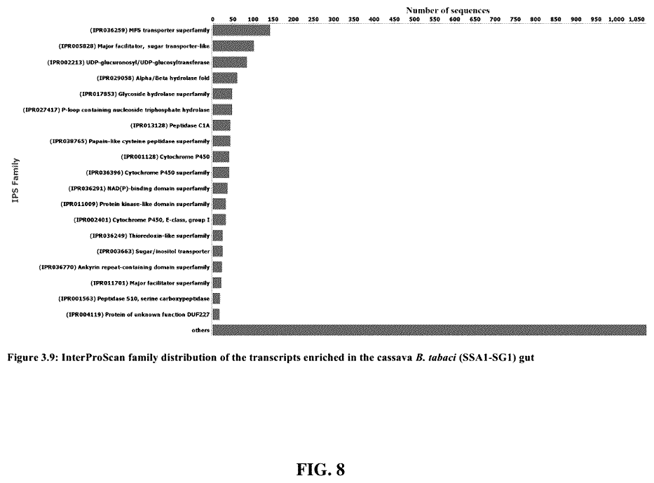

Based on InterProScan IDs of the transcripts enriched in cassava B. tabaci (SSA1-SG1) gut, a total of 19 major superfamilies were characterized. The highest number of transcripts belonged to Major Facilitator Superfamily (MFS) transporter superfamily (140 transcripts) and major facilitator, sugar transporter-like (100 transcripts). Of the top eight superfamilies with the highest abundance of transcripts, three relate to metabolism and in particular, sugar metabolic processes. These include: MFS transporter superfamily (IPR036259), major facilitator-sugar transport like (IPR005828), UDP-glucuronosyl/UDP-glucosyltransferase (IPR002213), Alpha/Beta hydrolase fold (IPR029058) and glycoside hydrolase superfamily (IPR017853) (FIG. 8).

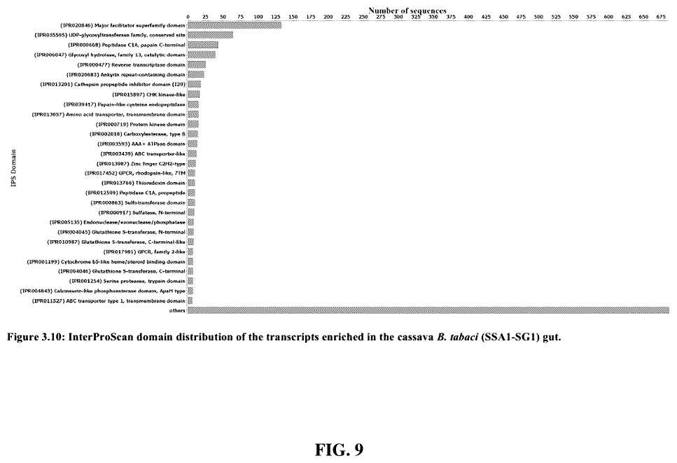

An InterProScan domain distribution analysis for all transcripts enriched in the gut revealed the common and functional domains in the cassava whitefly gut. Based on this analysis, glycosyl hydrolase family 13, catalytic domain (IPR006047), was identified among the top five InterProScan domain for the transcripts enriched in cassava B. tabaci (SSA1-SG1) gut (FIG. 9). Other InterProScan domains that relate to metabolism include; Major facilitator superfamily domain (IPR020846), peptidase CIA, papain C-terminal (IPR000668) and amino acid transporter, transmembrane (IPR013057).

Identification of Genes for Sucrose Metabolism and Water Recycling in Cassava B. tabaci (SSA1-SG1) Gut

Sucrose hydrolysis is mediated by α-glucosidase (EC 3.2.1.20), belonging to glycosyl hydrolase family 13 (IPR006047) (Jing et. al., 2016). Transcripts with glycosyl hydrolase family 13 (IPR006047) catalytic domain were selected for functional analysis. A total of 24 transcripts encoding for sugar processing enzymes belonging to glycosyl hydrolase, family 13 were identified and characterized. These genes had high expression in the B. tabaci gut relative to the whole-body with log 2 Counts Per Million (log CPM) ranging from 1.50-11.86 and fold difference ranging from 4.41-52.71-fold. Of these, gene ENSSSA1UGT025983 (Ssa01347), ENSSSA1UGT002057 (Ssa05154), ENSSSA1UGT024045 (Ssa07143), Ssa12230, ENSSSA1UGT006139 (Ssa02431), ENSSSA1UGT025983 (Ssa01347) and Ssa04510 had the highest expression with log CPM ranging from 10.18-11.86 and fold difference ranging from 17.15-29.65-fold in the B. tabaci gut relative to whole-body (Table 3). These genes corresponded to Bta07453, Bta05386, Bta12682, Bta15649, Bta03439, Bta04298 and Bta14419, orthologs of α-glucosidase gene in MEAM1 B. tabaci. Another gene, ENSSSA1UGT008267 (Ssa04743) had a relatively low expression (log CPM of 1.50) but with the highest fold differences (52.71-fold) in the B. tabaci gut relative to the whole-body. This gene corresponded to Bta06059, an α-glucosidase gene in MEAM1 B. tabaci species.

For water recycling in the gut, three genes encoding for major intrinsic protein (IPR000425) were identified. These were (i) ENSSSA1UGT022145 (Ssa034847), (ii) ENSSSA1UGT021259 (Ssa02238) and (iii) ENSSSA1UGT023163 (Ssa08000) which has been identified through NCBI blastx as AQP1, AQP4 and AQP12 respectively. Genes in parentheses correspond to the gene in the African cassava whitefly genome (Chen, et al., 2019). Of the three major intrinsic proteins, ENSSSA1UGT022145 had the highest expression in cassava B. tabaci (SSA1-SG1) gut with a log CPM of 8.49 and fold difference of 32.67-fold in B. tabaci gut relative to the whole-body (Table 4). This gene corresponded to Bta01973, a water specific AQP1 gene in MEAM1 B. tabaci species.

TABLE 4
Candidate osmoregulation genes selected from differentially expressed genes in cassava B. tabaci (SSA1-SG1) gut

| | | | | | Fold | | |
|---|---|---|---|---|---|---|---|
| | | | Domain | | enrichment | | |
| Gene ID*** | Description | Name | Interpro | logCPM | Gut | P-value | Gene** |
| ENSSSA1UGT025983 | Maltase A3-like | GH-13* | IPR006047 | 11.86 | 25.11 | 0.01 | Bta07453 |
| Ssa12230 | Maltase A3-like | GH-13* | IPR006047 | 11.25 | 17.15 | 0 | Bta15649 |
| ENSSSA1UGT024045 | Maltase 2-like | GH-13* | IPR006047 | 11.23 | 25.11 | 5e−278 | Bta12682 |
| ENSSSA1UGT002057 | Maltase 2-like | GH-13* | IPR006047 | 11.05 | 21.71 | 0.01 | Bta05386 |
| Ssa04510 | Maltase A3-like | GH-13* | IPR006047 | 10.44 | 29.65 | 0.01 | Bta14419 |
| ENSSSA1UGT025983 | Maltase 2-like | GH-13* | IPR006047 | 10.9 | 27.67 | 0 | Bta04298 |
| ENSSSA1UGT006139 | Maltase A3-like | GH-13* | IPR006047 | 10.18 | 24.93 | 4.6e−116 | Bta03439 |
| ENSSSA1UGT009050 | Maltase A3-like | GH-13* | IPR006047 | 9.96 | 23.92 | 1.7e−57 | Bta08425 |
| ENSSSA1UGT002261 | Maltase A3-like | GH-13* | IPR006047 | 9.93 | 23.42 | 1.5e−75 | Bta05396 |
| ENSSSA1UGT002066 | Maltase A3-like | GH-13* | IPR006047 | 9.43 | 25.99 | 6.8e−79 | Bta03818 |
| ENSSSA1UGT013702 | Maltase A3-like | GH-13* | IPR006047 | 9.12 | 20.25 | 0.01 | Bta06458 |
| Ssa04512 | Maltase A3-like | GH-13* | IPR006047 | 9.04 | 17.27 | 4.9e−306 | Bta14422 |
| ENSSSA1UGT001313 | Maltase A3-like | GH-13* | IPR006047 | 8.99 | 11.00 | 0.01 | Bta07452 |
| ENSSSA1UGT028740 | Maltase A3-like | GH-13* | IPR006047 | 8.14 | 30.06 | 1.3e−166 | Bta07377 |
| ENSSSA1UGT021237 | Maltase A3-like | GH-13* | IPR006047 | 7.35 | 25.46 | 0.01 | Bta07764 |
| ENSSSA1UGT006406 | Maltase A3-like | GH-13* | IPR006047 | 7.17 | 7.36 | 0.01 | Bta01478 |
| ENSSSA1UGT005553 | Maltase A3-like | GH-13* | IPR006047 | 7.12 | 22.63 | 0 | Bta08427 |
| ENSSSA1UGT001811 | Maltase A3-like | GH-13* | IPR006047 | 5.07 | 20.25 | 9.0e−192 | Bta09696 |
| ENSSSA1UGT031126 | Maltase A3-like | GH-13* | IPR006047 | 4.74 | 17.27 | 1.8e−146 | Bta10022 |
| ENSSSA1UGT021697 | Maltase | GH-13* | IPR006047 | 4.35 | 28.25 | 3.3e−127 | Bta09633 |
| ENSSSA1UGT000627 | Maltase A3-like | GH-13* | IPR006047 | 3.25 | 4.41 | 1.2e−24 | Bta13914 |
| ENSSSA1UGT003669 | Maltase A3-like | GH-13* | IPR006047 | 2.71 | 8.51 | 3.2e−11 | Bta11358 |
| ENSSSA1UGT010695 | Maltase A3-like | GH-13* | IPR006047 | 2.04 | 28.25 | 1.7e−23 | Bta05340 |
| ENSSSA1UGT008267 | Maltase A3-like | GH-13* | IPR006047 | 1.50 | 52.71 | 1.2e−19 | Bta06059 |
| ENSSSA1UGT022145 | Aquaporin 1 | Major intrinsic | IPR000425 | 8.49 | 32.67 | 0.01 | Bta01973 |
| ENSSSA1UGT021259 | Aquaporin 4 | Major intrinsic | IPR000425 | 5.17 | 2.20 | 3.9e−84 | Bta07505 |
| ENSSSA1UGT023163 | Aquaporin 12 | Aquaporin 11/12 | IPR016697 | 3.31 | 2.07 | 2.2e−08 | Bta14320 |

GH-13* — (Glycosyl hydrolase - Family 13)
Gene** — Ortholog in Bemisia tabaci (MEAM1)
Gene ID*** — (scaffold_xxxxxxF_transcript_id_xxxxx)

### Structural Analysis for Residues of the Hallmarks of the Water-Specific Aquaporins

In addition to sequence identity, the selected sequences for aquaporins were further analyzed to identify hallmarks of the water-specific aquaporins: the aromatic/arginine (ar/R) region and the asparagine-proline-alanine (NPA) boxes in loop B and Loop E. This analysis revealed that gene ENSSSA1UGT022145 had a similar structure of ar/R and NPA as BtAQP1, an experimentally validated water-specific gut aquaporin gene in B. tabaci MEAM1 (Mathew et. al., 2011). Gene ENSSSA1UGT022145 ar/R filter comprised of phenylalanine at position 71, histidine at position 198, alanine at position 207 and arginine at position 213 which is characteristic to water-specific aquaporin. Its NPA had an asparagine at position 91 in loop B and position 210 in loop E (Table 5).

Both ENSSSA1UGT021259 (Ssa02238) and ENSSSA1UGT023163 (Ssa08000) showed variation in their ar/R filter, with ENSSSA1UGT021259 having a leucine at position 196 instead of histidine at position 198 and serine at position 205 instead of alanine at position 207. It also showed differences in its NPA structure with asparagine at position 88 in loop B and at position 208 in loop E. This variation has been reported to be due to a mutation in the ar/R selectivity filter of water-selective channel, forming entomoglyceroporins (Eglps), which are more closely related to the classical aquaporin 4-type channel (Finn et al., 2015). Gene ENSSSA1UGT023163 was truncated, therefore it lacked both ar/R region and NPA boxes.

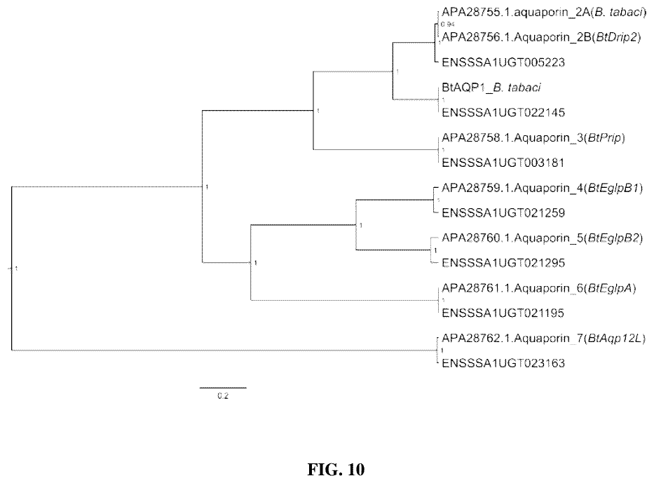

Phylogenetic analysis based on Bayesian approach showed that ENSSSA1UGT022145 clustered with BtAQP1, indicating that ENSSSA1UGT022145 is the water specific AQP1 gene enriched in the cassava B. tabaci (SSA1-SG1) gut (FIG. 10). Both ENSSSA1UGT021259 and ENSSSA1UGT023163 clustered with APA28759.1 and APA28762.1 respectively. This analysis confirmed that ENSSSA1UGT021259 (Ssa02238) is an entomoglyceroporins (AQP4) while ENSSSA1UGT023163 is an aquaporin 7 (BtAQP12L).

TABLE 5
NPA motif and ar/R filter of selected aquaporin protein
sequences of cassava B. tabaci (SSA1-SG1)

| | | | NPA signature motif | |
|---|---|---|---|---|
| Gene ID | Gene | Ar/R Filter | Loop B | Loop E |
| ENSSSA1UGT022145 | Bta01973 | F (71) H (198) A (207) R (213) | NPA (91) | NPA (210) |
| ENSSSA1UGT021259 | Bta07505 | F (68) L (196) S (205) R (211) | NPA (88) | NPA (208) |
| ENSSSA1UGT023163 | Bta14320 | — | — | — |
| BtAQP1* | | F (71) H (198) A (207) R (213) | NPA (91) | NPA (210) |

*Experimentally validated water-specific gut aquaporin gene of B. tabaci (Mathew, et. al., 2011)

The number in parenthesis is the position of the respective amino acid in Ar/R filter and asparagine of the NPA motif for each aquaporin protein sequence
Identification of α-Glucosidase Proteins with Both the Catalytic Residue and Predicted Signal Peptide

A total of 24 protein sequences belonging to glycosyl hydrolase family 13 were identified using sequence similarity methods. Analysis of amino acid residues was done to identify protein sequences with both predicted signal peptide and catalytic site residue, characteristic to α-glucosidase of GH-13. Of the 24 α-glucosidase protein sequences selected, only 14 had both a predicted signal peptide and catalytic site residue. These included: (i) Ssa04510 (Bta14419), (ii) ENSSSA1UGT005553 (Bta08427), (iii) ENSSSA1UGT002057 (Bta05386), (iv) ENSSSA1UGT010695 (Bta05340), (v) ENSSSA1UGT021697 (Bta09633), (vi) ENSSSA1UGT008267 (Bta06059), (vii) ENSSSA1UGT000627 (Bta13914), (viii) ENSSSA1UGT021237 (Bta07764), (ix) ENSSSA1UGT002066 (Bta03818), (x) Ssa12230 (Bta15649), (xi) ENSSSA1UGT006139 (Bta03439), (xii) Ssa04512 (Bta14422), (xiii) ENSSSA1UGT013702 (Bta06458) and (xiv) ENSSSA1UGT009050 (Bta08425). Genes in parentheses represent a corresponding ortholog in B. tabaci MEAM1.

All these protein sequences had aspartic acid as the nucleophile and glutamic acid as the proton donor located at varying positions in each protein sequence except for one sequence ENSSSA1UGT013702 (Bta06458) which had glutamic acid at position 229 and arginine as a proton donor at position 287 (Table 6).

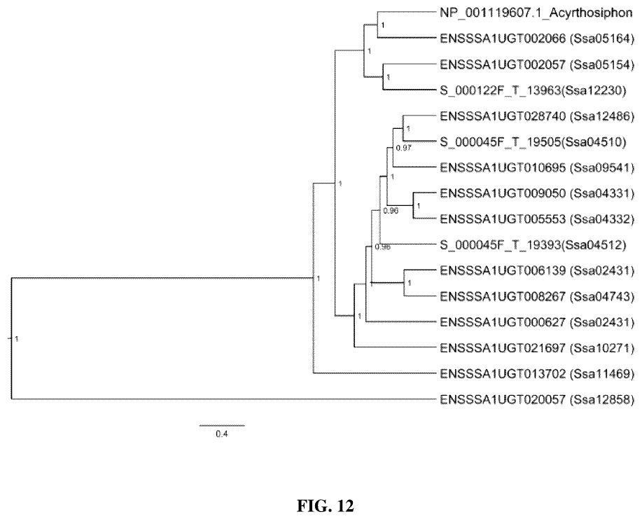

The phylogenetic analysis for the selected protein sequences with both predicted signal peptide and catalytic site residue also revealed three clusters (FIG. 12), which may relate to the substrate (sucrose, maltose and other sugars) specificity of these enzymes. A total of three genes clustered with an experimentally validated sucrase gene (Price et al., 2007). These included; Ssa12230, ENSSSA1UGT002057 and ENSSSA1UGT002066. All these had a very high level of expression with fold difference ranging between 17.15-25.99 in the cassava B. tabaci (SSA1-SG1) gut relative to whole-body. Based on NCBI conserved protein domain family, the three sequences; Ssa12230, ENSSSA1UGT002057 (Ssa05154) and ENSSSA1UGT002066 (Ssa05164) along with the experimentally validated sucrase had an AmyAc_Maltase (cd11328) catalytic domain. These are potential α-glucosidase genes in cassava B. tabaci (SSA1-SG1) that hydrolyze sucrose to glucose and fructose.

Identification of Sucrose Hydrolase in Cassava B. tabaci (SSA1-SG1) Gut

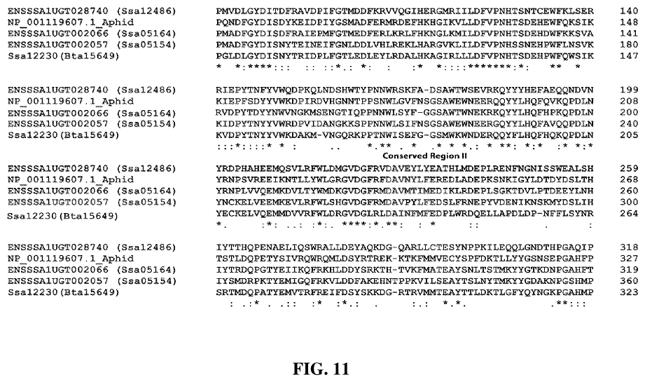

All selected protein sequences had variation in seven amino acid residues of the partial sequence of the conserved region II of α-glucosidase family 13. A conserved region II is a region on the protein sequence where the catalytic site of alpha-glucosidase begins. The protein sequences analyzed exhibited a sequence motif DAxxxxx except for ENSSSA1UGT013702 (Ssa11469) which had a different sequence motif ETxxxxx. Partial sequences of conserved region II of all the selected protein sequences were analyzed and compared with that of the experimentally validated sucrase to identify the enzyme that specifically hydrolyzes sucrose in the whitefly gut. Only one protein sequence ENSSSA1UGT002057 (Ssa05154) had the closest configuration of the seven amino acid (DAVPYLF; SEQ ID NO:49) to that of the experimentally validated sucrase/transglucosidase of the pea aphid (DAVNYLF; SEQ ID NO:57) (Price et al., 2007) (Table 6, FIG. 11).

TABLE 6
Genes encoding proteins with characteristic catalytic site residue and a signal peptide for α-glucosidase enzyme

| | | | | | | | Catalytic site | | |
|---|---|---|---|---|---|---|---|---|---|
| | | | | | | | residue*** | | |
| | | Fold | Signal | D- | Cleavage | | Proton | Conserved | SEQ ID |
| Gene ID | Gene* | difference | peptide | Score** | site | Nucleophile | Donor | region II | NO. |
| ENSSSA1UGT008267 | Bta06059 | 52.71 | + | 0.760 | A(19)F | D(224) | E(289) | DAPGWLM | 35 |
| ENSSSA1UGT028740 | Bta07377 | 30.06 | + | 0.768 | S(20)R | D(227) | E(295) | DAVEYLY | 36 |
| Ssa04510 | Bta14419 | 29.65 | + | 0.474 | S(20)H | D(230) | E(297) | DAVNHLL | 37 |
| ENSSSA1UGT010695 | Bta05340 | 28.25 | + | 0.739 | G(21)H | D(183) | E(250) | DAARHFF | 38 |
| ENSSSA1UGT021697 | Bta09633 | 28.25 | + | 0.774 | C(20)N | D(227) | E(295) | DAPEFIF | 39 |
| ENSSSA1UGT025983* | Bta04298 | 27.67 | − | 0.195 | — | D(19) | E(86) | DAVTYMY | 40 |
| ENSSSA1UGT002066 | Bta03818 | 25.99 | + | 0.843 | C(16)R | D(228) | E(296) | DAVMTIM | 41 |
| ENSSSA1UGT021237 | Bta07764 | 25.46 | + | 0.512 | E(20)E | D(257) | E(289) | DAVQILF | 42 |
| ENSSSA1UGT025983* | Bta07453 | 25.11 | − | 0.103 | — | D(65) | E(132) | DAVTYMY | 43 |
| ENSSSA1UGT024045 | Bta12682 | 25.11 | − | 0.119 | — | D(288) | E(344) | DAVPHLI | 44 |
| ENSSSA1UGT006139 | Bta03439 | 24.93 | + | 0.833 | A(22)Q | D(227) | E(232) | DAAKWLF | 45 |
| ENSSSA1UGT009050 | Bta08425 | 23.92 | + | 0.825 | G(20)I | D(231) | E(290) | DAVPWLY | 46 |
| ENSSSA1UGT002261 | Bta05396 | 23.43 | − | 0.329 | — | D(244) | E(312) | DAIKHLV | 47 |
| ENSSSA1UGT005553 | Bta08427 | 22.63 | + | 0.860 | G(20)V | D(227) | E(286) | DAVVCLY | 48 |
| ENSSSA1UGT002057 | Bta05386 | 21.71 | + | 0.743 | A(24)V | D(268) | E(337) | DAVPYLF | 49 |
| ENSSSA1UGT013702 | Bta06458 | 20.25 | + | 0.765 | G(20)G | E(229) | R(287) | ETVSYLF | 50 |
| ENSSSA1UGT001811 | Bta09696 | 20.25 | + | 0.742 | A(25)Q | — | — | — | |
| Ssa04512 | Bta14422 | 17.27 | + | 0.799 | Q(23)S | D(228) | E(296) | DAIKHVY | 51 |
| ENSSSA1UGT031126* | Bta10022 | 17.27 | − | 0.147 | — | — | — | — | |
| Ssa12230 | Bta15649 | 17.15 | + | 0.693 | E(21)F | D(233) | E(300) | DAINFMF | 52 |
| ENSSSA1UGT001313 | Bta07452 | 11.00 | − | 0.428 | — | D(227) | E(294) | DAVAYLF | 53 |
| ENSSSA1UGT003669* | Bta11358 | 8.51 | + | 0.810 | A(20)D | — | — | — | 54 |
| ENSSSA1UGT006406 | Bta01478 | 7.36 | − | 0.105 | — | D(281) | E(369) | DAVHTMF | 55 |
| ENSSSA1UGT000627 | Bta13914 | 4.41 | + | 0.805 | S(17)T | D(220) | E(288) | DAIPILF | 56 |
| NP_001119607.1_Aphid | — | — | + | 0.781 | S(21)E | D(236) | E(304) | DAVNYLF | 57 |

* Ortholog in Bemisia tabaci (MEAM1)
**Discrimination score (D-score) for a best-predicted signal peptide for predicted proteins of GH13 family
***Catalytic site residue in each GH13 sequence. The position of the Nucleophile residue of the partial sequence of conserved region II is indicated by the number in the parentheses.
Presence or lack of signal peptide denoted as + or − respectively

Identification of Symbiosis Genes in Cassava B. tabaci (SSA1-SG1)

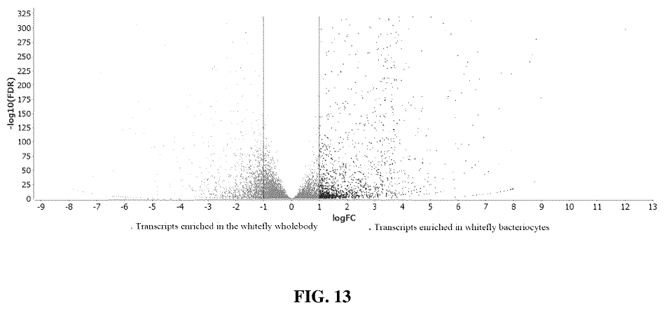

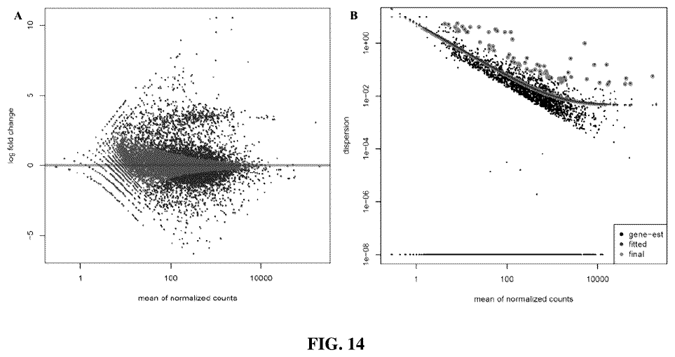

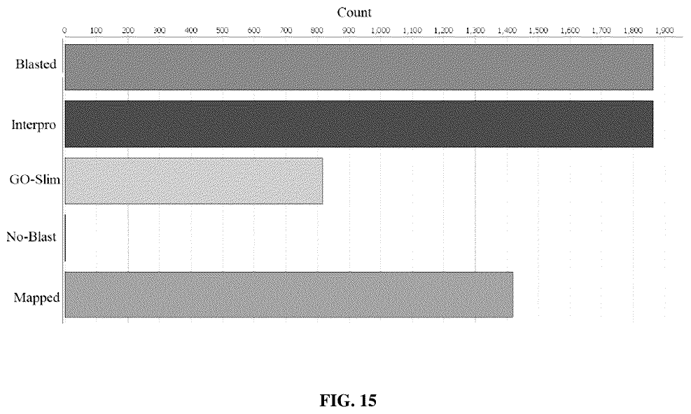

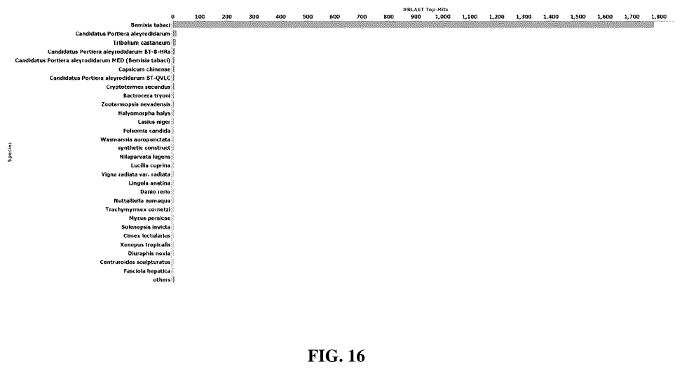

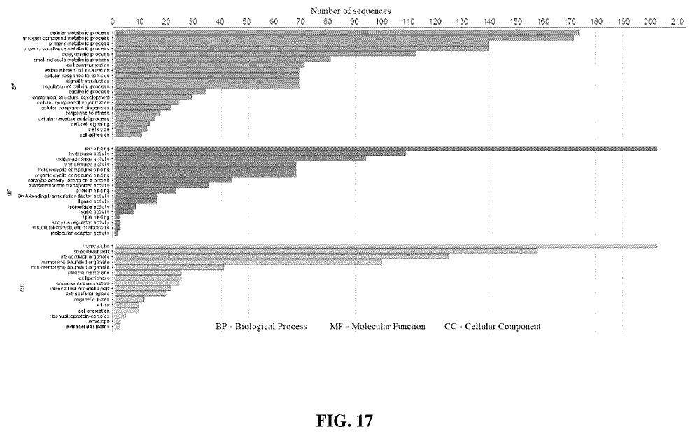

In B. tabaci, biosynthesis of essential amino acids requires interaction between the host and endosymbionts in the bacteriocyte. RNAseq analysis was conducted to identify critical genes that facilitate the shared metabolic interaction in B. tabaci SSA1-SG1. A total of 3212 transcripts were differentially expressed in both bacteriocyte and cassava B. tabaci (SSA1-SG1) whole-body (Table 3). Of these, a total of 1909 transcripts that are enriched in the cassava B. tabaci (SSA1-SG1) bacteriocyte were selected for further analysis. Both volcano and MA-plot showed a wide dispersion showing a high level of differences in gene expression between bacteriocytes and whole-body samples (FIG. 13, FIG. 14). Blastx search against NCBI non-redundant (nr) database was done to identify the transcripts enriched in the cassava B. tabaci (SSA1-SG1) bacteriocyte. Only one sequence did not have NCBI-blast hit (FIG. 15). Top-hit species distribution analysis revealed that a large percentage (93%) of the top hits belonged to Bemisia tabaci (FIG. 16). A small number of hits belonged to Candidatus Portiera aleyrodidarum, Candidatus P. aleyrodidarum BT-B-HRs, Candidatus P. aleyrodidarum MED and Candidatus P. aleyrodidarum BT-QVLC, resident endosymbiont in bacteriocytes of different B. tabaci species. Furthermore, the analysis was done to identify the functional signatures of the transcripts that are enriched in the B. tabaci bacteriocyte. A total of 1909 sequences were successfully scanned against Interpro signatures to identify and characterize functional domains and GO terms. The blasted protein sequences with hits were then mapped against extensively curated gene ontology annotated proteins. Of the 1909 Interpro scanned transcripts, only 1419 had functional labels and 817 had specific GO annotation terms at an annotation score cutoff of 55 (FIG. 17).

Gene Ontology Categorization for the Differentially Expressed Transcripts in Cassava B. tabaci (SSA1-SG1) Bacteriocyte

Gene ontology classification for transcripts enriched in cassava B. tabaci (SSA1-SG1) bacteriocyte was based on three aspects: molecular level activities performed by gene products (molecular function), biological processes and the location relative to cellular structure (cellular component). Based on the cellular component, many transcripts were expressed in the intracellular component (204 transcripts), intracellular part (158 transcripts), intracellular organelle (125 transcripts) and membrane-bounded organelle (100 transcripts) (FIG. 17). At the molecular level, most of the transcripts enriched in the B. tabaci bacteriocyte encoded for ion binding (203 transcripts), hydrolase activity (109 transcripts), oxidoreductase activity (94 transcripts) and transferase activity (68 transcripts). Several transcripts also showed molecular signatures for transmembrane transporter activities, catalytic activity-acting on protein and protein binding. These are important functions for amino acid biosynthesis and host-symbiont interaction in the bacteriocyte. Gene ontology for biological process showed that the largest percentage of transcripts are involved in metabolic processes (organic substance metabolic process, primary metabolic process, cellular metabolic process, nitrogen compound metabolic process). Other biological processes that relate to metabolism include regulation of cellular process, biosynthetic process, small molecule metabolism and regulation of metabolic process.

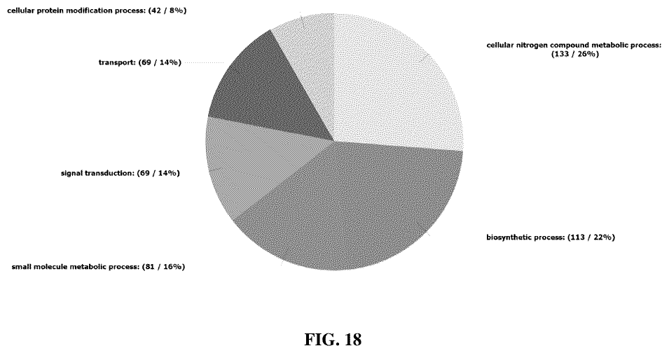

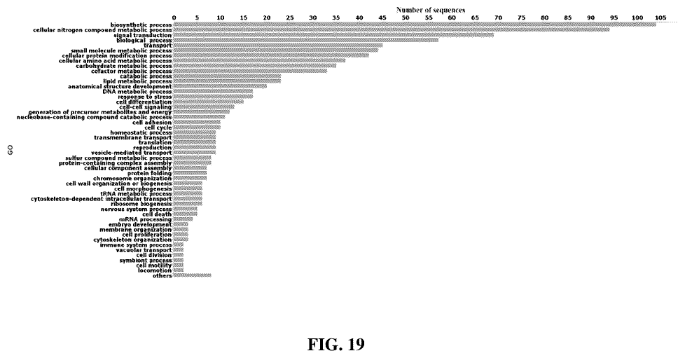

Biological processes were further categorized based on sequence distribution of the transcript that are enriched in cassava B. tabaci bacteriocyte. This categorization revealed that the top five biological processes in cassava B. tabaci (SSA1-SG1) bacteriocyte relate to protein metabolism (amino acid biosynthesis). These include cellular nitrogen compound metabolic process (26%), biosynthetic process (22%), transport (14%) and cellular protein modification process (8%) (FIG. 18). In addition, direct gene ontology count for the transcripts enriched in cassava B. tabaci bacteriocyte also showed several transcripts related to protein metabolic processes which include: biosynthetic process (104 transcripts), cellular nitrogen compound metabolic process (94 transcripts), cellular protein modification process (42 transcripts) and cellular amino acid metabolic process (37 transcripts) (FIG. 19).

Identification of Potential Symbiosis Genes Targets in Cassava B. tabaci (SSA1-SG1)

Transcripts enriched in B. tabaci SSA1-SG1 bacteriocyte were analyzed to identify proteins that facilitate essential amino acid biosynthesis, as potential symbiont gene targets. These included (i) aminotransferases, (ii) amino acid transporters, (iii) horizontally transferred genes and (iv) receptors of intermediate metabolites for essential amino acid biosynthesis across the symbiosome membrane, a special membrane that separates the endosymbiont and host in the bacteriocyte. A total of 20 genes encoding for amino acid transporters and aminotransferases were identified and selected. Amino acid transporters belonged to different families which included (i) Amino acid/polyamine transporter 1 (IPR002293), (ii) cationic amino acid transporter, C-terminal (IPR029485), (iii) proton-dependent oligopeptide transporter (IPR000109) and (iv) sodium: dicarboxylate symporter (IPR001991). Amino acid transferases belonged to (i) kynurenine—oxoglutarate transaminase 3, KAT_III (IPR034612), (ii) aminotransferase class 4 (IPR001544) and (iii) aminotransferase class V domain (IPR00192) (Table 7).

Genes (ENSSSA1UGT021004 (Ssa04473) and ENSSSA1UGT023671 (Ssa10714)) belonging to aminotransferase family had the highest gene expression among the selected genes in cassava B. tabaci bacteriocyte. These were identified as branched-chain-amino acid aminotransferase and kynurenine—oxoglutarate transaminase 3 with an expression of log (base 2) Counts Per Million (log CPM) of 8.89 and 6.79 and fold difference of 8.41 and 4.75-fold in bacteriocytes relative to the whole-body respectively. These two genes corresponded to Bta10673 and Bta05157 respectively, two aminotransferase orthologs in MEAM1 B. tabaci. Other aminotransferase included phosphoserine aminotransferase (ENSSSA1UGT011663 (Ssa14433)), which showed a relatively higher gene expression (log CPM of 7.30 and fold difference of 6.16) in the bacteriocyte compared to other selected genes.

Among the amino acid transporters, the highest number of potential gene targets (6 genes) belonged to amino acid transporter, transmembrane domain (IPR013057). Six targets belonging to amino acid/auxin permease family were also identified as amino acid permeases, amino acid transporters more specialized in transporting cation amino acids (lysine, arginine, leucine, methionine and glutamine) from the cell to the extracellular matrix. These include, cationic amino acid transporter 3 (ENSSSA1UGT002031), high affinity cationic AA transporter 1 (ENSSSA1UGT020942 and ENSSSA1UGT030948) and y⁺L amino acid transporter 2 (ENSSSA1UGT020386, ENSSSA1UGT011941 and ENSSSA1UGT019654). Of these, a y⁺L amino acid transporter 2, ENSSSA1UGT011941 (Ssa06643) had a relatively high expression with a log CPM of 7.39 and fold difference of 8.59-fold relative to whole-body. This gene corresponded to Bta11657, a y+L amino acid transporter 2 gene in MEAM1 B. tabaci.

Another set of amino acid transporters identified contained an amino acid transporter, transmembrane domain (Aa_trans, IPRO13057), which is unique for the amino acid polyamine organocation superfamily. These amino acids included (i) transmembrane protein 104 homolog (ii) three, proton-coupled amino acid transporter 1, (iii) proton-coupled amino acid transporter 4 and (iv) sodium-coupled neutral AA transporter 9 (Table 7). Other amino acid transporters identified included excitatory amino acid transporter 1 and peptide transporter family 1 belonging to proton-dependent oligopeptide transporter family.

In summary, based on the level of gene expression in the cassava B. tabaci bacteriocyte, eight genes (ENSSSA1UGT011663, ENSSSA1UGT021004, ENSSSA1UGT017527, ENSSSA1UGT023671, ENSSSA1UGT011941, ENSSSA1UGT030948, ENSSSA1UGT002031 and ENSSSA1UGT011431) were selected as targets whose disruption may cause significant effects on growth and development of cassava B. tabaci (SSA1-SG1).

TABLE 7
Potential gene targets selected from differentially expressed genes in cassava B. tabaci bacteriocytes

| | NCBI-BLAST | Domain | | | Fold | | |
|---|---|---|---|---|---|---|---|
| Gene ID | Hit | Name | Interpro | LogCPM | differences | P-value | Gene** |
| ENSSSA1UGT021004 | Branched-chain-amino-acid | Aminotrans_4 | IPR001544 | 8.89 | 6.79 | 0 | Bta10673 |
| | aminotransferase | | | | | | |
| ENSSSA1UGT023671 | Kynurenine--oxoglutarate | KAT_III | IPR034612 | 8.41 | 4.75 | 0 | Bta05157 |
| | transaminase 3 | | | | | | |
| ENSSSA1UGT011663 | Phosphoserine | Aminotran_5 | IPR000192 | 7.30 | 6.16 | 3.7e−85 | Bta04363 |
| | aminotransferase | | | | | | |
| ENSSSA1UGT011941 | y⁺L amino acid transporter 2 | AA_Permease_2 | IPR002293 | 7.39 | 8.59 | 0 | Bta11657 |
| ENSSSA1UGT030948 | High affinity cationic AA | AA_Permease_2 | IPR002293 | 6.57 | 3.19 | 1.01 | Bta01499 |
| | transporter 1 | | | | | | |
| ENSSSA1UGT002031 | Cationic amino acid | AA_Permease_C | IPR029485 | 6.36 | 4.07 | 1.0e−105 | Bta11613 |
| | transporter 3 | | | | | | |
| ENSSSA1UGT019654 | y⁺L amino acid transporter 2 | AA_Permease_2 | IPR002293 | 5.98 | 3.21 | 3.5e−258 | Bta02775 |
| ENSSSA1UGT020386 | y⁺L amino acid transporter 2 | AA_Permease_2 | IPR002293 | 5.96 | 5.24 | 0 | Bta08771 |
| ENSSSA1UGT020942 | High affinity cationic AA | AA_Permease_2 | IPR002293 | 5.18 | 12.25 | 0 | Bta13409 |
| | transporter 1 | | | | | | |
| ENSSSA1UGT011431 | Transmembrane protein 104 | Aa_trans | IPR013057 | 7.41 | 5.32 | 0 | Bta03456 |
| | homolog | | | | | | |
| ENSSSA1UGT000759 | Proton-coupled amino acid | Aa_trans | IPR013057 | 4.83 | 4.49 | 1.4e−202 | Bta08775 |
| | transporter 1 | | | | | | |
| ENSSSA1UGT001543 | Proton-coupled amino acid | Aa_trans | IPR013057 | 4.74 | 3.25 | 5.4e−127 | Bta08693 |
| | transporter 4 | | | | | | |
| ENSSSA1UGT024971 | Proton-coupled amino acid | Aa_trans | IPR013057 | 4.88 | 4.78 | 1.3e−222 | Bta01726 |
| | transporter 1 | | | | | | |
| ENSSSA1UGT000813 | Sodium-coupled neutral AA | Aa_trans | IPR013057 | 2.66 | 2.47 | 7.8e−21 | Bta11166 |
| | transporter 9 | | | | | | |
| ENSSSA1UGT025011 | Proton-coupled amino acid | Aa_trans | IPR013057 | 0.61 | 3.62 | 9.6e−08 | Bta01718 |
| | transporter 1 | | | | | | |
| ENSSSA1UGT017527 | vesicular glutamate | Major | IPR020846 | 6.95 | 11.97 | 0 | Bta06520 |
| | transporter 3 | facilitator | | | | | |
| ENSSSA1UGT008430 | Neutral and basic amino | GH 13 | IPR006589 | 6.26 | 2.92 | 2.2e−246 | Bta10614 |
| | acid transport protein rBAT | | | | | | |
| ENSSSA1UGT021057 | excitatory amino acid | PTR2 | IPR000109 | 5.02 | 7.09 | 4.3e−317 | Bta11487 |
| | transporter 1 | | | | | | |
| ENSSSA1UGT023619 | peptide transporter family 1 | PTR2 | IPR000109 | 3.40 | 8.99 | 0 | Bta04879 |
| ENSSSA1UGT012586 | Na-dependent excitatory | SDF | IPR001991 | 2.01 | 4.02 | 5.4e−28 | Bta09439 |
| | AA transporter glt-6 | | | | | | |

**ortholog in Bemisia tabaci (MEAM1)

Horizontally Transferred Genes of Bacterial Origin Expressed in Cassava (SSA1-SG1) B. tabaci Species

Previous studies by Luan et. al. (2015) showed that horizontally transferred genes (HTGs) are important for essential amino acid biosynthesis in B. tabaci. Candidate based search of HTGs previously identified in MEAM1 B. tabaci species (Chen et al., 2016) was done to identify HTGs expressed in cassava (SSA1-SG1) B. tabaci bacteriocytes. A reciprocal blast revealed a total of 43 HTGs of bacterial origin in SSA1-SG1 bacteriocytes. These genes had varying levels of gene expression in the bacteriocytes relative to the whole-body ranging from log CPM of −0.42-11.16 and fold difference ranging from −4.73-13.89-fold.

HTGs contributing to lysine, arginine and proline biosynthesis in the bacteriocytes had relatively high gene expression compared to other genes identified. These included (i) 20G-Fc (II) oxygenase (log CPM 11.16, 1.00-fold), (ii) 4-hydroxy-tetrahydrodipicolinate reductase (log CPM 7.88, 5.45-fold), (iii) allophanate hydrolase (log CPM 7.04, 1.13-fold), (iv) diaminopimelate decarboxylase (LysA) (log CPM 6.89. 13.89 fold), (v) arginosuccinate lyase (ArgH) (log CPM 6.68, 1.79 fold) and (vi) arginosuccinate synthase (ArgG) (log CPM 5.95, 2.10 fold). These genes corresponded to Bta20012, Bta20020, Bta13949, Bta03593, Bta00063 and Bta00062, all HTGs in MEAM1 B. tabaci species (Table 8). Other genes involved in amino acid biosynthesis in the bacteriocyte include (i) an Acetyltransferase, (ii) amidinotransferase and (iii) histidine ammonia-lyase (Table 7).

HTGs that are important in vitamin B (biotin) biosynthesis pathway also exhibited a relatively high gene expression in B. tabaci bacteriocytes. These included (i) adenosylmethionine—8-amino-7-oxononanoate aminotransferase (bioA) (log CPM 6.69, 7.11 fold), (ii) ATP-dependent dethiobiotin synthetase (bioD) (log CPM 6.69, 7.11-fold), (iii) adenosylmethionine-8-amino-7-oxononanoate aminotransferase (bioA) (log CPM 5.16, 1.50 fold), (iv) biotin synthase (bioB) (log CPM 5.21, 1.41-fold) and (v) ATP-dependent dethiobiotin synthetase (bioD) (log CPM 5.16, −1.50-fold), corresponding to Bta01937, Bta1938, Bta00841, Bta09725 and Bta00840, HTGs in MEAM1. SSA1-SG1 contained two copies of bioA and bioD like what was reported in MEAM1 (Chen et al., 2016). Gene 000024F_arrow: 4385697-4418725 (bioA) has a lower expression (1.50 compared to 7.11-fold of the second bioA) in the bacteriocyte. This gene corresponded to Bta00841, a truncated (Pseudogene) in MEAM1. For bioD, Bta01938 was reported as a pseudogene in MEAM1, however the corresponding gene in SSA1-SG1, ENSSSA1UGT012203 (Ssa01088) had a high expression (log CPM 6.69, 7.11-fold) in the bacteriocyte. Other HTGs with enriched gene expression in bacteriocytes and contributing to various pathways were cyclopropane-fatty-acyl-phospholipid synthase (log CPM 9.57, 11.07-fold), crossover junction endodeoxyribonuclease rusA (log CPM 7.89, 1.36-fold), BH1803 protein (log CPM 7.75, 1.54-fold), leucine rich repeat containing proteins (log CPM 7.75, 1.54 fold), ribosome recycling factor (log CPM 6.48, 10.77-fold) and L-galactonate dehydratase (log CPM 5.49, 1.9-fold).

A search for possible sources of all detected HTGs revealed several bacterial genera which included: Rickettsia, Planctomycetes, Klebsiella, Pantoea, Curtobacterium, Pseudomonas, Rahnella, Wolbachia, Cardinium, Photorhabdus, Bacillus, Sodalis, Cronobacter, Rhizobium, Methylobacterium, Parabarkholderia, Burholderia, Pectobacterium and Mucilaginibacter.

In addition, a total of 38 HTGs of fungal origin were also identified in the SSA1-SG1 B. tabaci bacteriocyte sequences. These genes had lower expression in the bacteriocytes compared to HTGs of bacterial origin identified in this study. Of the 38 genes identified, only four genes had enriched expression within the SSA1-SG1 bacteriocytes. These encoded for gamma-glutamyltranspeptidase (log CPM 3.02, 3.80-fold), aromatic peroxygenase (log CPM 7.17, 7.42-fold), major royal jelly-related protein (log CPM 4.73, 2.09-fold) and squalene synthase (log CPM 6.43, 2.20-fold) (Table 9). Function analysis revealed that gene, ENSSSA1UGT000931 (Ssa05748) encoding for gamma-glutamyltranspeptidase could be a potential target. This is because gamma-glutamyltranspeptidase has been reported to be key in the transfer of amino acids across cellular membranes (Taniguchi and Ikeda, 1998).

TABLE 8
Horizontally transferred genes of bacterial origin expressed in cassava B. tabaci (SSA1-SG1) bacteriocytes

| | | | Gene expression | | Possible origin: | |
|---|---|---|---|---|---|---|
| | | | | Fold | Bacteria | |
| Gene ID | Description | Pathway | LogCPM | difference | Genus | Gene*** |
| ENSSSA1UGT010158 | 20G-Fe (II) oxygenase | Arg, Proline | 11.16 | 1.00 | Pantoea | Bta20012 |
| ENSSSA1UGT008257 | 4-hydroxy-tetrahydrodipicolinate | L-lysine | 7.88 | 5.45 | Rickettsia | Bta20020 |
| | reductase dapB | | | | | |
| ENSSSA1UGT007134 | Diaminopimelate decarboxylase | L-lysine | 6.89 | 13.89 | Planctomyces | Bta03593 |
| | (LysA) | | | | | |
| ENSSSA1UGT012203 | ATP-dependent dethiobiotin | Biotin | 6.69 | 7.11 | Wolbachia | Bta01938 |
| | synthetase (BioD) | | | | | |
| ENSSSA1UGT003467 | Arginosuccinate lyase (ArgH) | L-arginine | 6.68 | 1.79 | Erwinia | Bta00063 |
| ENSSSA1UGT016543 | Ribosome recycling factor | | 6.48 | 10.77 | Not well defined | Bta15019 |
| ENSSSA1UGT003597 | Arginosuccinate synthase (ArgG) | L-arginine | 5.95 | 2.10 | Pantoea/Erwinia | Bta00062 |
| ENSSSA1UGT003262 | Phenazine biosynthesis-like | | 5.45 | −1.51 | Pseudomonas | Bta02987 |
| | domain-containing protein | | | | | |
| ENSSSA1UGT031701 | Biotin synthase (BioB) | Biotin | 5.21 | 1.41 | Not well defined | Bta09725 |
| ENSSSA1UGT012203 | ATP-dependent dethiobiotin | Biotin | 5.16 | −1.50 | Wolbachia | Bta00840 |
| | synthetase (BioD) | | | | | |
| ENSSSA1UGT025651 | Chorismate mutase (CM) | shikimate | 5.01 | 2.57 | Rahnella | Bta15103 |
| ENSSSA1UGT004633 | Leucine-rich repeat containing | | 4.71 | −1.87 | Rickettsia | Bta13776 |
| | proteins | | | | | |
| ENSSSA1UGT009294 | SCP-like extracellular protein | | 4.27 | 1.32 | Bacillus | Bta15713 |
| ENSSSA1UGT017373 | ATP-dependent DNA helicase Q- | | 4.04 | 1.24 | Photorhabdus | Bta00002 |
| | like 3 | | | | | |
| ENSSSA1UGT006636 | Squalene-hopene cyclase | | 3.77 | −4.73 | Burkholderia | Bta02625 |
| ENSSSA1UGT028316 | Squalene-hopene cyclase | | 3.72 | 1.05 | Paraburkholderia | Bta07125 |
| ENSSSA1UGT008515 | Ribosomal RNA subunit | | 3.65 | 8.17 | Methylobacterium | Bta03791 |
| | methyltransferase A | | | | | |
| ENSSSA1UGT002124 | 3-methyl-2-oxobutanoate | Pantothenate | 3.57 | 30.50 | Pseudomonas | Bta05339 |
| | hydroxymethyltransferase | | | | | |
| ENSSSA1UGT016889 | Tryptophan-tRNA ligase | | 3.32 | −1.31 | Pectobacterium | Bta06820 |
| ENSSSA1UGT001349 | Ribonuclease H | | 3.04 | 6.12 | Wolbachiae | Bta09186 |
| ENSSSA1UGT005350 | Urea amidolyase | | 2.86 | −2.15 | Pseudomonas | Bta04508 |
| ENSSSA1UGT018122 | Squalene-hopene cyclase | | 0.22 | 2.96 | Paraburkholderia | Bta07024 |
| ENSSSA1UGT013675 | Diaminopimelate epimerase (DapF) | L-lysine | 0.13 | 1.57 | Klebsiella | Bta06657 |
| Ssa09979 | Cyclopropane-fatty-acyl- | | 9.57 | 11.07 | Cronobacter | Bta06818 |
| | phospholipid synthase | | | | | |
| Ssa10244 | Crossover junction | | 7.89 | 1.36 | Sodalis | Bta03871 |
| | endodeoxyribonuclease rusA | | | | | |
| Ssa12239 | Leucine-rich repeat containing | | 7.06 | 1.15 | Rickettsia | Bta13226 |
| | proteins | | | | | |
| Ssa03692 | Allophanate hydrolase | L-arginine | 7.04 | 1.13 | Pantoea | Bta13949 |
| Ssa01553 | Adenosylmethionine--8-amino-7- | Biotin | 6.69 | 7.11 | Cardinium | Bta01937 |
| | oxononanoate aminotransferase (BioA) | | | | | |
| Ssa09927 | Methyltransferase | | 6.31 | 1.11 | Pseudomonas | Bta20014 |
| Ssa08027 | 4,5-DOPA dioxygenase extradiol | Betalain | 6.27 | −1.42 | Pantoea | Bta02812 |
| Ssa03128 | L-galactonate dehydratase | | 5.49 | 1.39 | Pseudomonas | Bta03200 |
| Ssa12718 | Cyclopropane-fatty-acyl- | | 5.36 | 1.08 | Cronobacter | Bta11912 |
| | phospholipid synthase | | | | | |
| Ssa01553 | adenosylmethionine--8-amino-7- | Biotin | 5.16 | 1.50 | Wolbachia | Bta00841 |
| | oxononanoate aminotransferase (BioA) | | | | | |
| Ssa00937 | Acetyltransferase | L-lysine | 5.14 | 0.86 | Klebsiella | Bta04431 |
| Ssa08712 | Amidohydrolase | | 4.92 | −1.14 | Mucilaginibacter | Bta14802 |
| Ssa02297 | SCP-like extracellular | | 4.27 | 1.32 | Bacillus | Bta15712 |
| Ssa10201 | tRNA-splicing ligase RtcB | | 4.17 | 6.61 | Cardinium | Bta14500 |
| Ssa15043 | Histidine ammonia-lyase | L-histidine | 3.48 | −1.04 | Pantoea | Bta08776 |
| Ssa00739 | Amidinotransferase | Thr, Ser, Arg | 2.90 | −3.83 | Curtobacterium | Bta04987 |
| Ssa00740 | Amidinotransferase | Thr, Ser, Arg | 2.90 | −3.83 | Curtobacterium | Bta04988 |
| Ssa10534 | Leucine-rich repeat containing | | 2.82 | −1.46 | Not well defined | Bta11397 |
| | proteins | | | | | |
| Ssa00993 | Cyclopropane-fatty-acyl- | | 0.47 | 1.37 | Not well defined | Bta04469 |
| | phospholipid synthase | | | | | |
| Ssa12577 | Oxidoreductase, 2OG-Fe (II) | | −0.42 | 1.78 | Pantoea | Bta15191 |
| | oxygenase | | | | | |

Gene*** - Ortholog in MEAM1

TABLE 9
Horizontally transferred genes of fungal origin expressed in cassava B. tabaci
(SSA1-SG1) bacteriocytes

| | | Gene expression | | |
|---|---|---|---|---|
| Gene ID | Description | LogCPM | Fold change | Gene*** |
| ENSSSA1UGT005001 | Major royal jelly-related protein | 8.04 | −1.37 | Bta10729 |
| ENSSSA1UGT003831 | NFX1-type zinc finger-containing | 7.53 | −1.08 | Bta08945 |
| | protein 1 | | | |
| ENSSSA1UGT023534 | cryptochrome | 6.83 | 1.37 | Bta20021 |
| ENSSSA1UGT009368 | Squalene synthase | 6.43 | 2.20 | Bta11043 |
| ENSSSA1UGT002200 | Serine/threonine dehydratase serine | 5.40 | −1.02 | Bta13117 |
| | racemase | | | |
| ENSSSA1UGT027113 | Adenosine deaminase | 5.37 | 1.49 | Bta03315 |
| ENSSSA1UGT002367 | MYND finger family protein | 5.03 | −2.43 | Bta00932 |
| ENSSSA1UGT016597 | Major royal jelly-related protein | 4.73 | 2.09 | Bta14896 |
| ENSSSA1UGT001581 | Major royal jelly-related protein | 4.54 | 1.63 | Bta07455 |
| ENSSSA1UGT000931 | Gamma-glutamyltranspeptidase | 3.02 | 3.80 | Bta03557 |
| ENSSSA1UGT003831 | NFX1-type zinc finger-containing | 2.73 | −1.38 | Bta08944 |
| | protein 1 | | | |
| ENSSSA1UGT011235 | Gamma-glutamyltranspeptidase | 0.19 | 4.92 | Bta05545 |
| ENSSSA1UGT009478 | Glucosylceramidase, putative | 0.16 | 1.19 | Bta02658 |
| ENSSSA1UGT007947 | Major royal jelly-related protein | 0.16 | −1.92 | Bta10760 |
| ENSSSA1UGT007941 | Major royal jelly-related protein | −0.89 | 3.29 | Bta10761 |
| Ssa04524 | 2OG—Fe (II) oxygenase superfamily | 7.18 | −1.04 | Bta14373 |
| | protein | | | |
| Ssa04526 | 2OG—Fe (II) oxygenase superfamily | 7.17 | −1.36 | Bta14374 |
| | protein | | | |
| Ssa12366 | Aromatic peroxygenase | 7.17 | 7.42 | Bta05753 |
| Ssa12366 | Aromatic peroxygenase | 5.98 | 1.26 | Bta15602 |
| Ssa10936 | Aromatic peroxygenase | 4.34 | −33.68 | Bta00041 |
| Ssa04914 | MYND finger family protein | 3.77 | −7.98 | Bta05475 |
| Ssa15364 | Aromatic peroxygenase | 3.67 | 9.23 | Bta11373 |
| Ssa09482 | Major royal jelly-related protein | 3.53 | −42.90 | Bta05467 |
| Ssa15384 | MYND finger family protein | 3.05 | −7.06 | Bta07433 |
| Ssa09478 | Major royal jelly protein | 2.49 | −35.17 | Bta05463 |
| Ssa02002 | Zinc finger MYND-type protein | 2.26 | −3.27 | Bta10703 |
| Ssa14298 | Uracil phosphoribosyltransferase | 2.09 | −1.51 | Bta14185 |
| Ssa14166 | Dextranase | 2.06 | −1.51 | Bta02250 |
| Ssa06839 | Aminotransferase family protein (LolT), | 1.73 | 1.12 | Bta02178 |
| | putative | | | |
| Ssa03924 | Galactose oxidase | 1.61 | −18.38 | Bta01072 |
| Ssa09479 | Major royal jelly protein | 1.37 | −622.58 | Bta05465 |
| Ssa06267 | Aromatic peroxygenase | 1.29 | −14.49 | Bta04808 |
| Ssa10522 | Late sexual development protein | 0.44 | 3.03 | Bta12908 |
| Ssa10745 | Major royal jelly protein | 0.33 | −288.36 | Bta10736 |
| Ssa07646 | Aromatic peroxygenase | 0.80 | 124.02 | Bta07721 |
| Ssa03925 | Aromatic peroxygenase | 0.05 | −5.27 | Bta01073 |
| Ssa02113 | Galactose oxidase | −0.56 | 4.77 | Bta05093 |
| Ssa02112 | Galactose oxidase | −0.56 | 4.77 | Bta05092 |

Gene***—Ortholog in MEAM1

### Validation of Expression of Selected Genes Using Real-Time Quantitative PCR

A total of eight genes were selected as critical gene targets with enriched expression in either the whitefly gut or the bacteriocyte compared to the whole-body. These were, (i) ENSSSA1UGT022145: Aquaporin 1 (AQP1) for water recycling in the gut, (ii) ENSSSA1UGT002057: Alpha-glucosidase (SUC1) for sucrose hydrolysis, (iii) ENSSSA1UGT002066: alpha-glucosidase (SUC2) for sucrose hydrolysis, (iv) ENSSSA1UGT007134: diaminopimelate decarboxylase (LysA), essential in lysine biosynthesis, (v) ENSSSA1UGT003467: arginosuccinate lyase (ArgH), essential in arginine biosynthesis, (vi) ENSSSA1UGT021004: branched-chain-amino-acid aminotransferase (BCAT), critical in isoleucine, leucine and valine biosynthesis, (vii) ENSSSA1UGT008852: aspartate aminotransferase (AAT), essential in phenylalanine biosynthesis and (viii) ENSSSA1UGT000364: 4-hydroxy-tetrahydrodipicolinate reductase (dapB), essential in lysine biosynthesis. The selected genes with enriched expression in the bacteriocyte were selected from both differentially expressed gene set and horizontally transferred genes and their essentiality confirmed through constraint-based metabolic modeling of the genome-scale model of cassava whitefly (SSA1-SG1).

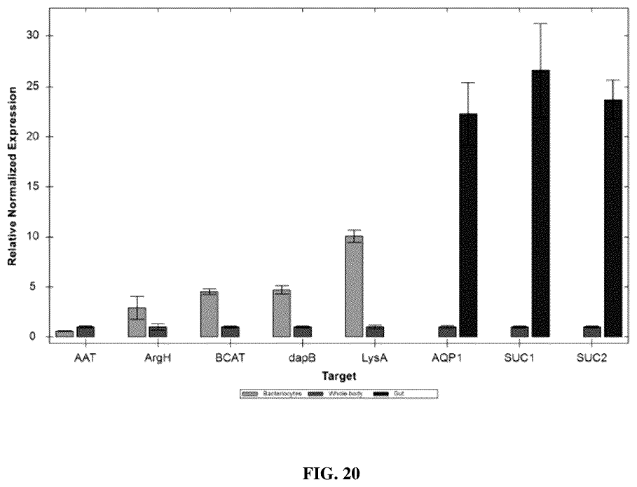

This analysis revealed that, of the eight selected genes, seven had enriched expression cither in the cassava whitefly gut (AQP1, SUC1 and SUC2) or bacteriocyte (ArgH, BCAT, dapB and LysA) compared to the whole-body (FIG. 20). The expression of AAT was more enriched in the whole-body compared to the bacteriocyte. Results from RNAseq analysis and RT-qPCR were similar with fold differences in the gut ranging from 22.30 to 26.54-folds for RT-qPCR compared to 21.71 to 32.67-folds from RNAseq analysis. In the bacteriocyte, gene expression for the selected gene ranged from 0.57 to 10.07-folds for RT-qPCR compared to 1.79 to 13.89-folds for RNAseq analysis.

In conclusion, essential gene targets encoding for proteins with different molecular but linked physiological functions have been identified and studied. Several factors have been considered, which include: (i) substrate specificity for sugar processing enzymes (ii) water specificity for water recycling mechanism in the gut (iii) reaction mediated in the essential amino acid biosynthesis pathways in the bacteriocyte and (iv) indispensability of the genes in the amino acid biosynthesis. Based on these factors, several genes have been selected as critical genes in cassava B. tabaci (SSA1-SG1). These include: ENSSSA1UGT022145: aquaporin (AQP1), ENSSSA1UGT002057: alpha glucosidase (SUC1), ENSSSA1UGT002066: alpha glucosidase (SUC2), dapB, dapF and lysA for lysine biosynthesis, branched chain amino-acid transferase for isoleucine, leucine, phenylalanine and valine biosynthesis, and argH for arginine biosynthesis. The expression of these genes has been validated using RT-qPCR (FIG. 20). It is envisaged that stacking these essential genes with similar physiological functions in combinatorial RNAi will further contribute to management of super abundant whiteflies in cassava growing areas in Sub-Saharan Africa.

### Example 2: Identification of Essential Genes Targets in Cassava B. tabaci Through Genome-Scale Metabolic Modeling

Whiteflies and other phloem-sap feeding insects depend on endosymbionts for essential amino acid and B vitamin biosynthesis. This example analyzes the interaction between cassava B. tabaci (SSA1-SG1) and its primary and secondary endosymbionts in the bacteriocyte using genome-scale metabolic modeling to identify key gene targets responsible for amino acid provisioning in cassava B. tabaci SSA1-SG1. The key symbiosis genes in SSA1-SG1 identified in this study may be downregulated using RNAi for suppression of cassava B. tabaci SSA1-SG1 and the cassava diseases (CMD and CBSD) they transmit.

Dissection of Bacteriocytes from Cassava B. tabaci

Bacteriocytes were dissected from B. tabaci SSA1-SG1 adult female flies as described in Example 1. RNA extraction, rRNA depletion, library preparation and sequencing were done as described above.

Identification of Metabolic Genes for Both SSA1-SG1 and Portiera aleyrodidarum

B. tabaci SSA1-SG1 reads were mapped using blastn to a Portiera aleyrodidarum reference assembly from NCBI, REfseq id NC_018677.1. Reads for the two SSA1-SG1-Portiera were mapped and the blast mappings were parsed and paired, taking the best hits in each case using the highest bit scores. Where there was a conflict, resulting from a read mapping equally to two locations because of the same bit score, the longest match length was picked. Matching read ids were then used to retrieve the original fastq reads which were paired and assembled using the Sassy assembler as described in (Kazakoff et al., 2012; Visendi et al., 2016). The generated Portiera genome was then uploaded to RAST (Rapid Annotation Using Subsystem Technology) server (Aziz et al., 2008) and annotated using RAST annotation scheme with a gene caller based on release70 FIGfam version. Metabolic genes with their respective enzyme commission numbers (EC numbers) were used for the reconstruction of the genome-scale metabolic model of Portiera.

For B. tabaci (SSA1-SG1), the coding sequences were obtained from the draft genome currently undergoing annotation. A Blastx search (Altschul et al., 1997) with a cutoff E-value of 1e-3 against NCBI protein reference non-redundant database (nr), mapping and annotation for all these sequences was done using Blast2GO suite (Conesa et al., 2005). Coding sequences with EC numbers were used to construct the genome-scale metabolic reconstruction for cassava B. tabaci (SSA1-SG1).

### Genome-Scale Reconstruction of Portiera and SSA1-SG1 Metabolic Models

Single compartment models for Portiera and B. tabaci (SSA1-SG1) were reconstructed following the procedures reported by Thiele & Palsson (2010). In brief, Enzyme Commission (EC) numbers for coding sequences of both Portiera and SSA1-SG1 were used to identify enzymes and pathways for the selected metabolic genes in Kyoto Encyclopedia of Genes and Genomes (KEGG) (Kanchisa & Goto, 2000). Reconstruction assembly was assembled manually in a pathway-pathway manner based on KEGG pathways using an Excel spreadsheet. Genes associated with each EC number were noted and used in constructing gene-protein-reaction (GPR) based on Boolean rules. Distinct proteins catalyzing the different enzymatic reaction to produce the same metabolite or product were considered as isozyme, therefore, an “OR” rule was applied while proteins from different transcripts acting together to catalyze a given reaction (complex reaction) were assigned with the “AND” rule. The selected enzyme was further analyzed using the BiGG Models, a knowledgebase of genome-scale metabolic network reconstructions (King et al., 2016) to determine the exact reaction stoichiometry of the biochemical reaction, chemical formula, changes of metabolites and reaction direction and reversibility. Other databases used to verify reaction stoichiometry of the biochemical reactions include EcoCyc (Keseler et al., 2013) and Braunschweig enzyme database (BRENDA) (Chang et al., 2015).

On completion, the reconstruction assembly for both Portiera and B. tabaci consisted of (i) reaction code, (ii) reaction equation, (iii) genes, (iv) gene-protein-reaction (GPR) association, (v) subsystem, (vi) reaction thermodynamics (reversibility), (vii) reaction constraints, (viii) confidence score, (ix) EC number for each reaction, (x) Biomass objective function and (xi) metabolite list with both charged and uncharged formula. Refining and manual curation of the model were done by (i) analyzing and verifying substrate and cofactor usage for each reaction, (ii) adding sink and demand reaction, (iii) verifying subcellular localization for each reaction and (iv) adding transport and exchange reactions which represent interaction and communication between Portiera and SSA1-SG1 B. tabaci. A pseudo reaction for biomass generation was developed as the objective function for the model as described by (Feist & Palsson, 2010; Ankrah et al., 2017). Briefly, each transcript expressed in cassava whitefly was translated and the number of each amino acid residue for each protein translated from each transcript was calculated. Transcript abundance for each transcript was multiplied by the total number of amino acid residues for each protein translated from each transcript to determine the total amount of each amino acid translated from each transcript. This was then used to calculate the total amount of each amino acid expressed in the cassava whitefly. The amino acid quantity for each amino acid was then used to determine the fractional contribution of each amino acid as a ratio of each amino acid to the total amino acid translated in the cassava whitefly. This fractional contribution was converted into fractional proportion of each amino acid (Mmolmonomer/gDw) required to fulfil the total protein requirement (Mmol/gDw) for cassava whitefly. These fractional proportions were then used to construct the objective function for the survival of cassava whitefly (SSA1-SG1). Stoichiometric coefficients in the objective function describe the relative abundance of essential building blocks, focusing on amino acid synthesis (amino acids, ATP and water) that enables Portiera and B. tabaci to grow and survive. This reaction was selected as the objective in the calculation of optimal flux distribution for the Portiera and B. tabaci SSA1-SG1 model.

The two single compartment models generated (iKT90 and iKT330) were combined to form a two-compartment model of B. tabaci SSA1-SG1 and Portiera, that was used for analysis and identification of essential symbiosis genes. The reactions in single models were renamed with a prefix SSA1_Por for Portiera model (iKT90) and SSA1_Bt for the B. tabaci SSA1-SG1 model (iKT330). Metabolites in iKT90 and iKT330 were localized in [p] and [c] compartments respectively. Transport reactions were assigned to ensure that dead-end metabolites from each compartment are exchanged between the two compartments (Portiera and bacteriocyte) and import essential metabolites into the compartment. Exchange reactions were also included so that the metabolites or products produced by the two compartments are exported to the external environment (B. tabaci haemolymph).

Conversion of the Metabolic Reconstruction into a Mathematical Model

The curated metabolic reconstruction was converted into the genome-scale metabolic model, a stoichiometric matrix using Constraint-based reconstruction and analysis (COBRA) toolbox (Schellenberger et al., 2011) running within MATLAB environment (Mathworks Inc., Natick, MA) using the Gurobi solver tool (Gurobi Optimization™). The stoichiometric matrix enables computation of metabolic capabilities and quantitative prediction of cellular behavior using constraint-based approaches. Constraints implemented on individual reaction fluxes included defining lower and upper bounds. Bounds define the space of allowable flux distribution that estimate the rate at which a metabolite is produced or consumed by a reaction. In the Portiera model, bounds for exchange reactions were set to zero and 1000 mmol gDw−1 h⁻¹ for lower and upper bound respectively to prevent metabolite uptake while leaving the rate of secretion of desired metabolite unconstrained. For irreversible reactions, the lower bound was constrained to 0 mmol gDw−h while reversible reactions were constrained to −1000 mmol gDw−h. In the B. tabaci metabolic model, RNAseq expression data were used to set the upper bounds. For reaction mediated by a single gene or many genes with ‘OR’ Boolean rule, the highest value of the expression (log 2CPM) was used, while the mean of expression values for all the genes was used for the reactions with ‘AND’ Boolean rule. Another constraint considered was a mass balance which assumed that each metabolite produced or transported into the cell is balanced by the amount of metabolite consumed within or secreted from the cell.

### Analysis and Evaluation of the Models

Several parameters of the model were assessed. These included the optimal steady state flux profile and essential reaction/genes in the Portiera and Portiera/B. tabaci model that would best predict the amino acid requirement for growth of Portiera and B. tabaci. The model was analyzed using flux balance analysis (Varma & Palsson, 1994) to determine the steady state flux profile that optimizes the objective function and to simulate gene essentiality of the two-compartment model. This also allowed identification of network gaps, blocked reactions or dead-end metabolite and determine whether the model generates the precursor metabolites for reactions.

The amino acid requirement for growth of Portiera and B. tabaci (SSA1-SG1) were determined using flux balance analysis (FBA) with simulations performed under aerobic conditions with a maximum oxygen uptake rate of 20 mmol gDW⁻¹h⁻¹, fructose and glucose as carbon sources and ammonia as the nitrogen source. The objective function for the two-compartment model was developed to optimize the amino acid requirement for both Portiera and B. tabaci. FBA was also used to determine the effect of gene/reaction deletion on the metabolic model. Each reaction was perturbed, one at a time, by constraining both the upper and lower bound to zero to restrict it from carrying flux. Gene or reaction knock out were implemented and growth simulated using SingleGeneDeletion or SingleReactionDelection function of COBRA Toolbox. A reaction was classified as essential or critical if when removed from the model, the resultant value of biomass flux is zero (the network does not support growth) or non-essential if perturbation caused no effect on the network (network supports growth). Genes mediating reactions in essential amino acid biosynthesis were selected from the set of identified essential genes as potential gene targets for controlling cassava B. tabaci through RNAi. Minimization of metabolic adjustment (MOMA) (Segre et al., 2002) and regulatory on/off minimization (ROOM) (Shlomi et al., 2005) were also used to confirm the essential genes selected.

Assembly and Functional Annotation of Portiera Genome from B. tabaci SSA1-SG1

A single compartment metabolic model for SSA1-SG1-Portiera was reconstructed using the assembled SSA1-SG1-Portiera genome. The assembled Portiera genome from cassava B. tabaci SSA1-SG1 was comparable in size, percentage GC content and number of coding sequences to Portiera genome from B. tabaci species MEAM1 and MED. It had a size of 347165 bp compared to 352068 bp and 357461 bp of Portiera from MEAM1 and MED respectively (Table 10).

TABLE 10
Comparison of genomic features of Portiera genome from
MEAM1, MED and SSA1-SG1

| Description | MEAM1 | MED | SSA1-SG1 |
|---|---|---|---|
| Size (bp) | 352068 | 357461 | 347165 |
| GC content (%) | 26.2 | 26.1 | 26.2 |
| Number of coding sequences | 282 | 290 | 291 |
| Number of subsystems | 66 | 71 | 65 |
| Number of RNAs | 36 | 36 | 36 |
| Number of unique hypothetical | 23* | 27** | 31*** |
| proteins | | | |

*present in MEAM1 but missing in SSA1-SG1
**present in MED but missing in SSA1-SG1
***present in SSA1-SG1 but missing in MEAM1

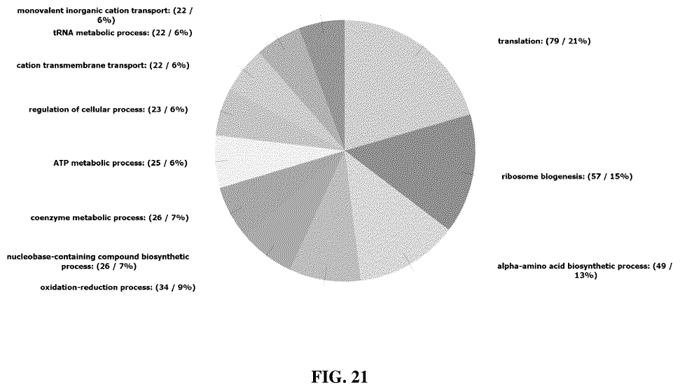

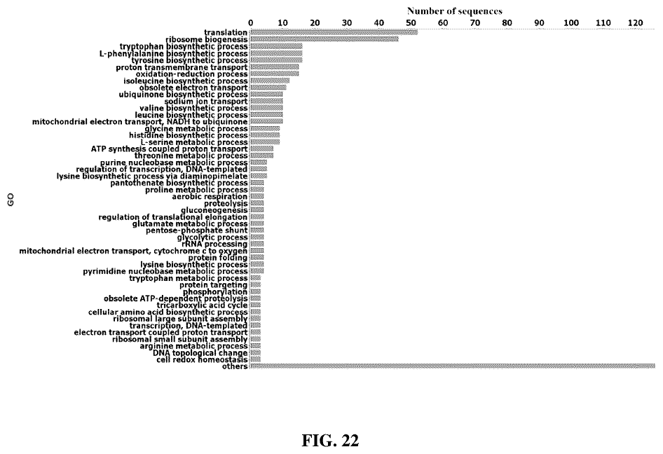

A total of 291 coding sequences were annotated using both RAST and Blast2GO to determine the function of the genes in the SSA1-SG1 Portiera genome. Sequence distribution of genes in the SSA1-SG1-Portiera genome revealed that the highest percentage of genes encoded for gene translation (21%), ribosome biogenesis (15%) and alpha-amino acid biosynthetic processes (13%) (FIG. 21). Direct Gene Ontology (GO) count of the biological processes for the coding sequences confirmed the contribution of Portiera in the biosynthesis and metabolism of amino acids in the bacteriocyte. Of the 20 top direct GO counts of biological processes, 10 related to amino acids biosynthesis and metabolic processes (FIG. 22).

Comparison of Portiera from B. tabaci SSA1-SG1, MEAM1 and MED

Although Portiera genome from MEAM1, MED and SSA1-SG1 were comparable in size, GC content, number of coding sequences and RNAs, comparative functional analysis was done to identify protein/function missing in the three Portiera genomes. A total of six unique proteins were identified, all of which were missing in SSA1-SG1-Portiera (Table 11). These included (i) 4-hydroxy-tetrahydrodipicolinate reductase DapB (EC 1.17.1.8), (ii) inner membrane protein translocase component YidC, (iii) RNA uridine 5-carboxymethylaminomethyl enzyme (GidA), (iv) cytochrome O ubiquinol oxidase subunit 1, (v) GTPase and tRNA-U34 5-formylation enzyme (TrmE) and (vi) SSU ribosomal protein S6p. Of these, only one metabolic enzyme (4-hydroxy-tetrahydrodipicolinate reductase—EC 1.17.1.8) was identified in Portiera from MEAM1 and MED but missing in Portiera from cassava B. tabaci SSA1-SG1 (Table 11). Previous study by (Luan et al., 2015) showed that the metabolic enzyme (4-hydroxy-tetrahydrodipicolinate reductase—EC 1.17.1.8) that is missing in SSA1-SG1-Portiera is encoded by dapB, a key gene in the lysine biosynthesis pathway.

TABLE 11
Missing protein/function in Portiera genome from
MEAM1, MED and SSA1-SG1

| Unique protein/function | MEAM1 | MED | SSA1-SG1 |
|---|---|---|---|
| 4-hydroxy-tetrahydrodipicolinate | + | + | − |
| reductase (EC 1.17.1.8) | | | |
| Inner membrane protein translocase | − | + | − |
| component YidC | | | |
| tRNA uridine | − | + | − |
| 5-carboxymethylaminomethyl | | | |
| enzyme (GidA) | | | |
| Cytochrome O ubiquinol | + | + | − |
| oxidase subunit 1 | | | |
| GTPase and tRNA-U34 5-formylation | − | + | − |
| enzyme TrmE | | | |
| SSU ribosomal protein S6p | − | + | − |

Single Compartment Models of SSA1-SG1-Portiera and B. tabaci SSA1-SG1

The reconstructed single compartment metabolic model for SSA1-SG1-Portiera comprised 76 intercellular reactions, 150 metabolites and a total of 90 genes supporting a metabolic network, iKT90. The model had an optimal solution based on flux balance analysis of 6.70, showing that the flux through the reconstructed metabolic network maximizes growth rate, resulting in a predicted exponential growth rate of 6.70 h⁻¹. The symbiosis system in both SSA1-SG1 (iKT90) and MEAM1 (iNA94) is quite conserved. Comparison of both single compartment models of Portiera from both SSA1-SG1 and MEAM1 showed that the two models are quite similar in the number of intercellular reactions, genes and metabolites (Table 12).

TABLE 12
Comparison of genome-scale metabolic models of B. tabaci SSA1-SG1 and MEAM1

| | B. tabaci SSA1-SG1 | | | B. tabaci MEAM1 | | |
|---|---|---|---|---|---|---|
| Description | iKT90 | iKT330 | iKT420** | iNA94 | iNA332 | iNA774*** |
| Number of metabolic genes | 90 | 330 | 420 | 94 | 332 | 774 |
| Number of intracellular reactions | 76 | 233 | 310 | 76 | 236 | 774 |
| Number of metabolites | 150 | 251 | 402 | 148 | 253 | 550 |
| Flux balance optimal solution (h⁻¹) | 6.70 | 25.38 | 0.31 | 12.02 | 26.55 | 0.20 |
| Total protein (mmol/gDw) | | | 6.17 | | | 6.20 |
| Total cost for protein synthesis | | | 26.58 | | | 26.71 |

**Two compartment model for B. tabaci SSA1-SG1 and Portiera
***Three-compartment model for B. tabaci MEAM1, Portiera and Hamiltonella (Ankrah et al., 2017)

The primary role of Portiera in the bacteriocyte relates to amino acid biosynthesis. The reconstructed metabolic model for Portiera was analyzed to determine essential amino acid provisioning and the genes mediating critical reaction in the essential amino acid biosynthesis in the cassava whitefly bacteriocyte. Portiera as a primary symbiont in B. tabaci SSA1-SG1 contributes final/terminal reactions for the synthesis of three essential amino acids. These include (i) threonine, (ii) methionine and (iii) tryptophan. Once synthesized, the three essential amino acids are exported to the bacteriocyte, where they are made available to the B. tabaci. Furthermore, Portiera also contributes to the synthesis of metabolites for intermediate reactions of other essential amino acids. For example, it possesses genes that encode for proteins required for chorismate biosynthesis, which is a precursor of aromatic amino acids (phenylalanine, tryptophan and tyrosine).

The bulk of reactions of the biosynthesis pathways of seven essential amino acids (arginine, histidine, lysine, phenylalanine, isoleucine, valine and leucine) are mediated by genes in Portiera. Metabolites produced in these reactions are exported across a symbiosome membrane into the bacteriocyte for amino acid biosynthesis. Metabolites (intermediate precursors) exported across the symbiosome membrane include, (i) N (Omega)-(L-Arginino) succinate for arginine biosynthesis, (ii) L-Histidinol phosphate for histidine biosynthesis, (iii) LL-2,6-Diaminoheptanedioate for lysine biosynthesis, (iv) phenylpyruvate for phenylalanine biosynthesis, (v) (5)-3-methyl-2-oxopentanoate for isoleucine biosynthesis, (vi) 3-methyl-2-oxobutanoate for valine biosynthesis and (vii) 3-carboxy-4-methyl-2-oxopentanoate for leucine biosynthesis.

In addition, the single compartment model of Portiera revealed that Portiera lacks genes that encode for metabolites required in the biosynthesis of non-essential amino acids (alanine, asparagine, aspartic acid, cysteine, glutamic acid, glutamine, glycine, proline, serine and tyrosine). This implies that Portiera depends on the B. tabaci SSA1-SG1 for the provision of non-essential amino acids. It also requires eight host-derived metabolites for the biosynthesis of essential amino acids. These include, (i) 2, 3, 4, 5-tetrahydrodipicolinate, (ii) L-aspartate, (iii) alpha-D-ribose 5-phosphate, (iv) 5-methyltetrahydrofolate, (v) L-glutamine, and (vi) L-serine. These metabolites are imported across the symbiosome membrane by Portiera as precursors in different essential amino acid biosynthesis pathways.

Two-Compartment Models of B. tabaci SSA1-SG1 and Portiera aleyrodidarum

### Identification of Endosymbionts in the SSA1-SG1 Bacteriocyte

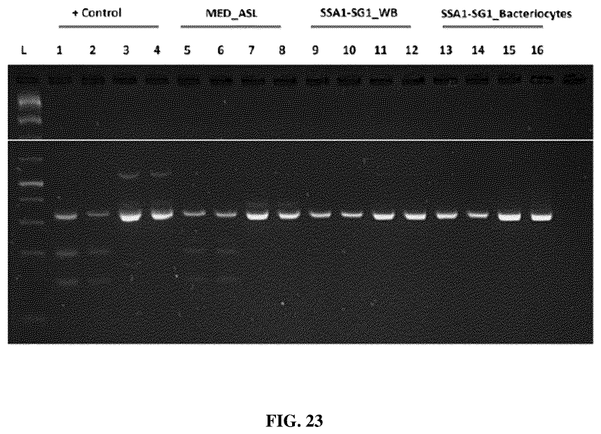

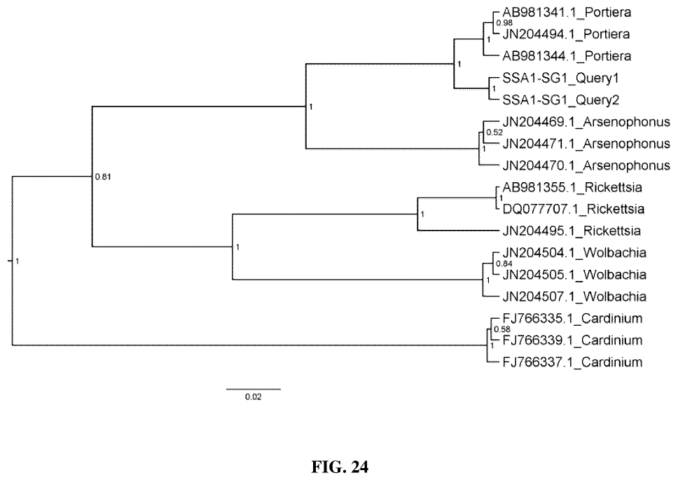

Secondary endosymbionts in the bacteriocytes also make a contribution to essential amino acid biosynthesis, albeit little (Ankrah et al., 2017). Therefore, a preliminary study was done to identify endosymbionts co-existing in the SSA1-SG1 bacteriocyte. Modified primers for 16S rRNA (907Fmod and 1237R) (Jing et al., 2014) were used to amplify the V6-V7 region of the all endosymbionts in the bacteriocytes. Prior to this analysis, in silico analysis of PacBio reads of SSA1-SG1 (used for assembly of SSA1-SG1 genome) revealed traces of Wolbachia, therefore both wholebody and bacteriocyte samples of SSA1-SG1 were compared with B. tabaci MED_ASL and a control, which were both positive for Wolbachia and other endosymbionts. Digestion of 16S rRNA amplicon of the V6-V7 region using Mwo1 restriction enzyme showed that MED_ASL had both Portiera (restriction bands of 331 kb) and Wolbachia (restriction band of 171 kb) while B. tabaci SSA1-SG1 had only Portiera in its bacteriocyte (FIG. 23). Phylogenetic analysis of digested 16S rRNA amplicon of the V6-V7 region from SSA1-SG1 bacteriocytes confirmed that the dominant endosymbiont was Portiera (FIG. 24). Based on these results, a two-compartment model for B. tabaci SSA1-SG1 and Portiera was constructed to identify the essential gene targets in the different essential amino acid biosynthetic pathways.

Therefore, a two-compartment model (iKT420) was reconstructed combining both the single compartment model for Portiera (iKT90) and for B. tabaci (iKT330). The naming of the two-compartment model (iKT420) for cassava whitefly (SSA1-SG1) followed the standard system biology procedure of naming metabolic models with i representing insilico metabolic model containing 420 metabolic genes (Table 12) generated by Kaweesi Tadeo (KT).

### Shared Metabolic Interaction Between Portiera and SSA1-SG1

Terminal reactions in the essential amino acid biosynthesis pathway are important because they influence the provisioning of amino acid. Genome-scale metabolic reconstruction of both B. tabaci SSA1-SG1 and its primary endosymbiont revealed that the terminal reaction of isoleucine, leucine, valine, histidine and phenylalanine are all mediated by genes of intrinsic origin in the host bacteriocyte. For example, for branched-chain amino acids, one enzyme, branched-chain amino acid transaminases (EC 2.6.1.42) encoded by BCAT, mediate the conversion of 3-methyl-2-oxobutanoate to valine, 4-methyl-2-oxopentanoate to leucine and(S)-3-Methyl-2-oxopentanoate to isoleucine. Other genes include, hisD (EC 1.1.1.23) and aspC (EC 2.6.1.58).

In Silico Prediction of Essential Amino Acid Synthesis Rate in B. tabaci SSA1-SG1

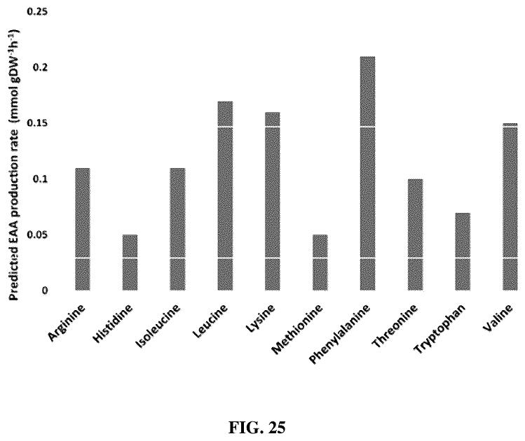

Flux balance analysis was used to predict the in-silico production rate of essential amino acids by the two-compartment genome-scale metabolic model of B. tabaci SSA1-SG1. The predicted production rate of different essential amino acids in B. tabaci SSA1-SG1 varied from 0.05 mmol gDW⁻¹h⁻¹ to 0.21 mmol gDW⁻¹h⁻¹. Three essential amino acids had a relatively high rate of production in the SSA1-SG1 bacteriocytes. These were (i) phenylalanine with 0.21 mmol gDW⁻¹h⁻¹, (ii) leucine with 0.17 mmol gDW⁻¹h⁻¹ and (iii) lysine with 0.16 mmol gDW⁻¹h⁻¹ (FIG. 25). Some essential amino acids, for example, histidine, methionine tryptophan and threonine were produced at a relatively low rate. Three of these amino acid (methionine, threonine and tryptophan) are produced mainly by Portiera.

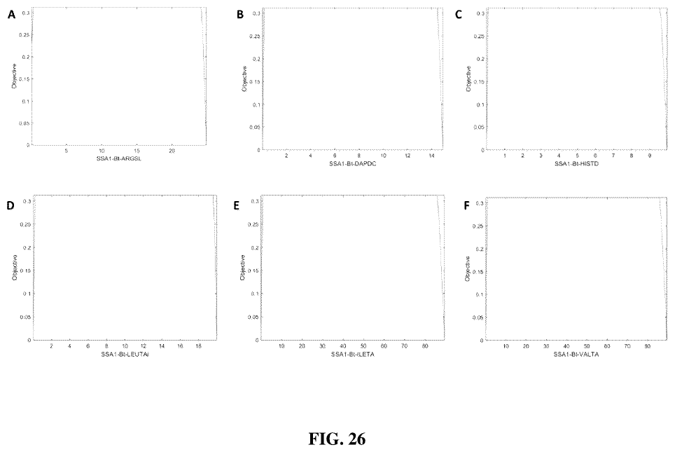

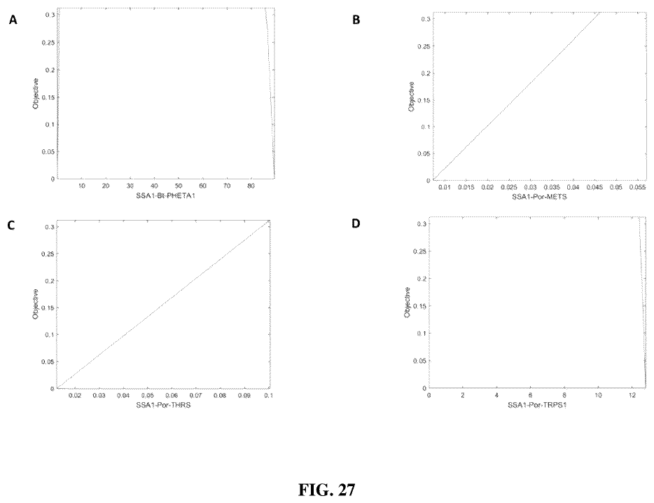

Robustness analysis to determine the systemic effect of varying metabolite flux through terminal reactions of different essential amino acids on the objective (growth of B. tabaci SSA1-SG1) revealed five reactions that are sensitive to fluctuation in the maximum allowable flux. These were (i) SSA1-Bt-HISTD for histidine synthesis (ii) SSA1-Por-TRPS1 for tryptophan synthesis (iii) SSA1-Bt-DAPDC for lysine synthesis (iv) SSA1-Bt-LEUTAi for leucine synthesis and (v) SSA1-Bt-ARGSL for arginine, all with maximum allowable flux ranging from 9.5-24 mmol gDW⁻¹h⁻¹ (FIG. 26 & FIG. 27). The most crucial point in this robustness analysis is the flux at which the optimal flux balance solution is attained and the flux at which the model begins to deviate from the optimal solution, which is the optimum growth rate (FIG. 26 and FIG. 27). Some reactions, especially terminal reactions within Portiera (SSA1-Por-METS, for methionine, SSA1-Por-THRS for threonine and SSA1-Por-TRPS1 for tryptophan) exhibit different patterns, with relatively very low flux required to attain the optimal solution (FIGS. 27 B, C and D).

### Essential Gene Targets in Different Essential Amino Acid Biosynthesis Pathways in SSA1-SG1

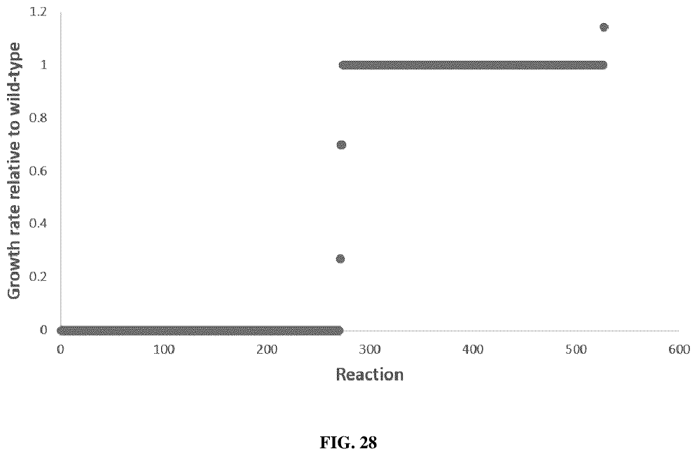

Genome-scale reconstruction of both B. tabaci SSA1-SG1 and Portiera demonstrated an interdependence of both cassava whitefly and its primary endosymbiont. Analysis of the shared metabolic interaction coupled with constraint-based metabolic modeling offered opportunities to identify and select critical/essential symbiosis genes that can be used as gene targets in the management of cassava whiteflies. Gene essentiality was simulated by constraining each reaction, one at a time, from carrying flux and the effect of this perturbation was assessed by comparing the growth rate of the perturbed network to that of wild type (FIG. 28). Based on the growth rate, a total of 270 reactions were characterized as essential. Of these, 129 reactions were mediated by genes in Portiera while 141 reactions were mediated by genes in B. tabaci SSA1-SG1. Essential amino acid biosynthesis pathways in B. tabaci SSA1-SG1 had a varying number of indispensable genes. Histidine pathway had the highest number of indispensable genes (10 genes) followed by lysine and leucine biosynthesis pathway with 9 and 6 essential genes, respectively.

Single gene deletion also revealed that seven of the ten biosynthesis pathway had essential genes mediating their terminal reactions. Therefore, these genes were characterized as accessory essential genes in the provisioning of essential amino acids in the cassava B. tabaci SSA1-SG1. These include, (i) argH (EC 4.3.2.1) for arginine biosynthesis, (ii) lysA (EC 4.1.1.20) for lysine biosynthesis, (iii) Branched-chain amino acid transaminase (EC 2.6.1.42) for isoleucine, valine and leucine biosynthesis, (iv) hisD (EC 1.1.1.23) for histidine and (v) aspC (EC 2.6.1.58) for phenylalanine biosynthesis. Of these, two genes (argH and lysA) are horizontally transferred genes of bacterial origin while three genes (BCAT, hisD and aspC) are of intrinsic origin (Table 13). All the selected reactions except PHETA 1 for phenylalanine biosynthesis were mediated by the protein encoded by a single gene. This is contrary to the genes that mediate metabolic inputs (metabolites that are exported across the symbiosome membrane) in different essential amino acid biosynthesis pathways (Table 14). Therefore, three factors, (i) indispensability of the gene, (ii) reaction mediated by a single gene and (iii) gene mediating a terminal reaction were considered in the selection of the best candidate gene targets for the management of cassava whitefly SSA1-SG1 (Table 13).

TABLE 13
Accessory essential genes mediating critical reactions of essential amino acid biosynthesis
in B. tabaci (SSA1-SG1) bacteriocytes simulated using FBA, MOMA and ROOM

| | | | | | | Number of | |
|---|---|---|---|---|---|---|---|
| Reaction | Gene name | Enzyme | EC number | Origin* | Flux | isoforms | Pathway |
| DHDPRy | dapB | Dihydrodipicolinate reductase | 1.17.1.8 | HTG | 0.16 | 1 | Lysine |
| DAPE | dapF | Diaminopimelate epimerase | 5.1.1.7 | HTG | 0.16 | 1 | Lysine |
| DAPDC | lysA | Diaminopimelate decarboxylase | 4.1.1.20 | HTG | 0.16 | 1 | Lysine |
| ARGSL | argH | Argininosuccinate lyase | 4.3.2.1 | HTG | 0.11 | 1 | Arginine |
| PHETA1 | aspC | Phenylalanine transaminase | 2.6.1.58 | Intrinsic | 0.21 | 2 | Phenylalanine |
| LEUTAi | BCAT | Leucine transaminase | 2.6.1.42 | Intrinsic | 0.17 | 1 | Leucine |
| ILETA | BCAT | Isoleucine transaminase | 2.6.1.42 | Intrinsic | 0.11 | 1 | Isoleucine |
| VALTA | BCAT | Valine transaminase | 2.6.1.42 | Intrinsic | 0.15 | 1 | Valine |
| HISTP | hisB | Histidinol phosphatase | 3.1.3.15 | Intrinsic | 0.05 | | Histidine |
| HISTD | hisD | Histidinol dehydrogenase | 1.1.1.23 | Intrinsic | 0.05 | | Histidine |

*HTG - Horizontally transferred genes. Intrinsic - Host genes
Flux - the amount of substrates/products produced in a certain reaction per unit time (mmol gDW⁻¹ h⁻¹)

TABLE 14
Essentiality of genes mediating synthesis of metabolic inputs in the synthesis of essential amino acids

| | | | | | | No of | |
|---|---|---|---|---|---|---|---|
| Metabolite | Reaction | Gene | Enzyme | Essentiality | EC No | genes | Pathway |
| mlthf | GHMT2r | GlyA | Glycine hydroxymethyl transferase | Essential | 2.1.2.1 | 2 | Methionine |
| | GLYCL | GcvT | Glycine cleavage system | Non-essential | 2.1.2.10 | 12 | Methionine |
| | MTHFD | | Methylenetetrahydrofolated e hydrogenase | Essential | 1.5.1.5 | 2 | Methionine |
| amet | METAT | metK | Methionine adenosyltransferase | Essential | 2.5.1.6 | 1 | Methionine |
| r5p | NRSPRT | PNP | Nicotinate D - ribonucleoside: orthophosphate | Non-essential | 2.4.2.1 | 2 | Histidine |
| | RP1 | RpiA | Ribose-5-phosphate isomerase | Essential | 5.3.1.6 | 1 | Histidine |
| | TKT1 | TKtA | Transketolase | Non-essential | 2.2.1.1 | 2 | Histidine |
| asp-L | ASPTA | AspC | Aspartate transaminase | Essential | 2.6.1.1 | 3 | Lysine, Threonine |
| | ASNN | AnsA | L-asparaginase | Non-essential | 3.5.1.1 | 6 | Lysine, Threonine |
| gln-L | GLNS | GlnA | Glutamine synthase | Essential | 6.3.1.2 | 3 | Arginine, Tryptophan |

In summary, four genes were selected as the potential gene targets to disrupt six essential amino acid biosynthesis pathways in the B. tabaci SSA1-SG1 bacteriocyte. These include (i) BCAT for valine, isoleucine and leucine biosynthesis, (ii) lysA for lysine biosynthesis, (iii) argH for arginine biosynthesis and (iv) hisD for histidine biosynthesis. Factors considered in the selection of these gene include (i) essentiality of the gene, (ii) genes that encode for a protein that mediate terminal reactions and (iii) the terminal reactions that are mediated by the protein encoded by a single gene. These target genes are useful not only in controlling the superabundant SSA1-SG1 population but also in managing the spread of cassava viral diseases they transmit.

### Example 3: Efficacy of RNA Interference Against Osmoregulation Genes in the Management of Bemisia tabaci

In B. tabaci, two candidate osmoregulation genes; Aquaporin 1 (AQP1), a major intrinsic protein and Sucrase 1 (SUC1) an alpha glucoside (family 13) have been identified in MEAM1 (Jing et. al., 2016). In this study, the efficacy of RNAi against AQP1 and SUC1, genes with different molecular but linked physiological function was evaluated. Tomato plants (Solanum lycopersicum L) stably transformed with dsRNA against these genes were used to determine the effect of these transgenic plants on survival and physiology of whiteflies.

### Plant Material and Insects

The B. tabaci species, Middle East-Asia Minor (MEAM1) (GenBank accession no KM507785) was collected originally from Ithaca, New York. The colony had been maintained at Cornell University on tomato (Solanum lycopersicum cv Florida Lanai) at 25±2° C. with 12:12 h L:D at 350 μmol^{m-2s-1} PAR. Six types of stably transformed tomato plant (cv Florida Lanai) generated by Luo et. al, (2017) were obtained from the Douglas laboratory, Department of Entomology, Cornell University. These plants had the following transformations: (i) dsRNA against RNase (dsRNase1 & 2) (NCBI Accession KX390872 and KX390873 respectively), (ii) dsRNA against GFP (dsGFP-dsRNA), (iii) dsRNA against sucrase (dsSUC) (NCBI Accession KX390871), (iv) dsRNA against aquaporin (NCBI Accession KX390870) and sucrase (dsAQP+dsSUC), (v) dsRNA against RNase 1 & 2, aquaporin and sucrase (dsRNase+dsAQP+dsSUC) and (vi) empty vector.

Due to the limited number of transformed tomato plants per line (T1), tomato seeds from different stably transformed plants were generated and treated with Rapidase (12 ml/L of water) for 1.5-2 hours. The treated seeds were established to generate seedlings from each line (second generation plants, T2) for the experiment. Seedlings were tested for the presence of target dsRNAs to ensure that only stably transformed plants were used. The plants were grown on compost (Cornell mix) supplemented with a water-soluble fertilizer and maintained in a climate-controlled greenhouse at 25±2° C., 60% relative humidity and a 12:12 h L:D for 6 weeks.

Evaluating B. tabaci Performance on Tomato Transgenic Plants

After 6 weeks, the transgenic tomato plants were transferred to the climate-controlled chamber (25±2° C., 60% relative humidity and a 12:12 h L:D with a light intensity of 350 μmol^{m-2s-1} PAR). The experiment was set up as a non-replicated single row, with each row consisting of five plants per transgenic line and two clip cages were attached to each plant. A tomato leaf containing only 3rd instar nymphs and pupae was placed in a cage containing a clean tomato plant, at two days before the experimental setup to ensure that only newly emerged whiteflies were used. Wet cotton wool was placed on the leaf stalk to avoid quick desiccation. After two days, all the emerged whiteflies had settled on the leaves and were acclimatized. Only whiteflies in courtship (pairs) were collected in glass vials. This was done to minimize stress or strain on whiteflies prior to the experiment. A total of 10 newly emerged whiteflies (5 males: 5 females) were transferred into each cage. Data on insect survival was taken daily for six days.

Analysis of SUC1 and AQP1 Gene Expression in MEAM1 B. tabaci

The effect of RNAi on the expression of both SUC1 and AQP1 genes was analyzed using quantitative real-time PCR (qRT-PCR). Total RNAs were extracted from MEAM 1 B. tabaci that survived after six days of feeding on the transgenic plants. Total RNA for all samples were normalized to ensure an equal amount of starting RNA template (100 ng). Normalized RNAs were then treated with 1 μl of the ezDNase™ enzyme (Invitrogen™) for 2 minutes at 37° C. to remove genomic DNA. The RNA samples were further incubated at 55° C. for 5 minutes in the presence of 10 mM DTT to inactivate the enzyme. First-strand cDNA synthesis was carried out using SuperScript™ II Reverse Transcriptase (Invitrogen™) according to manufacturer's instructions. A three-step thermocycling protocol was used with polymerase activation and DNA denaturation at 95° C. for 3 minutes followed by 40 cycles of both denaturation at 95° C. and annealing/extension at 55.2° C. for 45 seconds, performed on a C1000 Thermo cycler with CFX96 Real-time detection system (Bio-Rad), with 60S ribosomal protein L13a (RPL13) as the internal control. A total of 20 μl reaction mix containing 10 μl iQ™ SYBR® Green Supermix (2×), 0.5 μl of forward and reverse primer, 1 μl of cDNA and 8 μl of water was used. Melting curve analysis at the end of the reaction was done by increasing temperature from 55-953 C in increments of 0.5° C. every 5 seconds to assess the dissociation characteristics of dsDNA during heating.

### Honeydew Collection

The honeydew analysis was done to determine the physiological impact of RNAi on sugar transformation within the B. tabaci gut. Honeydew was collected from whiteflies feeding on transgenic plants, wild-type tomato, Arabidopsis thaliana (L.) Heynth cv Columbia and artificial diet. An artificial diet with 0.75 M sucrose was made following a modified protocol by Prosser & Douglas, 1992, and Douglas, et al., 2001. A total of 200 whiteflies were placed in each cage and honeydew was collected after 48 h of feeding. Each cage was placed at the same level (fourth branch) of the plant. Honeydew was collected on aluminum foil placed at the base of the cage. Before the collection, the aluminum foil was washed with 80% methanol to remove any contaminants before being placed in an oven at 60° C. for 12 h. The weight of the aluminum foil (X0) was taken before it was used in the cage (X0) using an ultrabalance (Toledo MX5). The weight of honeydew (y) was assessed by determining the difference in the weight of aluminum foil (X0) and the weight of aluminium foil after B. tabaci depositing honeydew (X1). Honeydew was then washed off the aluminum foil using 300-500 μl of deionized water. Aluminum foil was dried and weighed again (X2) to determine the amount of honeydew washed off the aluminum foil. Honeydew samples were then stored at −20° C. for sugar analysis. A negative control was included (foil from an empty clip cage) to correct for weight due to any other factors like moisture.

Analysis of Sugars in Honeydew Samples from Whiteflies

### Sucrose Assays

The amount of sucrose in the honeydew sample signified the activity of Sucrase (net hydrolysis of sucrose) that ensures osmotic balance within B. tabaci. Therefore, in this study, honeydew samples were analyzed to quantify the amount of sucrose in honeydew produced by B. tabaci feeding on different stably transformed tomato plants. Sucrose was hydrolyzed in vitro to its constituent monosaccharides, α-glucose and β-fructose by β-fructofuranosidase (Invertase, Sucrase, EC 3.2.1.26). Each sample was divided into two aliquots. One of the aliquots was treated with Invertase while the other was not. In principle, if the honeydew sample contains sucrose, the amount of glucose in the aliquoted sample treated with Invertase should be higher than the aliquoted sample that has not been treated with Invertase. A total of 20 μl honeydew was hydrolyzed to completion using 4 μl (0.4 U) Invertase (Sigma I-4504) in 50 mmol 1⁻¹ sodium acetate buffer, pH 4.5 at 37° C. for 30·min and the glucose produced was determined by the glucose method described below.

### Glucose Assay

Glucose assays were conducted using Sigma Glucose Oxidase kit (GAGO20). In brief, the kit contains a glucose oxidase assay reagent which is a mixture of glucose oxidase reagent and O-dianisidine reagent and a glucose assay standard. Glucose standards were made. The aliquoted honeydew samples were normalized by adding 4 μl water to 20 μl honeydew. Then 5 μl of the normalized sample, glucose standard and honeydew sample treated with Invertase were added to microtiter, flat-bottom 96 well plate in triplicates for each sample. This was followed by adding 150 μl assay reagent to each sample. The samples were then incubated at 37° C. for 30 minutes. After incubation, 150 μl of dilute sulphuric acid (12 N H2SO4) was added to each sample and then absorbance was read at 540 nm using a Bio-RAD xMark™ microplate spectrophotometer. A regression equation from the glucose standard curve was used to compute the content of glucose in each sample.

Analysis of variance on the amount of sucrose in honeydew samples collected from different transgenic lines and sucrose hydrolysis rate was used to determine the effect of dsRNAs on the sugar transformation within the B. tabaci gut.

### Quantification of Higher Sugars in Honeydew Samples

Each honeydew sample collected from different transgenic plants, tobacco plants and artificial diet was divided into aliquots. One of the aliquots was used to measure sucrose content using enzymatic methods (Invertase) while the other aliquot was sent to the Max Planck Institute for Chemical Ecology (Jena, Germany) for analysis of disaccharides and higher sugars. Disaccharides and higher sugars were analyzed using liquid chromatography-mass spectrometry. Both percentage molar ratio and sugar concentration for different sugar categories in each honeydew samples were computed. Sugar category analyses included were fructose, glucose, disaccharides, trisaccharides, tetrasaccharides and pentasaccharides. Sugar concentration was computed by calculating the hexose units (nmol/mg) for all the sugar categories measured for each milligram of honeydew.

Determination of Osmotic Pressure of B. tabaci Hemolymph

Haemolymph osmotic pressure of B. tabaci feeding on different transgenic tomato plants was measured to assess the physiological consequence of RNAi against the osmoregulation genes (aquaporin and sucrase). Third instar nymphs collected from different transgenic tomato plants (SUC1, dsAQP+SUC1, dsRNase+dsAQP+dsSUC, Empty vector, Wild-type and dsRNase) were used to measure the impact of RNAi on the osmotic pressure in whiteflies. This was done using a freezing point depression method, measured on the Otago™ nanoliter osmometer. Haemolymph was collected from individual third instar nymphs. Each nymph was placed in water saturated nondrying immersion oil (Cargille Laboratories, New Jersey, USA) on a microscope glass slide. Each nymph was pierced in-between the eyes using a very fine dissection pin to obtain the haemolymph. The capillary tube containing non-drying immersion oil A (Cargille laboratories, New Jersey, USA) was used to transfer collected haemolymph to non-drying immersion oil B (Cargille Laboratories, New Jersey, USA) on the cooling block of the nanoliter osmometer.

The sample in non-drying immersion oil B on the cooling block was frozen to −20° C. then the melting temperature of the sample was noted. A standard curve was done using 100 mOSm/kg, 500 mOSm/kg, 900 mOSm/kg, 1500 mOSm/kg and 2000 mOSm/kg osmolarity linear set (Advanced Instrument Inc, Norwood Massachusetts, USA). A linear equation from the standard curve was used to determine the haemolymph osmotic pressure for each sample. Analysis of variance was used to determine whether the dsRNAs in each test plant had a significant impact on the osmotic pressure of B. tabaci.

### Sucrose Hydrolysis in Whiteflies Feeding on Different Sources of Sucrose

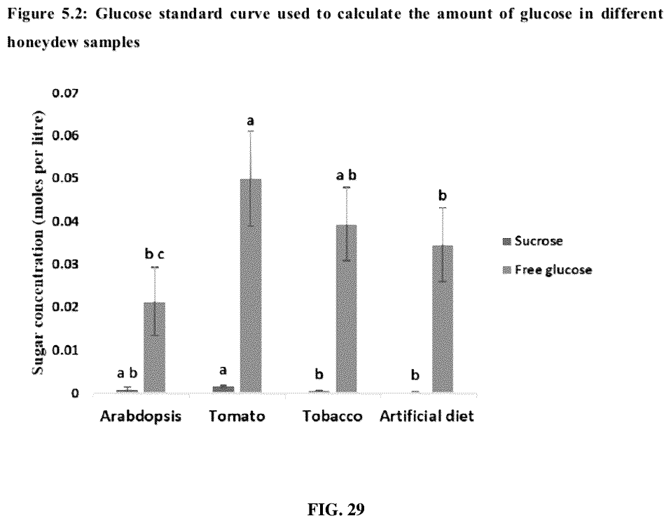

Before evaluating the efficacy of RNAi on the survival of whiteflies, preliminary studies were conducted to understand sucrose hydrolysis within whiteflies feeding on different sources of sucrose, the role of SUC1 gene in osmoregulation and the suitability of tomato plants (Solanum lycopersicum) as source for B. tabaci (MEAM1). Honey dew was collected from whiteflies feeding on four sugar sources. These were: (i) tomato (Solanum lycopersicum cv. Florida Lanai), (ii) tobacco (Nicotiana tabacum L), (iii) Arabidopsis thaliana and (iv) artificial diet with known sucrose concentration (0.75 M). Honeydew samples from whiteflies feeding on Arabidopsis and tomato had significantly higher sucrose content (0.001 M and 0.001 M, respectively) compared to the honeydew samples from whiteflies feeding on tobacco and artificial diet (0.5 mM and 0.3 mM respectively) (FIG. 29). Though honeydew samples from whiteflies feeding on Arabidopsis had significantly higher sucrose, it had significantly lower free glucose compared to honeydew samples from whiteflies feeding on tomato, tobacco and artificial diet. Honeydew samples from whiteflies feeding on tomato, tobacco and artificial diet had a relatively similar amount of free glucose, that was not statistically different from each other.

Sugar analysis from honeydew samples of whiteflies feeding on an artificial diet with known sucrose concentration (0.75 M) revealed that whiteflies hydrolyzed 97% ingested sucrose to monosaccharides; fructose and glucose. Differences therefore in sucrose hydrolysis in whiteflies feeding on different sucrose sources are attributed to the sucrose sources (tobacco, tomato, Arabidopsis and artificial diet) rather than the insect physiology.

### Comparison of Sucrose Hydrolysis in Aphids and Whiteflies

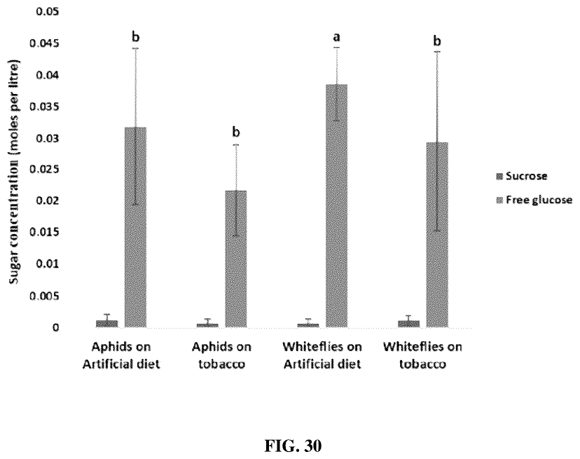

Honeydew samples from aphids (in this case, Myzus persicae Sulzer) and whiteflies (B. tabaci MEAM1) feeding on tobacco and artificial diet (0.75 M) were analyzed to compare sucrose hydrolysis in aphids and whiteflies. Tobacco plants were used in preference to tomato because tomato plants were not a suitable host for Myzus persicae. There was no significant difference in the amount of sucrose in the honeydew samples from both aphids and whiteflies feeding on tobacco and artificial diet. Nearly all the sucrose (0.74 M out of 0.75 M) was hydrolyzed by both the aphids and whiteflies. Sugar hydrolysis of honeydew samples from an artificial diet with known sucrose concentration (0.75 M) revealed that free glucose in honeydew from whiteflies was significantly higher than that from aphids, showing that the differences in sugar assimilation and isomerization is attributed to insect physiology rather than the sugar sources (FIG. 30).

B. tabaci Performance on Different Transgenic Tomato Lines

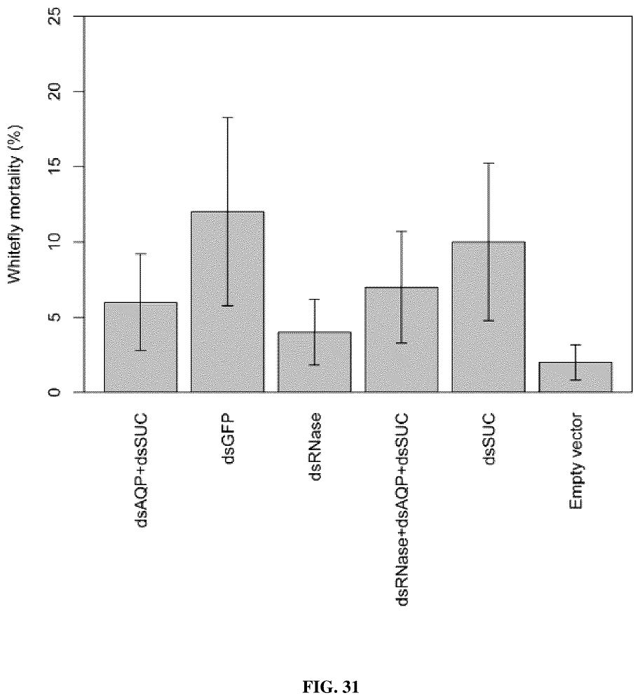

The efficacy of RNAi in the control of B. tabaci MEAM1 population on tomato plants was determined by evaluating the survival of B. tabaci on stably transformed tomato plants during a span of six days. Tomato plants were stably transformed to down-regulate the expression of either or both AQP1 and SUC1 genes to disrupt osmoregulation within B. tabaci, thereby affecting their survival. Analysis of mortality among the whiteflies feeding on different transgenic tomato using generalized linear model (negative binomial) revealed that there was no significant difference (P0.05=0.27) in the percentage mortality recorded in whiteflies feeding on all the tested tomato transgenic plants. A low mortality ranging from 2-12% was observed for all the tested transgenic tomato lines with the highest mortality on plants with dsGFP construct (12%) followed by plants with dsSUC construct (10%) and dsRNase+dsAQP+dsSUC construct (7%) (FIG. 31).

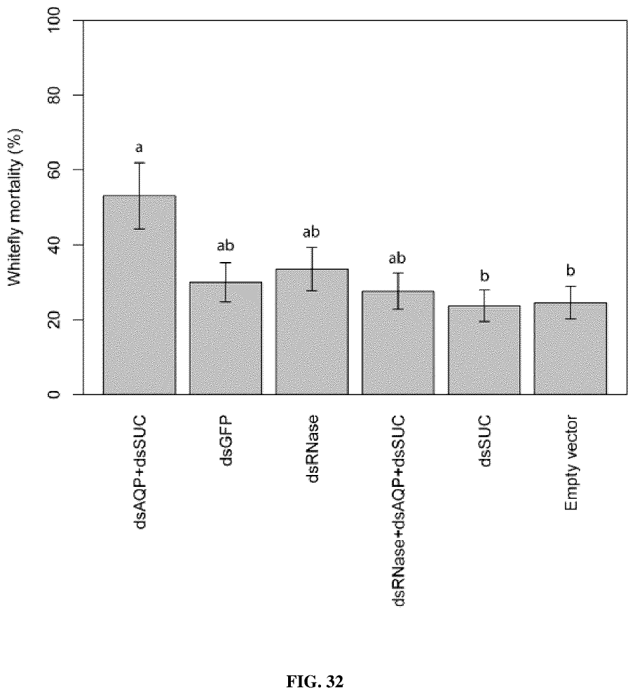

Percentage mortality of the whiteflies feeding on the tested tomato transgenic plants in a second experiment using modified clip cages ranged from 24-53%. Analysis of variance of whitefly mortality on different transgenic tomato plants revealed a statistically significant effect in whitefly mortality in whiteflies feeding on tomato plants with dsAQP+dsSUC construct (P0.05=0.007), with percentage mortality of 53%, based on generalized linear model (negative binomial) test. This performance was almost twice that recorded on tomato plants with dsSUC and dsRNase+dsAQP+dsSUC construct (FIG. 32).

Analysis of SUC1 and AQP1 Gene Expression in B. tabaci Feeding on Different Transgenic Tomato Plants

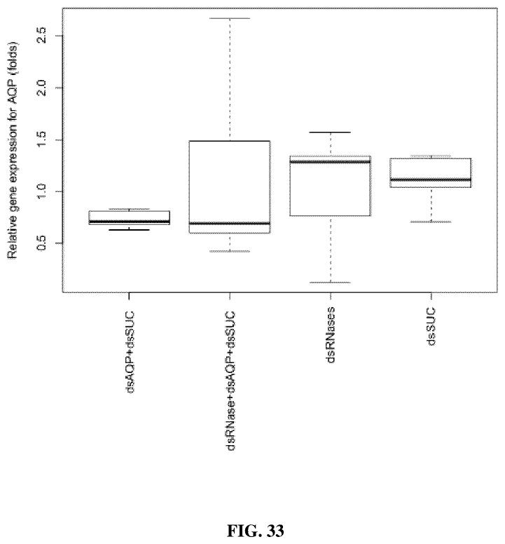

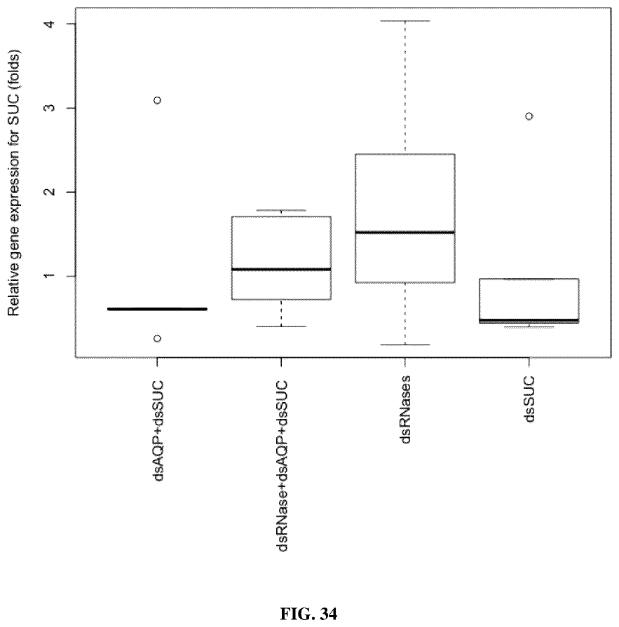

Expression of both AQP1 and SUC1 gene in the surviving whiteflies after six days of feeding was quantified using RT-qPCR. In the first experiment, transgenic lines transformed to knockdown SUC1 gene (dsSUC, dsAQP+dsSUC and dsRNase+dsAQP+dsSUC) had a minimal effect on the expression of SUC1 gene in whiteflies feeding on these tomato plants, with incomplete knockdown not exceeding 22%. Downregulation of SUC1 was observed in whiteflies feeding on tomato transgenic plants containing dsSUC construct and dsAQP+dsSUC construct. Tomato plants containing dsAQP+dsSUC construct were able to downregulate the expression of both AQP1 and SUC1 expression by 0.15±0.11 fold (15%) and 0.22±0.10 fold (22%), respectively (FIG. 33 and FIG. 34).

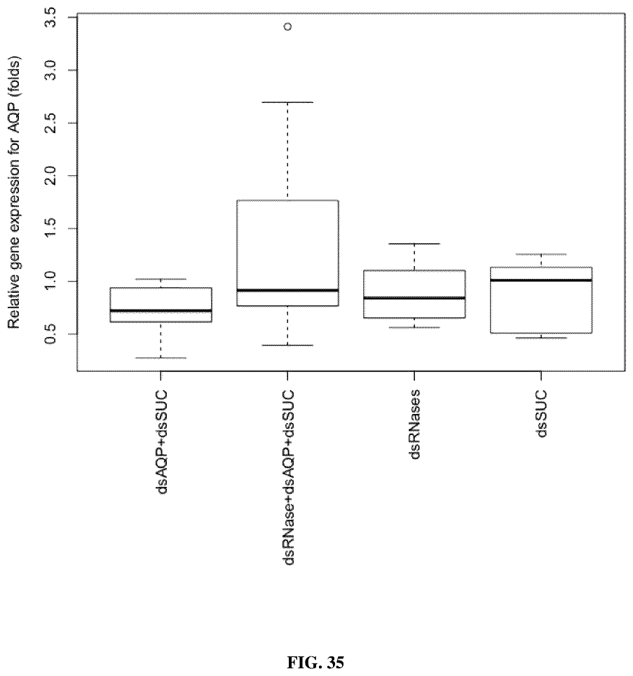

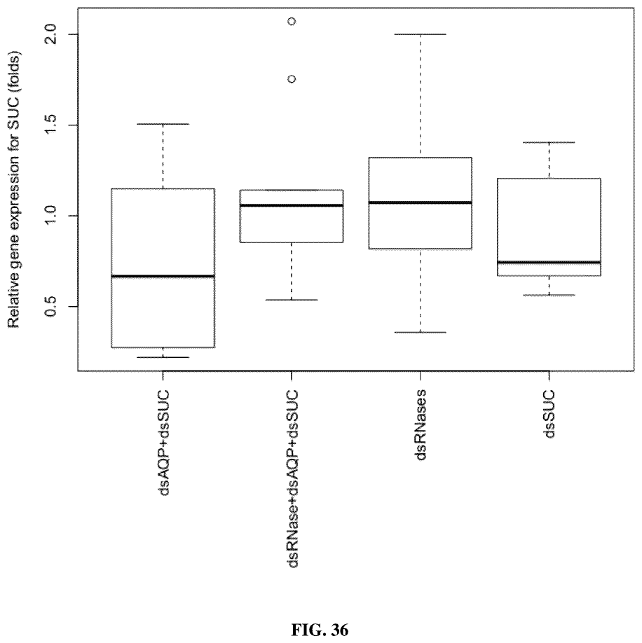

In the second experiment, there was no significant difference in the gene expression of SUC1 in whiteflies feeding on tomato plants with dsRNase construct, dsSUC construct, dsAQP+dsSUC construct and dsRNase+dsAQP+dsSUC construct. However, plants with dsAQP+dsSUC construct had the lowest gene expression of SUC1 of 0.08-fold compared to 0.04 fold and 1.22 fold in whiteflies feeding on tomato plants with dsSUC construct and dsRNase+dsAQP+dsSUC construct, respectively. Furthermore, no significant effect was observed in AQP1 gene expression in whiteflies feeding on the tested transgenic tomato line (Empty vector, dsRNase construct, dsSUC construct, dsAQP+dsSUC construct and dsRNase+dsAQP+dsSUC construct). However, whiteflies feeding on plants with the dsAQP+dsSUC construct had a reduction in AQP1 expression (0.09±0.28 fold) compared to an increase in the gene expression of 1.56±0.48 fold (56%) recorded on the empty vector (FIG. 35 and FIG. 36).

Effect of RNA Interference on B. tabaci Development

In the first experiment, adult whiteflies feeding on different transgenic tomato plants did not exhibit significant effects of RNAi within the six days as hypothesized. Crawlers and nymphs arising from these whiteflies, therefore, were monitored to determine whether RNAi was effective on the next generation. Crawlers (first instar nymphs of whiteflies) emerging from eggs deposited by the five female whiteflies in each cage were counted. These crawlers were monitored through second, third and fourth instar stages. The number of fourth instar nymphs was compared to the crawlers to determine the survival rate, which gave an inference on the effect of RNAi on the development of whiteflies.

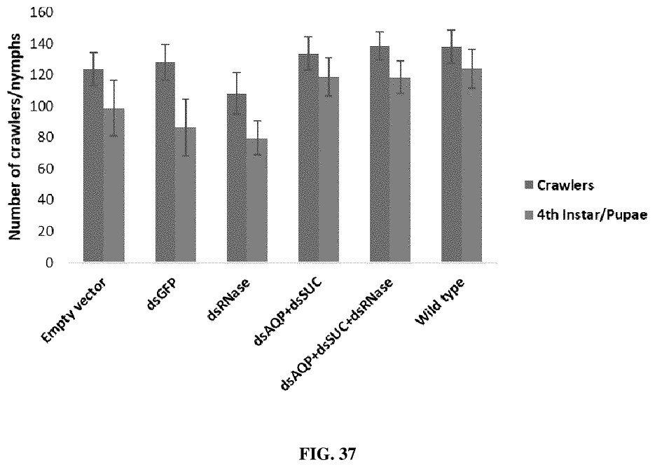

Analysis of variance revealed that there was no significant effect on the number of fourth instar nymphs compared to the crawlers on each test transgenic line. The number of crawlers ranged from 108.2-138.6 with the highest number of crawlers recorded on plants with dsRNase+dsAQP+dsSUC construct (138.6) followed by those with dsAQP+dsSUC construct (133.8). The transgenic plants with the lowest number of crawlers also had the highest level of instar mortality between crawler and 4th instar stage. Plants with the dsGFP-dsRNA construct had the highest instar mortality (32.8%) followed by plants with dsRNase construct (26.4%) and empty vector (20.2%). On average, 70% of the emerged whiteflies on each transgenic line developed to the adult stage (FIG. 37).

Effect of RNAi Against SUC1 and AQP1 Genes on B. tabaci Osmotic Pressure

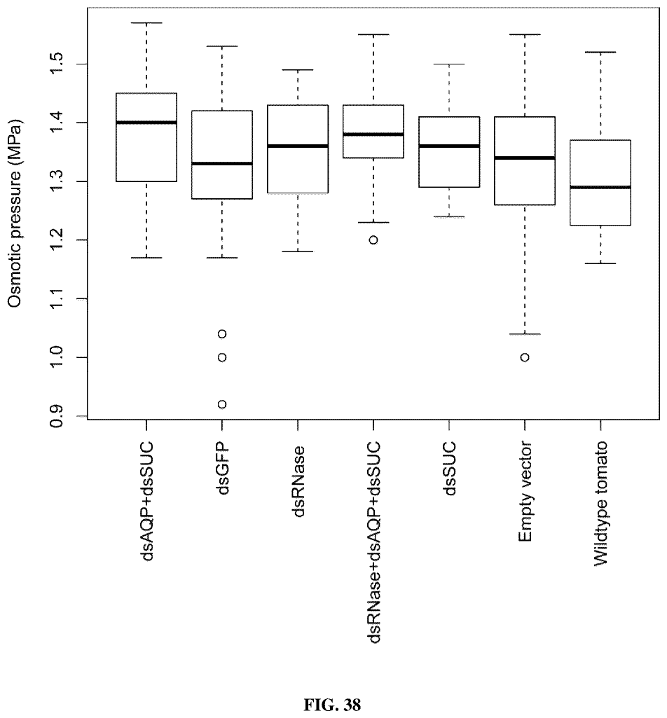

Knock down of osmoregulation gene(s) in phloem-sap feeding insects is expected to increase osmotic pressure within the insect haemolymph. Therefore, haemolymph samples from third instar nymphs feeding on different transgenic tomato lines, empty vector and wild-type tomato plants in the first experiment were analyzed. Results revealed that there was no significant difference (P0.05=1) in haemolymph osmotic pressure of nymphs feeding on all transgenic tomato lines compared to nymphs feeding on empty vector and wild-type tomato plants. Haemolymph osmotic pressure from nymphs feeding on all tested transgenic tomato lines ranged from 1.322-1.375 MPa. Nymphs feeding on transgenic plants containing double-stranded RNA against SUC1 and AQP1 genes (dsAQP+dsSUC and dsRNase+dsAQP+dsSUC) had relatively higher osmotic pressure (1.375 MPa and 1.373 MPa respectively) compared to nymphs feeding on other transgenic lines, empty vector and wild-type tomato plants (FIG. 38).

### Sucrose Hydrolysis in Whiteflies Feeding on Different Transgenic Tomato Lines

The two preliminary studies showed a general trend in sucrose hydrolysis in B. tabaci. It was hypothesized that knockdown of SUC1 would reverse this trend, in other words, knockdown of SUC1 should leave a significant amount of sucrose unhydrolyzed. Sugar analysis of honeydew samples from whiteflies feeding on different transgenic tomato plants with dsRNA against SUC1 (dsSUC) revealed that a significant amount of sucrose remained unhydrolyzed in tomato transgenic line dsSUC (6 M) compared to transgenic line with dsAQP+dsSUC construct and dsRNase+dsAQP+dsSUC construct with 0.4 mM and 0.8 mM respectively.

A reverse trend was observed for free glucose, with significantly lower free glucose in honeydew from whiteflies feeding on transgenic line dsSUC (20 mM) followed by plants with the dsAQP+dsSUC and dsRNase+dsAQP+dsSUC constructs with 30 M and 40 M respectively. In summary, sugar analysis revealed that as more dsRNAs were stacked together with dsSUC, sucrose hydrolysis efficiency increased, yielding more free glucose.

Effect of RNA Interference on Sugar Composition in B. tabaci Honeydew

Liquid chromatography-mass spectrometry sugar analysis was also conducted to determine the percentage molar ratio of monosaccharides, disaccharides and higher sugars in honeydew samples collected from whiteflies feeding on different transgenic tomato plants. There was no significant difference in the molar ratio of glucose, disaccharides and pentasaccharides in honeydew collected from whiteflies feeding on all tested transgenic plants, compared to honeydew collected from whiteflies feeding on plants with empty vector. Based on the percentage molar ratio, B. tabaci honeydew samples tested had more disaccharides in all samples compared to other sugar categories, with the highest amount of disaccharide recorded in honeydew collected from plants with dsRNase construct.

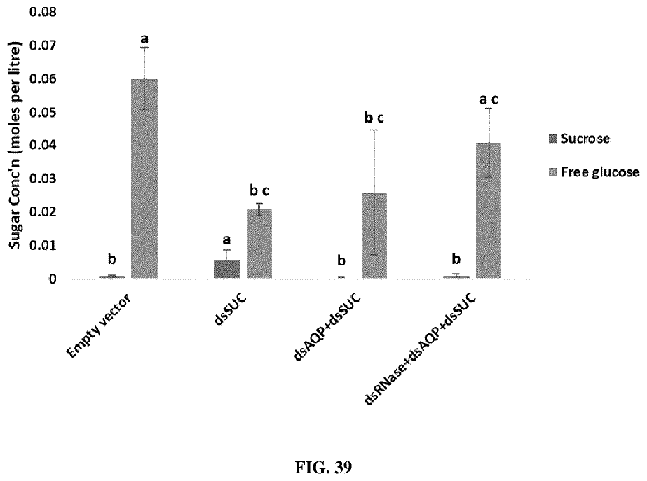

An inverse relation between the number of hexose units (degree of polymerization) and the molar ratio was observed for the oligosaccharides in the honeydew samples analyzed. As the number of hexose units increases, the percentage molar ratio of oligosaccharide reduces, therefore in all honeydew samples, the amount of trisaccharides was higher than tetrasaccharides and pentasaccharides. Analysis of variance of percentage molar ratio among the tested honeydew samples showed that honeydew from whiteflies feeding on plants with dsSUC had a significantly higher percentage of trisaccharides (P0.05=0.03) and tetrasaccharides (P0.05=2.21×10⁻⁶) with 14.03±2.44% and 4.01±0.26% respectively compared to all honeydew samples from whiteflies feeding on all other tested transgenic plants and plant with the empty vector. Furthermore, honeydew from whiteflies feeding on tomato plants with the dsSUC construct had a significantly lower amount of fructose (P0.05=0.006) compared to other transgenic plants and control plant (FIG. 39).

### Effect of RNAi on the Concentration of Different Sugar Categories in Honeydew Samples

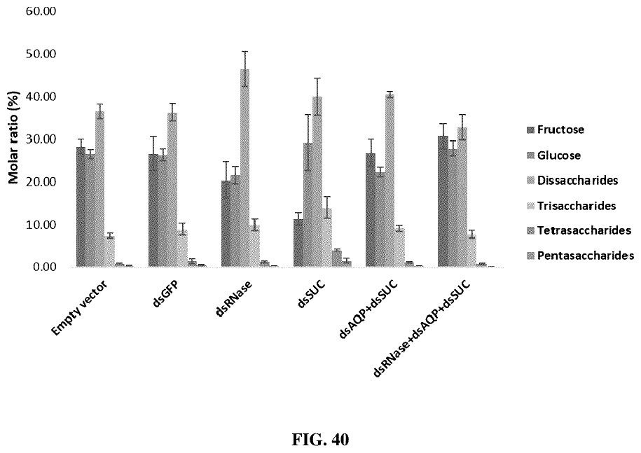

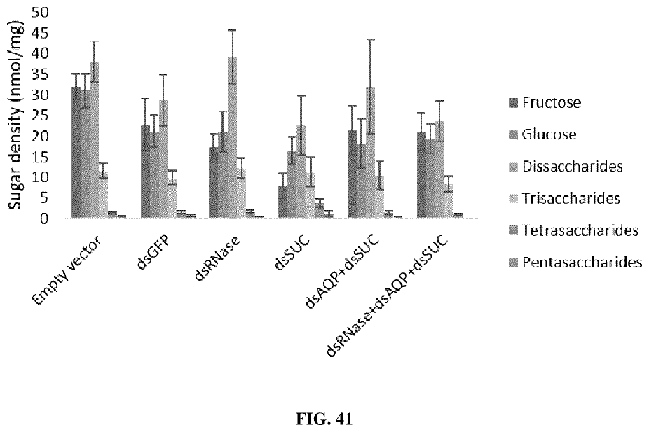

Analysis of concentration of honeydew samples from whiteflies feeding on different transgenic tomato plants revealed that there was no significant difference in concentration of fructose (P0.05=0.05), glucose (P0.05=0.26) and higher sugars in the honeydew samples tested. Honeydew samples from whiteflies feeding on tomato plants with dsSUC construct had relatively few hexose units per mg of honeydew for fructose, glucose and disaccharides but had relatively more hexose units per mg of honeydew for tetrasaccharides and pentasaccharides (FIG. 40 and FIG. 41).

### Comparison of Diet Sugar Utilization of Whiteflies and Aphids

### Assimilation Efficiency of Whiteflies and Aphids Feeding on Artificial Diet

A comparison of sugar concentration of honeydew from both B. tabaci and Aphis gossypii feeding on an artificial diet with 0.75M sucrose concentration showed that B. tabaci honeydew had significantly lower hexose units per mg of honeydew (313.1±51.1 nmol/mg) compared to A. gossypii (612.3±67.7 nmol/mg). Results also showed that B. tabaci MEAM1 assimilated more dietary sugar than aphids with an assimilation rate of 79.1% compared to 59.2% for aphids (Table 15). On tobacco, the sugar concentration in honeydew is much lower for both whiteflies and aphids. Sugar concentration pattern for honeydew from tobacco was similar to that of artificial diet, with significantly lower hexose units (100.9±12.6 nmol/mg) in B. tabaci honeydew, compared to that in aphid honeydew (445.8±99.2 nmol/mg).

TABLE 15
Sugar concentration per mg of honeydew collected from the
whitefly B. tabaci MEAM1 and the aphids
A. gossypii feeding on artificial diet

| | Sugar concentration | | Assimilation |
|---|---|---|---|
| | (Hexose units nmol/mg) | | efficiency |
| Insect | Artificial diet (0.75M) | Tobacco | (%) ** |
| B. tabaci (MEAM1) | 313.1 ± 51.1 | 100.9 ± 12.6 | 79.1 |
| A. gossypii (Aphid) | 612.3 ± 67.7 | 445.8 ± 99.2 | 59.2 |
| p-value | 0.002 | 0.003 | |

** Assimilation efficiency based on sugar concentration of honeydew from aphids and whiteflies feeding on an artificial diet with known sugar concentration

Comparison of Molar Ratio and Concentration of Sugars in Aphid and B. tabaci Honeydew Samples

Comparison of the molar ratios of sugars in honeydew collected from aphids and whiteflies feeding on tobacco and an artificial diet with known sucrose concentration revealed that whiteflies feeding on tobacco produced significantly higher amounts of monosaccharides; fructose and glucose with percentage molar ratio of 23.26±1.89 and 23.32±1.42, respectively compared to honeydew from aphids (14.41±2.71 and 9.42±1.82, respectively). On the contrary, aphids produced significantly higher amount of higher sugars (tetrasaccharides and pentasaccharides), with percentage molar ratio of 10.26±3.92 and 2.61±1.01, respectively compared to 1.14±0.15 and 0.09±0.02 respectively in B. tabaci honeydew.

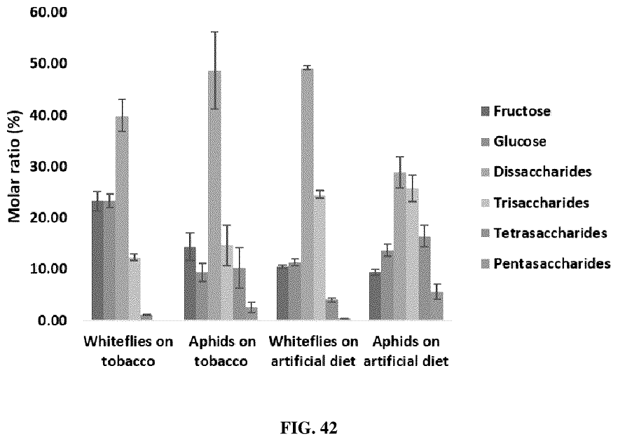

Analysis of honeydew collected from aphids and whiteflies feeding on an artificial diet with the same concentration of sucrose (0.75 M) revealed that there was no significant difference in the percentage molar ratio of fructose, glucose and trisaccharides in honeydew samples. Honeydew from whiteflies had a significantly higher amount of disaccharide, almost 50% compared to 28.9% in the honeydew from aphids. On the other hand, honeydew from aphids was much richer in higher sugars than that from whiteflies. Honeydew samples from aphids feeding on an artificial diet had the significantly higher amount of tetrasaccharides and pentasaccharides (16.5% and 5.6% respectively) compared to 4.1% and 0.4%, respectively from B. tabaci honeydew samples (FIG. 42). These results show that B. tabaci honeydew is composed of more monosaccharides and disaccharides (higher percentage of monomers), while aphids produce a high percentage of oligomers.

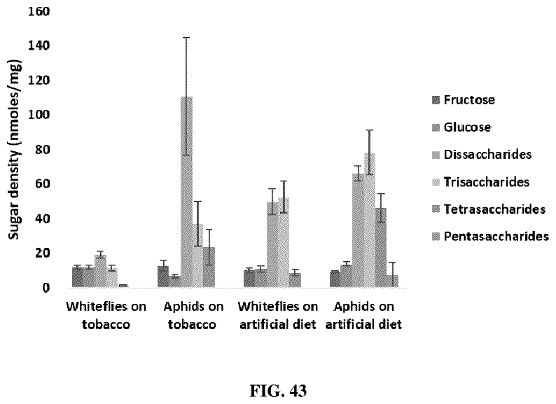

In addition, analysis of sugar concentration in honeydew samples collected from both aphids and whiteflies feeding on tobacco and an artificial diet showed that honeydew collected from aphids was much richer in sugars compared to honeydew from whiteflies (FIG. 43). Pairwise comparison of monosaccharides, disaccharides and higher sugars of aphid and whitefly honeydew sugars showed no significant difference in the concentration of fructose and trisaccharides per mg of honeydew, in both aphids and whiteflies honeydew samples from tobacco. Whiteflies feeding on tobacco produced honeydew with a significantly higher number of hexose units per mg of honeydew for glucose (11.87±1.52), compared to 6.74±1.18 in aphid honeydew sample. On the contrary, honeydew samples from aphids feeding on tobacco had significantly higher concentration for disaccharide (110.6 nmol/mg), tetrasaccharides (23.37 nmol/mg) and pentasaccharides (0.07 nmol/mg) compared to 19.18 per mg, 1.08 per mg and 0.02 per mg respectively, in B. tabaci honeydew.

On the artificial diet, the pattern of sugar concentration for monosaccharide, disaccharide and oligosaccharide changed, showing that the source of sugar influences the concentration of sugars in the resulting honeydew. Comparison of the concentration of different sugars in honeydew samples collected from whiteflies and aphids feeding on the same artificial diet with 0.75M sucrose showed that only tetrasaccharides in aphid honeydew sample were significant, with 46.2 nmol/mg compared to 8.64 nmol/mg in B. tabaci honeydew. In summary, aphids produce honeydew richer in sugars than the equivalent honeydew samples from whiteflies.

### Example 4: Enzymes from the Glycoside Hydrolase Family 13 of Bemisia tabaci Play an Important Role in Osmoregulation Processes

To further elucidate key genes involved in B. tabaci osmoregulation, GH13 enzymes involved in sucrose isomerization, especially the biosynthesis of trehalulose, were investigated. Trehalulose is synthesized by rearranging the glycosidic bond of sucrose from the two to the one position of fructose. Trehalulose has been found to be the main sugar present in the honeydew of B. tabaci, and its fractions can account for up to 70% of the total carbohydrates. Trehalulose is 10 times less susceptible to hydrolysis by sucrases than sucrose, and its production prevents the rapid rise in gut osmolarity due to the hydrolysis of sucrose to its monomers.

Transcriptomic data and phylogenetic analyses were used to select four “key” GH13 family members potentially involved in sucrose isomerization. All four selected genes were individually silenced by feeding adult insects on double-stranded RNA (dsRNA) diets, and the silencing effects on survival, fecundity, and progeny development were tested. In addition, metabolomic analyses were conducted on the honeydew secreted during the silencing period. This allowed determination of how the decreased expression levels of the target genes affected the metabolism of sucrose. These findings support the model that the isomerization of sucrose is vital for osmoregulation in B. tabaci and directly relates to the performance and fitness of the insect.

### Host Plant and Insect Population

Brussels sprout plants, Brassica oleracea L var. gemmifera L. ‘Franklin’ cultivar (Eden Seeds, Israel), were used throughout this study as host plants for B. tabaci, both for colony maintenance and performance assays. The Brussels sprout seeds were germinated in square plastic pots (6×6×6.5 cm³) containing peat, coconut, tuff, NPK fertilizer, and micronutrients (Bental 11, Tuff Merom Golan, Israel). Two to three weeks later, the seedlings were transplanted into larger pots (12 cm diameter). The plants were used for rearing and experimenting when they developed four “true” leaves (seven weeks from germination). Greenhouse conditions were 30±4° C. with 30-40% relative humidity and a 14:10 (L:D) photoperiod. The colony of the B. tabaci, MEAM1 species, was established using adults collected in Menahamya (North of the Jordan Valley, Israel). The colony was reared on Brussels sprout plants in fine mesh (160 μm) insect-rearing tents (47.5×47.5×93 cm³) under greenhouse conditions (28±4° C., 40-60% relative humidity, and a 14:10 (L:D) photoperiod).

Phylogenetic Analysis of the Glycoside Hydrolase Family 13 in B. tabaci:

Putative α-glucoside hydrolase family 13 (GH13; Pfam: PF00128, Finn et al., 2016) protein sequences of B. tabaci were downloaded from NCBI (https://www.ncbi.nlm.nih.gov/) and the whitefly genomics open databases (http://www.whiteflygenomics.org/; Chen et al., 2016). First, duplications and variant sequences with 98% sequence identity or higher were concatenated and de-replicated using CDHIT v4.6 with default parameters (Fu et al., 2012). The remaining 70 sequences were aligned using MAFFT v7.215 (Katoh and Standley, 2013; default parameters) and blocks of phylogenetically informative positions were chosen using Gblocks v0.91b (Castresana, 2000; most permissive parameters per alignment). A maximum-likelihood phylogenetic tree was built using the best-fit model of amino acid replacement selection and 5000 bootstraps in IQ-TREE v1.6.5 (Nguyen et al., 2015), with the following command: iqtree-s all_CDHIT098.faa.aln-gb-st AA-nt 14-m TEST-bb 5000-alrt 1000. Tree representation was performed using iTOL version 6 (https://itol.embl.de/).

dsRNA Design and Synthesis

The dsRNA molecules were designed using E-RNAi version 3.2 (http://www.e-rnai.org/; Horn and Boutros, 2010). Each designed dsRNA molecule was BLASTed using NCBI BLASTn (https://blast.ncbi.nlm.nih.gov/Blast.cgi/) and manually curated to make sure that no ≥14 nucleotide long fragments are identical between the designed dsRNA sequence and transcripts of Brussels sprout, beneficial insects, farm and household animals, and humans (Chen et al., 2021). The sequences were then sent to RNA Greentech LLC (Texas) for dsRNA synthesis. All dsRNA sequences are available in Table 16. As a control, dsRNA molecule targeting the gene coding for the coat protein of the Cassava Brown Streak virus (dsCBSV) was used, which is not present in the genome of B. tabaci.

### Artificial Diet Bioassay

One hundred newly emerged B. tabaci adults were placed in 30×200 mm glass vials and fed with 150 μl treatment solution containing autoclaved tap water, dsRNA at a final concentration of 0.5 μg/μl, and sucrose at a final concentration of 0.5M. The treatment solution was placed between two layers of 4×4 cm Parafilm pieces stretched over a self-designed (Fusion 360 modeling software, Autodesk Inc.), 3D-printed lid (35 mm diameter). Before each experiment was conducted, all vials, lids, and Parafilm pieces were sterilized for 15 min under UV-C light. All vials were wrapped with aluminium foil, and the lids were covered with green cellophane paper to mimic feeding on the abaxial side of the leaf. The artificial diet experiments were conducted in a rearing chamber under controlled conditions at 28±2° C., 50-55% relative humidity, and a 14:10 (L:D) photoperiod.

### Analysis of Survival Assays

After five days of feeding, the number of surviving and dead insects in each vial was counted separately, and the survival proportion was calculated. The artificial diet experiments were conducted in three blocks, each with five replicates for each dsRNA treatment and five replicates for the control treatment. To enable the analysis of the combined data, the survival proportion of each replicate in each block was normalized to the mean survival proportion of the block's control treatment. Finally, the significance of the differences between the survival means was determined by a one-way ANOVA model followed by pairwise comparisons. Prior to the analysis, the proportional data were arcsin-square root transformed. As multiple tests were conducted, a false discovery rate (FDR) correction was applied. Statistical significance was assumed at P≤0.05. All described statistical analyses in the manuscript were conducted using JMP Pro 17.0 (SAS Institute, Cary, NC).

### Gene Expression Analyses

Samples of 50 surviving adults from the artificial diet assays were collected in 1.5 ml Eppendorf tubes, immediately thrown into liquid nitrogen, and stored at −80° C. Next, the RNA was extracted using ISOLATE II RNA mini kit (Meridian Bioscience, Ohio) following the manufacturer's protocol. DNA contamination was removed using a PerfeCTa DNaseI (Quanta Bioscience, Massachusetts) treatment. RNA quality and quantity were evaluated using a NanoDrop 2000 spectrophotometer (Thermo Fisher Scientific, Massachusetts). Samples in which the 260/280 or 260/230 values were less than 1.8 were treated with RNA Clean & Concentrator kit (Zymo Research, California). Next, a reverse transcriptase reaction was performed using 500 ng RNA from each sample and the Verso cDNA synthesis kit with Oligo-dT primer (Thermo Fisher Scientific, Massachusetts). Finally, the expression level of a target gene was examined using quantitative real-time PCR (qRT-PCR). The reactions were performed using CFX Connect Real-Time PCR System (BIO-RAD, California). A set of primers was designed and calibrated for each target gene based on the Thornton and Basu (2011) protocol using Primer3 software (https://primer3.ut.ce/) (Table 17). The B. tabaci ribosomal protein L13a (RPL13A) was used as the reference gene. The qRT-PCR conditions were adjusted for the amplification efficiencies of the target and reference genes to be in the log range of 1.9-2.1; they were optimally set to a master mix containing 5 μl iTaq Universal SYBR Green Supermix (BIO-RAD, California), 0.5 μl of both forward and reverse primers (2 μmol/μl), 2 μl DDW and 2 μl cDNA template. The expression of target and reference genes was tested in triplicate for each sample to ensure the validity of the results. qRT-PCR thermal conditions consisted of one cycle of 95° C. for 2 min, followed by 40 cycles of 95° C. for 5 sec and 60° C. for 30 sec, and an ending cycle of 95° C. for 5 sec, 65° C. for 5 sec, and 95° C. for 30 sec. The homogeneity of the PCR products was confirmed by melting curves analyses. Quantification of the transcript's expression levels was conducted according to the ΔΔCt method (Applied Biosystems). A one-way ANOVA model was used to determine the significance of the differences between the expression means of the treatment and control samples (P≤0.05).

### Development Assays

Artificial diets were conducted as detailed above and on the fifth day, all vials of each treatment (silencing one specific gene) were collected and placed (after removing the lids) for 24 h in an insect-rearing tent containing an 8-week-old Brussels sprout plant. During this period, the surviving adults moved to the Brussels sprout plant, and the females laid “mature” eggs (i.e., eggs that were already ready to be laid before the beginning of the dsRNA feeding treatment or completed their development during the feeding period). After 24 h, samples of seven pairs (male and female) were collected into 2.5×5 cm glass clip-cages, and each clip-cage was placed on a new 8-weeks-old brussels sprout plant in a rearing tent. The females were allowed to lay eggs for 48 h, after which the clip-cages and all adults were gently removed. The development assays were conducted in greenhouse conditions (30±4° C. with 30-40% relative humidity and a 14:10 (L:D) photoperiod), and the plants were watered as needed every two to three days. Once newly emerged adults were detected in one of the treatments, all the infested leaves of all the treatments were removed from the plants, and a “snapshot” was conducted, meaning that we determined for each leaf the proportion of unhatched eggs, undeveloped nymphs (dried nymphs and nymphs with abnormal morphology), 2^{nd} instars, 3^{rd} instars, early 4^{th} instars, late 4^{th} instars (also known as “pupae”), and exuviae (indicating the successful completion of the development and emergence of an adult). Differences in the proportion of undeveloped progeny (undeveloped eggs and nymphs) and progeny at advanced developmental stages (early 4^{th} instars, “pupae”, and exuviae) between each treatment and the control were tested for significance using the log-likelihood ratio test. As multiple tests were conducted, a false discovery rate (FDR) correction was applied. Statistical significance was assumed at P≤0.05.

### Metabolomic Analysis

At the end of the five days of feeding on the dsRNA diets, the sugar profile of the honeydew that was excreted by the feeding adults was examined. First, the honeydew deposited on the glass vials was washed with two ml of deionized water (Bio-Lab ltd., Israel), preheated to 75±5° C. Next, the honeydew samples were filtered through Corning costar spin-x plastic centrifuge tube filters (cellulose acetate membranes with 0.22 μm pore size; Sigma-Aldrich, Missouri), vacuum dried, reconstituted in deionized water by 15 min sonication at 35° C. and stored frozen at −20° C. For analysis, the aqueous sample was diluted in an N, N-Dimethylformamide (DMF, 99.8% pure; Sigma-Aldrich, Missouri) acetonitrile mixture to a final composition of 9-aqueous: 10-DMF: 21-acetonitrile (volume base).

The composition of sucrose metabolites in the diluted honeydew samples was analyzed via liquid chromatography-mass spectrometry (LC-MS) using the ACQUITY UPLC H-Class system equipped with a Single Quadruple Detector (SQD2; Waters, Massachusetts). All analyses were performed as follows: 0.1-7 μl sample volumes were injected onto a BEH-amide column (2.1×50 mm, 1.7 μm), maintained at 36° C. Chromatographic separation was attained using a gradient of water (Solvent A), and acetonitrile (Solvent B) supplemented with 0.1% ammonium hydroxide, at a flow rate of 0.17 mL/min as follows: 80-75% B for 6 min, 75-65% B for 1.5 min, 65-58% B for 0.5 min, 58-55% B for 3 min, 55-80% B for 1 min, and a 10 min hold on 80% B. Monosaccharides to nonasaccharides sugars and sugar alcohols were detected by negative electrospray ionization, following predefined m/z values. The profiles were acquired and integrated using Empower 3 software (Waters, Massachusetts). The sugar reference materials were purchased from Sigma-Aldrich (fructose, glucose, sorbitol, mannitol, inositol, sucrose, turanose, isomaltulose, maltose, trehalose, α,β-trehalose, erlose, melezitose, raffinose, stachyose), TCI (isomaltose), Carbosynth (maltulose, maltohexaose, isomaltohexaose, maltononaose), and Muschem (trehalulose).

Peaks in each m/z (mass/charge) channel of the LC-MS separation were identified by comparing their retention times to reference materials of the same m/z or were given only a class identification based on their m/z values (e.g., “trisaccharide”) if their retention times did not fit any of the reference materials. In the three cases of reference material coelution-sorbitol with mannitol (m/z 181.2), trehalulose with maltose (m/z 341.3) and melezitose with erlose (m/z 503.4), peaks in honeydew profiles with the retention times and m/z values in question were identified as sorbitol, trehalulose and melezitose, respectively, for consistency with previous studies (Byrne et al., 2003; Costa et al., 1999; Davidson et al., 1994; Hendrix and Wei, 1994; Roopa et al., 2016; Salvucci et al., 1997; Salvucci et al., 1999; Wei et al., 1996; Wei et al., 1997; Wolfe et al., 1999). In addition, peak areas of the two sucrose isomers maltulose and trehalulose (both with m/z=341.3) that eluted sequentially were summed together and identified as trehalulose due to very high quantities of the latter compared to the former (typically at least 10-fold more), that prevented appropriate division of the peak areas. Areas were converted to nano-equivalents of sucrose using non-linear calibration curves generated using the reference materials. For compounds with only a class identification, average calibration parameters from the most chemically similar reference materials were used. Finally, the quantity of each compound in the original sample was calculated using the dilution data. To allow separate analysis of honeydew composition and sugar honeydew quantity, the sugars from monosaccharides and sugar alcohols up to hexasaccharides were summed to give the “yield” in each sample. Next, the fraction of each sugar out of the total honeydew was calculated by dividing its quantity by the yield. Sugars larger than hexasaccharides were not included in the calculations due to their inconsistent quantitation, resulting from a decrease in the sensitivity of the analysis method to the increasing sugar size.

The significance of the differences in the fraction size of sucrose, and the combined fraction sizes of glucose+fructose, disaccharide isomers (of sucrose) and oligosaccharides (trisaccharide to hexasaccharides), was determined by a one-way ANOVA model followed by pairwise comparisons. Prior to the analysis, the proportional data were arcsin-square root transformed. As multiple tests were conducted, a false discovery rate (FDR) correction was applied. Statistical significance was assumed at P≤0.05.

TABLE 16
dsRNA construct sequences directed against SUC3,
SUC4, SUC5, SUC7, SUC12, and CBSV

| Name | Construct Sequence |
|---|---|
| dsSUC3 | SEQ ID NO: 70 |
| dsSUC4 | SEQ ID NO: 71 |
| dsSUC5 | SEQ ID NO: 72 |
| dsSUC7 | SEQ ID NO: 73 |
| dsSUC12 | SEQ ID NO: 74 |
| dsCBSV | SEQ ID NO: 75 |

TABLE 17
Primers directed to RPL13A, SUC3, SUC4, SUC7, and SUC12.

| # | Name | Sequence (5′ -> 3′) |
|---|---|---|
| 1 | qRT-PCR:Bta04282 (RPL13A) Forward | SEQ ID NO: 76 |
| 2 | qRT-PCR:Bta04282 (RPL13A) Reverse | SEQ ID NO: 77 |
| 3 | qRT-PCR:Bta04298 (SUC3) Forward | SEQ ID NO: 78 |
| 4 | qRT-PCR:Bta04298 (SUC3) Reverse | SEQ ID NO: 79 |
| 5 | qRT-PCR:Bta15649 (SUC4) Forward | SEQ ID NO: 80 |
| 6 | qRT-PCR:Bta15649 (SUC4) Reverse | SEQ ID NO: 81 |
| 7 | qRT-PCR:Bta07453 (SUC7) Forward | SEQ ID NO: 82 |
| 8 | qRT-PCR:Bta07453 (SUC7) Reverse | SEQ ID NO: 83 |
| 9 | qRT-PCR:Bta12682 (SUC12) Forward | SEQ ID NO: 84 |
| 10 | qRT-PCR:Bta12682 (SUC12) Reverse | SEQ ID NO: 85 |

Bioinformatic Analysis, Identification and Selection of GH13 Enzymes with Putative Sucrose-Isomerization Activity

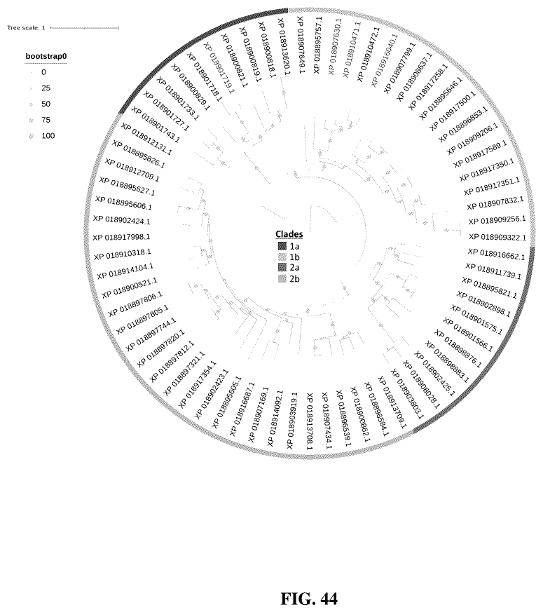

A phylogenetic tree of the GH13 enzyme family in B. tabaci was produced using 70 protein sequences containing the Pfam alpha-amylase domain (PF00128; Finn et al., 2016). This analysis resulted in two sub-trees (1 and 2), each containing two main clades (la, 1b, 2a, and 2b) (FIG. 44). SUC5, which was previously shown to express strong transglucosidation activity (Malka et al., 2020) was placed in sub-tree 2. Therefore, sucrose isomerization enzymes in sub-tree 1 was investigated, specifically in the 1b clade. This is because SUC2, which showed a mild ability to glycosylate glucosinolates in previous analysis, was placed in clade 1a. Moreover, clade 1b contained the three others previously analysed GH13 enzymes, SUC1, SUC3, and SUC4, which were shown not to produce transglucosidation products. In addition, the gut transcriptome of B. tabaci (SRX3230134) was investigated to choose the most highly expressed GH13 genes. After combining the data from the two bioinformatic approaches, BtSUC3 (XM_019052085.1), BtSUC4 (XM_019060495.1), and BtSUC7 (XM_019054926.1) were selected for further study. As a reference gene, BtSUC12 (XM_019046174.1) was chosen, which also showed high expression levels in the insect's gut but belonged to clade 1a.

### Silencing Selected GH13 Genes in Adults and the Effect on Survival

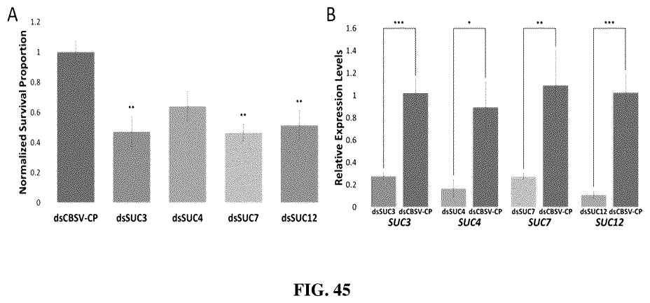

Five days of feeding on artificial diets containing dsSUC3, dsSUC4, dsSUC7, and dsSUC12 resulted in mortality rates of 78.75±4.51%, 71.02±4.58%, 78.85±2.53%, and 77.06±4.29%, respectively, which were significantly (excluding dsSUC4) higher than the mortality rates (54.75±4.24%) in the control assay (feeding on a diet containing dsCBSV) (FIG. 45, Panel A). To associate between the increased mortality rates and the silencing of the target genes, the expression levels of each one of them was examined using RT-PCR. The results indicated that the artificial diets were effective in reducing the expression levels of all four genes (3-9-fold), when compared to insects feeding on dsCBSV (FIG. 45, Panel B).

Plant Performance Assays: Silencing Effects on the Fecundity and Survival of Adults and their Progeny Development

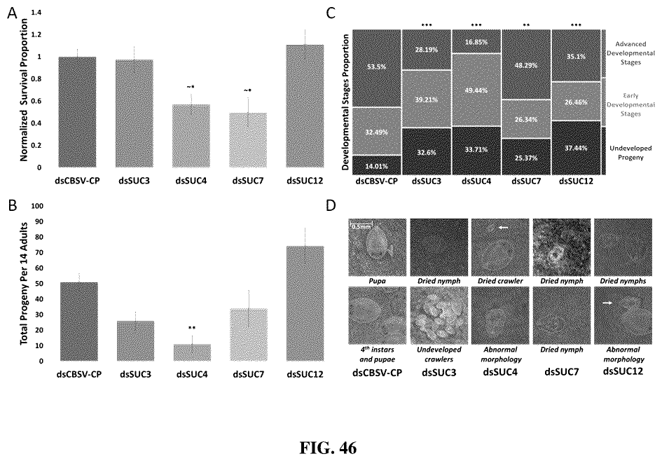

After feeding for five days on artificial diets of dsRNA, samples of surviving insects were collected and placed on Brussels sprout plants. It was observed that the dsSUC4 and dsSUC7 treatments have resulted in a nearly-significant increased mortality (when compared to the dsCBSV treatment) of the silenced adults after 48 hours (FIG. 46, Panel A), and that the dsSUC4 treatment also caused a significant reduced production of progeny (when compared to the dsCBSV treatment) (FIG. 46, Panel B). Next, it was determined whether targeting the GH13 genes of the mothers also affects the development rate and/or causes abnormal phenotypes in their progeny. These experiments showed that the progeny of the surviving insects in all treatments had a significantly delayed development compared to the progeny of adults fed on dsCBSV (FIG. 46, Panel C). This included a higher proportion of undeveloped progeny (i.e., unhatched eggs and dried nymphs) and a lower proportion of progeny in advanced developmental stages after 21 days of development (i.e., both 4^{th} instar nymphs, “pupae”, and progeny that completed their development and emerged). Moreover, the “undeveloped progeny” group which included nymphs displaying abnormal morphology, such as flat nymphs, distorted shapes, and pale color, was significantly higher in all treatments than the control (the various abnormal phenotypes are presented in FIG. 46, Panel D).

### Metabolomic Analyses

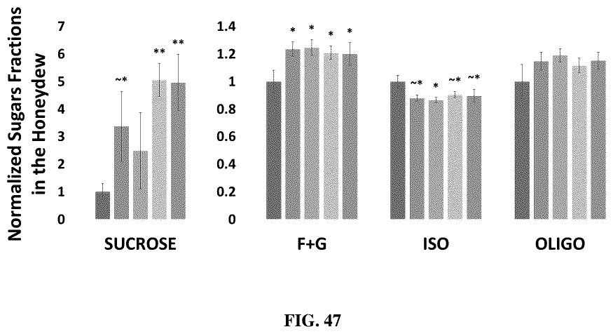

Examining the carbohydrates composition of the honeydew secreted by B. tabaci adults during the dsRNA feeding period, revealed how silencing each of the GH13 genes affects the sucrose metabolism of the insects (FIG. 47). A higher fraction of sucrose (significant effect during the silencing of SUC7 and SUC12, and nearly significant effect during the silencing of SUC3) was found in all dsRNA treatments when compared to feeding on the dsCBSV control. A significantly higher fraction of the sucrose monomers fructose and glucose was also observed in the treatment samples (when compared to the control). These two results indicated impaired sucrose hydrolysis and a lower ability to assimilate the hydrolysis products. Next, it was determined whether the GH13 silencing affected sucrose isomerization and transglucosidation. The combined fractions of oligosaccharides did not differ significantly between the dsGH13 diets and the control. In contrast, the combined fractions of all sucrose isomers, which were mainly composed of trehalulose (approximately 40-60% of all isomers), were significantly or nearly significantly lower in all dsRNA treatments compared to the dsCBSV control, suggesting that the GH13 genes targeted in this research are involved in sucrose isomerization in B. tabaci.

### Example 5: Field Trials Showing the Efficacy of RNA Interference Against Osmoregulation Genes and Symbiosis Genes in the Management of Bemisia tabaci

Two confined field trials (CFTs) were implemented and completed in Namulonge in Uganda to evaluate the resistance of transgenic cassava events to whiteflies. The first field trial tested only lines that target osmoregulation genes (see Examples 1-3). The second field trial tested an additional line that targets an osmoregulation gene (see Example 4). It also tested lines that disrupt the symbiotic relationship between B. tabaci (whitefly) and its obligate symbiotic bacterium Portiera, preventing whitefly-nymphal growth and adult egg production (see Example 1).

### Whitefly Resistance Trait Selection Trial I (WFR TST I)

The first whitefly resistance Trait Selection Trial (WFR TST I) was planted using tissue culture plantlets. The trial had 18 plots (14 RNAi transgenic events and 4 controls). The transgenic lines included plants with RNAi dsRNA targeting the aquaporin (AQP1, SEQ ID NO:2) and RNase genes in DWF10, aquaporin (AQP1) gene in DWF11, stacked aquaporin (AQP), sucrase (SUC1, SEQ ID NO:4) and RNase genes in DWF14 and aquaporin (AQP1) stacked with sucrase (SUC1) in DWF15. The lines included; DWF10-N13009, DWF10-N13011, DWF10-N13012, DWF11-N13001, DWF11-N13004, DWF11-N13008, DWF14-N06002, DWF14-N13006, DWF15-N06001, DWF15-N06002, DWF15-N06003, DWF15-N13001, DWF15-N13002, and DWF15-N13005. Controls included; 5001-N13006 (transgenic CBSD resistant NASE 13 control), 0000-N13001 (non-transgenic NASE 13 control), NASE 12 (non-transgenic whitefly susceptible control) and Mkumba (non-transgenic whitefly tolerant control). The field was planted in a Randomized Complete Block Design (RCBD) with 5 replications. Each plot consisted of 10 plants (5 rows, each with 2 plants). To determine the number of adult B. tabaci on each entry, in-field observations were made on the underside of the top five fully-expanded leaves of the tallest shoot from all the 10 plants in the plot. For the total B. tabaci nymph count, the 14^{th} leaf from the top, known to host the highest number of 3^{rd} and 4^{th} instar nymphs were also manually counted in the field (also called raw counts).

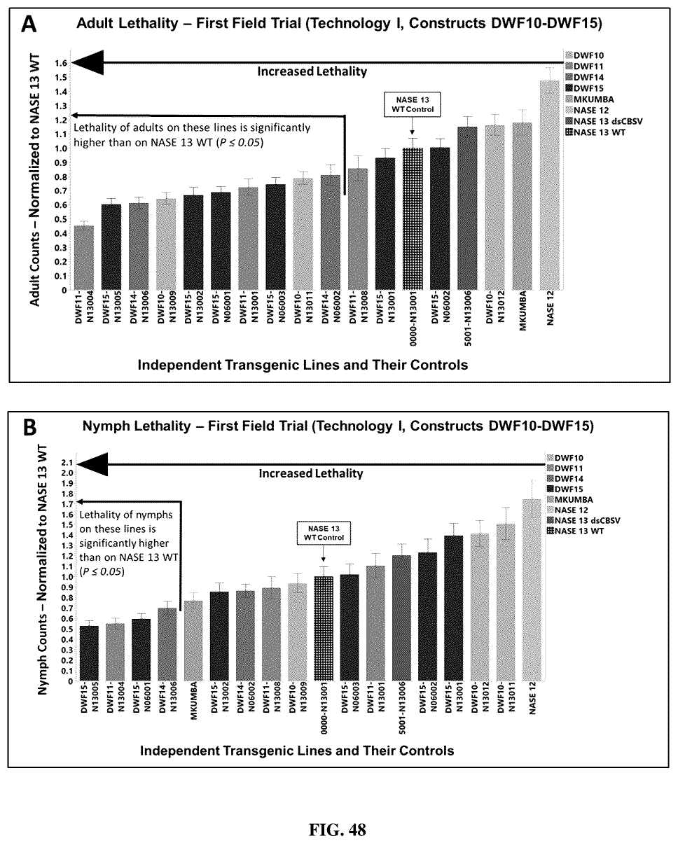

Data was collected over a five-month period after which the plants were too tall to allow accurate visual assessment. Data showed that ten transgenic events had significantly lower adult whitefly numbers on the top five leaves and four had lower nymph counts when compared to non-transgenic control variety, NASE 13 WT (FIG. 48, panels A and B).

### Whitefly Resistance Trait Selection Trial II (WFR TST II)

A second trait selection trial (WFR TST II) was planted in Namulonge. This trial was established using disease-free tissue culture-derived plants. It evaluated 11 transgenic events including 6 related to the osmoregulation and symbiosis technologies. The transgenic lines included plants with RNAi dsRNA targeting the osmoregulation sucrase 5 (SUC5—SEQ ID NO: 72) gene in DWF57, and two genes involved in the symbiotic relationship between whiteflies and the bacterium Portiera, Arginosuccinate lyase (ArgH—SEQ ID NO:8) in DWF61 and Chorismate mutase (CM)—SEQ ID NO:68) in DWF62. The lines included: DWF57-N13003, DWF57-N13008, DWF61-N13007, DWF61-N13009, DWF61-N13010 and DWF62-N13004. These were received as tissue-culture plantlets, micro-propagated, and planted alongside disease-free plants of 0000-N13001 (WT-NASE 13), and NASE 12 and Mkumba as whitefly susceptible and tolerant checks, respectively. Each plot consists of ten plants with four replications planted in a RCBD.

Plant establishment for this trial varied across different entries. The control, NASE 12, established from disease-free stakes, had the best-establishment, with a 100% survival rate. On average, the trial achieved a 67.6% establishment rate, with several entries surpassing the average. Notably, entries like DWF57-N13008 and DWF61-N13009 had an 86% establishment rate. To determine the number of adult B. tabaci on each entry, in-field observations were made on the underside of the top five fully-expanded leaves of the tallest shoot from all the 10 plants in the plot. For the total B. tabaci nymph count, the 14^{th} leaf from the top, known to host the highest number of 3^{rd} and 4^{th} instar nymphs were also manually counted in the field (also called raw counts).

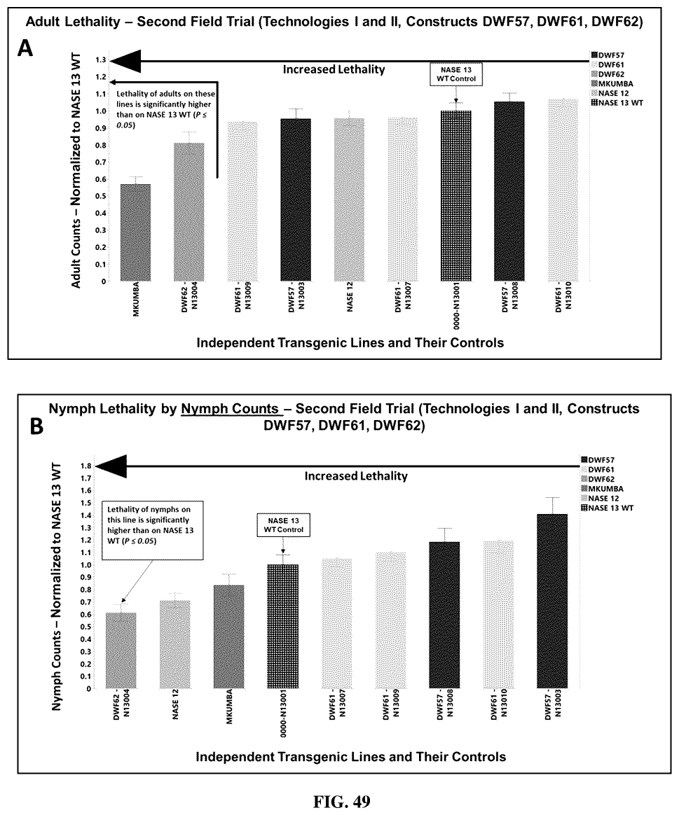

Data was collected over a five-month period after which the plants were too tall to allow accurate visual assessment. Data showed that only one transgenic event, DWF62-N13004, had significantly lower adult and nymphs whitefly numbers when compared to non-transgenic control variety, NASE 13 WT (FIG. 49, Panels A and B).

### Example 6: Laboratory Assays

Prior to sending the cassava plants of NASE 13 modified to express the RNAi technology to the field, they were tested under laboratory conditions using a SS1-SG1 whitefly colony. The transgenic lines that targeted the osmoregulation genes were: aquaporin (AQP1) and RNase genes in DWF10, the aquaporin (AQP1) gene in DWF11, the stacked aquaporin (AQP), sucrase (SUC1) and RNase genes in DWF14, the aquaporin (AQP1) stacked with sucrase (SUC1) in DWF15, the sucrase 5 (SUC5) gene in DWF57 and the sucrase 12 (SUC12, SEQ ID NO:74) gene in DWF59. The transgenic lines that targeted the symbiotic relationship between whiteflies and the bacterium Portiera were: DWF 61 (Arginosuccinate lyase, ArgH); DWF 62 (Chorismate mutase, CM); DWF 63 (Diaminopimelate decarboxylase, LysA, SEQ ID NO:9); DWF 64 (Diaminopimelate epimerase, DapF, SEQ ID NO:69) and DWF 65 (Biotin synthase, BioB, SEQ ID NO:67). We used two bioassays, adult survival after 7 days of exposure to the RNAi plants and nymph development rate (the development stage reached 25 days after the egg was laid).

In the adult survival assays, whiteflies were released onto six-week-old cassava plants in Lock-Lock cages within an insectary (25±2° C.; 14:10 h light: dark cycle at 40% relative humidity). Five plants (replications) per transgenic event/line were tested with fifteen pairs (male and female) of 1-2-day-old, whitefly adults. Survival was recorded on the 7th day after the release of the whiteflies.

In the nymph development assays, fifteen pairs (male and female) of 1-2-day-old whitefly adults were released onto six-week-old cassava plants in Lock-Lock cages within the NRI insectary (25±2° C.; 14:10 h light: dark cycle at 40% relative humidity) for a 24 h egg laying period. Six plants (replications) per transgenic event/line were tested. After 25 days, the development status of the progeny was determined in each replicate (number of 2nd and 3rd nymphs, number of red-eyed 4th nymphs, number of progeny that completed development and emerged as adults leaving the remains of their exoskeleton (exuviae) behind).

### Laboratory Adult Survival Assays—Osmoregulation Genes Targeted

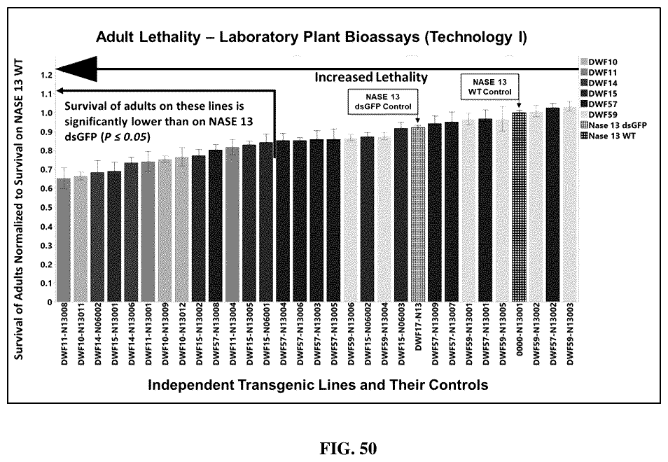

The best transgenic lines caused ˜35% increased adult mortality after seven days when compared to the control line NASE 13 WT. There were 13 transgenic lines that showed significant effect (increased adult mortality) when compared to a line expressing dsRNA against the GFP gene (WF17-GFP004) (FIG. 50).

### Laboratory Nymph Development Assays—Osmoregulation Genes Targeted

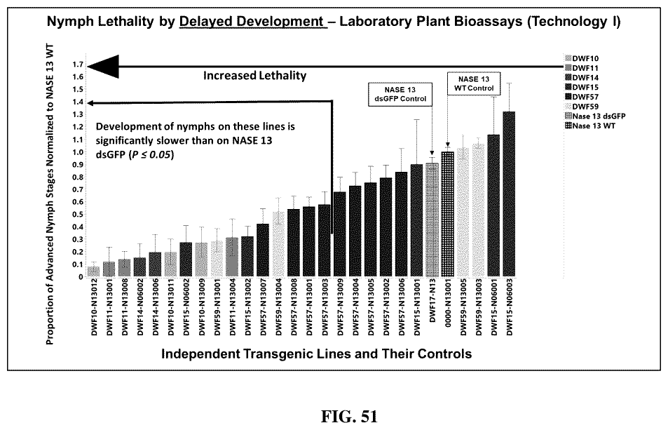

In the nymph development assays, the proportion of progeny that were in advanced development stage (red-eyed 4th nymphs) or completed their development (newly emerged adults) significantly differed between 16 of the transgenic lines and the controls (expressing dsRNA against the green fluorescent protein [GFP] or not expressing dsRNA at all [WT plants]. The best transgenic lines caused ˜90% delay in development when the normalized proportion of progeny that were in advanced stages or completed development was compared to control lines (FIG. 51). It Is likely that the vast majority of progeny are not only delayed but will actually never complete their development achieving significant whitefly population suppression.

### Laboratory Adult Survival Assays—Targeting Genes Involved in the Symbiotic Relationship Between Whiteflies and the Bacterium Portiera

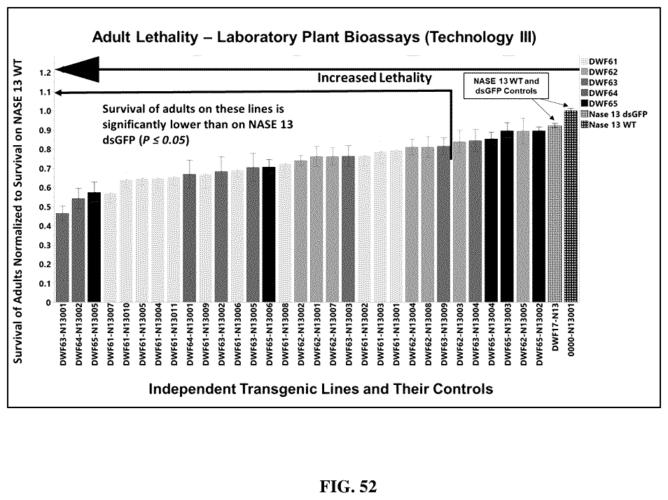

The best transgenic lines caused ˜50% increased adult mortality after seven days when compared to the control line NASE 13 WT. There were 25 transgenic lines from all the DWF 61-65 technologies that showed significant effect (Increased adult mortality) when compared to a line expressing dsRNA against the GFP gene (WF17-GFP004) (FIG. 52).

### Laboratory Nymph Development Assays—Targeting Genes Involved in the Symbiotic Relationship Between Whiteflies and the Bacterium Portiera

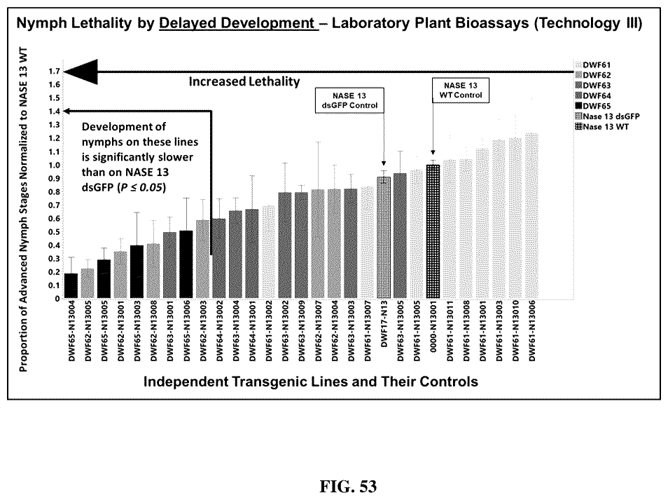

In the nymph development assays, the proportion of progeny that were in advanced development stage (red-eyed 4th nymphs) or completed their development (newly emerged adults) significantly differed between 9 of the transgenic lines and the controls (expressing dsRNA against the green fluorescent protein [GFP] or not expressing dsRNA at all [WT plants]. The best transgenic lines (mainly from DWF 62 AND 65) caused ˜80% delay in development when the normalized proportion of progeny that were in advanced stages or completed development was compared to control lines (FIG. 53). It is likely that the vast majority of progeny are not only delayed but will actually never complete their development achieving significant whitefly population suppression.

### Example 7: Gene Expression Silencing Assays and their Correlation with Performance

An RT-PCR system was established to further elucidate the mechanism/s underlying the negative effects on whiteflies of the transgenic RNAi cassava plants determine the relationship between the observed effects and downregulation of the target gene/s. For this, an assay was developed that allows RNA extraction from 50 adults and/or 50 nymphs. Once the RNA is extracted and cDNA is produced, a Real Time-PCR is performed. Importantly, it is more straightforward to use nymph samples than adult samples because in the assay for adults only surviving adults can be collected and all the individuals that died prior to adulthood from exposures to the dsRNA are lost.

Samples of 50 surviving adults (after 7 days of exposure to dsRNA producing plants) and/or nymphs (after a 25 days development period) were collected in 1.5 ml Eppendorf tubes, immediately thrown into liquid nitrogen, and stored at −80° C. Next, the RNA was extracted using ISOLATE II RNA mini kit (Meridian Bioscience, Ohio) following the manufacturer's protocol. DNA contamination was removed using a PerfeCTa DNaseI (Quanta Bioscience, Massachusetts) treatment. RNA quality and quantity were evaluated using a NanoDrop 2000 spectrophotometer (Thermo Fisher Scientific, Massachusetts). Samples in which the 260/280 or 260/230 values were less than 1.8 were treated with RNA Clean & Concentrator kit (Zymo Research, California). Next, a reverse transcriptase reaction was performed using 500 ng RNA from each sample and the Verso cDNA synthesis kit with Oligo-dT primer (Thermo Fisher Scientific, Massachusetts). Finally, the expression level of a target gene was examined using quantitative real-time PCR (qRT-PCR). The reactions were performed using CFX Connect Real-Time PCR System (BIO-RAD, California). A set of primers was designed and calibrated for each target gene based on the Thornton and Basu (2011) protocol using Primer3 software (https://primer3.ut.ce/) (see Table at the end of this document). The B. tabaci ribosomal protein L13a (RPL13A) was used as the reference gene. The qRT-PCR conditions were adjusted for the amplification efficiencies of the target and reference genes to be in the log range of 1.9-2.1; they were optimally set to a master mix containing 5 μl iTaq Universal SYBR Green Supermix (BIO-RAD, California), 0.5 μl of both forward and reverse primers (2 μmol/μl), 2 μl DDW and 2 μl cDNA template. The expression of target and reference genes was tested in triplicate for each sample to ensure the validity of the results. qRT-PCR thermal conditions consisted of one cycle of 95° C. for 2 min, followed by 40 cycles of 95° C. for 5 sec and 60° C. for 30 sec, and an ending cycle of 95° C. for 5 sec, 65° C. for 5 sec, and 95° C. for 30 sec. The homogeneity of the PCR products was confirmed by melting curves analyses. Quantification of the transcript's expression levels was conducted according to the ΔΔCt method (Applied Biosystems). A one-way ANOVA model was used to determine the significance of the differences between the expression means of the treatment and control samples (P≤0.05).

### Adult Gene Silencing Assays

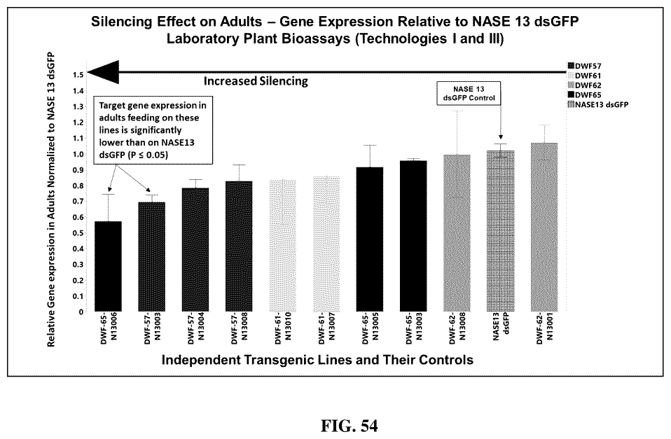

The expression levels of the targeted genes were up-to 43% lower in treated whitefly adults feeding on the RNAi plants when compared to the control adults feeding on NASE 13 plants expressing dsGFP (FIG. 54). Two lines, one from DWF65 (targeting the Biotin synthase gene [symbiotic relationship between whiteflies and the bacterium Portiera]) and one from DWF57 (targeting the Sucrase 5 gene [osmoregulation]) were significantly downregulated when compared to the gene expression level in adults feeding on NASE 13 dsGFP plants.

### Nymph Gene Silencing Assays

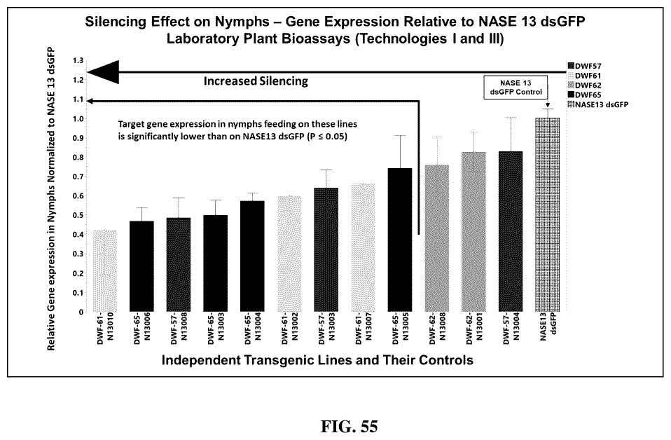

The expression levels of the targeted genes were up-to 57% lower (2.34-fold) in treated whitefly nymphs feeding on the RNAi plants when compared to the control nymphs feeding on NASE 13 plants expressing dsGFP (FIG. 55). Nine lines, four from DWF65 (targeting the Biotin synthase gene [symbiotic relationship between whiteflies and the bacterium Portiera]), two from DWF57 (targeting the Sucrase 5 gene [osmoregulation]) and three from DWF61 targeting the Arginosuccinate lyase gene [symbiotic relationship between whiteflies and the bacterium Portiera]) were significantly downregulated when compared to the gene expression level in nymphs feeding on NASE 13 dsGFP plants.

### Correlation Between Gene Silencing and Adults/Nymphs Performance Assays

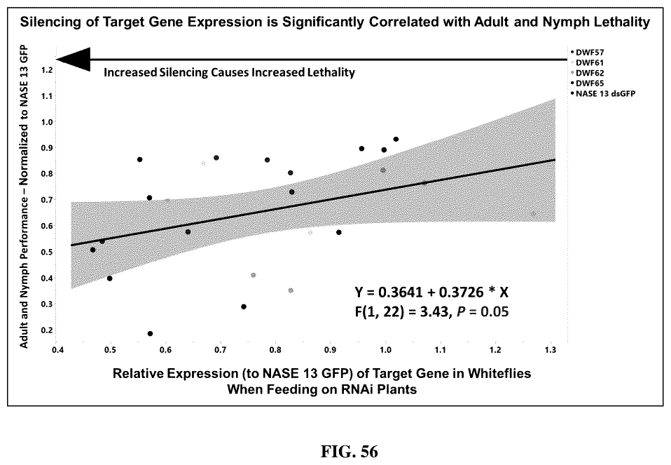

At a final stage, a correlation was evaluated (FIG. 56) between the gene silencing assays (presented in FIGS. 54 and 55) and adult/nymph lethality (presented in FIGS. 50-53). A significant positive correlation (P=0.05) was found which suggests that the more the target gene expression is suppressed, the lower the performance of immature and adult stages of the Insect.

TABLE 18
Primers used in qRT-PCR analyses.

| Primer Name | Sequence Identifier |
|---|---|
| DWF_57_F | SEQ ID NO: 86 |
| DWF_57_R | SEQ ID NO: 87 |
| DWF_61_F | SEQ ID NO: 88 |
| DWF_61_R | SEQ ID NO: 89 |
| DWF62_9_F | SEQ ID NO: 90 |
| DWF62_9_R | SEQ ID NO: 91 |
| 65_B_Forward | SEQ ID NO: 92 |
| 65_B_Reverse | SEQ ID NO: 93 |

What is claimed is:

1. A recombinant polynucleotide molecule comprising a first polynucleotide sequence having at least about 85% sequence identity to at least 18 contiguous nucleotides of a target nucleotide sequence, wherein said target nucleotide sequence:
a) encodes a polypeptide having a sequence selected from the group consisting of SEQ ID NO: 1, SEQ ID NO:3, SEQ ID NO:5, SEQ ID NO:7, SEQ ID NO:9, SEQ ID NO:11, and SEQ ID NO:13; or
b) has a sequence selected from the group consisting of SEQ ID NO:57, 59, 51, 63, 65, and 67-74;
wherein said recombinant polynucleotide molecule disrupts the activity of said polypeptide when provided in the diet of an invertebrate pest.

2. The recombinant polynucleotide molecule of claim 1, wherein said first polynucleotide sequence is operably linked to a heterologous promoter.

3. The recombinant polynucleotide molecule of claim 1, wherein said first polynucleotide sequence encodes an interfering RNA molecule.

4. The recombinant polynucleotide molecule of claim 1, wherein said first polynucleotide sequence encodes a ssRNA molecule or a dsRNA molecule.

5. The recombinant polynucleotide molecule of claim 1, wherein the first polynucleotide sequence has at least about 90%, at least about 95%, or 100% sequence identity to at least 18 contiguous nucleotides of said target sequence.

6. The recombinant polynucleotide molecule of claim 1, wherein the first polynucleotide sequence has at least about 85% sequence identity to at least 19 contiguous nucleotides, at least 20 contiguous nucleotides, or at least 21 contiguous nucleotides of said target sequence.

7. The recombinant polynucleotide molecule of claim 1, wherein the invertebrate pest is a pest of the order Hemiptera.

8. The recombinant polynucleotide molecule of claim 7, wherein the pest of the order Hemiptera is a Bemisia species pest.

9. A plant, plant part, plant cell, seed, or commodity product comprising the recombinant polynucleotide molecule of claim 1.

10. A composition comprising the recombinant polynucleotide molecule of claim 1.

11. A composition comprising a polynucleotide molecule comprising a first polynucleotide sequence having at least about 85% sequence identity to at least 18 contiguous nucleotides of a target nucleotide sequence, wherein said target nucleotide sequence:
a) encodes a polypeptide having a sequence selected from the group consisting of SEQ ID NO: 1, SEQ ID NO:3, SEQ ID NO:5, SEQ ID NO:7, SEQ ID NO:9, SEQ ID NO:11, and SEQ ID NO:13; or
b) has a sequence selected from the group consisting of SEQ ID NO:57, 59, 51, 63, 65, and 67-74;
wherein said composition disrupts the activity of said polypeptide when provided in the diet of an invertebrate pest.

12. The composition of claim 11, wherein the polynucleotide molecule is an interfering RNA molecule or encodes an interfering RNA molecule.

13. The composition of claim 11, wherein the polynucleotide molecule is a ssRNA molecule or a dsRNA molecule or encodes a ssRNA molecule or a dsRNA molecule.

14. A method for controlling invertebrate pest infestation, the method comprising providing a polynucleotide molecule comprising a first polynucleotide sequence having at least 85% sequence identity to at least 18 contiguous nucleotides of a target nucleotide sequence in the diet of an invertebrate pest, wherein said polynucleotide molecule disrupts the activity of a polypeptide encoded by said target nucleotide sequence, wherein said target nucleotide sequence:
a) encodes a polypeptide having a sequence selected from the group consisting of SEQ ID NO: 1, SEQ ID NO:3, SEQ ID NO:5, SEQ ID NO:7, SEQ ID NO:9, SEQ ID NO:11, and SEQ ID NO:13; or
b) has a sequence selected from the group consisting of SEQ ID NO:57, 59, 51, 63, 65, and 67-74.

15. The method of claim 14, wherein the polynucleotide molecule is an interfering RNA molecule or encodes an interfering RNA molecule.

16. The method of claim 14, wherein the polynucleotide molecule is a ssRNA molecule or a dsRNA molecule or encodes a ssRNA molecule or a dsRNA molecule.

17. The method of claim 14, wherein providing the polynucleotide molecule comprises providing a plant, plant part, plant cell, seed, or composition comprising said polynucleotide molecule in the diet of the invertebrate pest.

18. The method of claim 14, wherein the invertebrate pest is a pest of the order Hemiptera.

19. The method of claim 18, wherein the pest of the order Hemiptera is a Bemisia species pest.
